(19) United States
(12) Patent Application Publication
(10) Pub. No.: US 2025/0325602 A1
(43) Pub. Date: Oct. 23, 2025

(54) METHODS FOR IMPROVING TREATMENT OF EQUINE COLIC BY ADMINISTRATION OF A SYNTHETIC BIOENSEMBLE OR PURIFIED STRAINS THEREOF

(71) Applicant: Native Microbials, Inc., San Diego, CA (US)

(72) Inventors: Mallory EMBREE, San Diego, CA (US); Cameron MARTINO, Solana Beach, CA (US); Fan YANG, San Diego, CA (US); Jordan EMBREE, San Diego, CA (US); James GAFFNEY, San Diego, CA (US)

(73) Assignee: Native Microbials, Inc., San Diego, CA (US)

(21) Appl. No.: 18/959,890

(22) Filed: Nov. 26, 2024

Related U.S. Application Data
(63) Continuation of application No. 17/609,138, filed on Nov. 5, 2021, now abandoned.
(60) Provisional application No. 62/843,689, filed on May 6, 2019.

(51) Int. Cl. A61K 35/744; A61K 35/741; A61K 35/742; A61K 45/06; A61P 1/14

(52) U.S. Cl. CPC A61K 35/744; A61K 35/741; A61K 35/742; A61K 45/06; A61P 1/14

(57) ABSTRACT

The disclosure relates to isolated microorganisms, including novel strains of the microorganisms, synthetic bioensembles, and compositions comprising the same. Furthermore, the disclosure teaches methods of utilizing the described microorganisms, synthetic bioensembles, and compositions comprising the same, in methods for modulating the health of equine animals. In particular aspects, the disclosure provides methods of treating and/or preventing colic and shifting the gut microbiome.

### CROSS-REFERENCE TO RELATED APPLICATIONS

This application is a Continuaion of U.S. application Ser. No. 17/609,138, filed Nov. 5, 2021, which is a U.S. National Stage Application under 35 U.S.C. § 371 of International PCT Application No. PCT/US2020/031724, filed May 6, 2020 which claims priority to and benefit of U.S. Provisional Application 62/843,689, filed May 6, 2019, the contents of which are incorporated herein by reference in their entirety.

### STATEMENT REGARDING SEQUENCE LISTING

The contents of the electronic sequence listing (ASBI_013_03US_SeqList_ST26.xml; Size: 663,962 bytes; and Date of Creation: Oct. 9, 2024) are herein incorporated by reference in its entirety.

### FIELD

The present disclosure relates to isolated and biologically pure microorganisms that have applications, inter alia, in the treatment of colic in equines. The disclosed microorganisms can be utPCT already filed-no further filingsPCT already filed-no further filingsilized in their isolated and biologically pure states, as well as being formulated into compositions.

### BACKGROUND

The equine industry is a vital economic component of our economy, which produces horses for aid in work, show, entertainment, racing, rodeo, and companionship. There are over 70 different types of equine colic. Many of these are attributed to microbes. Others are likely but not confirmed to be microbial and some have no link to microbes. Some early stage microbial-based forms of colic are the precipice for many other forms of colic. This is caused by a healthy horse or other equine being induced through internal or external pressures to colic-like state. This temporal colic can either correct naturally back to the healthy state or progress into the many other types of symptomatic colic. Induction of colic can often occur through emotional, physical, and/or immunomodulatory stress as well as poor diet.

### SUMMARY

In some embodiments, the present disclosure provides a microbial composition comprising: one or more bacteria with a 16S nucleic acid sequence that is at least 97% identical to a nucleic acid sequence selected from SEQ ID NOs: 1-574; and a carrier suitable for equine administration.

In some embodiments, the microbial composition comprises one or more bacteria with a 16S nucleic acid sequence that is at least 97%, at least 98%, or at least 99% identical to a nucleic acid sequence selected from the group consisting of SEQ ID NO: 5, SEQ ID NO: 141, SEQ ID NO: 319, SEQ ID NO: 426, and SEQ ID NO: 475. In some embodiments, the microbial composition comprises one or more bacteria with a 16S nucleic acid sequence comprising or consisting of a nucleic acid sequence selected from the group consisting of SEQ ID NO: 5, SEQ ID NO: 141, SEQ ID NO: 319, SEQ ID NO: 426, and SEQ ID NO: 475. In some embodiments, the microbial composition comprises one or more bacteria with a 16S nucleic acid sequence that is at least 97%, at least 98%, or at least 99% identical to a nucleic acid sequence selected from the group consisting of SEQ ID NO: 11, SEQ ID NO: 142, SEQ ID NO: 320, SEQ ID NO: 433, and SEQ ID NO: 476. In some embodiments, the microbial composition comprises one or more bacteria with a 16S nucleic acid sequence comprising or consisting of a nucleic acid sequence selected from the group consisting of SEQ ID NO: 11, SEQ ID NO: 142, SEQ ID NO: 320, SEQ ID NO: 433, and SEQ ID NO: 476.

In some embodiments, the microbial composition comprises two, three, four, five, or more bacteria with a 16S nucleic acid sequence that is at least 97% identical to a nucleic acid sequence selected from SEQ ID NOs: 1-574. In some embodiments, the microbial composition comprises two, three, four, or five bacteria with a 16S nucleic acid sequence that is at least 97%, at least 98%, or at least 99% identical to a nucleic acid sequence selected from the group consisting of SEQ ID NO: 5, SEQ ID NO: 141, SEQ ID NO: 319, SEQ ID NO: 426, and SEQ ID NO: 475. In some embodiments, the microbial composition comprises two, three, four, or five bacteria with a 16S nucleic acid sequence comprising or consisting of a nucleic acid sequence selected from the group consisting of SEQ ID NO: 5, SEQ ID NO: 141, SEQ ID NO: 319, SEQ ID NO: 426, and SEQ ID NO: 475. In some embodiments, the microbial composition comprises two, three, four, or five bacteria with a 16S nucleic acid sequence that is at least 97%, at least 98%, or at least 99% identical to a nucleic acid sequence selected from the group consisting of SEQ ID NO: 11, SEQ ID NO: 142, SEQ ID NO: 320, SEQ ID NO: 433, and SEQ ID NO: 476. In some embodiments, the microbial composition comprises two, three, four, or five bacteria with a 16S nucleic acid sequence comprising or consisting of a nucleic acid sequence selected from the group consisting of SEQ ID NO: 11, SEQ ID NO: 142, SEQ ID NO: 320, SEQ ID NO: 433, and/or SEQ ID NO: 476.

In some embodiments, the present disclosure provides a microbial composition comprising: one or more bacterium selected from a Clostridium spp. bacterium; a Streptococcus spp. bacterium; an Escheria spp. bacterium; and an Atlantibacter spp. bacterium; and a carrier suitable for equine administration.

In some embodiments, the present disclosure provides a microbial composition comprising: one or more bacterium selected from a Clostridium butyricum bacterium; a Streptococcus equinis bacterium; an Escheria coli bacterium; a Clostridium maximum bacterium; and an Atlantibacter hermannii bacterium; and a carrier suitable for equine administration.

In some embodiments, the Clostridium butyricum bacterium comprises a 16S nucleic acid sequence that is at least 97%, 98%, or 99% identical to one of SEQ ID NOs: 5-13; the Streptococcus equinis bacterium comprises a 16S nucleic acid sequence that is at least 97%, 98%, or 99% identical to one of SEQ ID NOs: 143-150; theEscheria coli bacterium comprises a 16S nucleic acid sequence that is at least 97%, 98%, or 99% identical to one of SEQ ID NOs: 321-328; the Clostridium maximum bacterium comprises a 16S nucleic acid sequence that is at least 97%, 98%, or 99% identical to one of SEQ ID NOs: 430-437; and/or the Atlantibacter hermannii bacterium comprises a 16S nucleic acid sequence that is at least 97%, 98%, or 99% identical to one of SEQ ID NOs: 480-486.

In some embodiments, the Clostridium butyricum bacterium comprises a 16S nucleic acid sequence comprising or consisting of one of SEQ ID NOs: 5-13; the Streptococcus equinis bacterium comprises a 16S nucleic acid sequence comprising or consisting of one of SEQ ID NOs: 143-150; theEscheria coli bacterium comprises a 16S nucleic acid sequence comprising or consisting of one of SEQ ID NOs: 321-328; the Clostridium maximum bacterium comprises a 16S nucleic acid sequence comprising or consisting of one of SEQ ID NOs: 430-437; and/or the Atlantibacter hermannii bacterium comprises a 16S nucleic acid sequence comprising or consisting of one of SEQ ID NOs: 480-486.

In some embodiments, the Clostridium butyricum bacterium comprises a 16S nucleic acid sequence that is at least 97%, 98%, or 99% identical to SEQ ID NO: 5 or SEQ ID NO: 11; the Streptococcus equinis bacterium comprises a 16S nucleic acid sequence that is at least 97%, 98%, or 99% identical to SEQ ID NO: 141 or SEQ ID NO: 142; theEscheria coli bacterium comprises a 16S nucleic acid sequence that is at least 97%, 98%, or 99% identical to SEQ ID NO: 319 or SEQ ID NO: 320; the Clostridium maximum bacterium comprises a 16S nucleic acid sequence that is at least 97%, 98%, or 99% identical to SEQ ID NO: 426 or SEQ ID NO: 433; and/or the Atlantibacter hermannii bacterium comprises a 16S nucleic acid sequence that is at least 97%, 98%, or 99% identical SEQ ID NO: 475 or SEQ ID NO: 476.

In some embodiments, the Clostridium butyricum bacterium comprises a 16S nucleic acid sequence comprising or consisting of SEQ ID NO: 5 or SEQ ID NO: 11; the Streptococcus equinis bacterium comprises a 16S nucleic acid sequence comprising or consisting of SEQ ID NO: 141 or SEQ ID NO: 142; theEscheria coli bacterium comprises a 16S nucleic acid sequence comprising or consisting of SEQ ID NO: 319 or SEQ ID NO: 320; the Clostridium maximum bacterium comprises a 16S nucleic acid sequence comprising or consisting of SEQ ID NO: 426 or SEQ ID NO: 433; and/or the Atlantibacter hermannii bacterium comprises a 16S nucleic acid sequence comprising or consisting of SEQ ID NO: 475 or SEQ ID NO: 476.

In some embodiments, the one or more bacteria has a MIC score of at least about 0.2. In some embodiments, the equine is a domesticated equine or a wild equine. In some embodiments, the equine is selected from a horse, a zebra, a mule, and a donkey.

In some embodiments, the carrier comprises a solidification agent and a sweeting agent. In some embodiments, the solidification agent is selected from xantham gum, agar, and gelatin. In some embodiments, the sweeting agent is selected from corn syrup, molasses, cane molasses, brewer's yeast, and honey.

In some embodiments, the composition is formulated as a gel, a liquid, a powder, a tablet, a capsule, a pill, a feed additive, a food ingredient, a food supplement, a water additive, a heat-stabilized additive, a moisture-stabilized additive, a pelleted feed additive, a pre-pelleted applied feed additive, a post-pelleted applied feed additive, or a spray additive.

In some embodiments, the composition is formulated for administration by injection, direct application to target organ, bolus administration, oral administration (such as with or as part of food), fecal enema, fecal microbiota transplant via nasogastric intubation

In some embodiments, the microbial composition comprises the one or more bacteria in an amount effective to treat one or more symptoms of colic in an equine or to reduce the frequency of colic episodes.

In some embodiments, the present disclosure provides a method for preventing and/or treating colic in an equine comprising administering a microbial composition described herein to an equine in need thereof in an amount effective to prevent and/or treat colic in the equine administered the microbial composition compared to an equine that was not administered the microbial composition.

In some embodiments, the equine is a domesticated equine or a wild equine. In some embodiments, the equine is selected from a horse, a zebra, a mule, and a donkey.

In some embodiments, the microbial composition is administered daily for at least 1, 2, 3, 4, 5, 6, 7 days, or longer. In some embodiments, the microbial composition is administered daily for at least 1 week, at least 2 weeks, at least 3 weeks, at least 4 weeks, at least 5 weeks, at least 6 weeks, at least 7 weeks, at least 8 weeks, or longer. In some embodiments, the microbial composition is administered daily for at least 1 month, at least 2 months, at least 3 months, at least 4 months, at least 5 months, at least 6 months, or longer.

In some embodiments, the microbial composition is administered to the equine with an antibiotic, a proton pump inhibitor, and/or food. In some embodiments, the microbial composition is administered to the equine after administration of an antibiotic, a proton pump inhibitor, and/or food.

In some embodiments, the administration of the microbial composition reduces one or more symptoms of colic selected from abdominal pain, stomach irritation, rolling, kicking at stomach, distension of GI organs, and decreased eating. In some embodiments, the administration of the microbial composition reduces the frequency of colic episodes in an equine administered the microbial composition compared to an equine that has not been administered the microbial composition.

### Budapest Treaty on the International Recognition of the Deposit of Microorganisms for the Purpose of Patent Procedures

Some microorganisms described in this application were deposited with the United States Department of Agriculture (USDA) Agricultural Research Service (ARS) Culture Collection (NRRL®), located at 1815 N. University St., Peoria, IL 61604, USA. Some microorganisms described in this application were deposited with the Bigelow National Center for Marine Algae and Microbiota, located at 60 Bigelow Drive, East Boothbay, Maine 04544, USA.

The deposits were made under the terms of the Budapest Treaty on the International Recognition of the Deposit of Microorganisms for the Purposes of Patent Procedure. The NRRL® and Bigelow National Center for Marine Algae and Microbiota accession numbers and corresponding dates of deposit for the microorganisms described in this application are provided in Table 1.

The strains designated in the below table have been deposited in the labs of Ascus Biosciences, Inc. since at least February 2019.

TABLE 1
Microbial Deposits

| | Strain | SEQ | | Accession |
|---|---|---|---|---|
| BLAST taxonomy | Designation | ID | Depository | No. |
| Clostridium butyricum | AscusEQ_4A | 6 | Bigelow | 202004045 |
| | AscusEQ_4B | 7 | Bigelow | 202004042 |
| | AscusEQ_4C | 8 | Bigelow | 202004042 |
| | AscusEQ_4D | 9 | Bigelow | 202004043 |
| | AscusEQ_4E | 10 | Bigelow | 202004043 |
| | AscusEQ_4F | 11 | Bigelow | 202004043 |
| | | | NRRL | NRRL B-67941 |
| | AscusEQ_4G | 12 | Bigelow | 202004043 |
| | AscusEQ_4H | 13 | Bigelow | 202004044 |
| Terrisporobacter mayombei | AscusEQ_5A | 15 | Bigelow | 202004042 |
| | AscusEQ_5B | 16 | Bigelow | 202004042 |
| | AscusEQ_5C | 17 | Bigelow | 202004042 |
| | AscusEQ_5D | 18 | Bigelow | 202004044 |
| Mogibacterium pumilum | AscusEQ_7A | 20 | Bigelow | 202004042 |
| | AscusEQ_7B | 21 | Bigelow | 202004043 |
| | AscusEQ_7C | 22 | Bigelow | 202004043 |
| | AscusEQ_7D | 23 | Bigelow | 202004043 |
| Catabacter hongkongensis | AscusEQ_9A | 26 | Bigelow | 202004043 |
| Clostridium nitritogenes | AscusEQ_12A | 30 | Bigelow | 202004045 |
| | AscusEQ_12B | 31 | Bigelow | 202004044 |
| | AscusEQ_12C | 32 | Bigelow | 202004044 |
| | AscusEQ_12D | 33 | Bigelow | 202004044 |
| | AscusEQ_12E | 34 | Bigelow | 202004044 |
| | AscusEQ_12F | 35 | Bigelow | 202004044 |
| | AscusEQ_12G | 36 | Bigelow | 202004044 |
| Oribacterium sp. | AscusEQ_14A | 39 | Bigelow | 202004043 |
| | AscusEQ_14B | 40 | Bigelow | 202004043 |
| Lachnospiraceae bacterium | AscusEQ_15A | 42 | Bigelow | 202004044 |
| | AscusEQ_15B | 43 | Bigelow | 202004044 |
| | AscusEQ_15C | 44 | Bigelow | 202004044 |
| | AscusEQ_15D | 45 | Bigelow | 202004044 |
| | AscusEQ_15E | 46 | Bigelow | 202004044 |
| Lachnospiraceae bacterium | AscusEQ_16A | 48 | Bigelow | 202004043 |
| | AscusEQ_16B | 49 | Bigelow | 202004043 |
| | AscusEQ_16C | 50 | Bigelow | 202004043 |
| | AscusEQ_16D | 51 | Bigelow | 202004043 |
| Odoribacter splanchnicus | AscusEQ_17A | 53 | Bigelow | 202004042 |
| | AscusEQ_17B | 54 | Bigelow | 202004043 |
| | AscusEQ_17C | 55 | Bigelow | 202004044 |
| | AscusEQ_17D | 56 | Bigelow | 202004044 |
| | AscusEQ_17E | 57 | Bigelow | 202004044 |
| | AscusEQ_17F | 58 | Bigelow | 202004044 |
| Terrisporobacter glycolicus | AscusEQ_21A | 63 | Bigelow | 202004045 |
| | AscusEQ_21B | 64 | Bigelow | 202004044 |
| | AscusEQ_21C | 65 | Bigelow | 202004044 |
| | AscusEQ_21D | 66 | Bigelow | 202004044 |
| | AscusEQ_21E | 67 | Bigelow | 202004044 |
| | AscusEQ_21F | 68 | Bigelow | 202004044 |
| | AscusEQ_21G | 69 | Bigelow | 202004044 |
| Eubacterium barkeri | AscusEQ_24A | 73 | Bigelow | 202004043 |
| | AscusEQ_24B | 74 | Bigelow | 202004043 |
| | AscusEQ_24C | 75 | Bigelow | 202004043 |
| | AscusEQ_24D | 76 | Bigelow | 202004043 |
| Lachnospiraceae bacterium | AscusEQ_32A | 78 | Bigelow | 202004043 |
| Cellulosilyticum ruminicola | AscusEQ_31A | 81 | Bigelow | 202004043 |
| | AscusEQ_31B | 82 | Bigelow | 202004043 |
| | AscusEQ_31C | 83 | Bigelow | 202004043 |
| | AscusEQ_31D | 84 | Bigelow | 202004043 |
| Erysipelotrichaceae bacterium | AscusEQ_28A | 86 | Bigelow | 202004043 |
| | AscusEQ_28B | 87 | Bigelow | 202004043 |
| | AscusEQ_28C | 88 | Bigelow | 202004043 |
| | AscusEQ_28D | 89 | Bigelow | 202004043 |
| | AscusEQ_28E | 90 | Bigelow | 202004043 |
| | AscusEQ_28F | 91 | Bigelow | 202004043 |
| | AscusEQ_28G | 92 | Bigelow | 202004043 |
| Howardella ureilytica | AscusEQ_29A | 94 | Bigelow | 202004043 |
| | AscusEQ_29B | 95 | Bigelow | 202004043 |
| Butyrivibrio fibrisolvens | AscusEQ_33A | 98 | Bigelow | 202004043 |
| | AscusEQ_33B | 99 | Bigelow | 202004043 |
| | AscusEQ_33C | 100 | Bigelow | 202004043 |
| | AscusEQ_33D | 101 | Bigelow | 202004043 |
| | AscusEQ_33E | 102 | Bigelow | 202004043 |
| Lachnospiraceae bacterium | AscusEQ_35A | 105 | Bigelow | 202004043 |
| | AscusEQ_35B | 106 | Bigelow | 202004043 |
| | AscusEQ_35C | 107 | Bigelow | 202004043 |
| | AscusEQ_35D | 108 | Bigelow | 202004043 |
| | AscusEQ_35E | 109 | Bigelow | 202004043 |
| Frisingicoccus caecimuris | AscusEQ_37A | 112 | Bigelow | 202004044 |
| | AscusEQ_37B | 113 | Bigelow | 202004044 |
| | AscusEQ_37C | 114 | Bigelow | 202004044 |
| | AscusEQ_37D | 115 | Bigelow | 202004044 |
| | AscusEQ_37E | 116 | Bigelow | 202004044 |
| | AscusEQ_37F | 117 | Bigelow | 202004044 |
| | AscusEQ_37G | 118 | Bigelow | 202004044 |
| | AscusEQ_37H | 119 | Bigelow | 202004044 |
| Ruminiclostridium thermocellum | AscusEQ_38A | 120 | Bigelow | 202004043 |
| Spiroplasma sp. | AscusEQ_39A | 122 | Bigelow | 202004043 |
| Algoriphagus ratkowskyi | AscusEQ_130A | 126 | Bigelow | 202004044 |
| | AscusEQ_130B | 127 | Bigelow | 202004044 |
| | AscusEQ_130C | 128 | Bigelow | 202004044 |
| | AscusEQ_130D | 129 | Bigelow | 202004044 |
| | AscusEQ_130E | 130 | Bigelow | 202004044 |
| | AscusEQ_130F | 131 | Bigelow | 202004044 |
| | AscusEQ_130G | 132 | Bigelow | 202004044 |
| Prevotella copri | AscusEQ_344A | 134 | Bigelow | 202004044 |
| | AscusEQ_344B | 135 | Bigelow | 202004044 |
| | AscusEQ_344C | 136 | Bigelow | 202004044 |
| | AscusEQ_344D | 137 | Bigelow | 202004044 |
| | AscusEQ_344E | 138 | Bigelow | 202004044 |
| | AscusEQ_344F | 139 | Bigelow | 202004044 |
| | AscusEQ_344G | 140 | Bigelow | 202004044 |
| Streptococcus equinus | AscusEQ_140A | 142 | Bigelow | 202004045 |
| | | | NRRL | NRRL B-67943 |
| | AscusEQ_140B | 143 | Bigelow | 202004045 |
| | AscusEQ_140C | 144 | Bigelow | 202004044 |
| | AscusEQ_140D | 145 | Bigelow | 202004044 |
| | AscusEQ_140E | 146 | Bigelow | 202004044 |
| | AscusEQ_140F | 147 | Bigelow | 202004044 |
| | AscusEQ_140G | 148 | Bigelow | 202004044 |
| Prevotella copri | AscusEQ_484A | 150 | Bigelow | 202004044 |
| | AscusEQ_484B | 151 | Bigelow | 202004044 |
| | AscusEQ_484C | 152 | Bigelow | 202004044 |
| | AscusEQ_484D | 153 | Bigelow | 202004044 |
| | AscusEQ_484E | 154 | Bigelow | 202004044 |
| | AscusEQ_484F | 155 | Bigelow | 202004044 |
| | AscusEQ_484G | 156 | Bigelow | 202004044 |
| Duodembacillus massiliensis | AscusEQ_1187A | 158 | Bigelow | 202004045 |
| | AscusEQ_1187B | 159 | Bigelow | 202004044 |
| | AscusEQ_1187C | 160 | Bigelow | 202004044 |
| | AscusEQ_1187D | 161 | Bigelow | 202004044 |
| Bacteroidales bacterium | AscusEQ_200A | 163 | Bigelow | 202004042 |
| | AscusEQ_200B | 164 | Bigelow | 202004043 |
| | AscusEQ_200C | 165 | Bigelow | 202004043 |
| | AscusEQ_200D | 166 | Bigelow | 202004043 |
| | AscusEQ_200E | 167 | Bigelow | 202004043 |
| | AscusEQ_200F | 168 | Bigelow | 202004044 |
| | AscusEQ_200G | 169 | Bigelow | 202004044 |
| Bacteroidia bacterium | AscusEQ_183A | 171 | Bigelow | 202004045 |
| | AscusEQ_183B | 172 | Bigelow | 202004043 |
| | AscusEQ_183C | 173 | Bigelow | 202004043 |
| | AscusEQ_183D | 174 | Bigelow | 202004043 |
| | AscusEQ_183E | 175 | Bigelow | 202004043 |
| | AscusEQ_183F | 176 | Bigelow | 202004043 |
| | AscusEQ_183G | 177 | Bigelow | 202004044 |
| Cecembia lonarensis | AscusEQ_226A | 179 | Bigelow | 202004042 |
| | AscusEQ_226B | 180 | Bigelow | 202004043 |
| | AscusEQ_226C | 181 | Bigelow | 202004043 |
| | AscusEQ_226D | 182 | Bigelow | 202004044 |
| | AscusEQ_226E | 183 | Bigelow | 202004044 |
| | AscusEQ_226F | 184 | Bigelow | 202004044 |
| | AscusEQ_226G | 185 | Bigelow | 202004044 |
| Bacteroidia bacterium | AscusEQ_369A | 187 | Bigelow | 202004043 |
| | AscusEQ_369B | 188 | Bigelow | 202004044 |
| | AscusEQ_369C | 189 | Bigelow | 202004044 |
| | AscusEQ_369D | 190 | Bigelow | 202004044 |
| | AscusEQ_369E | 191 | Bigelow | 202004044 |
| | AscusEQ_369F | 192 | Bigelow | 202004044 |
| | AscusEQ_369G | 193 | Bigelow | 202004044 |
| Bacteroidales bacterium | AscusEQ_820A | 195 | Bigelow | 202004042 |
| | AscusEQ_820B | 196 | Bigelow | 202004043 |
| | AscusEQ_820C | 197 | Bigelow | 202004044 |
| | AscusEQ_820D | 198 | Bigelow | 202004044 |
| | AscusEQ_820E | 199 | Bigelow | 202004044 |
| Clostridium maximum | AscusEQ_253A | 201 | Bigelow | 202004043 |
| | AscusEQ_253B | 202 | Bigelow | 202004043 |
| | AscusEQ_253C | 203 | Bigelow | 202004043 |
| | AscusEQ_253D | 204 | Bigelow | 202004043 |
| | AscusEQ_253E | 205 | Bigelow | 202004043 |
| | AscusEQ_253F | 206 | Bigelow | 202004044 |
| | AscusEQ_253G | 207 | Bigelow | 202004044 |
| Bacteroidales bacterium | AscusEQ_126A | 210 | Bigelow | 202004042 |
| | AscusEQ_126B | 211 | Bigelow | 202004043 |
| | AscusEQ_126C | 212 | Bigelow | 202004043 |
| | AscusEQ_126D | 213 | Bigelow | 202004043 |
| | AscusEQ_126E | 214 | Bigelow | 202004043 |
| | AscusEQ_126F | 215 | Bigelow | 202004043 |
| | AscusEQ_126G | 216 | Bigelow | 202004044 |
| Bacteroidales bacterium | AscusEQ_1029A | 218 | Bigelow | 202004044 |
| | AscusEQ_1029B | 219 | Bigelow | 202004044 |
| | AscusEQ_1029C | 220 | Bigelow | 202004044 |
| Bacteroidales bacterium | AscusEQ_980A | 223 | Bigelow | 202004043 |
| | AscusEQ_980B | 224 | Bigelow | 202004043 |
| | AscusEQ_980C | 225 | Bigelow | 202004043 |
| | AscusEQ_980D | 226 | Bigelow | 202004043 |
| | AscusEQ_980E | 227 | Bigelow | 202004043 |
| | AscusEQ_980F | 228 | Bigelow | 202004044 |
| Bacteroidia bacterium | AscusEQ_1166A | 230 | Bigelow | 202004043 |
| | AscusEQ_1166B | 231 | Bigelow | 202004043 |
| | AscusEQ_1166C | 232 | Bigelow | 202004043 |
| | AscusEQ_1166D | 233 | Bigelow | 202004043 |
| | AscusEQ_1166E | 234 | Bigelow | 202004043 |
| | AscusEQ_1166F | 235 | Bigelow | 202004044 |
| | AscusEQ_1166G | 236 | Bigelow | 202004044 |
| Acidaminococcus sp. | AscusEQ_105A | 238 | Bigelow | 202004042 |
| | AscusEQ_105B | 239 | Bigelow | 202004043 |
| | AscusEQ_105C | 240 | Bigelow | 202004043 |
| | AscusEQ_105D | 241 | Bigelow | 202004043 |
| | AscusEQ_105E | 242 | Bigelow | 202004043 |
| | AscusEQ_105F | 243 | Bigelow | 202004043 |
| | AscusEQ_105G | 244 | Bigelow | 202004043 |
| Bacteroidales bacterium | AscusEQ_145A | 246 | Bigelow | 202004042 |
| | AscusEQ_145B | 247 | Bigelow | 202004043 |
| | AscusEQ_145C | 248 | Bigelow | 202004043 |
| Bacteroides graminisolvens | AscusEQ_669A | 251 | Bigelow | 202004043 |
| | AscusEQ_669B | 252 | Bigelow | 202004043 |
| | AscusEQ_669C | 253 | Bigelow | 202004043 |
| | AscusEQ_669D | 254 | Bigelow | 202004043 |
| | AscusEQ_669E | 255 | Bigelow | 202004043 |
| | AscusEQ_669F | 256 | Bigelow | 202004043 |
| | AscusEQ_669G | 257 | Bigelow | 202004043 |
| Bacteroidales bacterium | AscusEQ_703A | 259 | Bigelow | 202004043 |
| | AscusEQ_703B | 260 | Bigelow | 202004044 |
| | AscusEQ_703C | 261 | Bigelow | 202004044 |
| | AscusEQ_703D | 262 | Bigelow | 202004044 |
| Parabacteroides distasonis | AscusEQ_436A | 264 | Bigelow | 202004042 |
| | AscusEQ_436B | 265 | Bigelow | 202004042 |
| | AscusEQ_436C | 266 | Bigelow | 202004042 |
| | AscusEQ_436D | 267 | Bigelow | 202004044 |
| | AscusEQ_436E | 268 | Bigelow | 202004044 |
| | AscusEQ_436F | 269 | Bigelow | 202004044 |
| | AscusEQ_436G | 270 | Bigelow | 202004044 |
| Bacteroides coprophilus | AscusEQ_680A | 272 | Bigelow | 202004043 |
| | AscusEQ_680B | 273 | Bigelow | 202004044 |
| | AscusEQ_680C | 274 | Bigelow | 202004044 |
| | AscusEQ_680D | 275 | Bigelow | 202004044 |
| | AscusEQ_680E | 276 | Bigelow | 202004044 |
| | AscusEQ_680F | 277 | Bigelow | 202004044 |
| Bacteroidales bacterium | AscusEQ_762A | 280 | Bigelow | 202004043 |
| | AscusEQ_762B | 281 | Bigelow | 202004043 |
| | AscusEQ_762C | 282 | Bigelow | 202004043 |
| | AscusEQ_762D | 283 | Bigelow | 202004043 |
| | AscusEQ_762E | 284 | Bigelow | 202004043 |
| | AscusEQ_762F | 285 | Bigelow | 202004043 |
| | AscusEQ_762G | 286 | Bigelow | 202004043 |
| Phascolarctobacterium sp. | AscusEQ_773A | 289 | Bigelow | 202004044 |
| | AscusEQ_773B | 290 | Bigelow | 202004044 |
| | AscusEQ_773C | 291 | Bigelow | 202004044 |
| | AscusEQ_773D | 292 | Bigelow | 202004044 |
| | AscusEQ_773E | 293 | Bigelow | 202004044 |
| | AscusEQ_773F | 294 | Bigelow | 202004044 |
| | AscusEQ_773G | 295 | Bigelow | 202004044 |
| Phascolarctobacterium sp | AscusEQ_625A | 298 | Bigelow | 202004042 |
| | AscusEQ_625B | 299 | Bigelow | 202004043 |
| | AscusEQ_625C | 300 | Bigelow | 202004044 |
| | AscusEQ_625D | 301 | Bigelow | 202004044 |
| | AscusEQ_625E | 302 | Bigelow | 202004044 |
| | AscusEQ_625F | 303 | Bigelow | 202004044 |
| Bacteroides graminisolvens | AscusEQ_783A | 306 | Bigelow | 202004043 |
| | AscusEQ_783B | 307 | Bigelow | 202004043 |
| | AscusEQ_783C | 308 | Bigelow | 202004043 |
| | AscusEQ_783D | 309 | Bigelow | 202004043 |
| | AscusEQ_783E | 310 | Bigelow | 202004043 |
| Phascolarctobacterium sp. | AscusEQ_1316A | 312 | Bigelow | 202004042 |
| | AscusEQ_1316B | 313 | Bigelow | 202004044 |
| | AscusEQ_1316C | 314 | Bigelow | 202004044 |
| | AscusEQ_1316D | 315 | Bigelow | 202004044 |
| | AscusEQ_1316E | 316 | Bigelow | 202004044 |
| | AscusEQ_1316F | 317 | Bigelow | 202004044 |
| | AscusEQ_1316G | 318 | Bigelow | 202004044 |
| Escherichia coli | AscusEQ_61A | 320 | Bigelow | 202004045 |
| | | | NRRL | NRRL B-67942 |
| | AscusEQ_61B | 321 | Bigelow | 202004044 |
| | AscusEQ_61C | 322 | Bigelow | 202004044 |
| | AscusEQ_61D | 323 | Bigelow | 202004044 |
| | AscusEQ_61E | 324 | Bigelow | 202004044 |
| | AscusEQ_61F | 325 | Bigelow | 202004044 |
| | AscusEQ_61G | 326 | Bigelow | 202004044 |
| Bacteroidetes bacterium | AscusEQ_1022A | 328 | Bigelow | 202004042 |
| | AscusEQ_1022B | 329 | Bigelow | 202004043 |
| | AscusEQ_1022C | 330 | Bigelow | 202004043 |
| | AscusEQ_1022D | 331 | Bigelow | 202004044 |
| Phascolarctobacterium sp. | AscusEQ_607A | 333 | Bigelow | 202004045 |
| | AscusEQ_607B | 334 | Bigelow | 202004045 |
| | AscusEQ_607C | 335 | Bigelow | 202004043 |
| | AscusEQ_607D | 336 | Bigelow | 202004043 |
| | AscusEQ_607E | 337 | Bigelow | 202004043 |
| Alloprevotella sp. | AscusEQ_412A | 339 | Bigelow | 202004042 |
| | AscusEQ_412B | 340 | Bigelow | 202004043 |
| | AscusEQ_412C | 341 | Bigelow | 202004043 |
| | AscusEQ_412D | 342 | Bigelow | 202004043 |
| | AscusEQ_412E | 343 | Bigelow | 202004044 |
| | AscusEQ_412F | 344 | Bigelow | 202004044 |
| | AscusEQ_412G | 345 | Bigelow | 202004044 |
| Muribaculaceae bacterium | AscusEQ_1362A | 347 | Bigelow | 202004044 |
| Bacteroidia bacterium | AscusEQ_696A | 349 | Bigelow | 202004043 |
| | AscusEQ_696B | 350 | Bigelow | 202004043 |
| | AscusEQ_696C | 351 | Bigelow | 202004043 |
| | AscusEQ_696D | 352 | Bigelow | 202004043 |
| | AscusEQ_696E | 353 | Bigelow | 202004043 |
| | AscusEQ_696F | 354 | Bigelow | 202004043 |
| Bacteroides massiliensis | AscusEQ_835A | 357 | Bigelow | 202004043 |
| | AscusEQ_835B | 358 | Bigelow | 202004044 |
| | AscusEQ_835C | 359 | Bigelow | 202004044 |
| | AscusEQ_835D | 360 | Bigelow | 202004044 |
| Oscillibacter valericigenes | AscusEQ_595A | 362 | Bigelow | 202004043 |
| | AscusEQ_595B | 363 | Bigelow | 202004044 |
| | AscusEQ_595C | 364 | Bigelow | 202004044 |
| | AscusEQ_595D | 365 | Bigelow | 202004044 |
| | AscusEQ_595E | 366 | Bigelow | 202004044 |
| | AscusEQ_595F | 367 | Bigelow | 202004044 |
| | AscusEQ_595G | 368 | Bigelow | 202004044 |
| TM7 bacterium | AscusEQ_1850A | 370 | Bigelow | 202004042 |
| | AscusEQ_1850B | 371 | Bigelow | 202004043 |
| | AscusEQ_1850C | 372 | Bigelow | 202004043 |
| Phascolarctobacterium succinatutens | AscusEQ_298A | 374 | Bigelow | 202004044 |
| | AscusEQ_298B | 375 | Bigelow | 202004044 |
| | AscusEQ_298C | 376 | Bigelow | 202004044 |
| | AscusEQ_298D | 377 | Bigelow | 202004044 |
| | AscusEQ_298E | 378 | Bigelow | 202004044 |
| Bacteroides graminisolvens | AscusEQ_743A | 380 | Bigelow | 202004043 |
| | AscusEQ_743B | 381 | Bigelow | 202004043 |
| | AscusEQ_743C | 382 | Bigelow | 202004043 |
| | AscusEQ_743D | 383 | Bigelow | 202004043 |
| | AscusEQ_743E | 384 | Bigelow | 202004043 |
| Alphaproteobacteria bacterium | AscusEQ_722A | 386 | Bigelow | 202004043 |
| | AscusEQ_722B | 387 | Bigelow | 202004043 |
| | AscusEQ_722C | 388 | Bigelow | 202004043 |
| | AscusEQ_722D | 389 | Bigelow | 202004043 |
| | AscusEQ_722E | 390 | Bigelow | 202004044 |
| | AscusEQ_722F | 391 | Bigelow | 202004044 |
| | AscusEQ_722G | 392 | Bigelow | 202004044 |
| Cecembia lonarensis | AscusEQ_603A | 395 | Bigelow | 202004043 |
| | AscusEQ_603B | 396 | Bigelow | 202004043 |
| | AscusEQ_603C | 397 | Bigelow | 202004043 |
| | AscusEQ_603D | 398 | Bigelow | 202004044 |
| | AscusEQ_603E | 399 | Bigelow | 202004044 |
| | AscusEQ_603F | 400 | Bigelow | 202004044 |
| | AscusEQ_603G | 401 | Bigelow | 202004044 |
| Phascolarctobacterium sp. | AscusEQ_1567A | 404 | Bigelow | 202004042 |
| | AscusEQ_1567B | 405 | Bigelow | 202004042 |
| | AscusEQ_1567C | 406 | Bigelow | 202004042 |
| | AscusEQ_1567D | 407 | Bigelow | 202004044 |
| | AscusEQ_1567E | 408 | Bigelow | 202004044 |
| | AscusEQ_1567F | 409 | Bigelow | 202004044 |
| | AscusEQ_1567G | 410 | Bigelow | 202004044 |
| Bacteroidia bacterium | AscusEQ_1921A | 412 | Bigelow | 202004042 |
| | AscusEQ_1921B | 413 | Bigelow | 202004044 |
| | AscusEQ_1921C | 414 | Bigelow | 202004044 |
| | AscusEQ_1921D | 415 | Bigelow | 202004044 |
| | AscusEQ_1921E | 416 | Bigelow | 202004044 |
| | AscusEQ_1921F | 417 | Bigelow | 202004044 |
| | AscusEQ_1921G | 418 | Bigelow | 202004044 |
| Bacteroides graminisolvens | AscusEQ_476A | 420 | Bigelow | 202004043 |
| | AscusEQ_476B | 421 | Bigelow | 202004043 |
| | AscusEQ_476C | 422 | Bigelow | 202004043 |
| | AscusEQ_476D | 423 | Bigelow | 202004043 |
| | AscusEQ_476E | 424 | Bigelow | 202004043 |
| | AscusEQ_476F | 425 | Bigelow | 202004043 |
| Clostridium maximum | AscusEQ_414A | 427 | Bigelow | 202004043 |
| | AscusEQ_414B | 428 | Bigelow | 202004043 |
| | AscusEQ_414C | 429 | Bigelow | 202004043 |
| | AscusEQ_414D | 430 | Bigelow | 202004043 |
| | AscusEQ_414E | 431 | Bigelow | 202004043 |
| | AscusEQ_414F | 432 | Bigelow | 202004044 |
| | AscusEQ_414G | 433 | Bigelow | 202004044 |
| | | | NRRL | NRRL B-67940 |
| Bacteroidia bacterium | AscusEQ_1253A | 435 | Bigelow | 202004043 |
| | AscusEQ_1253B | 436 | Bigelow | 202004043 |
| | AscusEQ_1253C | 437 | Bigelow | 202004044 |
| Bacteroides mediterraneensis | AscusEQ_324A | 439 | Bigelow | 202004045 |
| | AscusEQ_324B | 440 | Bigelow | 202004045 |
| | AscusEQ_324C | 441 | Bigelow | 202004042 |
| | AscusEQ_324D | 442 | Bigelow | 202004043 |
| | AscusEQ_324E | 443 | Bigelow | 202004043 |
| Phascolarctobacterium sp. | AscusEQ_1536A | 445 | Bigelow | 202004043 |
| | AscusEQ_1536B | 446 | Bigelow | 202004044 |
| | AscusEQ_1536C | 447 | Bigelow | 202004044 |
| | AscusEQ_1536D | 448 | Bigelow | 202004044 |
| | AscusEQ_1536E | 449 | Bigelow | 202004044 |
| | AscusEQ_1536F | 450 | Bigelow | 202004044 |
| | AscusEQ_1536G | 451 | Bigelow | 202004044 |
| Bacteroides massiliensis | AscusEQ_971A | 453 | Bigelow | 202004043 |
| | AscusEQ_971B | 454 | Bigelow | 202004043 |
| | AscusEQ_971C | 455 | Bigelow | 202004044 |
| | AscusEQ_971D | 456 | Bigelow | 202004044 |
| | AscusEQ_971E | 457 | Bigelow | 202004044 |
| Algoriphagus taiwanensis | AscusEQ_257A | 459 | Bigelow | 202004044 |
| | AscusEQ_257B | 460 | Bigelow | 202004044 |
| | AscusEQ_257C | 461 | Bigelow | 202004044 |
| | AscusEQ_257D | 462 | Bigelow | 202004044 |
| | AscusEQ_257E | 463 | Bigelow | 202004044 |
| | AscusEQ_257F | 464 | Bigelow | 202004044 |
| Bacteroidales bacterium | AscusEQ_452A | 466 | Bigelow | 202004042 |
| | AscusEQ_452B | 467 | Bigelow | 202004042 |
| | AscusEQ_452C | 468 | Bigelow | 202004043 |
| | AscusEQ_452D | 469 | Bigelow | 202004043 |
| | AscusEQ_452E | 470 | Bigelow | 202004043 |
| | AscusEQ_452F | 471 | Bigelow | 202004043 |
| | AscusEQ_452G | 472 | Bigelow | 202004043 |
| Atlantibacter hermannii | AscusEQ_109A | 476 | Bigelow | 202004045 |
| | | | NRRL | NRRL B-67944 |
| | AscusEQ_109B | 477 | Bigelow | 202004045 |
| | AscusEQ_109C | 478 | Bigelow | 202004045 |
| | AscusEQ_109D | 479 | Bigelow | 202004042 |
| | AscusEQ_109E | 480 | Bigelow | 202004044 |
| | AscusEQ_109F | 481 | Bigelow | 202004044 |
| | AscusEQ_167A | 482 | Bigelow | 202004045 |
| | AscusEQ_167B | 483 | Bigelow | 202004043 |
| | AscusEQ_167C | 484 | Bigelow | 202004043 |
| Prevotella copri | AscusEQ_363A | 486 | Bigelow | 202004042 |
| | AscusEQ_363B | 487 | Bigelow | 202004044 |
| | AscusEQ_363C | 488 | Bigelow | 202004044 |
| | AscusEQ_363D | 489 | Bigelow | 202004044 |
| | AscusEQ_363E | 490 | Bigelow | 202004044 |
| | AscusEQ_363F | 491 | Bigelow | 202004044 |
| | AscusEQ_363G | 492 | Bigelow | 202004044 |
| Bacteroidales bacterium | AscusEQ_299A | 494 | Bigelow | 202004042 |
| | AscusEQ_299B | 495 | Bigelow | 202004043 |
| | AscusEQ_299C | 496 | Bigelow | 202004043 |
| | AscusEQ_299D | 497 | Bigelow | 202004043 |
| | AscusEQ_299E | 498 | Bigelow | 202004043 |
| | AscusEQ_299F | 499 | Bigelow | 202004043 |
| | AscusEQ_299G | 500 | Bigelow | 202004043 |
| Prevotella oris | AscusEQ_456A | 502 | Bigelow | 202004043 |
| | AscusEQ_456B | 503 | Bigelow | 202004043 |
| | AscusEQ_456C | 504 | Bigelow | 202004043 |
| | AscusEQ_456D | 505 | Bigelow | 202004043 |
| | AscusEQ_456E | 506 | Bigelow | 202004043 |
| | AscusEQ_456F | 507 | Bigelow | 202004043 |
| Bacteroides caecicola | AscusEQ_232A | 509 | Bigelow | 202004045 |
| | AscusEQ_232B | 510 | Bigelow | 202004043 |
| | AscusEQ_232C | 511 | Bigelow | 202004043 |
| Algoriphagus ratkowskyi | AscusEQ_235A | 513 | Bigelow | 202004043 |
| | AscusEQ_235B | 514 | Bigelow | 202004044 |
| | AscusEQ_235C | 515 | Bigelow | 202004044 |
| | AscusEQ_235D | 516 | Bigelow | 202004044 |
| | AscusEQ_235E | 517 | Bigelow | 202004044 |
| | AscusEQ_235F | 518 | Bigelow | 202004044 |
| | AscusEQ_235G | 519 | Bigelow | 202004044 |
| Parabacteroides distasonis | AscusEQ_490A | 521 | Bigelow | 202004042 |
| | AscusEQ_490B | 522 | Bigelow | 202004042 |
| | AscusEQ_490C | 523 | Bigelow | 202004043 |
| | AscusEQ_490D | 524 | Bigelow | 202004044 |
| | AscusEQ_490E | 525 | Bigelow | 202004044 |
| | AscusEQ_490F | 526 | Bigelow | 202004044 |
| | AscusEQ_490G | 527 | Bigelow | 202004044 |
| Bacteroidales bacterium | AscusEQ_588A | 530 | Bigelow | 202004043 |
| | AscusEQ_588B | 531 | Bigelow | 202004043 |
| | AscusEQ_588C | 532 | Bigelow | 202004043 |
| | AscusEQ_588D | 533 | Bigelow | 202004043 |
| | AscusEQ_588E | 534 | Bigelow | 202004043 |
| Alphaproteobacteria bacterium | AscusEQ_1234A | 536 | Bigelow | 202004045 |
| | AscusEQ_1234B | 537 | Bigelow | 202004043 |
| | AscusEQ_1234C | 538 | Bigelow | 202004043 |
| | AscusEQ_1234D | 539 | Bigelow | 202004043 |
| | AscusEQ_1234E | 540 | Bigelow | 202004043 |
| Anaerocella delicata | AscusEQ_962A | 542 | Bigelow | 202004045 |
| | AscusEQ_962B | 543 | Bigelow | 202004042 |
| | AscusEQ_962C | 544 | Bigelow | 202004042 |
| Alphaproteobacteria bacterium | AscusEQ_916A | 546 | Bigelow | 202004045 |
| | AscusEQ_916B | 547 | Bigelow | 202004043 |
| | AscusEQ_916C | 548 | Bigelow | 202004043 |
| | AscusEQ_916D | 549 | Bigelow | 202004043 |
| | AscusEQ_916E | 550 | Bigelow | 202004043 |
| Prevotella sp. | AscusEQ_1095A | 552 | Bigelow | 202004042 |
| | AscusEQ_1095B | 553 | Bigelow | 202004043 |
| | AscusEQ_1095C | 554 | Bigelow | 202004043 |
| | AscusEQ_1095D | 555 | Bigelow | 202004043 |
| | AscusEQ_1095E | 556 | Bigelow | 202004043 |
| | AscusEQ_1095F | 557 | Bigelow | 202004043 |
| | AscusEQ_1095G | 558 | Bigelow | 202004043 |
| Phascolarctobacterium sp. | AscusEQ_614A | 560 | Bigelow | 202004044 |
| | AscusEQ_614B | 561 | Bigelow | 202004044 |
| | AscusEQ_614C | 562 | Bigelow | 202004044 |
| | AscusEQ_614D | 563 | Bigelow | 202004044 |
| | AscusEQ_614E | 564 | Bigelow | 202004044 |
| | AscusEQ_614F | 565 | Bigelow | 202004044 |
| | AscusEQ_614G | 566 | Bigelow | 202004044 |
| Bacteroidales bacterium | AscusEQ_698A | 569 | Bigelow | 202004044 |
| | AscusEQ_698B | 570 | Bigelow | 202004044 |
| | AscusEQ_698C | 571 | Bigelow | 202004044 |
| | AscusEQ_698D | 572 | Bigelow | 202004044 |
| | AscusEQ_698E | 573 | Bigelow | 202004044 |
| | AscusEQ_698F | 574 | Bigelow | 202004044 |
| | AscusEQ_698G | 575 | Bigelow | 202004044 |

### BRIEF DESCRIPTION OF THE DRAWINGS

FIG. 1 illustrates the significant difference in beta diversity (left) and alpha diversity (right) between colic and healthy states in equines.

FIG. 2 illustrates the differences in microbial load and microbial populations (total cells/ml) as well as taxonomic differences at the phylum level in the fecal microbiome of colic vs. no colic equines.

FIG. 3 shows that binary classification algorithms can utilize the microbial composition of fecal samples to determine if the source patient is in a colic or non-colic state.

FIG. 4 shows that multiclass classification algorithms can utilize the microbial composition of fecal samples to determine if the source patient is in a symptomatic colic, asymptomatic colic, or non-colic state.

FIG. 5 shows a principal coordinate analysis of samples classified as either no colic/healthy; colic; temporal/transient colic (colicing/symptomatic); or temporal/transient colic (not colicing/asymptomatic).

FIG. 6 shows the alpha diversity of of samples classified as either no colic/healthy; colic; temporal/transient colic (colicing/symptomatic); or temporal/transient colic (not colicing/asymptomatic).

FIG. 7 illustrates that the MIC score network and ranking based on colic are anti-correlated.

FIG. 8 shows cross validation scores of machine learning models to accurately diagnose microbial-mediated colic.

FIG. 9 shows heat maps of the fecal microbial abundances (y-axis) in healthy and colicking states over time (x-axis).

FIG. 10 shows that principal coordinate analysis can be used to determine the efficacy of fecal transplant.

FIG. 11 provides heat maps of the fecal microbial abundances (y-axis) between the healthy and colicking states over time (x-axis).

FIG. 12 illustrates that the network generated from MIC scores can be used to select target microorganisms to use as a supplement to prevent and treat colic.

FIG. 13 represents the taxonomies of colic-associated microbes (-MIC) and healthy-associated microbes (+MIC) identified through the platform analysis.

FIG. 14 illustrates the fecal microbiome of horses with large colon volvulus colic and healthy horses.

FIG. 15 shows the abundance of microorganisms in Patient #1's fecal microbiome before (left), after administration of native microorganisms for 2 weeks (center), and 1 month after the administration stopped (right).

FIG. 16 shows the alpha diversity of Patient #1's fecal microbiome before (left) after administration of native microorganisms for 2 weeks (center), and 1 month after the administration stopped (right).

FIG. 17 shows the abundance of microorganisms in Patient #2's fecal microbiome before and after administration of native microorganisms for 2 weeks. The microbiome undergoes a shift from a healthy (left) to an even more healthy (right) state.

FIG. 18 shows the alpha diversity of Patient #2's fecal microbiome before (left) and after administration (right) of native microorganisms for 2 weeks.

FIG. 19 shows the abundance of microorganisms in Patient #3's fecal microbiome before and after administration of native microorganisms for 2 weeks. The microbiome undergoes a shift from a healthy (left) to an even more healthy (right) state.

FIG. 20 shows the alpha diversity of Patient #3's fecal microbiome before (left) and after administration (right) of native microorganisms for 2 weeks.

FIG. 21 shows the abundance of microorganisms in Patient #4's fecal microbiome before and after administration of native microorganisms for 2 weeks. The microbiome undergoes a shift from a colic state (left) to a healthy (right) state.

FIG. 22 shows shows the alpha diversity of Patient #4's fecal microbiome before (left) and after administration (right) of native microorganisms for 2 weeks.

FIG. 23 shows the abundance of microorganisms in Patient #5's fecal microbiome before and after administration of native microorganisms for 2 weeks. The microbiome undergoes a shift from a colic state (left) to a more healthy (right) state.

FIG. 24 shows the alpha diversity of Patient #5's fecal microbiome before (left), during (center) and after administration (right) of native microorganisms for 2 weeks.

FIG. 25 shows the abundance of microorganisms in Patient #6's fecal microbiome before, after administration of native microorganisms for 2 weeks (center), and 1 month after administration (right). The microbiome undergoes a shift from a colic state (left) to a healthy (right) state.

FIG. 26 shows the alpha diversity of Patient #6's fecal microbiome before (left), after administration of native microorganisms for 2 weeks (center), and 1 month after administration (right).

FIG. 27 shows the abundance of microorganisms in Patient #7's fecal microbiome before and after administration of native microorganisms for 2 weeks. The microbiome undergoes a shift from a colic state (left) to a healthy (right) state.

FIG. 28 shows the alpha diversity of Patient #7's fecal microbiome before (left) and after administration (right) of native microorganisms for 2 weeks.

FIG. 29 shows the relative abundance of healthy-associated microorganisms with respect to alpha diversity in several horses.

FIG. 30 illustrates microbiome co-clustering of healthy, transitional, and colic health states.

FIG. 31 shows a general workflow of a method for determining the absolute abundance of one or more active microorganism strains.

FIG. 32 shows a general workflow of a method for determining the co-occurrence of one or more, or two or more, active microorganism strains in a sample with one or more metadata (environmental) parameters, followed by leveraging cluster analysis and community detection methods on the network of determined relationships.

### DETAILED DESCRIPTION

The ability to return to a healthy state after perturbation is in part regulated by the microbial populations and biochemical functions of the gut. A supplemented group of microbes isolated from the horse or other equine gut can regulate the community to a healthy state. The ability to properly regulate the gut microbial community to a healthy state can prevent early-stage colic from negative internal or external pressures as well as prevent the downward spiral from early stage-colic to other forms of severe colic. A supplemented group of microbes can be administered daily via animal feed, supplement, or water to prevent disease onset and progression. Such a supplement can also be administered by fecal transplant and/or directly to target organs during pre/post/during surgery to treat a horse or other equine in a pre-existing chronic state.

The disclosure is generally drawn to methods of administering one or more microbes of the present disclosure to equines. In some aspects, the disclosure is generally drawn to methods for treating or preventing colic in equines, the method comprising: administering to an equine an effective amount of a microbial composition comprising: (i) any one or more of the bacteria set forth in Table 2; and (ii) a carrier suitable for equine administration.

In some aspects, the disclosure is generally drawn to a microbial composition capable of treating or preventing colic in an equine, comprising: (i) a purified population of bacteria comprising one or more bacteria selected from Table 2; and (ii) a carrier suitable for equine administration, wherein the purified population of bacteria is present in the composition in an amount effective to reduce colic symptoms and/or shift the gut microbiome, as compared to an equine not having been administered the composition. In some embodiments, the bacteria are encapsulated. In some embodiments, the microbial composition is shelf stable.

In some aspects, the microbial composition is administered via a fecal transplant from a healthy equine. In some aspects, the microbial composition is administered in addition to a fecal transplant from a healthy equine. In some aspects, the microbial composition is administered orally. In further aspects, the oral administration includes administering the microbial composition sprayed onto or mixed into food/feed. In some aspects, the microbial composition is administered rectally. In further aspects, rectal administration includes administering the microbial composition as a suppository. In some aspects, the microbial composition is administered during equine surgery. In some aspects, the microbial composition is administered after equine surgery. In some aspects, the microbial composition is administered before equine surgery.

### Equines and Colic

In some embodiments, the present disclosure provides microbial compositions suitable for administration to an equine. In some embodiments, the present disclosure provides methods of preventing and/or treating colic in an equine. Herein, the term “equine animal” may be used interchangeably with the term “equine” and encompasses any member of the genus Equus. It encompasses, e.g., any horse or pony, the taxonomic designations Equus ferus and/or Equus caballus, and/or the subspecies Equus ferus caballus. The equine animal may, e.g., be a domestic or wild horse, zebra, mule, or donkey.

The term colic generally refers to abdominal pain. Throughout the years, it has become a broad term for a variety of conditions that cause a horse to exhibit clinical signs of abdominal pain. Consequently, it is used to refer to conditions of widely varying etiologies and severity. Numerous clinical signs are associated with colic. The most common include one or more of pawing repeatedly with a front foot, looking back at the flank region, curling the upper lip and arching the neck, repeatedly raising a rear leg or kicking at the abdomen, lying down, rolling from side to side, sweating, stretching out as if to urinate, straining to defecate, distention of the abdomen, loss of appetite, depression, and/or decreased number of bowel movements.

A colic diagnosis can be made and appropriate treatment begun after examination of the horse, considering the history of any previous problems or treatments, determining which part of the intestinal tract is involved, and identifying the cause of the particular episode of colic. The physical examination should include assessment of the cardiopulmonary and GI systems. The oral mucous membranes should be evaluated for color, moistness, and capillary refill time. The mucous membranes may become cyanotic or pale in horses with acute cardiovascular compromise and eventually hyperemic or muddy as peripheral vasodilation develops later in shock. The capillary refill time (normal ˜1.5 sec) may be shortened early but usually becomes prolonged as vascular stasis (venous pooling) develops. The membranes become dry as the horse becomes dehydrated. The heart rate increases due to pain, hemoconcentration, and hypotension; therefore, higher heart rates have been associated with more severe intestinal problems (strangulating obstruction). However, it is important to note that not all conditions requiring surgery are accompanied by a high heart rate.

An important aspect of the physical examination is the response to passing a nasogastric tube. Because horses can neither regurgitate nor vomit, adynamic ileus, obstructions involving the small intestine, or distention of the stomach with gas or fluid may result in gastric rupture. Passing a stomach tube may, therefore, save the horse's life and assist in diagnosis of these conditions. If fluid reflux occurs, the volume and color of the fluid should be noted. In healthy horses, it is common to retrieve <1 L of fluid from the stomach.

The most definitive part of the examination is the rectal examination. The veterinarian should develop a consistent method of palpating for the following: aorta, cranial mesenteric artery, cecal base and ventral cecal band, bladder, peritoneal surface, inguinal rings in stallions and geldings or the ovaries and uterus in mares, pelvic flexure, spleen, and left kidney. The intestine should be palpated for size, consistency of contents (gas, fluid, or impacted ingesta), distention, edematous walls, and pain on palpation. In healthy horses, the small intestine cannot be palpated; with small-intestinal obstruction, strangulating obstruction, or enteritis, the distended duodenum can be palpated dorsal to the base of the cecum on the right side of the abdomen, and distended loops of jejunum can be identified in the middle of the abdomen.

A sample of peritoneal fluid (obtained via paracentesis performed aseptically on midline) often reflects the degree of intestinal damage. The color, cell count and differential, and total protein concentration should be evaluated. Normal peritoneal fluid is clear to yellow, contains <5,000 WBCs/μL (most of which are mononuclear cells), and <2.5 g of protein/dL.

The age of the horse is important, because a number of age-related conditions cause colic. The more common of these include the following: in foals—atresia coli, meconium retention, uroperitoneum, and gastroduodenal ulcers; in yearlings—ascarid impaction; in the young—small-intestinal intussusception, nonstrangulating infarction, and foreign body obstruction; in the middle-aged—cecal impaction, enteroliths, and large-colon volvulus; and in the aged—pedunculated lipoma and mesocolic rupture.

In most instances, colic develops for one of four reasons: 1) The wall of the intestine is stretched excessively by either gas, fluid, or ingesta. This stimulates the stretch-sensitive nerve endings located within the intestinal wall, and pain impulses are transmitted to the brain. 2) Pain develops due to excessive tension on the mesentery, as might occur with an intestinal displacement. 3) Ischemia develops, most often as a result of incarceration or severe twisting of the intestine. 4) Inflammation develops and may involve either the entire intestinal wall (enteritis) or the covering of the intestine (peritonitis). Under such circumstances, pro-inflammatory mediators in the wall of the intestine decrease the threshold for painful stimuli.

The list of possible conditions that cause colic is long, and it is reasonable first to determine the most likely type of disease and begin appropriate treatments and then to make a more specific diagnosis, if possible. The general types of disease that cause colic include excessive gas in the intestinal lumen (flatulent colic), simple obstruction of the intestinal lumen, obstruction of both the intestinal lumen and the blood supply to the intestine (strangulating obstruction), interruption of the blood supply to the intestine alone (nonstrangulating infarction), inflammation of the intestine (enteritis), inflammation of the lining of the abdominal cavity (peritonitis), erosion of the intestinal lining (ulceration), and “unexplained colic.”

Horses with colic may need either medical or surgical treatments. Almost all require some form of medical treatment, but only those with certain mechanical obstructions of the intestine need surgery. The type of medical treatment is determined by the cause of colic and the severity of the disease. In some instances, the horse may be treated medically first and the response evaluated; this is particularly appropriate if the horse is mildly painful and the cardiovascular system is functioning normally. Ultrasonography can be used to evaluate the effectiveness of nonsurgical treatment. If necessary, surgery can be used for diagnosis as well as treatment.

If evidence of intestinal obstruction with dry ingesta is found on rectal examination, a primary aim of treatment is to rehydrate and evacuate the intestinal contents. If the horse is severely painful and has clinical signs indicating loss of fluid from the bloodstream (high heart rate, prolonged capillary refill time, and discoloration of the mucous membranes), the initial aims of treatment are to relieve pain, restore tissue perfusion, and correct any abnormalities in the composition of the blood and body fluids. If damage to the intestinal wall (as a result of either severe inflammation or a displacement or strangulating obstruction) is suspected, steps should be taken to prevent or counteract the ill effects of bacterial endotoxins that cross the damaged intestinal wall and enter the bloodstream. Finally, if there is evidence the colic episode is caused by parasites, one aim of treatment is to eliminate the parasites.

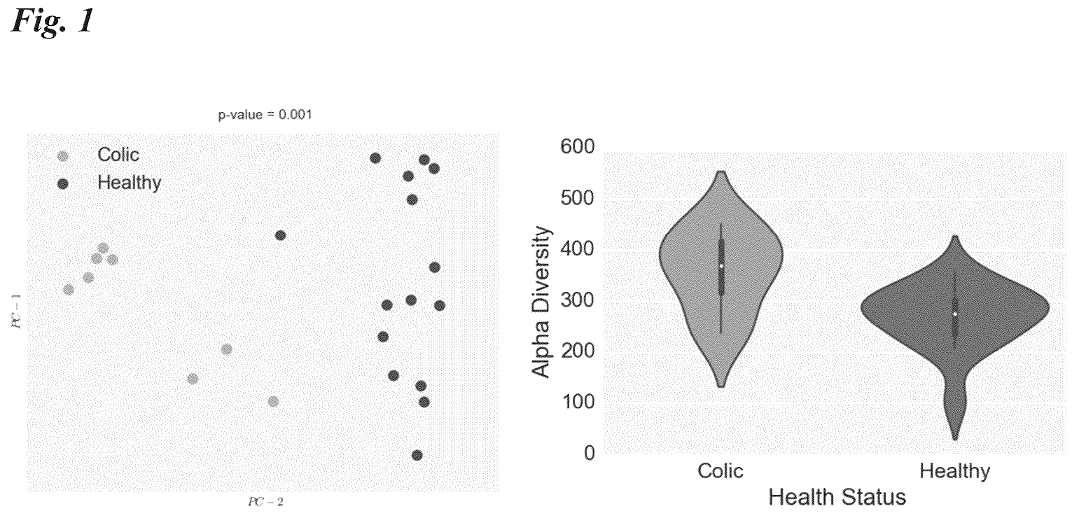

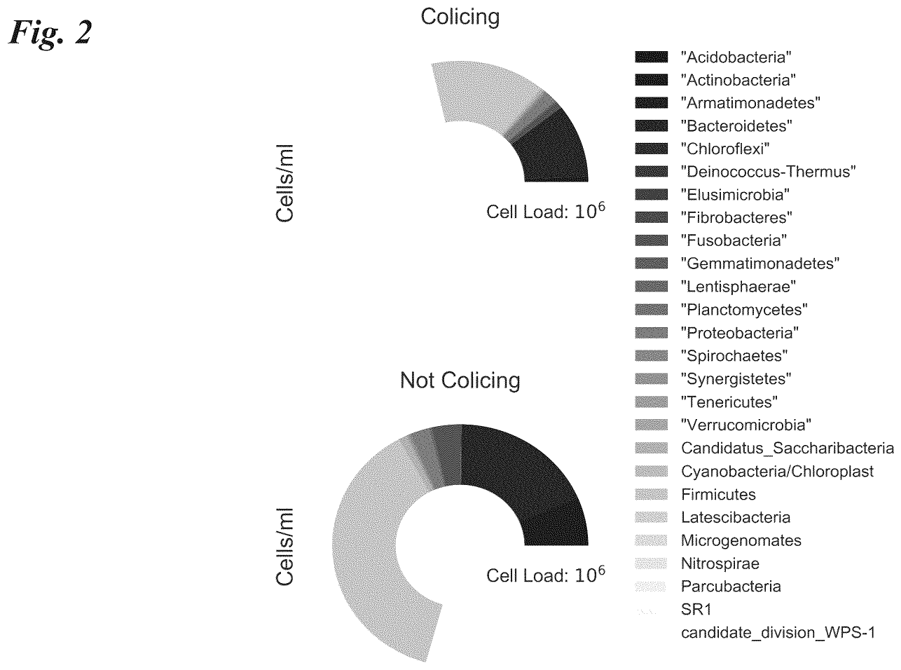

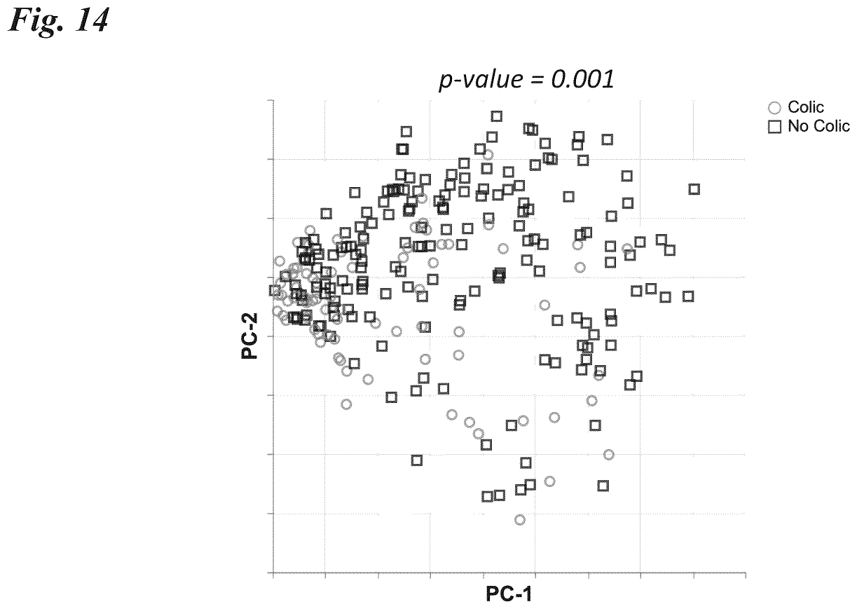

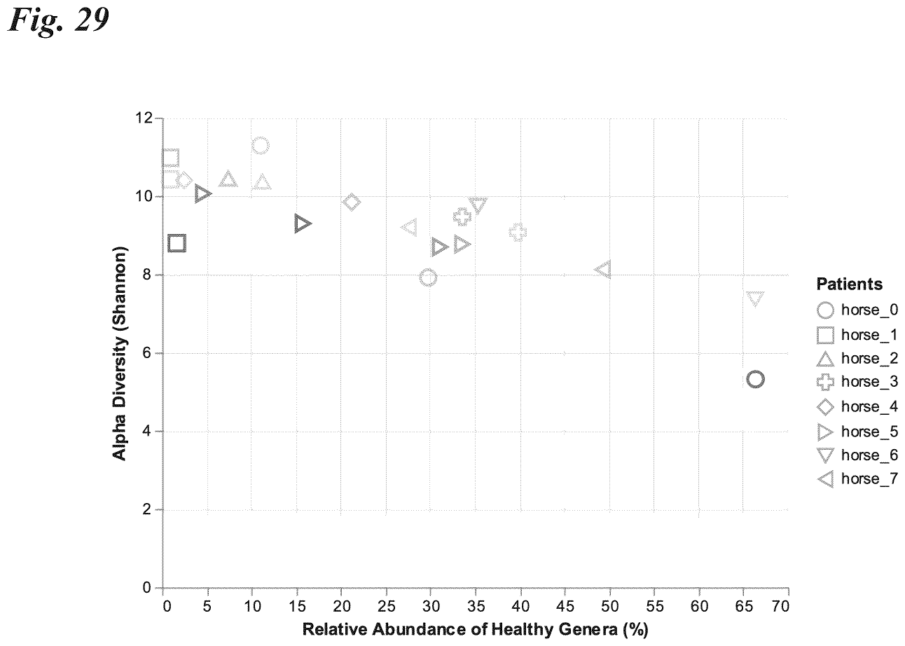

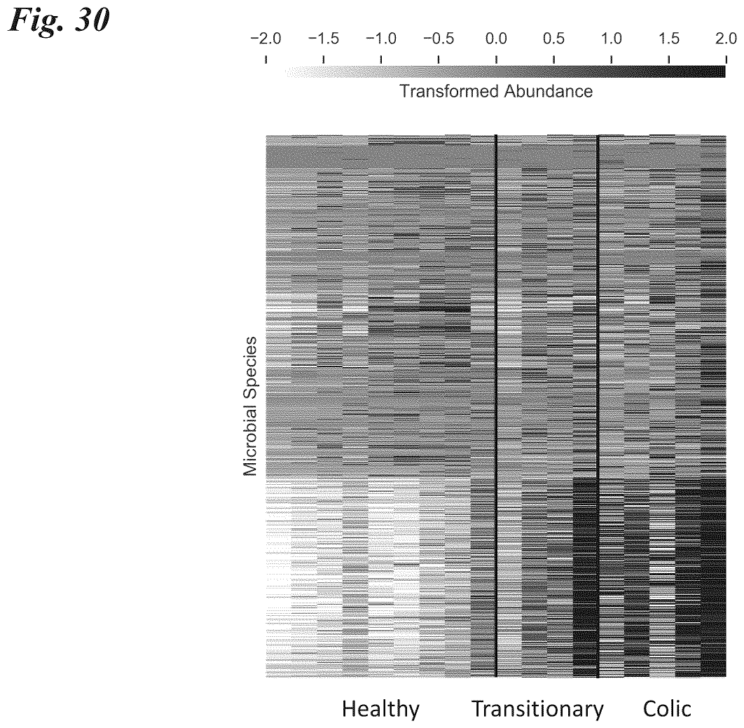

The microbiomes of healthy and colicking equines differ significantly. FIG. 1 shows the significant differences in beta diversity (left) and alpha diversity (right) between colic and healthy states in equines. In the left panel, each dot represents a microbiome sample from either a vet-diagnosed colicking horse (light gray) or healthy horse (black). The clear separation of samples (p-value=0.001) suggests clear microbiome differences between the healthy and colicking states. The right panel represents differences in alpha diversity between colicking (left) and healthy (right) animals with violin plots. As shown, colicking animals tend to have higher alpha diversity/more species diversity than healthy animals. FIG. 29 shows the relative abundance of healthy-associated microorganisms with respect to alpha diversity in several horses. The differences in the microbiomes are further illustrated in FIG. 2, which shows the differences in microbial load and microbial populations (total cells/ml) as well as taxonomic differences at the phylum level in the fecal microbiome of colic vs. no colic equines. Additional data demonstrating the differences in the fecal microbiome of colicking and healthy horses is provided in FIG. 14. The principal coordinate analysis of horses with large colon volvulus colic and health horses show distinct separation between colicing and healthy animals (p-value=0.001). Additional data demonstrating the differences in the fecal microbiome of colicking and healthy horses is provided in FIG. 30, which microbiome co-clustering of healthy, transitional, and colic health states.

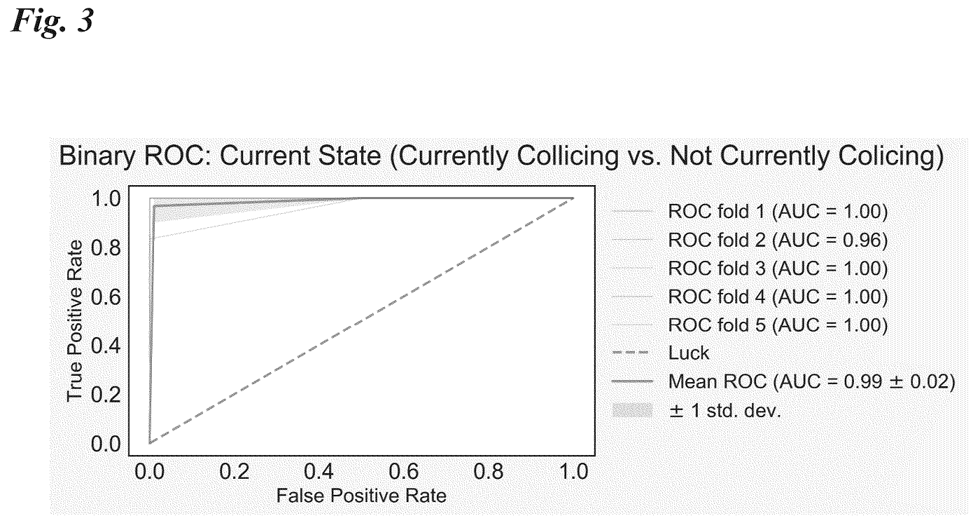

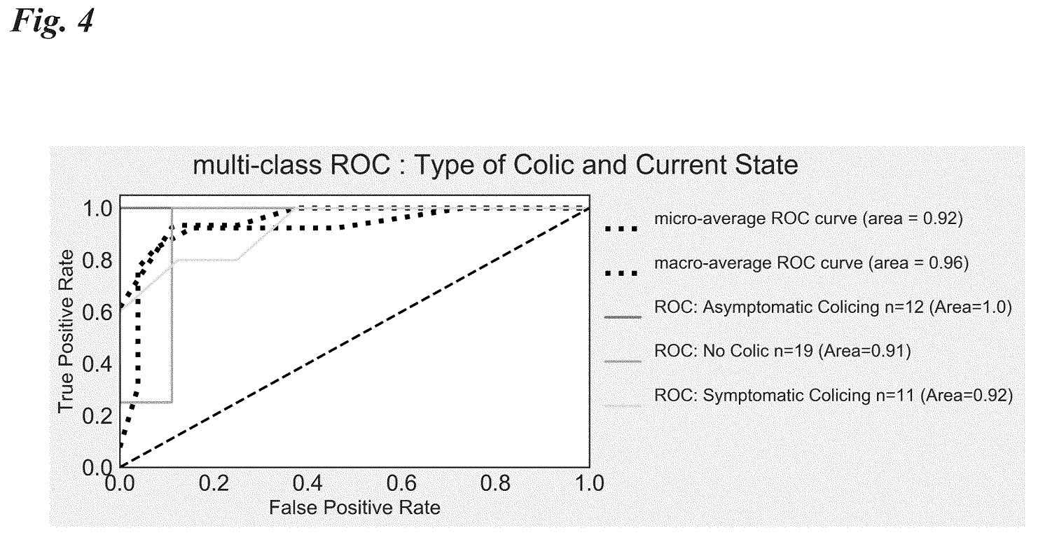

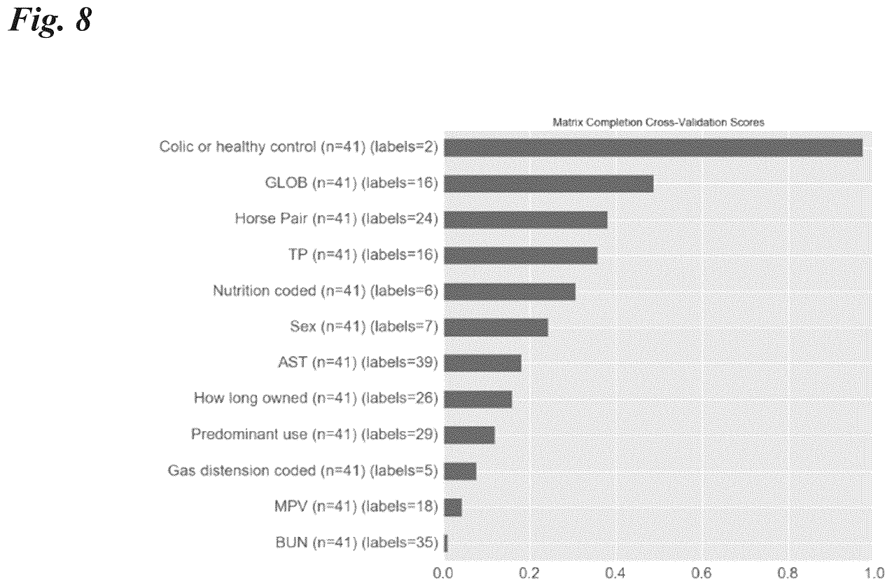

Machine learning algorithms can be used to determine if a patient is in a colic or non-colic state. FIG. 3 shows the receiver operator characteristic (ROC) curve for the performance of the binary classifier Machine learning between colic and healthy states has an accuracy of 99.99% using 5-fold 80:20 train: test split. Further, Multiclass classification algorithms can utilize the microbial composition of fecal samples to determine if the source patient is in a symptomatic colic, asymptomatic colic, or non-colic state. FIG. 4 shows the receiver operator characteristic (ROC) curve for the performance of the multiclass classifier. The machine learning between all states has a macro-average of 96% accuracy. The cross validation scores of machine learning models in FIG. 8 show that fecal microbiome data can be used to accurately diagnose microbial-mediated colic.

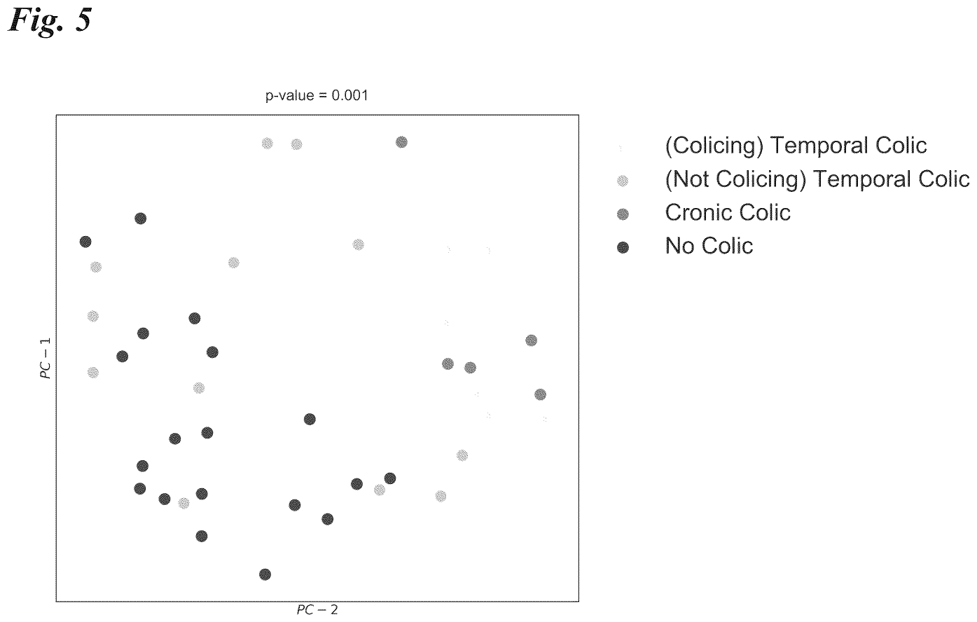

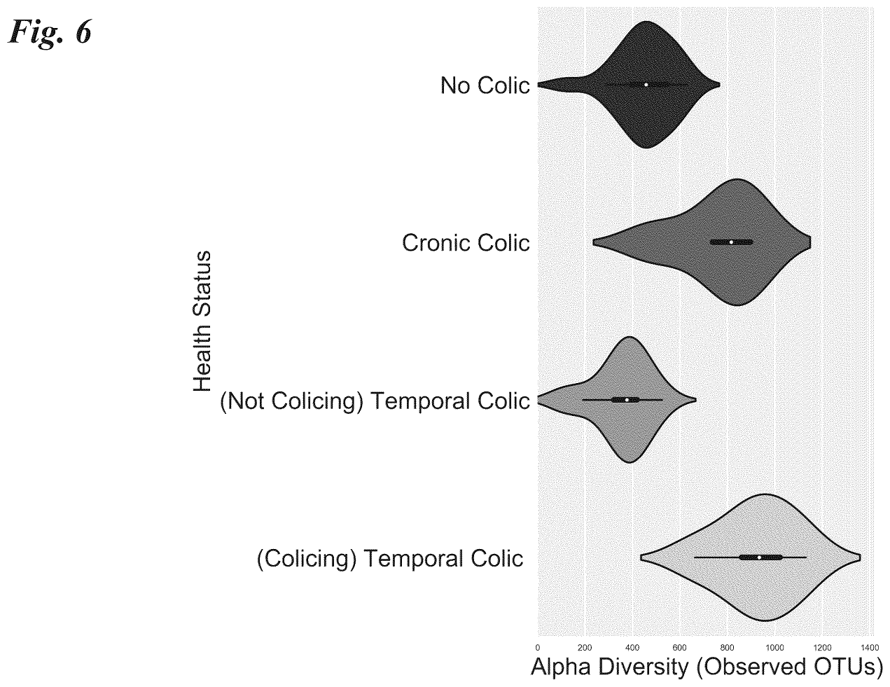

The differences in the microbiome of healthy equines compared to equines in various states of colic is further illustrated in FIG. 5. The principal coordinate analysis of samples classified as either no colic/healthy; colic; temporal/transient colic (colicing/symptomatic); or temporal/transient colic (not colicing/asymptomatic) suggests clear distinction between the β diversity of patients in each state (p-value=0.001). Similar data are provided in FIG. 6 for a diversity. Some overlap is observed between the transient colics and other states, but this is expected as it represents a temporary, actively shifting state to either a true colicking state or healthy state.

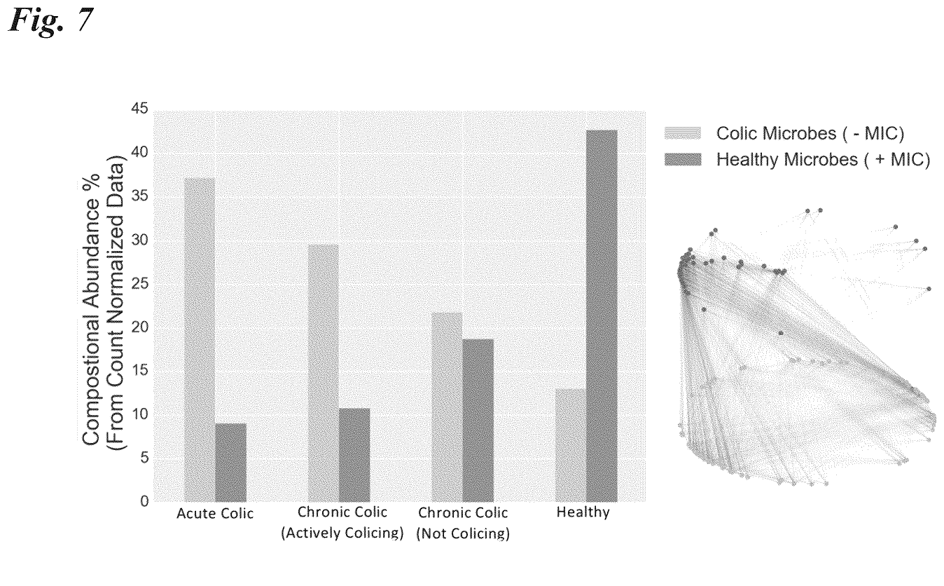

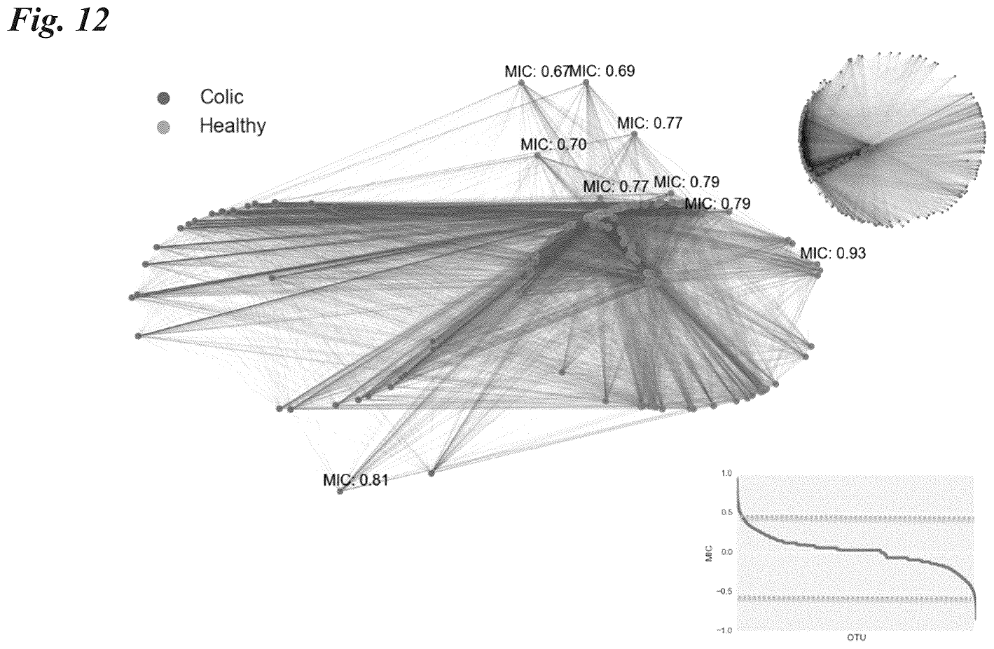

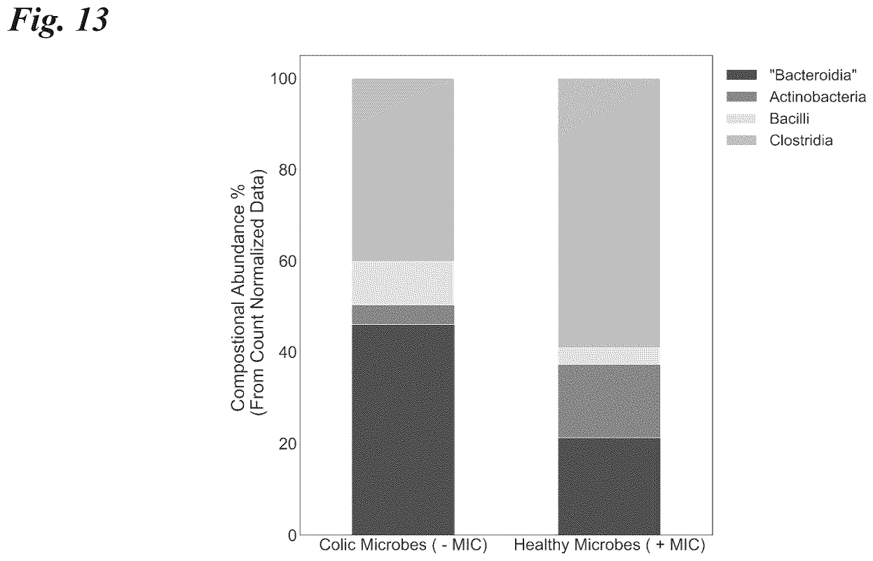

Additional differences in microbiomes of healthy and colic horses are illustrated by MIC scores. As shown in FIG. 7, the MIC score network and ranking based on colic are anti-correlated. Microorganisms with positive MIC scores (healthy state) are more abundant in healthy and transient/not colicing states. Microorganisms with negative MIC scores (colic state) are more abundant in colicing and transient/colicing states. The network generated from MIC scores was used to select target microorganisms to use as a supplement to prevent and treat colic, illustrated in FIG. 12. FIG. 13 shows the taxonomies of colic-associated microbes (-MIC) and healthy-associated microbes (+MIC) identified through the platform analysis.

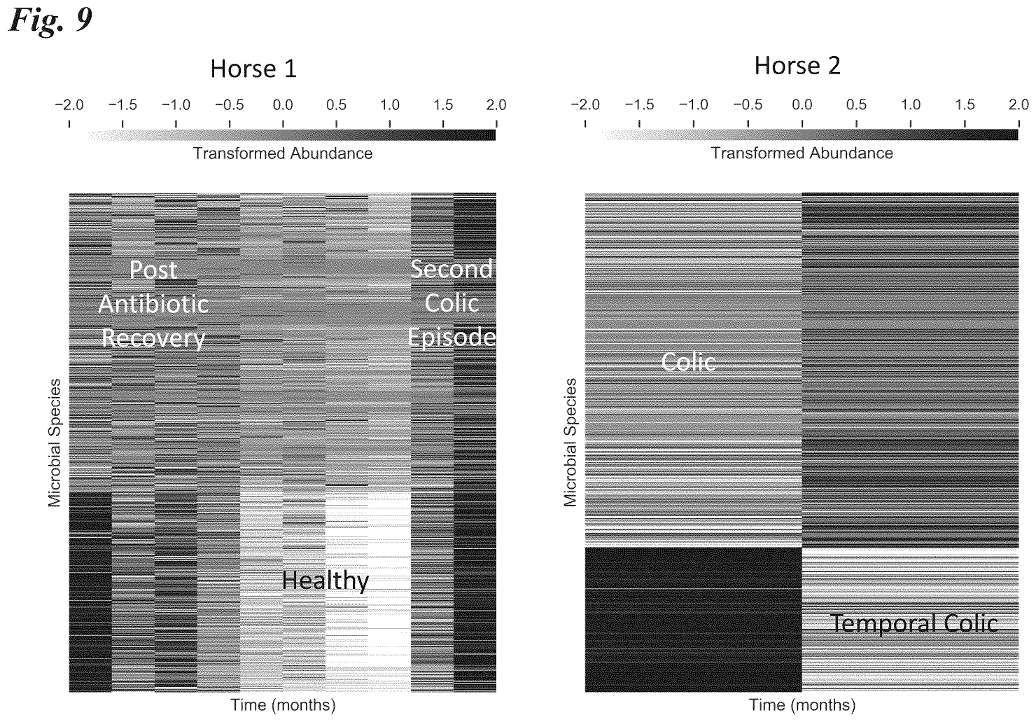

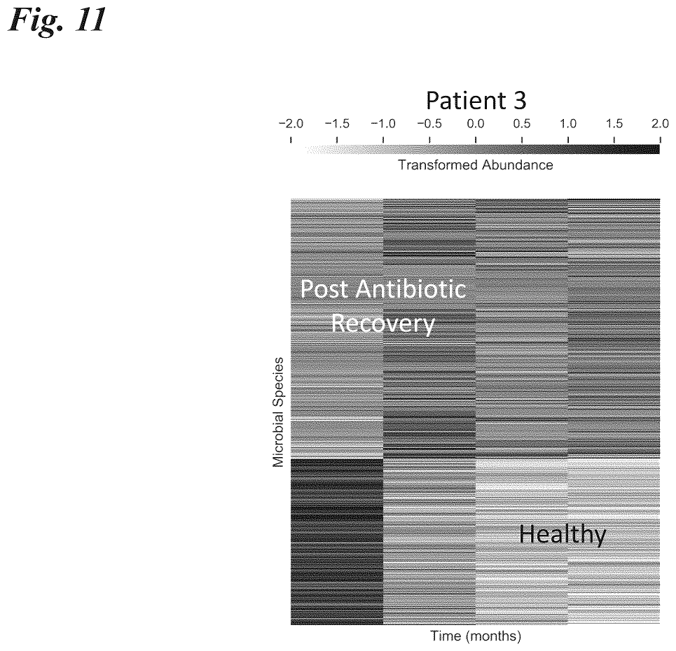

The microbial state of horses over time through periods of colic and non-colic further emphasize the differences between the microbiomes of healthy and colic horses. As shown in FIG. 9, heat maps of the fecal microbial abundances (y-axis) reveal clear differences between the healthy and colicking states over time (x-axis). Patient 1 was diagnosed with colic (far left) and underwent treatment with antibiotics. The patient seemed to recover and entered a healthy state for a few months. However, patient 1 experienced a second colic episode (far right), where the orginal colic-related microbes reemerged and caused colic symptoms in the patient. Patient 2 was diagnosed with colic (far left). Treatment started to push the patient's fecal microbiome towards a more healthy state, however, the patient relapsed and was ultimately euthanized. Similar results are shown in FIG. 11.

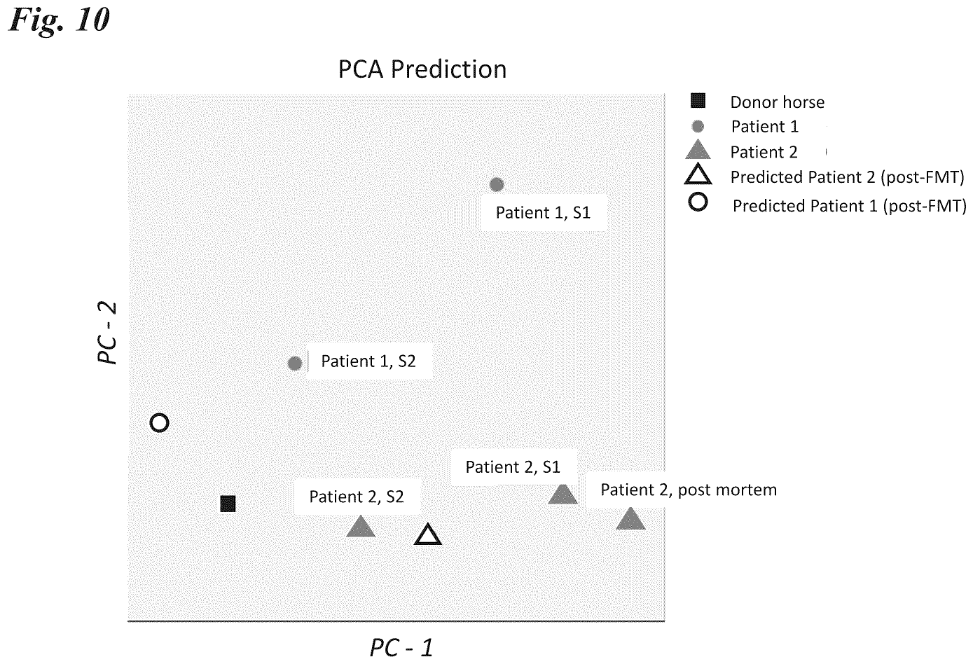

Principal coordinate analysis can also be used to determine the efficacy of fecal transplant, as shown in FIG. 10. Donor horse fecal material was used as a fecal transplant for Patient 1 and 2. To predict the efficacy of the procedure, Patient 1 and 2 fecal microbiome compositions prior to transplant (S1) were averaged with the donor horse microbiome composition. Predicted microbiomes are shown as a white circle (Patient 1) and white triangle (Patient 2). The direction/location of the predicted microbes are similar to the actual samples post fecal transplant (S2). Patient 1 had a successful transplant. Patient 2 relapsed, and was eventually euthanized (post mortem sample).

### Microbial Compositions

In some embodiments, the present disclosure provides microbial compositions comprising one or more target microbes. The target microbe may be any microorganisms suitable for use according to the present disclosure. As used herein the term “microorganism” should be taken broadly. It includes, but is not limited to, the two prokaryotic domains, Bacteria and Archaea, as well as eukaryotic fungi, protists, and viruses. By way of example, the microorganisms may include species of the genera of: Arcanobacterium, Acidaminococcus, Phascolarctobacterium, Bacteroides, Sutterella, Sutterella, Duodenibacillus, Catabacter, Christensenella, Clostridium, Clostridium sensu stricto, Anaerococcus, Finegoldia, Parvimonas, and Helcococcus, Anaerovorax, Ihubacter, Mogibacterium, Corynebacterium, Algoriphagus, Cecembia, Flavobacterium, Atlantibacter, Escherichia, Shigella, Erysipelothrix, Spiroplasma, Eubacterium, Flavobacterium, Devosia, Maritalea, Anaerocolumna, Anaerostipes, Butyrivibrio, Coprococcus, Cellulosilyticum, Clostridium XIVa, Frisingicoccus, Howardella, Oribacterium, Pediococcus, Peptococcus, Peptoniphilus, Terrisporobacter Peptostreptococcus, Barnesiella, Butyricimonas, Parabacteroides, Porphyromonas, Prevotella, Odoribacter, Alloprevotella, Bacteroides, Prevotella, Anaerocella Alistipe, Oscillibacter, Clostridium III, Intestinimonas, Ruminiclostridium, Monoglobus, Pedobacter, Streptococcus, Schwartzia, Selenomonas, Phascolarctobacterium, and Negativicoccus.

In some embodiments, the microbes are obtained from animals (e.g., mammals, reptiles, birds, and the like), soil (e.g., rhizosphere), air, water (e.g., marine, freshwater, wastewater sludge), sediment, oil, plants (e.g., roots, leaves, stems), agricultural products, and extreme environments (e.g., acid mine drainage or hydrothermal systems). In some embodiments, the microbes are obtained from marine or freshwater environments such as an ocean, river, or lake. In some embodiments, the microbes can be from the surface of the body of water, or any depth of the body of water (e.g., a deep sea sample).

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either administered to the GI tract of an ungulate, or concentrated by filtering or centrifugation, diluted to an appropriate concentration and administered to the GI tract of an ungulate with the bulk of the salt removed. By way of further example, microorganisms from mineralized or toxic sources may be similarly treated to recover the microbes for application to the ungulate to minimize the potential for damage to the animal.

In some embodiments, the microorganisms are used in a crude form, in which they are not isolated from the source material in which they naturally reside. For example, the microorganisms are provided in combination with the source material in which they reside; for example, fecal matter, cud, or other composition found in the gastrointestinal tract. In such embodiments, the source material may include one or more species of microorganisms.

In some embodiments, a mixed population of microorganisms is used in the methods of the disclosure. In embodiments of the disclosure where the microorganisms are isolated from a source material (for example, the material in which they naturally reside), any one or a combination of a number of standard techniques which will be readily known to skilled persons may be used. However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

In some embodiments, the material containing the microorganisms may be pre-treated prior to the isolation process in order to either multiply all microorganisms in the material. Microorganisms can then be isolated from the enriched materials.

The target microbes can be derived from any sample type that includes a microbial community. For example, samples for use with the present disclosure encompass without limitation, an animal sample (e.g., mammal, reptile, bird), soil, air, water (e.g., marine, freshwater, wastewater sludge), sediment, oil, plant, agricultural product, plant, soil (e.g., rhizosphere) and extreme environmental sample (e.g., acid mine drainage, hydrothermal systems). In the case of marine or freshwater samples, the sample can be from the surface of the body of water, or any depth of the body water, e.g., a deep sea sample. The water sample, in one embodiment, is an ocean, river, or lake sample.

In some embodiments, the animal sample is a body fluid. In some embodiments, the animal sample is a tissue sample. Non-limiting animal samples include tooth, perspiration, fingernail, skin, hair, feces, urine, semen, mucus, saliva, gastrointestinal tract. The animal sample can be, for example, a human, primate, bovine, porcine, canine, feline, rodent (e.g., mouse or rat), equine, or bird sample. In some embodiments, the bird sample comprises a sample from one or more chickens. In some embodiments, the sample is a human sample. The human microbiome comprises the collection of microorganisms found on the surface and deep layers of skin, in mammary glands, saliva, oral mucosa, conjunctiva, and gastrointestinal tract. The microorganisms found in the microbiome include bacteria, fungi, protozoa, viruses, and archaca. Different parts of the body exhibit varying diversity of microorganisms. The quantity and type of microorganisms may signal a healthy or diseased state for an individual. The number of bacteria taxa are in the thousands, and viruses may be as abundant. The bacterial composition for a given site on a body varies from person to person, not only in type, but also in abundance or quantity.

In another embodiment, the sample is a soil sample (e.g., bulk soil or rhizosphere sample). It has been estimated that 1 gram of soil contains tens of thousands of bacterial taxa, and up to 1 billion bacteria cells as well as about 200 million fungal hyphae (Wagg et al. (2010). Proc Natl. Acad. Sci. USA 111, pp. 5266-5270, incorporated by reference in its entirety for all purposes). Bacteria, actinomycetes, fungi, algae, protozoa, and viruses are all found in soil. Soil microorganism community diversity has been implicated in the structure and fertility of the soil microenvironment, nutrient acquisition by plants, plant diversity and growth, as well as the cycling of resources between above- and below-ground communities. Accordingly, assessing the microbial contents of a soil sample over time and the co-occurrence of active microorganisms (as well as the number of the active microorganisms) provides insight into microorganisms associated with an environmental metadata parameter such as nutrient acquisition and/or plant diversity.

The soil sample in one embodiment is a rhizosphere sample, i.e., the narrow region of soil that is directly influenced by root secretions and associated soil microorganisms. The rhizosphere is a densely populated area in which elevated microbial activities have been observed and plant roots interact with soil microorganisms through the exchange of nutrients and growth factors (San Miguel et al. (2014). Appl. Microbiol. Biotechnol. DOI 10.1007/s00253-014-5545-6, incorporated by reference in its entirety for all purposes). As plants secrete many compounds into the rhizosphere, analysis of the organism types in the rhizosphere may be useful in determining features of the plants which grow therein.

In another embodiment, the sample is a marine or freshwater sample. Ocean water contains up to one million microorganisms per milliliter and several thousand microbial types. These numbers may be an order of magnitude higher in coastal waters with their higher productivity and higher load of organic matter and nutrients. Marine microorganisms are crucial for the functioning of marine ecosystems; maintaining the balance between produced and fixed carbon dioxide; production of more than 50% of the oxygen on Earth through marine phototrophic microorganisms such as Cyanobacteria, diatoms and pico- and nanophytoplankton; providing novel bioactive compounds and metabolic pathways; ensuring a sustainable supply of seafood products by occupying the critical bottom trophic level in marine foodwebs. Organisms found in the marine environment include viruses, bacteria, archaca, and some eukarya. Marine viruses may play a significant role in controlling populations of marine bacteria through viral lysis. Marine bacteria are important as a food source for other small microorganisms as well as being producers of organic matter. Archaea found throughout the water column in the ocean are pelagic Archaea and their abundance rivals that of marine bacteria.

In another embodiment, the sample comprises a sample from an extreme environment, i.e., an environment that harbors conditions that are detrimental to most life on Earth. Organisms that thrive in extreme environments are called extremophiles. Though the domain Archaca contains well-known examples of extremophiles, the domain bacteria can also have representatives of these microorganisms. Extremophiles include: acidophiles which grow at pH levels of 3 or below; alkaliphiles which grow at pH levels of 9 or above; anaerobes such as Spinoloricus Cinzia which does not require oxygen for growth; cryptoendoliths which live in microscopic spaces within rocks, fissures, aquifers and faults filled with groundwater in the deep subsurface; halophiles which grow in about at least 0.2M concentration of salt; hyperthermophiles which thrive at high temperatures (about 80-122° C.) such as found in hydrothermal systems; hypoliths which live underneath rocks in cold deserts; lithoautotrophs such as Nitrosomonas europaea which derive energy from reduced mineral compounds like pyrites and are active in geochemical cycling; metallotolerant organisms which tolerate high levels of dissolved heavy metals such as copper, cadmium, arsenic and zinc; oligotrophs which grow in nutritionally limited environments; osmophiles which grow in environments with a high sugar concentration; piezophiles (or barophiles) which thrive at high pressures such as found deep in the ocean or underground; psychrophiles/cryophiles which survive, grow and/or reproduce at temperatures of about −15° C. or lower; radioresistant organisms which are resistant to high levels of ionizing radiation; thermophiles which thrive at temperatures between 45-122° C.; xerophiles which can grow in extremely dry conditions. Polyextremophiles are organisms that qualify as extremophiles under more than one category and include thermoacidophiles (prefer temperatures of 70-80° C. and pH between 2 and 3). The Crenarchacota group of Archaca includes the thermoacidophiles.

The sample can include microorganisms from one or more domains. For example, in some embodiments, the sample comprises a heterogeneous population of bacteria and/or fungi (also referred to herein as bacterial or fungal strains). For example, the one or more microorganisms can be from the domain Bacteria, Archaca, Eukarya or a combination thereof. Bacteria and Archaca are prokaryotic, having a very simple cell structure with no internal organelles. Bacteria can be classified into gram positive/no outer membrane, gram negative/outer membrane present and ungrouped phyla. Archaea constitute a domain or kingdom of single-celled microorganisms. Although visually similar to bacteria, archaea possess genes and several metabolic pathways that are more closely related to those of eukaryotes, notably the enzymes involved in transcription and translation. Other aspects of archacal biochemistry are unique, such as the presence of ether lipids in their cell membranes. The Archaca are divided into four recognized phyla: Thaumarchacota, Aigarchacota, Crenarchacota, and Korarchacota.

The domain of Eukarya comprises eukaryotic organisms, which are defined by membrane-bound organelles, such as the nucleus. Protozoa are unicellular eukaryotic organisms. All multicellular organisms are eukaryotes, including animals, plants, and fungi. The eukaryotes have been classified into four kingdoms: Protista, Plantae, Fungi, and Animalia. However, several alternative classifications exist. Another classification divides Eukarya into six kingdoms: Excavata (various flagellate protozoa); amoebozoa (lobose amoeboids and slime filamentous fungi); Opisthokonta (animals, fungi, choanoflagellates); Rhizaria (Foraminifera, Radiolaria, and various other amocboid protozoa); Chromalveolata (Stramenopiles (brown algae, diatoms), Haptophyta, Cryptophyta (or cryptomonads), and Alveolata); Archaeplastida/Primoplantae (Land plants, green algae, red algae, and glaucophytes).

Within the domain of Eukarya, fungi are microorganisms that are predominant in microbial communities. Fungi include microorganisms such as yeasts and filamentous fungi as well as the familiar mushrooms. Fungal cells have cell walls that contain glucans and chitin, a unique feature of these organisms. The fungi form a single group of related organisms, named the Eumycota that share a common ancestor. The kingdom Fungi has been estimated at 1.5 million to 5 million species, with about 5% of these having been formally classified. The cells of most fungi grow as tubular, elongated, and filamentous structures called hyphae, which may contain multiple nuclei. Some species grow as unicellular yeasts that reproduce by budding or binary fission. The major phyla (sometimes called divisions) of fungi have been classified mainly on the basis of characteristics of their sexual reproductive structures. Currently, seven phyla are proposed: Microsporidia, Chytridiomycota, Blastocladiomycota, Neocallimastigomycota, Glomeromycota, Ascomycota, and Basidiomycota.

Microorganisms for detection and quantification by the methods described herein can also be viruses. A virus is a small infectious agent that replicates only inside the living cells of other organisms. Viruses can infect all types of life forms in the domains of Eukarya, Bacteria, and Archaca. Virus particles (known as virions) consist of two or three parts: (i) the genetic material which can be either DNA or RNA; (ii) a protein coat that protects these genes; and in some cases (iii) an envelope of lipids that surrounds the protein coat when they are outside a cell. Seven orders have been established for viruses: the Caudovirales, Herpesvirales, Ligamenvirales, Mononegavirales, Nidovirales, Picornavirales, and Tymovirales. Viral genomes may be single-stranded (ss) or double-stranded (ds), RNA or DNA, and may or may not use reverse transcriptase (RT). In addition, ssRNA viruses may be either sense (+) or antisense (−). This classification places viruses into seven groups: I: dsDNA viruses (such as Adenoviruses, Herpesviruses, Poxviruses); II: (+) ssDNA viruses (such as Parvoviruses); III: dsRNA viruses (such as Reoviruses); IV: (+) ssRNA viruses (such as Picornaviruses, Togaviruses); V: (−) ssRNA viruses (such as Orthomyxoviruses, Rhabdoviruses); VI: (+) ssRNA-RT viruses with DNA intermediate in life-cycle (such as Retroviruses); VII: dsDNA-RT viruses (such as Hepadnaviruses).

Microorganisms for detection and quantification by the methods described herein can also be viroids. Viroids are the smallest infectious pathogens known, consisting solely of short strands of circular, single-stranded RNA without protein coats. They are mostly plant pathogens, some of which are of economic importance. Viroid genomes are extremely small in size, ranging from about 246 to about 467 nucleobases.

### Isolated Microbes

As used herein, “isolate”, “isolated”, “isolated microbe”, and like terms, are intended to mean that the one or more microorganisms has been separated from at least one of the materials with which it is associated in a particular environment (for example soil, water, animal tissue). Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an acceptable carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often necessarily differ from less pure or impure materials. See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S. D. N. Y. 1911) (Learned Hand discussing purified adrenaline), aff′d in part, rev′d in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

In some embodiments, the present disclosure provides microbial compositions comprising isolated microbial species belonging to taxonomic families of Actinomycetaceae, Acidaminococcaceae, Bacteroidaceae, Burkholderiaceae, Catabacteriaceae, Christensenellaceae, Clostridiaceae, Clostridiales Incertae Sedis XI, Clostridiales Incertae Sedis XIII, Corynebacteriaceae, Cyclobacteriaceae, Enterobacteriaceae, Erysipelotrichaceae, Eubacteriaceae, Flavobacteriaceae, Lactobacilluseae, Lachnospiraceae, Hyphomicrobiaceae,
Peptoniphilaceae, Peptococcaceae, Peptostreptococcaceae, Porphyromonadaceae, Prevotellaceae, Rikenellaceae, Ruminococcaceae, Sphingobacteriaceae, Spiroplasmataceae, Streptococcaceae, Sutterellaceae, and Veillonellaceae.

In some embodiments, the present disclosure provides microbial compositions comprising isolated microbial species from the genera Arcanobacterium of family Actinomycetaceae.

In some embodiments, the present disclosure provides microbial compositions comprising isolated microbial species selected from genera of family Acidaminococcaceae, including Acidaminococcus and Phascolarctobacterium.

In some embodiments, the present disclosure provides microbial compositions comprising isolated microbial species from the genera Bacteroides of family Bacteroidaceae.

In some embodiments, the present disclosure provides microbial compositions comprising isolated microbial species from the genera Sutterella of family Burkholderiaceae.

In some embodiments, the present disclosure provides microbial compositions comprising isolated microbial species selected from genera of family Sutterellaceae, including Sutterella and Duodenibacillus.

In some embodiments, the present disclosure provides microbial compositions comprising isolated microbial species from the genera Catabacter of family Catabacteriaceae.

In some embodiments, the present disclosure provides microbial compositions comprising isolated microbial species from the genera Christensenella of family Christensenellaceae.

In some embodiments, the present disclosure provides microbial compositions comprising isolated microbial species selected from genera of family Clostridiaceae, including Clostridium and Clostridium sensu stricto.

In some embodiments, the present disclosure provides microbial compositions comprising isolated microbial species selected from genera of family Clostridiales Incertae Sedis XI, including Anaerococcus, Finegoldia, Parvimonas, and Helcococcus.

In some embodiments, the present disclosure provides microbial compositions comprising isolated microbial species selected from genera of family Clostridiales Incertae Sedis XIII, including Anaerovorax, Ihubacter, and Mogibacterium.

In some embodiments, the present disclosure provides microbial compositions comprising isolated microbial species from the genera Corynebacterium of family Corynebacteriaceae.

In some embodiments, the present disclosure provides microbial compositions comprising isolated microbial species selected from genera of family Cyclobacteriaceae, including Algoriphagus, Cecembia, and Flavobacterium.

In some embodiments, the present disclosure provides microbial compositions comprising isolated microbial species selected from genera of family Enterobacteriaceae, including Atlantibacter, Escherichia, and Shigella.

In some embodiments, the present disclosure provides microbial compositions comprising isolated microbial species from the genera Erysipelothrix of family Erysipelotrichaceae.

In some embodiments, the present disclosure provides microbial compositions comprising isolated microbial species from the genera Spiroplasma of family Spiroplasmataceae.

In some embodiments, the present disclosure provides microbial compositions comprising isolated microbial species from the genera Eubacterium of family Eubacteriaceae.

In some embodiments, the present disclosure provides microbial compositions comprising isolated microbial species from the genera Flavobacterium of family Flavobacteriaceae.

In some embodiments, the present disclosure provides microbial compositions comprising isolated microbial species selected from genera of family Hyphomicrobiaceae, including Devosia, and Maritalea.

In some embodiments, the present disclosure provides microbial compositions comprising isolated microbial species selected from genera of family Lachnospiraceae, including Anaerocolumna, Anaerostipes, Butyrivibrio, Coprococcus, Cellulosilyticum, Clostridium XIVa, Frisingicoccus, Howardella, and Oribacterium.

In some embodiments, the present disclosure provides microbial compositions comprising isolated microbial species from the genera Pediococcus of family Lactobacilluseae.

In some embodiments, the present disclosure provides microbial compositions comprising isolated microbial species from the genera Peptococcus of family Peptococcaceae.

In some embodiments, the present disclosure provides microbial compositions comprising isolated microbial species from the genera Peptoniphilus of family Peptoniphilaceae.

In some embodiments, the present disclosure provides microbial compositions comprising isolated microbial species selected from genera of family Peptostreptococcaceae, including Terrisporobacter and Peptostreptococcus.

In some embodiments, the present disclosure provides microbial compositions comprising isolated microbial species selected from genera of family Porphyromonadaceae, including Barnesiella, Butyricimonas, Parabacteroides, Porphyromonas, Prevotella, and Odoribacter.

In some embodiments, the present disclosure provides microbial compositions comprising isolated microbial species selected from genera of family Prevotellaceae, including Alloprevotella, Bacteroides, and Prevotella.

In some embodiments, the present disclosure provides microbial compositions comprising isolated microbial species selected from genera of family Rikenellaceae, including Anaerocella and Alistipes.

In some embodiments, the present disclosure provides microbial compositions comprising isolated microbial species selected from genera of family Ruminococcaceae, including Oscillibacter, Clostridium III, Intestinimonas, Ruminiclostridium, and Monoglobus.

In some embodiments, the present disclosure provides microbial compositions comprising isolated microbial species from the genera Pedobacter of family Sphingobacteriaceae.

In some embodiments, the present disclosure provides microbial compositions comprising isolated microbial species from the genera Streptococcus of family Streptococcaceae.

In some embodiments, the present disclosure provides microbial compositions comprising isolated microbial species selected from genera of family Veillonellaceae, including Schwartzia, Selenomonas, Phascolarctobacterium, and Negativicoccus.

In some embodiments, the present disclosure provides microbial compositions comprising isolated microbial species selected from genera of: Clostridium, Sarcina, Streptococcus, Escheria, Atlantibacter, and Shigella.

In some embodiments, the isolated microbial strains in the compositions described herein have been genetically modified. In some embodiments, the genetically modified or recombinant microbes comprise polynucleotide sequences which do not naturally occur in said microbes. In some embodiments, the microbes may comprise heterologous polynucleotides. In further embodiments, the heterologous polynucleotides may be operably linked to one or more polynucleotides native to the microbes.

In some embodiments, the heterologous polynucleotides may be reporter genes or selectable markers. In some embodiments, reporter genes may be selected from any of the family of fluorescence proteins (e.g., GFP, RFP, YFP, and the like), β-galactosidase, or luciferase. In some embodiments, selectable markers may be selected from neomycin phosphotransferase, hygromycin phosphotransferase, aminoglycoside adenyltransferase, dihydrofolate reductase, acetolactase synthase, bromoxynil nitrilase, β-glucuronidase, dihydrogolate reductase, and chloramphenicol acetyltransferase. In some embodiments, the heterologous polynucleotide may be operably linked to one or more promoter.

In some embodiments, the isolated microbes are identified by ribosomal nucleic acid sequences. Ribosomal RNA genes (rDNA), especially the small subunit ribosomal RNA genes, i.e., 18S rRNA genes (18S rDNA) in the case of eukaryotes and 16S rRNA (16S rDNA) in the case of prokaryotes, have been the predominant target for the assessment of organism types and strains in a microbial community. However, the large subunit ribosomal RNA genes, 28S rDNAs, have been also targeted. rDNAs are suitable for taxonomic identification because: (i) they are ubiquitous in all known organisms; (ii) they possess both conserved and variable regions; (iii) there is an exponentially expanding database of their sequences available for comparison. In community analysis of samples, the conserved regions serve as annealing sites for the corresponding universal PCR and/or sequencing primers, whereas the variable regions can be used for phylogenetic differentiation. In addition, the high copy number of rDNA in the cells facilitates detection from environmental samples.

The internal transcribed spacer (ITS), located between the 18S rDNA and 28S rDNA, has also been targeted. The ITS is transcribed but spliced away before assembly of the ribosomes. The ITS region is composed of two highly variable spacers, ITS1 and ITS2, and the intercalary 5.8S gene. This rDNA operon occurs in multiple copies in genomes. Because the ITS region does not code for ribosome components, it is highly variable. In some embodiments, the unique RNA marker can be an mRNA marker, an siRNA marker, or a ribosomal RNA marker.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaca (Yarza et al. 2014. Nature Rev. Micro. 12:635-45)

In some embodiments, a sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

Exemplary isolated microbes for use according to the present disclosure are provided below in Table 2. Designation of a strain with the Ascus identifier followed by a letter (e.g., AscusEQ_4A, AscusEQ_4B, AscusEQ_4C, etc.) indicates that these strains are variants of the parental strain with the corresponding Ascus identifier. For example, AscusEQ_4A, AscusEQ_4B, AscusEQ_4C, etc. are all variants of the AscusEQ_4 parental strain. Each variant strain shares at least 97% sequence identity with the parental strain.

TABLE 2
Exemplary Isolated Microbes

| | | | | % Identity to | % Identity to | |
|---|---|---|---|---|---|---|
| Strain | | | | BLAST | parental | SEQ |
| Designation | MIC | Predicted Taxonomy (Utax) | Predicted Taxonomy (BLAST) | taxonomic hit | strain | ID: |
| AscusEQ_0 | 0.99885 | Arcanobacterium hippocoleae | Arcanobacterium hippocolea | 95 | | 1 |
| AscusEQ_1 | 0.99885 | Anaerovorax odorimutans | Ihubacter massiliensis | 90.73 | | 2 |
| AscusEQ_2 | 0.99885 | Peptoniphilus indolicus | Peptoniphilus asaccharolyticus | 91.95 | | 3 |
| AscusEQ_3 | 0.99885 | Anaerococcus octavius | Anaerococcus vaginalis | 89.37 | | 4 |
| AscusEQ_4 | 0.99885 | Clostridium butyricum | Clostridium butyricum | 99.67 | | 5 |
| AscusEQ_4A | | Clostridium butyricum | Clostridium butyricum | | 97.3 | 6 |
| AscusEQ_4B | | Clostridium butyricum | Clostridium butyricum | | 98.7 | 7 |
| AscusEQ_4C | | Clostridium butyricum | Clostridium butyricum | | 99.1 | 8 |
| AscusEQ_4D | | Clostridium butyricum | Clostridium butyricum | | 99.6 | 9 |
| AscusEQ_4E | | Clostridium butyricum | Clostridium butyricum | | 98.2 | 10 |
| AscusEQ_4F | | Clostridium butyricum | Clostridium butyricum | | 100 | 11 |
| AscusEQ_4G | | Clostridium butyricum | Clostridium butyricum | | 97.8 | 12 |
| AscusEQ_4H | | Clostridium butyricum | Clostridium butyricum | | 97.4 | 13 |
| AscusEQ_5 | 0.99885 | Terrisporobacter mayombei | Terrisporobacter mayombei | 97.66 | | 14 |
| AscusEQ_5A | | Terrisporobacter mayombei | Terrisporobacter mayombei | | 98.2 | 15 |
| AscusEQ_5B | | Terrisporobacter mayombei | Terrisporobacter mayombei | | 98.7 | 16 |
| AscusEQ_5C | | Terrisporobacter mayombei | Terrisporobacter mayombei | | 99.6 | 17 |
| AscusEQ_5D | | Terrisporobacter mayombei | Terrisporobacter mayombei | | 97.3 | 18 |
| AscusEQ_7 | 0.91885 | Mogibacterium neglectum | Mogibacterium pumilum | 91.06 | | 19 |
| AscusEQ_7A | | Mogibacterium neglectum | Mogibacterium pumilum | | 99.1 | 20 |
| AscusEQ_7B | | Mogibacterium neglectum | Mogibacterium pumilum | | 97.3 | 21 |
| AscusEQ_7C | | Mogibacterium neglectum | Mogibacterium pumilum | | 98.2 | 22 |
| AscusEQ_7D | | Mogibacterium neglectum | Mogibacterium pumilum | | 97.8 | 23 |
| AscusEQ_8 | 0.84284 | Arcanobacterium phocae | Arcanobacterium phocae | 97.37 | | 24 |
| AscusEQ_9 | 0.83885 | Christensenella minuta | Catabacter hongkongensis | 85.81 | | 25 |
| AscusEQ_9A | | Christensenella minuta | Catabacter hongkongensis | | 99.1 | 26 |
| AscusEQ_10 | 0.83885 | Parabacteroides merdae | Parabacieraides distasonis | 97 | | 27 |
| AscusEQ_11 | 0.83318 | Finegoldia magna | Finegoldia magna | 99.33 | | 28 |
| AscusEQ_12 | 0.82490 | Clostridium baratii | Clostridium nitritogenes | 98.67 | | 29 |
| AscusEQ_12A | | Clostridium baratii | Clostridium nitritogenes | | 100 | 30 |
| AscusEQ_12B | | Clostridium baratii | Clostridium nitritogenes | | 99.6 | 31 |
| AscusEQ_12C | | Clostridium baratii | Clostridium nitritogenes | | 99.1 | 32 |
| AscusEQ_12D | | Clostridium baratii | Clostridium nitritogenes | | 97.8 | 33 |
| AscusEQ_12E | | Clostridium baratii | Clostridium nitritogenes | | 98.7 | 34 |
| AscusEQ_12F | | Clostridium baratii | Clostridium nitritogenes | | 97.3 | 35 |
| AscusEQ_12G | | Clostridium baratii | Clostridium nitritogenes | | 98.2 | 36 |
| AscusEQ_13 | 0.81767 | Corynebacterium pyruviciproducens | Corynebacterium glaucum | 94.3 | | 37 |
| AscusEQ_14 | 0.80865 | Clostridium xylanovorans | Oribacterium sp. | 87.21 | | 38 |
| AscusEQ_14A | | Clostridium xylanovorans | Oribacterium sp. | | 99.1 | 39 |
| AscusEQ_14B | | Clostridium xylanovorans | Oribacterium sp. | | 99.6 | 40 |
| AscusEQ_15 | 0.80865 | Clostridium symbiosum | Lachnospiraceae bacterium | 96.32 | | 41 |
| AscusEQ_15A | | Clostridium symbiosum | Lachnospiraceae bacterium | | 99.6 | 42 |
| AscusEQ_15B | | Clostridrum symbiosum | Lachnospiraceae bacterium | | 98.2 | 43 |
| AscusEQ_15C | | Clostridium symbiosum | Lachnospiraceae bacterium | | 98.7 | 44 |
| AscusEQ_15D | | Clostridium symbiosum | Lachnospiraceae bacterium | | 97.3 | 45 |
| AscusEQ_15E | | Clostridium symbiosum | Lachnospiraceae bacterium | | 100 | 46 |
| AscusEQ_16 | 0.80865 | Eubacterium hadrum | Lachnospiraceae bacterium | 92.69 | | 47 |
| AscusEQ_16A | | Eubacterium hadrum | Lachnospiraceae bacterium | | 98.7 | 48 |
| AscusEQ_16B | | Eubacterium hadrum | Lachnospiraceae bacterium | | 99.6 | 49 |
| AscusEQ_16C | | Eubacterium hadrum | Lachnospiraceae bacterium | | 100 | 50 |
| AscusEQ_16D | | Eubacterium hadrum | Lachnospiraceae bacterium | | 99.1 | 51 |
| AscusEQ_17 | 0.75885 | Parabacteroides chinchillae | Odoribacler splanchnicus | 80.58 | | 52 |
| AscusEQ_17A | | Parabacteroides chinchillae | Odoribacler splanchnicus | | 97.3 | 53 |
| AscusEQ_17B | | Parabacteroides chinchillae | Odoribacler splanchnicus | | 98.2 | 54 |
| AscusEQ_17C | | Parabacteroides chinchillae | Odoribacler splanchnicus | | 99.1 | 55 |
| AscusEQ_17D | | Parabacteroides chinchillae | Odoribacler splanchnicus | | 99.6 | 56 |
| AscusEQ_17E | | Parabacteraides chinchillae | Odoribacler splanchnicus | | 98.7 | 57 |
| AscusEQ_17F | | Parabacteraides chinchillae | Odoribacler splanchnicus | | 100 | 58 |
| AscusEQ_18 | 0.75717 | Bacteroides thetaiotaomicron | Bacteroides thetaiotaomicron | 99.67 | | 59 |
| AscusEQ_19 | 0.73924 | Peptoniphilus coxii | Peptoniphilus sp. | 86.29 | | 60 |
| AscusEQ_20 | 0.72865 | Intestinimonas butyriciproducens | Intestinimonas butyriciproducens | 99.67 | | 61 |
| AscusEQ_21 | 0.72547 | Terrisporobacter glycolicus | Terrisporobacter glycolicus | 98.33 | | 62 |
| AscusEQ_21A | | Terrisporobacter glycolicus | Terrisporobacter glycolicus | | 97.3 | 63 |
| AscusEQ_21B | | Terrisporobacter glycolicus | Terrisporobacter glycohcus | | 99.1 | 64 |
| AscusEQ_21C | | Terrisporobacter glycolicus | Terrisporobacter glycohcus | | 100 | 65 |
| AscusEQ_21D | | Terrisporobacter glycolicus | Terrisporobacter glycolicus | | 97.8 | 66 |
| AscusEQ_21E | | Terrisporobacter glycolicus | Terrisporobacter glycolicus | | 98.2 | 67 |
| AscusEQ_21F | | Terrisporobacter glycolicus | Terrisporobacter glycolicus | | 98.7 | 68 |
| AscusEQ_21G | | Terrisporobacter glycolicus | Terrisporobacter glycolicus | | 99.6 | 69 |
| AscusEQ_22 | 0.72373 | Porphyromonas somerae | Porphyromonas canoris | 87 | | 70 |
| AscusEQ_23 | 0.72373 | Peptococcus niger | Peptococcus sp. | 86.6 | | 71 |
| AscusEQ_24 | 0.70904 | Eubacterium barkeri | Eubacterium barkeri | 92.08 | | 72 |
| AscusEQ_24A | | Eubacterium barkeri | Eubacterium barkeri | | 97.8 | 73 |
| AscusEQ_24B | | Eubacterium barkeri | Eubacterium barkeri | | 99.6 | 74 |
| AscusEQ_24C | | Eubacterium barkeri | Eubacterium barkeri | | 99.1 | 75 |
| AscusEQ_24D | | Eubacterium barkeri | Eubacterium barkeri | | 100 | 76 |
| AscusEQ_32 | 0.69845 | Coprococcus eutactus | Lachnospiraceae bacterium | 95 | | 77 |
| AscusEQ_32A | | Coprococcus eutactus | Lachnospiraceae bacterium | | 100 | 78 |
| AscusEQ_26 | 0.68284 | Clostridium alkahcellulosi | Monoglobus pectinilyticus | 84.11 | | 79 |
| AscusEQ_31 | 0.64865 | Cellulosilyticum ruminicola | Cellulosilyticum ruminicola | 99.33 | | 80 |
| AscusEQ_31A | | Cellulosilyticum ruminicola | Cellulosilyticum ruminicola | | 99.1 | 81 |
| AscusEQ_31B | | Cellulosilyticum ruminicola | Cellulosilyticum ruminicola | | 98.7 | 82 |
| AscusEQ_31C | | Cellulosilyticum ruminicola | Cellulosilyticum ruminicola | | 97.8 | 83 |
| AscusEQ_31D | | Cellulosilyticum ruminicola | Cellulosilyticum ruminicola | | 98.2 | 84 |
| AscusEQ_28 | 0.64865 | Schwartzia succinivorans | Erysipelotrichaceae bacterium | 82.12 | | 85 |
| AscusEQ_28A | | Schwartzia succinivorans | Erysipelotrichaceae bacterium | | 97.3 | 86 |
| AscusEQ_28B | | Schwartzia succinivorans | Erysipelotrichaceae bacterium | | 98.7 | 87 |
| AscusEQ_28C | | Schwartzia succinivorans | Erysipelotrichaceae bacterium | | 100 | 88 |
| AscusEQ_28D | | Schwartzia succinivorans | Erysipelotrichaceae bacterium | | 99.1 | 89 |
| AscusEQ_28E | | Schwartzia succinivorans | Erysipelotrichaceae bacterium | | 98.2 | 90 |
| AscusEQ_28F | | Schwartzia succinivorans | Erysipelotrichaceae bacterium | | 99.6 | 91 |
| AscusEQ_28G | | Sch wartzia succinivorans | Erysipelotrichaceae bacterium | | 97.8 | 92 |
| AscusEQ_29 | 0.64865 | Howardella ureilytica | Howardella ureilytica | 86.84 | | 93 |
| AscusEQ_29A | | Howardella ureilytica | Howardella ureilytica | | 99.6 | 94 |
| AscusEQ_29B | | Howardella ureilytica | Howardella ureilytica | | 100 | 95 |
| AscusEQ_30 | 0.64633 | Negativicoccus succinicivorans | Negativicoccus massiliensis | 88 | | 96 |
| AscusEQ_33 | 0.64298 | Clostridium lavalense | Biityrivibrio fibrisolvens | 86.56 | | 97 |
| AscusEQ_33A | | Clostridium lavalense | Butyrivibrio fibrisolvens | | 100 | 98 |
| AscusEQ_33B | | Clostridium lavalense | Butyrivibrio fibrisolvens | | 98.2 | 99 |
| AscusEQ_33C | | Clostridium lavalense | Butyrivibrio fibrisolvens | | 98.7 | 100 |
| AscusEQ_33D | | Clostridium lavalense | Butyrivibrio fibrisolvens | | 99.1 | 101 |
| AscusEQ_33E | | Clostridium lavalense | Butyrivibrio fibrisolvens | | 99.6 | 102 |
| AscusEQ_34 | 0.64206 | Peptostreptococcus anaerobius | Peptostreptococcus anaerobius | 95.39 | | 103 |
| AscusEQ_35 | 0.62904 | Clostridium fimetarium | Lachnospiraceae bacterium | 92.72 | | 104 |
| AscusEQ_35A | | Clostridium fimetarium | Lachnospiraceae bacterium | | 99.1 | 105 |
| AscusEQ_35B | | Clostridium fimetarium | Lachnospiraceae bacterium | | 99.6 | 106 |
| AscusEQ_35C | | Clostridium fimetarium | Lachnospiraceae bacterium | | 100 | 107 |
| AscusEQ_35D | | Clostridium fimetarium | Lachnospiraceae bacterium | | 98.7 | 108 |
| AscusEQ_35E | | Clostridium fimetarium | Lachnospiraceae bacterium | | 98.2 | 109 |
| AscusEQ_36 | 0.61446 | Lachnoanaerobaculum orale | Anaerocolumna sp. | 87.54 | | 110 |
| AscusEQ_37 | 0.61343 | Ruminococcus gnavus | Frisingicoccus caecimuris | 93.02 | | 111 |
| AscusEQ_37A | | Ruminococcus gnavus | Frisingicoccus caecimuris | | 98.2 | 112 |
| AscusEQ_37B | | Ruminococcus gnavus | Frisingicoccus caecimuris | | 97.4 | 113 |
| AscusEQ_37C | | Ruminococcus gnavus | Frisingicoccus caecimuris | | 99.6 | 114 |
| AscusEQ_37D | | Ruminococcus gnavus | Frisingicoccus caecimuris | | 99.1 | 115 |
| AscusEQ_37E | | Ruminococcus gnavus | Frisingicoccus caecimuris | | 98.7 | 116 |
| AscusEQ_37F | | Ruminococcus gnavus | Frisingicoccus caecimuris | | 100 | 117 |
| AscusEQ_37G | | Ruminococcus gnavus | Frisingicoccus caecimuris | | 97.3 | 118 |
| AscusEQ_37H | | Ruminococcus gnavus | Frisingicoccus caecimuris | | 97.8 | 119 |
| AscusEQ_38 | 0.61279 | Calabacter hongkongensis | Ruminiclostridium thermocellum | 84.19 | | 120 |
| AscusEQ_38A | | Catabacter hongkongensis | Ruminiclostridium thermocellum | | 97.3 | 121 |
| AscusEQ_39 | 0.57924 | Erysipelothrix tonsillarum | Spiroplasma sp. | 81.46 | | 122 |
| AscusEQ_39A | | Erysipelothrix tonsillarum | Spiroplasma sp. | | 98.2 | 123 |
| AscusEQ_62 | 0.35661 | Finegoldia magna | Finegoldia magna | 99.67 | | 124 |
| AscusEQ_130 | 0.31894 | Parabacteroides merdae | Algoriphagus ratkowskyi | 83.17 | | 125 |
| AscusEQ_130A | | Parabacteroides merdae | Algoriphagus ratkowskyi | | 97.3 | 126 |
| AscusEQ_130B | | Parabacteroides merdae | Algoriphagus ratkowskyi | | 100 | 127 |
| AscusEQ_130C | | Parabacteroides merdae | Algoriphagus ratkowskyi | | 99.6 | 128 |
| AscusEQ_130D | | Parabacteroides merdae | Algoriphagus ratkowskyi | | 98.7 | 129 |
| AscusEQ_130E | | Parabacteroides merdae | Algoriphagus ratkowskyi | | 97.8 | 130 |
| AscusEQ_130F | | Parabacteroides merdae | Algoriphagus ratkowskyi | | 99.1 | 131 |
| AscusEQ_130G | | Parabacteroides merdae | Algoriphagus ratkowskyi | | 98.2 | 132 |
| AscusEQ_344 | 0.31222 | Parabacteroides merdae | Prevotella copri | 82.7 | | 133 |
| AscusEQ_344A | | Parabacteroides merdae | Prevotella copri | | 100 | 134 |
| AscusEQ_344B | | Parabacteroides merdae | Prevotella copri | | 97.8 | 135 |
| AscusEQ_344C | | Parabacteroides merdae | Prevotella copri | | 98.2 | 136 |
| AscusEQ_344D | | Parabacteroides merdae | Prevotella copri | | 99.1 | 137 |
| AscusEQ_344E | | Parabacteroides merdae | Prevotella copri | | 97.3 | 138 |
| AscusEQ_344F | | Parabacteroides merdae | Prevotella copri | | 99.6 | 139 |
| AscusEQ_344G | | Parabacteroides merdae | Prevotella copri | | 98.7 | 140 |
| AscusEQ_140 | 0.30829 | Streptococcus infantarius subsp. coli | Streptococcus equinus | 99.67 | | 141 |
| AscusEQ_140A | | Streptococcus infantarius subsp. coli | Streptococcus equinus | | 100 | 142 |
| AscusEQ_140B | | Streptococcus infantarius subsp. coli | Streptococcus equinus | | 97.8 | 143 |
| AscusEQ_140C | | Streptococcus infantarius subsp. coli | Streptococcus equinus | | 98.2 | 144 |
| AscusEQ_140D | | Streptococcus infantarius subsp. coli | Streptococcus equinus | | 99.1 | 145 |
| AscusEQ_140E | | Streptococcus infantarius subsp. coli | Streptococcus equinus | | 97.3 | 146 |
| AscusEQ_140F | | Streptococcus infantarius subsp. coli | Streptococcus equinus | | 99.6 | 147 |
| AscusEQ_140G | | Streptococcus infantarius subsp. coli | Streptococcus equinus | | 98.7 | 148 |
| AscusEQ_484 | 0.30764 | Parabacteroides merdae | Prevotella copri | 82.39 | | 149 |
| AscusEQ_484A | | Parabacteroides merdae | Prevotella copri | | 97.3 | 150 |
| AscusEQ_484B | | Parabacteroides merdae | Prevotella copri | | 99.1 | 151 |
| AscusEQ_484C | | Parabacteroides merdae | Prevotella copri | | 98.7 | 152 |
| AscusEQ_484D | | Parabacteroides merdae | Prevotella copri | | 99.6 | 153 |
| AscusEQ_484E | | Parabacteroides merdae | Prevotella copri | | 97.8 | 154 |
| AscusEQ_484F | | Parabacteroides merdae | Prevotella copri | | 100 | 155 |
| AscusEQ_484G | | Parabacteroides merdae | Prevotella copri | | 98.2 | 156 |
| AscusEQ_1187 | 0.29874 | Sutterella stercoricanis | Duodenibacillus massiliensis | 93.36 | | 157 |
| AscusEQ_1187A | | Sutterella stercoricanis | Duodenibacillus massiliensis | | 99.1 | 158 |
| AscusEQ_1187B | | Sutterella stercoricanis | Duodenibacillus massiliensis | | 100 | 159 |
| AscusEQ_1187C | | Sutterella stercoricanis | Duodenibacillus massiliensis | | 98.2 | 160 |
| AscusEQ_1187D | | Sutterella stercoricanis | Duodenibacillus massiliensis | | 99.6 | 161 |
| AscusEQ_200 | 0.29193 | Pedobacter bauzanensis | Bacteroidales bacterium | 88.08 | | 162 |
| AscusEQ_200A | | Pedobacter bauzanensis | Bacteroidales bacterium | | 98.7 | 163 |
| AscusEQ_200B | | Pedobacter bauzanensis | Bacteroidales bacterium | | 100 | 164 |
| AscusEQ_200C | | Pedobacter bauzanensis | Bacteroidales bacterium | | 99.6 | 165 |
| AscusEQ_200D | | Pedobacter bauzanensis | Bacteroidales bacterium | | 99.1 | 166 |
| AscusEQ_200E | | Pedobacter bauzanensis | Bacteroidales bacterium | | 97.3 | 167 |
| AscusEQ_200F | | Pedobacter bauzanensis | Bacteroidales bacterium | | 97.8 | 168 |
| AscusEQ_200G | | Pedobacter bauzanensis | Bacteroidales bacterium | | 98.2 | 169 |
| AscusEQ_183 | 0.27773 | Pedobacter bauzanensis | Bacteroidia bacterium | 85.62 | | 170 |
| AscusEQ_183A | | Pedobacter bauzanensis | Bacteroidia bacterium | | 100 | 171 |
| AscusEQ_183B | | Pedobacter bauzanensis | Bacteroidia bacterium | | 99.1 | 172 |
| AscusEQ_183C | | Pedobacter bauzanensis | Bacteroidia bacterium | | 98.2 | 173 |
| AscusEQ_183D | | Pedobacter bauzanensis | Bacteroidia bacterium | | 97.3 | 174 |
| AscusEQ_183E | | Pedobacter bauzanensis | Bacteroidia bacterium | | 97.8 | 175 |
| AscusEQ_183F | | Pedobacter bauzanensis | Bacteroidia bacterium | | 98.7 | 176 |
| AscusEQ_183G | | Pedobacter bauzanensis | Bacteroidia bacterium | | 99.6 | 177 |
| AscusEQ_226 | 0.27722 | Prevotella bryantii | Cecembia lonarensis | 83.71 | | 178 |
| AscusEQ_226A | | Prevotella bryantii | Cecembia lonarensis | | 97.3 | 179 |
| AscusEQ_226B | | Prevotella bryantii | Cecembia lonarensis | | 99.6 | 180 |
| AscusEQ_226C | | Prevotella bryantii | Cecembia lonarensis | | 98.2 | 181 |
| AscusEQ_226D | | Prevotella bryantii | Cecembia lonarensis | | 97.8 | 182 |
| AscusEQ_226E | | Prevotella bryantii | Cecembia lonarensis | | 98.7 | 183 |
| AscusEQ_226F | | Prevotella bryantii | Cecembia lonarensis | | 100 | 184 |
| AscusEQ_226G | | Prevotella bryantii | Cecembia lonarensis | | 99.1 | 185 |
| AscusEQ_369 | 0.27637 | Alistipes shahii | Bacteroidia bacterium | 85.33 | | 186 |
| AscusEQ_369A | | Alistipes shahii | Bacteroidia bacterium | | 99.1 | 187 |
| AscusEQ_369B | | Alistipes shahii | Bacteroidia bacterium | | 97.3 | 188 |
| AscusEQ_369C | | Alistipes shahii | Bacteroidia bacterium | | 100 | 189 |
| AscusEQ_369D | | Alistipes shahii | Bacteroidia bacterium | | 98.2 | 190 |
| AscusEQ_369E | | Alistipes shahii | Bacteroidia bacterium | | 99.6 | 191 |
| AscusEQ_369F | | Alistipes shahii | Bacteroidia bacterium | | 97.8 | 192 |
| AscusEQ_369G | | Alistipes shahii | Bacteroidia bacterium | | 98.7 | 193 |
| AscusEQ_820 | 0.2734 | Pedobacter bauzanensis | Bacteroidales bacterium | 88.93 | | 194 |
| AscusEQ_820A | | Pedobacter bauzanensis | Bacteroidales bacterium | | 97.8 | 195 |
| AscusEQ_820B | | Pedobacter bauzanensis | Bacteroidales bacterium | | 99.1 | 196 |
| AscusEQ_820C | | Pedobacter bauzanensis | Bacteroidales bacterium | | 100 | 197 |
| AscusEQ_820D | | Pedobacter bauzanensis | Bacteroidales bacterium | | 98.7 | 198 |
| AscusEQ_820E | | Pedobacter bauzanensis | Bacteroidales bacterium | | 99.6 | 199 |
| AscusEQ_253 | 0.25933 | Sarcina maxima | Clostridium maximum | 99.64 | | 200 |
| AscusEQ_253A | | Sarcina maxima | Clostridium maximum | | 97.3 | 201 |
| AscusEQ_253B | | Sarcina maxima | Closiridium maximum | | 98.2 | 202 |
| AscusEQ_253C | | Sarcina maxima | Closiridium maximum | | 97.8 | 203 |
| AscusEQ_253D | | Sarcina maxima | Closiridium maximum | | 99.6 | 204 |
| AscusEQ_253E | | Sarcina maxima | Clostridium maximum | | 98.7 | 205 |
| AscusEQ_253F | | Sarcina maxima | Closiridium maximum | | 100 | 206 |
| AscusEQ_253G | | Sarcina maxima | Closiridium maximum | | 99.1 | 207 |
| AscusEQ_111 | 0.25846 | Finegoldia magna | Finegoldia magna | 99.33 | | 208 |
| AscusEQ_126 | 0.25749 | Pedobacter bauzanensis | Bacteroidales bacterium | 86.09 | | 209 |
| AscusEQ_126A | | Pedobacter bauzanensis | Bacteroidales bacterium | | 99.6 | 210 |
| AscusEQ_126B | | Pedobacter bauzanensis | Bacteroidales bacterium | | 98.7 | 211 |
| AscusEQ_126C | | Pedobacter bauzanensis | Bacteroidales bacterium | | 97.8 | 212 |
| AscusEQ_126D | | Pedobacter bauzanensis | Bacteroidales bacterium | | 97.3 | 213 |
| AscusEQ_126E | | Pedobacter bauzanensis | Bacteroidales bacterium | | 99.1 | 214 |
| AscusEQ_126F | | Pedobacter bauzanensis | Bacteroidales bacterium | | 98.2 | 215 |
| AscusEQ_126G | | Pedobacter bauzanensis | Bacteroidales bacterium | | 100 | 216 |
| AscusEQ_1029 | 0.25642 | Butyricimonas paravirosa | Bacteroidales bacterium | 88.22 | | 217 |
| AscusEQ_1029A | | Butyricimonas paravirosa | Bacteroidales bacterium | | 99.1 | 218 |
| AscusEQ_1029B | | Butyricimonas paravirosa | Bacteroidales bacterium | | 100 | 219 |
| AscusEQ_1029C | | Butyricimonas paravirosa | Bacteroidales bacterium | | 99.6 | 220 |
| AscusEQ_980 | 0.25083 | Pedobacter bauzanensis | Bacteroidales bacterium | 85.76 | | 222 |
| AscusEQ_980A | | Pedobacter bauzanensis | Bacteroidales bacterium | | 99.1 | 223 |
| AscusEQ_980B | | Pedobacter bauzanensis | Bacteroidales bacterium | | 98.2 | 224 |
| AscusEQ_980C | | Pedobacter bauzanensis | Bacteroidales bacterium | | 97.3 | 225 |
| AscusEQ_980D | | Pedobacter bauzanensis | Bacteroidales bacterium | | 99.6 | 226 |
| AscusEQ_980E | | Pedobacter bauzanensis | Bacteroidales bacterium | | 97.8 | 227 |
| AscusEQ_980F | | Pedobacter bauzanensis | Bacteroidales bacterium | | 98.7 | 228 |
| AscusEQ_1166 | 0.24945 | Pedobacter bauzanensis | Bacteroidia bacterium | 88.7 | | 229 |
| AscusEQ_1166A | | Pedobacter bauzanensis | Bacteroidia bacterium | | 97.3 | 230 |
| AscusEQ_1166B | | Pedobacter bauzanensis | Bacteroidia bacterium | | 100 | 231 |
| AscusEQ_1166C | | Pedobacter bauzanensis | Bacteroidia bacterium | | 98.2 | 232 |
| AscusEQ_1166D | | Pedobacter bauzanensis | Bacteroidia bacterium | | 97.8 | 233 |
| AscusEQ_1166E | | Pedobacter bauzanensis | Bacteroidia bacterium | | 99.6 | 234 |
| AscusEQ_1166F | | Pedobacter bauzanensis | Bacteroidia bacterium | | 98.7 | 235 |
| AscusEQ_1166G | | Pedobacter bauzanensis | Bacteroidia bacterium | | 99.1 | 236 |
| AscusEQ_105 | 0.24427 | Selenomonas sputigena | Acidaminococcus sp. | 88.89 | | 237 |
| AscusEQ_105A | | Selenomonas sputigena | Acidaminococcus sp. | | 97.3 | 238 |
| AscusEQ_105B | | Selenomonas sputigena | Acidaminococcus sp. | | 100 | 239 |
| AscusEQ_105C | | Selenomonas sputigena | Acidaminococcus sp. | | 98.7 | 240 |
| AscusEQ_105D | | Selenomonas sputigena | Acidaminococcus sp. | | 99.1 | 241 |
| AscusEQ_105E | | Selenomonas sputigena | Acidaminococcus sp. | | 99.6 | 242 |
| AscusEQ_105F | | Selenomonas sputigena | Acidaminococcus sp. | | 97.8 | 243 |
| AscusEQ_105G | | Selenomonas sputigena | Acidaminococcus sp. | | 98.2 | 244 |
| AscusEQ_145 | 0.24394 | Butyricimonas paravirosa | Bacteroidales bacterium | 88.63 | | 245 |
| AscusEQ_145A | | Butyricimonas paravirosa | Bacteroidales bacterium | | 99.1 | 246 |
| AscusEQ_145B | | Butyricimonas paravirosa | Bacteroidales bacterium | | 100 | 247 |
| AscusEQ_145C | | Butyricimonas paravirosa | Bacteroidales bacterium | | 99.6 | 248 |
| AscusEQ_128 | 0.24327 | Streptococcus orisasini | Streptococcus orisasini | 94.37 | | 249 |
| AscusEQ_669 | 0.23805 | Prevotella brevis | Bacteroides graminisolvens | 83.33 | | 250 |
| AscusEQ_669A | | Prevotella brevis | Bacteroides graminisolvens | | 100 | 251 |
| AscusEQ_669B | | Prevotella brevis | Bacteroides graminisolvens | | 97.3 | 252 |
| AscusEQ_669C | | Prevotella brevis | Bacteroides graminisolvens | | 98.7 | 253 |
| AscusEQ_669D | | Prevotella brevis | Bacteroides graminisolvens | | 98.2 | 254 |
| AscusEQ_669E | | Prevotella brevis | Bacteroides graminisolvens | | 99.6 | 255 |
| AscusEQ_669F | | Prevotella brevis | Bacteroides graminisolvens | | 97.8 | 256 |
| AscusEQ_669G | | Prevotella brevis | Bacteroides graminisolvens | | 99.1 | 257 |
| AscusEQ_703 | 0.23805 | Pedobacter bauzanensis | Bacteroidales bacterium | 89.26 | | 258 |
| AscusEQ_703A | | Pedobacter bauzanensis | Bacteroidales bacterium | | 99.1 | 259 |
| AscusEQ_703B | | Pedobacter bauzanensis | Bacteroidales bacterium | | 99.6 | 260 |
| AscusEQ_703C | | Pedobacter bauzanensis | Bacteroidales bacterium | | 98.2 | 261 |
| AscusEQ_703D | | Pedobacter bauzanensis | Bacteroidales bacterium | | 100 | 262 |
| AscusEQ_436 | 0.23361 | Bacteroides plebeius | Parabacteroides distasonis | 83.71 | | 263 |
| AscusEQ_436A | | Bacteroides plebeius | Parabacteroides distasonis | | 97.8 | 264 |
| AscusEQ_436B | | Bacteroides plebeius | Parabacteroides distasonis | | 98.7 | 265 |
| AscusEQ_436C | | Bacteroides plebeius | Parabacteroides distasonis | | 98.2 | 266 |
| AscusEQ_436D | | Bacteroides plebeius | Parabacteroides distasonis | | 99.6 | 267 |
| AscusEQ_436E | | Bacteroides plebeius | Parabacteroides distasonis | | 100 | 268 |
| AscusEQ_436F | | Bacteroides plebeius | Parabacteroides distasonis | | 99.1 | 269 |
| AscusEQ_436G | | Bacteroides plebeius | Parabacteroides distasonis | | 97.3 | 270 |
| AscusEQ_680 | 0.23329 | Alloprevotella rava | Bacteroides coprophilus | 85.86 | | 271 |
| AscusEQ_680A | | Alloprevotella rava | Bacteroides coprophilus | | 97.8 | 272 |
| AscusEQ_680B | | Alloprevotella rava | Bacteroides coprophilus | | 100 | 273 |
| AscusEQ_680C | | Alloprevotella rava | Bacteroides coprophilus | | 98.7 | 274 |
| AscusEQ_680D | | Alloprevotella rava | Bacteroides coprophilus | | 99.1 | 275 |
| AscusEQ_680E | | Alloprevotella rava | Bacteroides coprophilus | | 99.6 | 276 |
| AscusEQ_680F | | Alloprevotella rava | Bacteroides coprophilus | | 97.3 | 277 |
| AscusEQ_533 | 0.233 | Phascolarctobacterium succinatutens | Acidaminococcus fermentans | 88.31 | | 278 |
| AscusEQ_762 | 0.23246 | Prevotella stercorea | Bacteroidales bacterium | 89.8 | | 279 |
| AscusEQ_762A | | Prevotella stercorea | Bacteroidales bacterium | | 99.6 | 280 |
| AscusEQ_762B | | Prevotella stercorea | Bacteroidales bacterium | | 98.2 | 281 |
| AscusEQ_762C | | Prevotella stercorea | Bacteroidales bacterium | | 100 | 282 |
| AscusEQ_762D | | Prevotella stercorea | Bacteroidales bacterium | | 98.7 | 283 |
| AscusEQ_762E | | Prevotella stercorea | Bacteroidales bacterium | | 99.1 | 284 |
| AscusEQ_762F | | Prevotella stercorea | Bacteroidales bacterium | | 97.3 | 285 |
| AscusEQ_762G | | Prevotella stercorea | Bacteroidales bacterium | | 97.8 | 286 |
| AscusEQ_67 | 0.23225 | Streptococcus saliviloxodontae | Streptococcus sp. | 99.66 | | 287 |
| AscusEQ_773 | 0.23225 | Phascolarctobacterium succinatutens | Phascolarctobacterium sp. | 91.72 | | 288 |
| AscusEQ_773A | | Phascolarctobacterium succinatutens | Phascolarctobacterium sp. | | 100 | 289 |
| AscusEQ_773B | | Phascolarctobacterium succinatutens | Phascolarctobacterium sp. | | 97.8 | 290 |
| AscusEQ_773C | | Phascolarctobacterium succinatutens | Phascolarctobacterium sp. | | 99.6 | 291 |
| AscusEQ_773D | | Phascolarctobacterium succinatutens | Phascolarctobacterium sp. | | 99.1 | 292 |
| AscusEQ_773E | | Phascolarctobacterium succinatutens | Phascolarctobacterium sp. | | 97.3 | 293 |
| AscusEQ_773F | | Phascolarctobacterium succinatutens | Phascolarctobacterium sp. | | 98.2 | 294 |
| AscusEQ_773G | | Phascolarctobacterium succinatutens | Phascolarctobacterium sp. | | 98.7 | 295 |
| AscusEQ_100 | 0.23179 | Bacteroides | Bacteroides vulgatus | 100 | | 296 |
| AscusEQ_625 | 0.23167 | Phascolarctobacterium succinatutens | Phascolarctobacterium sp | 93.07 | | 297 |
| AscusEQ_625A | | Phascolarctobacterium succinatutens | Phascolarctobacterium sp | | 98.7 | 298 |
| AscusEQ_625B | | Phascolarctobacterium succinatutens | Phascolarctobacterium sp | | 98.2 | 299 |
| AscusEQ_625C | | Phascolarctobacterium succinatutens | Phascolarctobacterium sp | | 97.8 | 300 |
| AscusEQ_625D | | Phascolarctobacterium succinatutens | Phascolarctobacterium sp | | 99.6 | 301 |
| AscusEQ_625E | | Phascolarctobacterium succinatutens | Phascolarctobacterium sp | | 99.1 | 302 |
| AscusEQ_625F | | Phascolarctobacterium succinatutens | Phascolarctobacterium sp | | 100 | 303 |
| AscusEQ_121 | 0.23115 | Streptococcus orisasini | Streptococcus orisasini | 94.04 | | 304 |
| AscusEQ_783 | 0.2294 | Prevotella brevis | Bacteroides graminisolvens | 83.99 | | 305 |
| AscusEQ_783A | | Prevotella brevis | Bacteroides graminisolvens | | 99.1 | 306 |
| AscusEQ_783B | | Prevotella brevis | Bacteroides graminisolvens | | 97.8 | 307 |
| AscusEQ_783C | | Prevotella brevis | Bacteroides graminisolvens | | 97.3 | 308 |
| AscusEQ_783D | | Prevotella brevis | Bacteroides graminisolvens | | 98.7 | 309 |
| AscusEQ_783E | | Prevotella brevis | Bacteroides graminisolvens | | 98.2 | 310 |
| AscusEQ_1316 | 0.22636 | Phascolarctobacterium succinatutens | Phascolarctobacterium sp. | 93.02 | | 311 |
| AscusEQ_1316A | | Phascolarctobacterium succinatutens | Phascolarctobacterium sp. | | 98.2 | 312 |
| AscusEQ_1316B | | Phascolarctobacterium succinatutens | Phascolarctobacterium sp. | | 100 | 313 |
| AscusEQ_1316C | | Phascolarctobacterium succinatutens | Phascolarctobacterium sp. | | 97.3 | 314 |
| AscusEQ_1316D | | Phascolarctobacterium succinatutens | Phascolarctobacterium sp. | | 98.7 | 315 |
| AscusEQ_1316E | | Phascolarctobacterium succinatutens | Phascolarctobacterium sp. | | 99.1 | 316 |
| AscusEQ_1316F | | Phascolarctobacterium succinatutens | Phascolarctobacterium sp. | | 97.8 | 317 |
| AscusEQ_1316G | | Phascolarctobacterium succinatutens | Phascolarctobacterium sp. | | 99.6 | 318 |
| AscusEQ_61 | 0.22822 | Escherichia coli | Escherichia coli | 99.56 | | 319 |
| AscusEQ_61A | | Escherichia coli | Escherichia coli | | 100 | 320 |
| AscusEQ_61B | | Escherichia coli | Escherichia coli | | 99.6 | 321 |
| AscusEQ_61C | | Escherichia coli | Escherichia coli | | 97.3 | 322 |
| AscusEQ_61D | | Escherichia coli | Escherichia coli | | 98.2 | 323 |
| AscusEQ_61E | | Escherichia coli | Escherichia coli | | 97.8 | 324 |
| AscusEQ_61F | | Escherichia coli | Escherichia coli | | 99.1 | 325 |
| AscusEQ_61G | | Escherichia coli | Escherichia coli | | 98.7 | 326 |
| AscusEQ_1022 | 0.22542 | Butyricimonas paravirosa | Bacteroidetes bacterium | 85.62 | | 327 |
| AscusEQ_1022A | | Butyricimonas paravirosa | Bacteroidetes bacterium | | 100 | 328 |
| AscusEQ_1022B | | Butyricimonas paravirosa | Bacteroidetes bacterium | | 99.6 | 329 |
| AscusEQ_1022C | | Butyricimonas paravirosa | Bacteroidetes bacterium | | 98.7 | 330 |
| AscusEQ_1022D | | Butyricimonas paravirosa | Bacteroidetes bacterium | | 99.1 | 331 |
| AscusEQ_607 | 0.22533 | Phascolarctobacterium succinatutens | Phascolarctobacterium sp. | 92.08 | | 332 |
| AscusEQ_607A | | Phascolarctobacterium succinatutens | Phascolarctobacterium sp. | | 98.2 | 333 |
| AscusEQ_607B | | Phascolarctobacterium succinatutens | Phascolarctobacterium sp. | | 98.7 | 334 |
| AscusEQ_607C | | Phascolarctobacterium succinatutens | Phascolarctobacterium sp. | | 99.1 | 335 |
| AscusEQ_607D | | Phascolarctobacterium succinatutens | Phascolarctobacterium sp. | | 100 | 336 |
| AscusEQ_607E | | Phascolarctobacterium succinatutens | Phascolarctobacterium sp. | | 99.6 | 337 |
| AscusEQ_412 | 0.22322 | Bacteroides plebeius | Alloprevotella sp. | 87.83 | | 338 |
| AscusEQ_412A | | Bacteroides plebeius | Alloprevotella sp. | | 98.7 | 339 |
| AscusEQ_412B | | Bacteroides plebeius | Alloprevotella sp. | | 97.8 | 340 |
| AscusEQ_412C | | Bacteroides plebeius | Alloprevotella sp. | | 98.2 | 341 |
| AscusEQ_412D | | Bacteroides plebeius | Alloprevotella sp. | | 97.3 | 342 |
| AscusEQ_412E | | Bacteroides plebeius | Alloprevotella sp. | | 100 | 343 |
| AscusEQ_412F | | Bacteroides plebeius | Alloprevotella sp. | | 99.1 | 344 |
| AscusEQ_412G | | Bacteroides plebeius | Alloprevotella sp. | | 99.6 | 345 |
| AscusEQ_1362 | 0.22297 | Barnesiella viscericola | Muribaculaceae bacterium | 89.7 | | 346 |
| AscusEQ_1362A | | Barnesiella viscericola | Muribaculaceae bacterium | | 98.2 | 347 |
| AscusEQ_696 | 0.22246 | Cyclobacterium lianum | Bacteroidia bacterium | 85.34 | | 348 |
| AscusEQ_696A | | Cyclobacterium lianum | Bacteroidia bacterium | | 97.3 | 349 |
| AscusEQ_696B | | Cyclobacterium lianum | Bacteroidia bacterium | | 98.2 | 350 |
| AscusEQ_696C | | Cyclobacterium lianum | Bacteroidia bacterium | | 99.1 | 351 |
| AscusEQ_696D | | Cyclobacterium lianum | Bacteroidia bacterium | | 99.6 | 352 |
| AscusEQ_696E | | Cyclobacterium lianum | Bacteroidia bacterium | | 100 | 353 |
| AscusEQ_696F | | Cyclobacterium lianum | Bacteroidia bacterium | | 98.7 | 354 |
| AscusEQ_53 | 0.22093 | Finegoldia magna | Bacteroides massiliensis | 99.33 | | 355 |
| AscusEQ_835 | 0.22041 | Bacteroides massiliensis | Bacteroides massiliensis | 82.52 | | 356 |
| AscusEQ_835A | | Bacteroides massiliensis | Bacteroides massiliensis | | 97.8 | 357 |
| AscusEQ_835B | | Bacteroides massilicrisis | Bacteroides massiliensis | | 99.1 | 358 |
| AscusEQ_835C | | Bacteroides massiliensis | Bacteroides massiliensis | | 99.6 | 359 |
| AscusEQ_835D | | Bacteroides massiliensis | Bacteroides massiliensis | | 100 | 360 |
| AscusEQ_595 | 0.22036 | Oscillibacter valericigenes | Oscillibacter valericigenes | 90.13 | | 361 |
| AscusEQ_595A | | Oscillibacter valericigenes | Oscillibacter valericigenes | | 98.7 | 362 |
| AscusEQ_595B | | Oscillibacter valericigenes | Oscillibacter valericigenes | | 100 | 363 |
| AscusEQ_595C | | Oscillibacter valericigenes | Oscillibacter valericigenes | | 97.8 | 364 |
| AscusEQ_595D | | Oscillibacter valericigenes | Oscillibacter valericigenes | | 99.6 | 365 |
| AscusEQ_595E | | Oscillibacter valericigenes | Oscillibacter valericigenes | | 97.3 | 366 |
| AscusEQ_595F | | Oscillibacter valericigenes | Oscillibacter valericigenes | | 99.1 | 367 |
| AscusEQ_595G | | Oscillibacter valericigenes | Oscillibacter valericigenes | | 98.2 | 368 |
| AscusEQ_1850 | 0.21965 | Pediococcus ethanolidurans | TM7 bacterium | 81.79 | | 369 |
| AscusEQ_1850A | | Pediococcus ethanolidurans | TM7 bacterium | | 97.3 | 370 |
| AscusEQ_1850B | | Pediococcus ethanolidurans | TM7 bacterium | | 99.6 | 371 |
| AscusEQ_1850C | | Pediococcus ethanolidurans | TM7 bacterium | | 100 | 372 |
| AscusEQ_298 | 0.21933 | Phascolarctobacterium succinatutens | Phascolarctobacterium succinatutens | 80.48 | | 373 |
| AscusEQ_298A | | Phascolarctobacterium succinatutens | Phascolarctobacterium succinatutens | | 99.6 | 374 |
| AscusEQ_298B | | Phascolarctobacterium succinatutens | Phascolarctobacterium succinatutens | | 99.1 | 375 |
| AscusEQ_298C | | Phascolarctobacterium succinatutens | Phascolarctobacterium succinatutens | | 100 | 376 |
| AscusEQ_298D | | Phascolarctobacterium succinatutens | Phascolarctobacterium succinatutens | | 98.7 | 377 |
| AscusEQ_298E | | Phascolarctobacterium succinatutens | Phascolarctobacterium succinatutens | | 98.2 | 378 |
| AscusEQ_743 | 0.2193 | Prevotella brevis | Bacteroides graminisolvens | 83.66 | | 379 |
| AscusEQ_743A | | Prevotella brevis | Bacteroides graminisolvens | | 97.3 | 380 |
| AscusEQ_743B | | Prevotella brevis | Bacteroides graminisolvens | | 97.8 | 381 |
| AscusEQ_743C | | Prevotella brevis | Bacteroides graminisolvens | | 99.1 | 382 |
| AscusEQ_743D | | Prevotella brevis | Bacteroides graminisolvens | | 98.2 | 383 |
| AscusEQ_743E | | Prevotella brevis | Bacteroides graminisolvens | | 98.7 | 384 |
| AscusEQ_722 | 0.219 | Maritalea mobilis | Alphaproteobacteria bacterium | 91.72 | | 385 |
| AscusEQ_722A | | Maritalea mobilis | Alphaproteobacteria bacterium | | 98.7 | 386 |
| AscusEQ_722B | | Maritalea mobilis | Alphaproteobacteria bacterium | | 98.2 | 387 |
| AscusEQ_722C | | Maritalea mobilis | Alphaproteobacteria bacterium | | 97.3 | 388 |
| AscusEQ_722D | | Maritalea mobilis | Alphaproteobacteria bacterium | | 97.8 | 389 |
| AscusEQ_722E | | Maritalea mobilis | Alphaproteobacteria bacterium | | 100 | 390 |
| AscusEQ_722F | | Maritalea mobilis | Alphaproteobacteria bacterium | | 99.6 | 391 |
| AscusEQ_722G | | Maritalea mobilis | Alphaproteobacteria bacterium | | 99.1 | 392 |
| AscusEQ_50 | 0.21805 | Parvimonas micra | Parvimonas sp. | 92.64 | | 393 |
| AscusEQ_603 | 0.21616 | Prevotella copri | Cecembia lonarensis | 84.36 | | 394 |
| AscusEQ_603A | | Prevotella copri | Cecembia lonarensis | | 97.8 | 395 |
| AscusEQ_603B | | Prevotella copri | Cecembia lonarensis | | 100 | 396 |
| AscusEQ_603C | | Prevotella copri | Cecembia lonarensis | | 97.3 | 397 |
| AscusEQ_603D | | Prevotella copri | Cecembia lonarensis | | 98.7 | 398 |
| AscusEQ_603E | | Prevotella copri | Cecembia lonarensis | | 99.6 | 399 |
| AscusEQ_603F | | Prevotella copri | Cecembia lonarensis | | 99.1 | 400 |
| AscusEQ_603G | | Prevotella copri | Cecembia lonarensis | | 98.2 | 401 |
| AscusEQ_91 | 0.21505 | Streptococcus minor | Streptococcus pluranimalium | 98.66 | | 402 |
| AscusEQ_1567 | 0.21432 | Phascolarctobacterium succinatutens | Phascolarctobacterium sp. | 93.02 | | 403 |
| AscusEQ_1567A | | Phascolarctobacterium succinatutens | Phascolarctobacterium sp. | | 98.7 | 404 |
| AscusEQ_1567B | | Phascolarctobacterium succinatutens | Phascolarctobacterium sp. | | 97.3 | 405 |
| AscusEQ_1567C | | Phascolarctobacterium succinatutens | Phascolarctobacterium sp. | | 98.2 | 406 |
| AscusEQ_1567D | | Phascolarctobacterium succinatutens | Phascolarctobacterium sp. | | 99.1 | 407 |
| AscusEQ_1567E | | Phascolarctobacterium succinatutens | Phascolarctobacterium sp. | | 100 | 408 |
| AscusEQ_1567F | | Phascolarctobacterium succinatutens | Phascolarctobacterium sp. | | 97.8 | 409 |
| AscusEQ_1567G | | Phascolarctobacterium succinatutens | Phascolarctobacterium sp. | | 99.6 | 410 |
| AscusEQ_1921 | 0.21344 | Alistipes shahii | Bacteroidia bacterium | 86 | | 411 |
| AscusEQ_1921A | | Alistipes shahii | Bacteroidia bacterium | | 98.7 | 412 |
| AscusEQ_1921B | | Alistipes shahii | Bacteroidia bacterium | | 99.1 | 413 |
| AscusEQ_1921C | | Alistipes shahii | Bacteroidia bacterium | | 98.2 | 414 |
| AscusEQ_1921D | | Alistipes shahii | Bacteroidia bacterium | | 100 | 415 |
| AscusEQ_1921E | | Alistipes shahii | Bacteroidia bacterium | | 97.8 | 416 |
| AscusEQ_1921F | | Alistipes shahii | Bacteroidia bacterium | | 99.6 | 417 |
| AscusEQ_1921G | | Alistipes shahii | Bacteroidia bacterium | | 97.3 | 418 |
| AscusEQ_476 | 0.21308 | Prevotella brevis | Bacteroides graminisolvens | 83.01 | | 419 |
| AscusEQ_476A | | Prevotella brevis | Bacteroides graminisolvens | | 97.8 | 420 |
| AscusEQ_476B | | Prevotella brevis | Bacteroides graminisolvens | | 98.2 | 421 |
| AscusEQ_476C | | Prevotella brevis | Bacteroides graminisolvens | | 98.7 | 422 |
| AscusEQ_476D | | Prevotella brevis | Bacteroides graminisolvens | | 100 | 423 |
| AscusEQ_476E | | Prevotella brevis | Bacteroides graminisolvens | | 97.3 | 424 |
| AscusEQ_476F | | Prevotella brevis | Bacteroides graminisolvens | | 99.6 | 425 |
| AscusEQ_414 | 0.21285 | Sarcina maxima | Clostridium maximum | 99.64 | | 426 |
| AscusEQ_414A | | Sarcina maxima | Clostridium maximum | | 98.2 | 427 |
| AscusEQ_414B | | Sarcina maxima | Clostridium maximum | | 98.7 | 428 |
| AscusEQ_414C | | Sarcina maxima | Clostridium maximum | | 97.8 | 429 |
| AscusEQ_414D | | Sarcina maxima | Clostridium maximum | | 99.6 | 430 |
| AscusEQ_414E | | Sarcina maxima | Clostridium maximum | | 97.3 | 431 |
| AscusEQ_414F | | Sarcina maxima | Clostridium maximum | | 99.1 | 432 |
| AscusEQ_414G | | Sarcina maxima | Clostridium maximum | | 100 | 433 |
| AscusEQ_1253 | 0.21205 | Pedobacter bauzanensis | Bacteroidia bacterium | 88.96 | | 434 |
| AscusEQ_1253A | | Pedobacter bauzanensis | Bacteroidia bacterium | | 100 | 435 |
| AscusEQ_1253B | | Pedobacter bauzanensis | Bacteroidia bacterium | | 99.6 | 436 |
| AscusEQ_1253C | | Pedobacter bauzanensis | Bacteroidia bacterium | | 99.1 | 437 |
| AscusEQ_324 | 0.21122 | Bacteroides coprocola | Bacteroides mediterraneensis | 81.93 | | 438 |
| AscusEQ_324A | | Bacteroides coprocola | Bacteroides mediterraneensis | | 98.2 | 439 |
| AscusEQ_324B | | Bacteroides coprocola | Bacteroides mediterraneensis | | 98.7 | 440 |
| AscusEQ_324C | | Bacteroides coprocola | Bacteroides mediterraneensis | | 100 | 441 |
| AscusEQ_324D | | Bacteroides coprocola | Bacteroides mediterraneensis | | 99.6 | 442 |
| AscusEQ_324E | | Bacteroides coprocola | Bacteroides mediterraneensis | | 99.1 | 443 |
| AscusEQ_1536 | 0.21109 | Phascolarctobacterium succinatutens | Phascolarctobacterium sp. | 93.05 | | 444 |
| AscusEQ_1536A | | Phascolarctobacterium succinatutens | Phascolarctobacterium sp. | | 98.7 | 445 |
| AscusEQ_1536B | | Phascolarctobacterium succinatutens | Phascolarctobacterium sp. | | 98.2 | 446 |
| AscusEQ_1536C | | Phascolarctobacterium succinatutens | Phascolarctobacterium sp. | | 100 | 447 |
| AscusEQ_1536D | | Phascolarctobacterium succinatutens | Phascolarctobacterium sp. | | 99.6 | 448 |
| AscusEQ_1536E | | Phascolarctobacterium succinatutens | Phascolarctobacterium sp. | | 97.8 | 449 |
| AscusEQ_1536F | | Phascolarctobacterium succinatutens | Phascolarclobacterium sp. | | 97.3 | 450 |
| AscusEQ_1536G | | Phascolarctobacterium succinatutens | Phascolarclobacterium sp. | | 99.1 | 451 |
| AscusEQ_971 | 0.21101 | Bacteroides massiliensis | Bacteroides massiliensis | 82.79 | | 452 |
| AscusEQ_971A | | Bacteroides massiliensis | Bacteroides massiliensis | | 98.2 | 453 |
| AscusEQ_971B | | Bacteroides massiliensis | Bacteroides massiliensis | | 98.7 | 454 |
| AscusEQ_971C | | Bacteroides massiliensis | Bacteroides massiliensis | | 100 | 455 |
| AscusEQ_971D | | Bacteroides massiliensis | Bacteroides massiliensis | | 99.1 | 456 |
| AscusEQ_971E | | Bacteroides massiliensis | Bacteroides massiliensis | | 99.6 | 457 |
| AscusEQ_257 | 0.21002 | Anaerophaga thermohalophila | Algoriphagus taiwanensis | 82.85 | | 458 |
| AscusEQ_257A | | Anaerophaga thermohalophila | Algoriphagus taiwanensis | | 98.2 | 459 |
| AscusEQ_257B | | Anaerophaga thermohalophila | Algoriphagus taiwanensis | | 99.6 | 460 |
| AscusEQ_257C | | Anaerophaga thermohalophila | Algoriphagus taiwanensis | | 98.7 | 461 |
| AscusEQ_257D | | Anaerophaga thermohalophila | Algoriphagus taiwanensis | | 97.3 | 462 |
| AscusEQ_257E | | Anaerophaga thermohalophila | Algoriphagus taiwanensis | | 99.1 | 463 |
| AscusEQ_257F | | Anaerophaga thermohalophila | Algoriphagus taiwanensis | | 97.8 | 464 |
| AscusEQ_452 | 0.2092 | Prevotella stercorea | Bacteroidales bacterium | 90.13 | | 465 |
| AscusEQ_452A | | Prevotella stercorea | Bacteroidales bacterium | | 97.8 | 466 |
| AscusEQ_452B | | Prevotella stercorea | Bacteroidales bacterium | | 97.3 | 467 |
| AscusEQ_452C | | Prevotella stercorea | Bacteroidales bacterium | | 100 | 468 |
| AscusEQ_452D | | Prevotella stercorea | Bacteroidales bacterium | | 99.6 | 469 |
| AscusEQ_452E | | Prevotella stercorea | Bacteroidales bacterium | | 99.1 | 470 |
| AscusEQ_452F | | Prevotella stercorea | Bacteroidales bacterium | | 98.7 | 471 |
| AscusEQ_452G | | Prevotella stercorea | Bacteroidales bacterium | | 98.2 | 472 |
| AscusEQ_167 | 0.20793 | Butyricimonas paravirosa | Bacteroidales bacterium | 88.29 | | 473 |
| AscusEQ_167A | | Shigella sonnei | Atlantibacter hermannii | | 99.1 | 482 |
| AscusEQ_167B | | Shigella sonnei | Atlantibacter hermannii | | 99.6 | 483 |
| AscusEQ_167C | | Shigella sonnei | Atlantibacter hermannii | | 100 | 484 |
| AscusEQ_197 | 0.20789 | Parabacteroides merdae | Parabacteroides distasonis | 96.67 | | 474 |
| AscusEQ_109 | 0.20839 | Shigella sonnei | Atlantibacter hermannii | 100 | | 475 |
| AscusEQ_109A | | Shigella sonnei | Atlantibacter hermannii | | 100 | 476 |
| AscusEQ_109B | | Shigella sonnei | Atlantibacter hermannii | | 99.1 | 477 |
| AscusEQ_109C | | Shigella sonnei | Atlantibacter hermannii | | 99.6 | 478 |
| AscusEQ_109D | | Shigella sonnei | Atlantibacter hermannii | | 98.2 | 479 |
| AscusEQ_109E | | Shigella sonnei | Atlantibacter hermannii | | 97.8 | 480 |
| AscusEQ_109F | | Shigella sonnei | Atlantibacter hermannh | | 97.3 | 481 |
| AscusEQ_363 | 0.20779 | Anaerophaga thermohalophila | Prevotella copri | 82.08 | | 485 |
| AscusEQ_363A | | Anaerophaga thermohalophila | Prevotella copri | | 100 | 486 |
| AscusEQ_363B | | Anaerophaga thermohalophila | Prevotella copri | | 98.7 | 487 |
| AscusEQ_363C | | Anaerophaga thermohalophila | Prevotella copri | | 97.8 | 488 |
| AscusEQ_363D | | Anaerophaga thermohalophila | Prevotella copri | | 99.1 | 489 |
| AscusEQ_363E | | Anaerophaga thermohalophila | Prevotella copri | | 98.2 | 490 |
| AscusEQ_363F | | Anaerophaga thermohalophila | Prevotella copri | | 97.3 | 491 |
| AscusEQ_363G | | Anaerophaga thermohalophila | Prevotella copri | | 99.6 | 492 |
| AscusEQ_299 | 0.20694 | Prevotella stercorea | Bacteroidales bacterium | 89.8 | | 493 |
| AscusEQ_299A | | Prevotella stercorea | Bacteroidales bacterium | | 97.3 | 494 |
| AscusEQ_299B | | Prevotella stercorea | Bacteroidales bacterium | | 99.1 | 495 |
| AscusEQ_299C | | Prevotella stercorea | Bacteroidales bacterium | | 100 | 496 |
| AscusEQ_299D | | Prevotella stercorea | Bacteroidales bacterium | | 97.8 | 497 |
| AscusEQ_299E | | Prevotella stercorea | Bacteroidales bacterium | | 99.6 | 498 |
| AscusEQ_299F | | Prevotella stercorea | Bacteroidales bacterium | | 98.2 | 499 |
| AscusEQ_299G | | Prevotella stercorea | Bacteroidales bacterium | | 98.7 | 500 |
| AscusEQ_456 | 0.20595 | Prevotella oulorum | Prevotella oris | 84.21 | | 501 |
| AscusEQ_456A | | Prevotella oulorum | Prevotella oris | | 97.3 | 502 |
| AscusEQ_456B | | Prevotella oulorum | Prevotella oris | | 99.1 | 503 |
| AscusEQ_456C | | Prevotella oulorum | Prevotella oris | | 100 | 504 |
| AscusEQ_456D | | Prevotella oulorum | Prevotella oris | | 99.6 | 505 |
| AscusEQ_456E | | Prevotella oulorum | Prevotella oris | | 98.2 | 506 |
| AscusEQ_456F | | Prevotella oulorum | Prevotella oris | | 98.7 | 507 |
| AscusEQ_232 | 0.20589 | Bacteroides coprocola | Bacteroides caecicola | 81.82 | | 508 |
| AscusEQ_232A | | Bacteroides coprocola | Bacteroides caecicola | | 98.2 | 509 |
| AscusEQ_232B | | Bacteroides coprocola | Bacteroides caecicola | | 100 | 510 |
| AscusEQ_232C | | Bacteroides coprocola | Bacteroides caecicola | | 99.6 | 511 |
| AscusEQ_235 | 0.20536 | Parabacteroides merdae | Algoriphagus ratkowskyi | 83.17 | | 512 |
| AscusEQ_235A | | Parabacteroides merdae | Algoriphagus ratkowskyi | | 98.7 | 513 |
| AscusEQ_235B | | Parabacteroides merdae | Algoriphagus ratkowskyi | | 100 | 514 |
| AscusEQ_235C | | Parabacteroides merdae | Algoriphagus ratkowskyi | | 97.8 | 515 |
| AscusEQ_235D | | Parabacteroides merdae | Algoriphagus ratkowskyi | | 98.2 | 516 |
| AscusEQ_235E | | Parabacteroides merdae | Algoriphagus ratkowskyi | | 99.1 | 517 |
| AscusEQ_235F | | Parabacteroides merdae | Algoriphagus ratkowskyi | | 97.3 | 518 |
| AscusEQ_235G | | Parabacteroides merdae | Algoriphagus ratkowskyi | | 99.6 | 519 |
| AscusEQ_490 | 0.20524 | Bacteroides plebeius | Parabacteroides distasonis | 83.39 | | 520 |
| AscusEQ_490A | | Bacteroides plebeius | Parabacteroides distasonis | | 98.2 | 521 |
| AscusEQ_490B | | Bacteroides plebeius | Parabacteroides distasonis | | 97.8 | 522 |
| AscusEQ_490C | | Bacteroides plebeius | Parabacteroides distasonis | | 98.7 | 523 |
| AscusEQ_490D | | Bacteroides plebeius | Parabacteroides distasonis | | 100 | 524 |
| AscusEQ_490E | | Bacteroides plebeius | Parabacteroides distasonis | | 99.1 | 525 |
| AscusEQ_490F | | Bacteroides plebeius | Parabacteroides distasonis | | 97.3 | 526 |
| AscusEQ_490G | | Bacteroides plebeius | Parabacteroides distasonis | | 99.6 | 527 |
| AscusEQ_106 | 0.20492 | Helcococcus ovis | Helcococcus ovis | 92.38 | | 528 |
| AscusEQ_588 | 0.2049 | Pedobacter bauzanensis | Bacteroidales bacterium | 88.22 | | 529 |
| AscusEQ_588A | | Pedobacter bauzanensis | Bacteroidales bacterium | | 98.2 | 530 |
| AscusEQ_588B | | Pedobacter bauzanensis | Bacteroidales bacterium | | 99.1 | 531 |
| AscusEQ_588C | | Pedobacter bauzanensis | Bacteroidales bacterium | | 100 | 532 |
| AscusEQ_588D | | Pedobacter bauzanensis | Bacteroidales bacterium | | 99.6 | 533 |
| AscusEQ_588E | | Pedobacter bauzanensis | Bacteroidales bacterium | | 98.7 | 534 |
| AscusEQ_1234 | 0.20382 | Devosia pacifica | Alphaproteobacteria bacterium | 93.26 | | 535 |
| AscusEQ_1234A | | Devosia pacifica | Alphaproteobacteria bacterium | | 99.1 | 536 |
| AscusEQ_1234B | | Devosia pacifica | Alphaproteobacteria bacterium | | 100 | 537 |
| AscusEQ_1234C | | Devosia pacifica | Alphaproteobacteria bacterium | | 97.8 | 538 |
| AscusEQ_1234D | | Devosia pacifica | Alphaproteobacteria bacterium | | 99.6 | 539 |
| AscusEQ_1234E | | Devosia pacifica | Alphaproteobacteria bacterium | | 97.3 | 540 |
| AscusEQ_962 | 0.20356 | Anaerocella delicata | Anaerocella delicata | 85.86 | | 541 |
| AscusEQ_962A | | Anaerocella delicata | Anaerocella delicata | | 99.1 | 542 |
| AscusEQ_962B | | Anaerocella delicata | Anaerocella delicata | | 99.6 | 543 |
| AscusEQ_962C | | Anaerocella delicata | Anaerocella delicata | | 100 | 544 |
| AscusEQ_916 | 0.20303 | Sphingomicrobium astaxanthinifaciens | Alphaproteobacteria bacterium | 93.26 | | 545 |
| AscusEQ_916A | | Sphingomicrobium astaxanthinifaciens | Alphaproteobacteria bacterium | | 99.1 | 546 |
| AscusEQ_916B | | Sphingomicrobium astaxanthinifaciens | Alphaproteobacteria bacterium | | 99.6 | 547 |
| AscusEQ_916C | | Sphingomicrobium astaxanthinifaciens | Alphaproteobacteria bacterium | | 97.3 | 548 |
| AscusEQ_916D | | Sphingomicrobium astaxanthinifactens | Alphaproteobacteria bacterium | | 100 | 549 |
| AscusEQ_916E | | Sphingomicrobium astaxanthinifaciens | Alphaproteobacteria bacterium | | 97.8 | 550 |
| AscusEQ_1095 | 0.20119 | Prevotella stercorea | Prevotella sp. | 89.87 | | 551 |
| AscusEQ_1095A | | Prevotella stercorea | Prevotella sp. | | 97.3 | 552 |
| AscusEQ_1095B | | Prevotella stercorea | Prevotella sp. | | 98.7 | 553 |
| AscusEQ_1095C | | Prevotella stercorea | Prevotella sp. | | 99.1 | 554 |
| AscusEQ_1095D | | Prevotella stercorea | Prevotella sp. | | 99.6 | 555 |
| AscusEQ_1095E | | Prevotella stercorea | Prevotella sp. | | 97.8 | 556 |
| AscusEQ_1095F | | Prevotella stercorea | Prevotella sp. | | 100 | 557 |
| AscusEQ_1095G | | Prevotella stercorea | Prevotella sp. | | 98.2 | 558 |
| AscusEQ_614 | 0.20074 | Phascolarctobacterium succinatutens | Phascolarctobacterium sp. | 92.05 | | 559 |
| AscusEQ_614A | | Phascolarctobacterium succinatutens | Phascolarctobacterium sp. | | 99.1 | 560 |
| AscusEQ_614B | | Phascolarctobacterium succinatutens | Phascolarctobacterium sp. | | 98.7 | 561 |
| AscusEQ_614C | | Phascolarctobacterium succinatutens | Phascolarctobacterium sp. | | 100 | 562 |
| AscusEQ_614D | | Phascolarctobacterium succinatutens | Phascolarctobacterium sp. | | 99.6 | 563 |
| AscusEQ_614E | | Phascolarctobacterium succinatutens | Phascolarctobacterium sp. | | 97.8 | 564 |
| AscusEQ_614F | | Phascolarctobacterium succinatutens | Phascolarctobacterium sp. | | 98.2 | 565 |
| AscusEQ_614G | | Phascolarctobacterium succinatutens | Phascolarctobacterium sp. | | 97.3 | 566 |
| AscusEQ_698 | 0.20029 | Phascolarctobacterium succinatutens | Phascolarctobacterium sp. | 91.36 | | 567 |
| AscusEQ_678 | 0.2002 | Pedobacter bauzanensis | Bacteroidales bacterium | 88.33 | | 568 |
| AscusEQ_698A | | Pedobacter bauzanensis | Bacteroidales bacterium | | 100 | 569 |
| AscusEQ_698B | | Pedobacter bauzanensis | Bacteroidales bacterium | | 98.7 | 570 |
| AscusEQ_698C | | Pedobacter bauzanensis | Bacteroidales bacterium | | 98.2 | 571 |
| AscusEQ_698D | | Pedobacter bauzanensis | Bacteroidales bacterium | | 97.3 | 572 |
| AscusEQ_698E | | Pedobacter bauzanensis | Bacteroidales bacterium | | 99.1 | 573 |
| AscusEQ_698F | | Pedobacter bauzanensis | Bacteroidales bacterium | | 99.6 | 574 |
| AscusEQ_698G | | Pedobacter bauzanensis | Bacteroidales bacterium | | 97.8 | 575 |

In some embodiments, the present disclosure provides microbial compositions comprising isolated microbial species selected from a Clostridium spp. bacterium, a Sarcina spp. bacterium, a Streptococcus spp. bacterium, an Escheria spp. bacterium, an Atlantibacter spp. bacterium, and a Shigella spp. bacterium.

In some embodiments, the present disclosure provides microbial compositions comprising isolated microbial species selected from a Clostridium butyricum bacterium, a Streptococcus infantarius subsp. coli bacterium, a Streptococcus equinius bacterium, an Escheria coli bacterium, a Sacina maxima bacterium, a Clostridium maximum bacterium, a Shigella sonnei bacterium, and an Atlantibacter hermannii bacterium.

In some embodiments, the present disclosure provides microbial compositions comprising a Clostridium butyricum bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 5-13.

In some embodiments, the present disclosure provides microbial compositions comprising a Streptococcus infantarius subsp. coli bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 143-150.

In some embodiments, the present disclosure provides microbial compositions comprising a Streptococcus equinis bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 143-150.

In some embodiments, the present disclosure provides microbial compositions comprising anEscheria coli bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 321-328.

In some embodiments, the present disclosure provides microbial compositions comprising a Sarcina maxima bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 430-437.

In some embodiments, the present disclosure provides microbial compositions comprising a Clostridium maximum bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 430-437.

In some embodiments, the present disclosure provides microbial compositions comprising a Shigella sonnei bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 480-486.

In some embodiments, the present disclosure provides microbial compositions comprising an Atlantibacter hermannii bacterium comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 480-486.

In some embodiments, the present disclosure provides microbial compositions comprising one or more isolated bacteria comprising a 16S nucleic acid sequence selected from SEQ ID NOs: 1-574. In some embodiments, the microbial composition comprises one or more isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to a nucleic acid sequence selected from SEQ ID NOs: 1-574. In some embodiments, the microbial composition comprises one or more isolated bacteria comprising a 16S nucleic acid sequence comprising or consisting of a nucleic acid sequence selected from SEQ ID NOs: 1-574.

In some embodiments, the present disclosure provides a microbial composition comprising one or more of:

(a) AscusEQ_4 (SEQ ID NO: 5) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 5;
(b) AscusEQ_140 (SEQ ID NO: 141) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 141;
(c) AscusEQ_61 (SEQ ID NO: 319) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 319;
(d) AscusEQ_414 (SEQ ID NO: 426) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 426;
(e) AscusEQ_109 (SEQ ID NO: 475) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 475;

In some embodiments, the present disclosure provides a microbial composition comprising one or more of:

(a) AscusEQ_4F (SEQ ID NO: 11) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 11;

(b) AscusEQ_140A (SEQ ID NO: 142) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 142;

(c) AscusEQ_61A (SEQ ID NO: 320) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 320;

(d) AscusEQ_414G (SEQ ID NO: 433) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 433;

(e) AscusEQ_109A (SEQ ID NO: 476) or an isolated bacteria comprising a 16S nucleic acid sequence that is at least 95.0%, at least 95.1%, at least 95.2%, at least 95.3%, at least 95.4%, at least 95.5%, at least 95.6%, at least 95.7%, at least at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 476.

Tables 3-6 provide the various combinations of (a)-(e) above that are contemplated according to the present disclosure.

TABLE 3
Compositions comprising 5 isolated bacteria

(a)(b)(c)(d)(e)

TABLE 4
Compositions comprising 4 isolated bacteria

(a)(b)(c)(d)
(a)(b)(c)(e)
(a)(b)(d)(e)
(a)(c)(d)(e)
(b)(c)(d)(e)

TABLE 5
Compositions comprising 3 isolated bacteria

(a)(b)(c)
(a)(b)(d)
(a)(b)(e)
(a)(c)(d)
(a)(c)(e)
(b)(c)(d)
(b)(c)(e)
(b)(d)(e)
(c)(d)(e)

TABLE 6
Compositions comprising 2 isolated bacteria

(a)(b)
(a)(c)
(a)(d)
(a)(e)
(b)(c)
(b)(d)
(b)(e)
(c)(d)
(c)(e)
(d)(e)

In some embodiments, the microbial compositions include feed, such as cereals (barley, maize, oats, and the like); starches (tapioca and the like); oilseed cakes; and vegetable wastes. In some embodiments, the microbial compositions include vitamins, minerals, trace elements, emulsifiers, aromatizing products, binders, colorants, odorants, thickening agents, and the like. In some embodiments, the microbial compositions include one or more of an ionophore; vaccine; antibiotic; antihelmintic; virucide; nematicide; amino acids such as methionine, glycine, and arginine; fish oil; oregano; and biologically active molecules such as enzymes.

In some embodiments, the microbial compositions of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium sulfate; magnesium sulfate; magnesium oxide; zeolites, calcium carbonate; magnesium carbonate; trehalose; chitosan; shellac; albumins; starch; skim-milk powder; sweet-whey powder; maltodextrin; lactose; inulin; dextrose; products of vegetable origin such as cereal meals, tree bark meal, wood meal, and nutshell meal.

In some embodiments, the microbial compositions of the present disclosure are liquid. In further embodiments, the liquid comprises a solvent that may include water or an alcohol or a saline or carbohydrate solution, and other animal-safe solvents. In some embodiments, the microbial compositions of the present disclosure include binders such as animal-safe polymers, carboxymethylcellulose, starch, polyvinyl alcohol, and the like.

In some embodiments, the microbial compositions of the present disclosure comprise thickening agents such as silica, clay, natural extracts of seeds or seaweed, synthetic derivatives of cellulose, guar gum, locust bean gum, agar, gelatin, xantham gum, alginates, and methylcelluloses. In some embodiments, the microbial compositions comprise anti-settling agents such as modified starches, polyvinyl alcohol, xanthan gum, and the like.

In some embodiments, the microbial compositions of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some embodiments, the microbial compositions of the present disclosure comprise trace nutrients such as salts of iron, manganese, boron, copper, cobalt, molybdenum, and zinc. In some embodiments, the microbial compositions comprise dyes, both natural and artificial. In some embodiments, the dye is green in color.

In some embodiments, the microbial compositions of the present disclosure comprise an animal-safe virucide, bacteriocide, or nematicide.

In some embodiments, microbial compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts, and combinations thereof. In a further embodiment, microbial compositions comprise polymers of agar, agarose, gelrite, and gellan gum, and the like. In some embodiments, microbial compositions comprise plastic capsules, emulsions (e.g., water and oil), membranes, and artificial membranes. In some embodiments, emulsions or linked polymer solutions may comprise microbial compositions of the present disclosure. See Harel and Bennett (U.S. Pat. No. 8,460,726B2). In one embodiment, the microbial composition comprises glucose. In one embodiment, formulations of the microbial composition comprise glucose.

In some embodiments, microbial compositions of the present disclosure comprise one or more oxygen scavengers, denitrifiers, nitrifiers, heavy metal chelators, and/or dechlorinators; and combinations thereof. In one embodiment, the one or more oxygen scavengers, denitrifiers, nitrifiers, heavy metal chelators, and/or dechlorinators are not chemically active once the microbial compositions are mixed with food and/or water to be administered to the equine. In one embodiment, the one or more oxygen scavengers, denitrifiers, nitrifiers, heavy metal chelators, and/or dechlorinators are not chemically active when administered to the equine.

In some embodiments, the microbial compositions of the present disclosure comprise a solidification agent and a sweetening agent. In some embodiments, the sweetening agent is selected from corn syrup, molasses, cane molasses, brewer's yeast, and honey. In some embodiments, the sweetening agent is molasses. In some embodiments, the solidification agent is selected from gelatin, xantham gum, agar, a starch, alginin, guar gum, collagen, pectin, and carboxymethyl cellulose. In some embodiments, the solidification agent is gelatin. In some embodiments, the microbial composition comprises between about 0.1% to about 1.5% gelatin. In some embodiments, the solidification agent is xantham gum. In some embodiments, the microbial composition comprises between about 0.2% and about 2.0% of xantham gum. In some embodiments, the microbial composition comprises greater than 1.4% xantham gum. In some embodiments, the solidification agent is agar. In some embodiments, the microbial composition comprises between about 0.25% and about 2.5% agar. In some embodiments, the microbial composition comprises greater than about 1.0% agar.

In some embodiments, microbial compositions of the present disclosure occur in a solid form (e.g., dispersed lyophilized spores) or a liquid or gel form (microbes interspersed in a storage medium). In some embodiments, microbial compositions of the present disclosure are added in dry form to a liquid or gel to form a suspension prior to administration.

In some embodiments, microbial compositions of the present disclosure are formulated as a gel, a liquid, a powder, a tablet, a capsule, a pill, a feed additive, a food ingredient, a food supplement, a water additive, a heat-stabilized additive, a moisture-stabilized additive, a pelleted feed additive, a pre-pelleted applied feed additive, a post-pelleted applied feed additive, or a spray additive.

In some embodiments, microbial compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono- and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some embodiments, microbial compositions of the present disclosure include bacterial and/or fungal cells in spore form, vegetative cell form, dormant cell form, and/or lysed form. In one embodiment, the lysed cell form acts as a mycotoxin binder, e.g. mycotoxins binding to dead cells.

In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of about 1 to 100, about 1 to 95, about 1 to 90, about 1 to 85, about 1 to 80, about 1 to 75, about 1 to 70, about 1 to 65, about 1 to 60, about 1 to 55, about 1 to 50, about 1 to 45, about 1 to 40, about 1 to 35, about 1 to 30, about 1 to 25, about 1 to 20, about 1 to 15, about 1 to 10, about 1 to 5, about 5 to 100, about 5 to 95, about 5 to 90, about 5 to 85, about 5 to 80, about 5 to 75, about 5 to 70, about 5 to 65, about 5 to 60, about 5 to 55, about 5 to 50, about 5 to 45, about 5 to 40, about 5 to 35, about 5 to 30, about 5 to 25, about 5 to 20, about 5 to 15, about 5 to 10, about 10 to 100, about 10 to 95, about 10 to 90, about 10 to 85, about 10 to 80, about 10 to 75, about 10 to 70, about 10 to 65, about 10 to 60, about 10 to 55, about 10 to 50, about 10 to 45, about 10 to 40, about 10 to 35, about 10 to 30, about 10 to 25, about 10 to 20, about 10 to 15, about 15 to 100, about 15 to 95, about 15 to 90, about 15 to 85, about 15 to 80, about 15 to 75, about 15 to 70, about 15 to 65, about 15 to 60, about 15 to 55, about 15 to 50, about 15 to 45, about 15 to 40, about 15 to 35, about 15 to 30, about 15 to 25, about 15 to 20, about 20 to 100, about 20 to 95, about 20 to 90, about 20 to 85, about 20 to 80, about 20 to 75, about 20 to 70, about 20 to 65, about 20 to 60, about 20 to 55, about 20 to 50, about 20 to 45, about 20 to 40, about 20 to 35, about 20 to 30, about 20 to 25, about 25 to 100, about 25 to 95, about 25 to 90, about 25 to 85, about 25 to 80, about 25 to 75, about 25 to 70, about 25 to 65, about 25 to 60, about 25 to 55, about 25 to 50, about 25 to 45, about 25 to 40, about 25 to 35, about 25 to 30, about 30 to 100, about 30 to 95, about 30 to 90, about 30 to 85, about 30 to 80, about 30 to 75, about 30 to 70, about 30 to 65, about 30 to 60, about 30 to 55, about 30 to 50, about 30 to 45, about 30 to 40, about 30 to 35, about 35 to 100, about 35 to 95, about 35 to 90, about 35 to 85, about 35 to 80, about 35 to 75, about 35 to 70, about 35 to 65, about 35 to 60, about 35 to 55, about 35 to 50, about 35 to 45, about 35 to 40, about 40 to 100, about 40 to 95, about 40 to 90, about 40 to 85, about 40 to 80, about 40 to 75, about 40 to 70, about 40 to 65, about 40 to 60, about 40 to 55, about 40 to 50, about 40 to 45, about 45 to 100, about 45 to 95, about 45 to 90, about 45 to 85, about 45 to 80, about 45 to 75, about 45 to 70, about 45 to 65, about 45 to 60, about 45 to 55, about 45 to 50, about 50 to 100, about 50 to 95, about 50 to 90, about 50 to 85, about 50 to 80, about 50 to 75, about 50 to 70, about 50 to 65, about 50 to 60, about 50 to 55, about 55 to 100, about 55 to 95, about 55 to 90, about 55 to 85, about 55 to 80, about 55 to 75, about 55 to 70, about 55 to 65, about 55 to 60, about 60 to 100, about 60 to 95, about 60 to 90, about 60 to 85, about 60 to 80, about 60 to 75, about 60 to 70, about 60 to 65, about 65 to 100, about 65 to 95, about 65 to 90, about 65 to 85, about 65 to 80, about 65 to 75, about 65 to 70, about 70 to 100, about 70 to 95, about 70 to 90, about 70 to 85, about 70 to 80, about 70 to 75, about 75 to 100, about 75 to 95, about 75 to 90, about 75 to 85, about 75 to 80, about 80 to 100, about 80 to 95, about 80 to 90, about 80 to 85, about 85 to 100, about 85 to 95, about 85 to 90, about 90 to 100, about 90 to 95, or 95 to 100 weeks

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10, 1 to 5, 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, 5 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, 15 to 25, 15 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 weeks.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 months.

In some embodiments, the microbial compositions of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%.

Moisture content is a measurement of the total amount of water in a composition, usually expressed as a percentage of the total weight. The moisture content is a usefull measurement for determining the dry weight of a composition, and it can be used to confirm whether the desiccation/drying process of a composition is complete. The moisture content is calculated by dividing the (wet weight of the composition minus the weight after desiccating/drying) by the wet weight of the composition, and multiplying by 100.

Moisture content defines the amount of water in a composition, but water activity explains how the water in the composition will react with microorganisms. The greater the water activity, the faster microorganisms are able to grow. Water activity is calculated by finding the ratio of the vapor pressure in a composition to the vapor pressure of pure water. More specifically, the water activity is the partial vapor pressure of water in a composition divided by the standard state partial vapor pressure of pure water. Pure distilled water has a water activity of 1. A determination of water activity of a composition is not the amount of water in a composition, rather it is the amount of excess amount of water that is available for microorganisms to use. Microorganisms have a minimal and optimal water activity for growth.

In some embodiments, the microbial compositions of the present disclosure are desiccated. A microbial composition is desiccated if the moisture content of the composition is between 0% and 20%.

In some embodiments, the microbial compositions of the present disclosure have a moisture content of about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the microbial compositions of the present disclosure have a moisture content of less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, less than 50%, less than 51%, less than 52%, less than 53%, less than 54%, less than 55%, less than 56%, less than 57%, less than 58%, less than 59%, less than 60%, less than 61%, less than 62%, less than 63%, less than 64%, less than 65%, less than 66%, less than 67%, less than 68%, less than 69%, less than 70%, less than 71%, less than 72%, less than 73%, less than 74%, less than 75%, less than 76%, less than 77%, less than 78%, less than 79%, less than 80%, less than 81%, less than 82%, less than 83%, less than 84%, less than 85%, less than 86%, less than 87%, less than 88%, less than 89%, less than 90%, less than 91%, less than 92%, less than 93%, less than 94%, less than 95%, less than 96%, less than 97%, less than 98%, less than 99%, or less than 100%.

In some embodiments, the microbial compositions of the present disclosure have a moisture content of less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, less than about 50%, less than about 51%, less than about 52%, less than about 53%, less than about 54%, less than about 55%, less than about 56%, less than about 57%, less than about 58%, less than about 59%, less than about 60%, less than about 61%, less than about 62%, less than about 63%, less than about 64%, less than about 65%, less than about 66%, less than about 67%, less than about 68%, less than about 69%, less than about 70%, less than about 71%, less than about 72%, less than about 73%, less than about 74%, less than about 75%, less than about 76%, less than about 77%, less than about 78%, less than about 79%, less than about 80%, less than about 81%, less than about 82%, less than about 83%, less than about 84%, less than about 85%, less than about 86%, less than about 87%, less than about 88%, less than about 89%, less than about 90%, less than about 91%, less than about 92%, less than about 93%, less than about 94%, less than about 95%, less than about 96%, less than about 97%, less than about 98%, less than about 99%, or less than about 100%.

In some embodiments, the microbial compositions of the present disclosure have a moisture content of 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 100%, 15% to 95%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 100%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 100%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 100%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 100%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 100%, 40% to 95%, 40% to 90%, 40% to 85%, 40% to 80%, 40% to 75%, 40% to 70%, 40% to 65%, 40% to 60%, 40% to 55%, 40% to 50%, 40% to 45%, 45% to 100%, 45% to 95%, 45% to 90%, 45% to 85%, 45% to 80%, 45% to 75%, 45% to 70%, 45% to 65%, 45% to 60%, 45% to 55%, 45% to 50%, 50% to 100%, 50% to 95%, 50% to 90%, 50% to 85%, 50% to 80%, 50% to 75%, 50% to 70%, 50% to 65%, 50% to 60%, 50% to 55%, 55% to 100%, 55% to 95%, 55% to 90%, 55% to 85%, 55% to 80%, 55% to 75%, 55% to 70%, 55% to 65%, 55% to 60%, 60% to 100%, 60% to 95%, 60% to 90%, 60% to 85%, 60% to 80%, 60% to 75%, 60% to 70%, 60% to 65%, 65% to 100%, 65% to 95%, 65% to 90%, 65% to 85%, 65% to 80%, 65% to 75%, 65% to 70%, 70% to 100%, 70% to 95%, 70% to 90%, 70% to 85%, 70% to 80%, 70% to 75%, 75% to 100%, 75% to 95%, 75% to 90%, 75% to 85%, 75% to 80%, 80% to 100%, 80% to 95%, 80% to 90%, 80% to 85%, 85% to 100%, 85% to 95%, 85% to 90%, 90% to 100%, 90% to 95%, or 95% to 100%.

### Encapsulated Microbes

In some embodiments, the microbes or microbial compositions of the disclosure are encapsulated in an encapsulating composition. An encapsulating composition protects the microbes from external stressors prior to entering the gastrointestinal tract of equines. In some embodiments, external stressors include thermal, desiccating, and physical stressors associated with pelleting and extrusion. In some embodiments, external stressors include chemicals present in the compositions to which Encapsulating compositions further create an environment that may be beneficial to the microbes, such as minimizing the oxidative stresses of an aerobic environment on anaerobic microbes, preserving the viability of the microbes wherein vegetative cells or spores form during the pelleting/extrusion process, etc., See Kalsta et al. (U.S. Pat. No. 5,104,662A), Ford (U.S. Pat. No. 5,733,568A), and Mosbach and Nilsson (U.S. Pat. No. 4,647,536A) for encapsulation compositions of microbes, and methods of encapsulating microbes.

In one embodiment, the compositions of the present disclosure exhibit a thermal tolerance, which is used interchangeably with heat tolerance and heat resistance. In one embodiment, thermal tolerant compositions of the present disclosure are tolerant of the high temperatures associated with feed manufacturing, mixing of feed and compositions of the present disclosure, storage in high heat environments, etc. In one embodiment, thermal tolerant compositions of the present disclosure are resistant to heat-killing and denaturation of the cell wall components and the intracellular environment. In one embodiment, the compositions of the present disclosure is tolerant or resistant to dessication/water loss.

In one embodiments, the encapsulation is a reservoir-type encapsulation. In one embodiment, the encapsulation is a matrix-type encapsulation. In one embodiment, the encapsulation is a coated matrix-type encapsulation. Burgain et al. (2011. J. Food Eng. 104:467-483) discloses numerous encapsulation embodiments and techniques, all of which are incorporated by reference.

In some embodiments, the compositions of the present disclosure are encapsulated in one or more of the following: gellan gum, xanthan gum, K-Carrageenan, cellulose acetate phthalate, chitosan, starch, milk fat, whey protein, Ca-alginate, raftilose, raftiline, pectin, saccharide, glucose, maltodextrin, gum arabic, guar, seed flour, alginate, dextrins, dextrans, celluloase, gelatin, gelatin, albumin, casein, gluten, acacia gum, tragacanth, wax, paraffin, stearic acid, monodiglycerides, and diglycerides. In some embodiments, the compositions of the present disclosure are encapsulated by one or more of a polymer, carbohydrate, sugar, plastic, glass, polysaccharide, lipid, wax, oil, fatty acid, or glyceride. In one embodiment, the microbial composition is encapsulated by a glucose. In one embodiment, the microbial composition is encapsulated by a glucose-containing composition. In one embodiment, formulations of the microbial composition comprise a glucose encapsulant. In one embodiment, formulations of the microbial composition comprise a glucose-encapsulated composition.

In some embodiments, the encapsulation of the compositions of the present disclosure is carried out by an extrusion, emulsification, coating, agglomeration, lyophilization, vacuum-drying, or spray-drying.

In one embodiment, the encapsulating composition comprises microcapsules having a multiplicity of liquid cores encapsulated in a solid shell material. For purposes of the disclosure, a “multiplicity” of cores is defined as two or more.

A first category of useful fusible shell materials is that of normally solid fats, including fats which are already of suitable hardness and animal or vegetable fats and oils which are hydrogenated until their melting points are sufficiently high to serve the purposes of the present disclosure. Depending on the desired process and storage temperatures and the specific material selected, a particular fat can be either a normally solid or normally liquid material. The terms “normally solid” and “normally liquid” as used herein refer to the state of a material at desired temperatures for storing the resulting microcapsules. Since fats and hydrogenated oils do not, strictly speaking, have melting points, the term “melting point” is used herein to describe the minimum temperature at which the fusible material becomes sufficiently softened or liquid to be successfully emulsified and spray cooled, thus roughly corresponding to the maximum temperature at which the shell material has sufficient integrity to prevent release of the choline cores. “Melting point” is similarly defined herein for other materials which do not have a sharp melting point.

Specific examples of fats and oils useful herein (some of which require hardening) are as follows: animal oils and fats, such as beef tallow, mutton tallow, lamb tallow, lard or pork fat, fish oil, and sperm oil; vegetable oils, such as canola oil, cottonseed oil, peanut oil, corn oil, olive oil, soybean oil, sunflower oil, safflower oil, coconut oil, palm oil, linseed oil, tung oil, and castor oil; fatty acid monoglycerides and diglycerides; free fatty acids, such as stearic acid, palmitic acid, and oleic acid; and mixtures thereof. The above listing of oils and fats is not meant to be exhaustive, but only exemplary.

Specific examples of fatty acids include linoleic acid, γ-linoleic acid, dihomo-γ-linolenic acid, arachidonic acid, docosatetraenoic acid, vaccenic acid, nervonic acid, mead acid, crucic acid, gondoic acid, elaidic acid, oleic acid, palitoleic acid, stearidonic acid, eicosapentaenoic acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecyclic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

Another category of fusible materials useful as encapsulating shell materials is that of waxes. Representative waxes contemplated for use herein are as follows: animal waxes, such as beeswax, lanolin, shell wax, and Chinese insect wax; vegetable waxes, such as carnauba, candelilla, bayberry, and sugar cane; mineral waxes, such as paraffin, microcrystalline petroleum, ozocerite, ceresin, and montan; synthetic waxes, such as low molecular weight polyolefin (e.g., CARBOWAX), and polyol ether-esters (e.g., sorbitol); Fischer-Tropsch process synthetic waxes; and mixtures thereof. Water-soluble waxes, such as CARBOWAX and sorbitol, are not contemplated herein if the core is aqueous.

Still other fusible compounds useful herein are fusible natural resins, such as rosin, balsam, shellac, and mixtures thereof.

Various adjunct materials are contemplated for incorporation in fusible materials according to the present disclosure. For example, antioxidants, light stabilizers, dyes and lakes, flavors, essential oils, anti-caking agents, fillers, pH stabilizers, sugars (monosaccharides, disaccharides, trisaccharides, and polysaccharides) and the like can be incorporated in the fusible material in amounts which do not diminish its utility for the present disclosure.

The core material contemplated herein constitutes from about 0.1% to about 50%, about 1% to about 35%, or about 5% to about 30% by weight of the microcapsules. In some embodiments, the core material contemplated herein constitutes no more than about 30% by weight of the microcapsules. In some embodiments, the core material contemplated herein constitutes about 5% by weight of the microcapsules. The core material is contemplated as either a liquid or solid at contemplated storage temperatures of the microcapsules.

The cores may include other additives well-known in the pharmaceutical art, including edible sugars, such as sucrose, glucose, maltose, fructose, lactose, cellobiose, monosaccharides, disaccharides, trisaccharides, and polysaccharides, and mixtures thereof; artificial sweeteners, such as aspartame, saccharin, cyclamate salts, and mixtures thereof; edible acids, such as acetic acid (vinegar), citric acid, ascorbic acid, tartaric acid, and mixtures thereof; edible starches, such as corn starch; hydrolyzed vegetable protein; water-soluble vitamins, such as Vitamin C; water-soluble medicaments; water-soluble nutritional materials, such as ferrous sulfate; flavors; salts; monosodium glutamate; antimicrobial agents, such as sorbic acid; antimycotic agents, such as potassium sorbate, sorbic acid, sodium benzoate, and benzoic acid; food grade pigments and dyes; and mixtures thereof. Other potentially useful supplemental core materials will be apparent to those of ordinary skill in the art.

Emulsifying agents may be employed to assist in the formation of stable emulsions. Representative emulsifying agents include glyceryl monostearate, polysorbate esters, ethoxylated mono- and diglycerides, and mixtures thereof.

For ease of processing, and particularly to enable the successful formation of a reasonably stable emulsion, the viscosities of the core material and the shell material should be similar at the temperature at which the emulsion is formed. In particular, the ratio of the viscosity of the shell to the viscosity of the core, expressed in centipoise or comparable units, and both measured at the temperature of the emulsion, should be from about 22:1 to about 1:1, desirably from about 8:1 to about 1:1, and preferably from about 3:1 to about 1:1. A ratio of 1:1 would be ideal, but a viscosity ratio within the recited ranges is useful.

Encapsulating compositions are not limited to microcapsule compositions as disclosed above. In some embodiments encapsulating compositions encapsulate the microbial compositions in an adhesive polymer that can be natural or synthetic without toxic effect. In some embodiments, the encapsulating composition may be a matrix selected from sugar matrix, gelatin matrix, polymer matrix, silica matrix, starch matrix, foam matrix, glass/glassy matrix etc. See Pirzio et al. (U.S. Pat. No. 7,488,503). In some embodiments, the encapsulating composition may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; monosaccharides; fats; fatty acids, including oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

In some embodiments, the microbial composition or a subcomponent thereof is encapsulated in a solid glass matrix or a flexible glass matrix (rubber matrix) comprising one or more polysaccharides, one or more saccharides, and/or one or more sugar alcohols. In some embodiments, the matrix comprises a monosaccharide or a disaccharide. In some embodiments, the disaccharide may be selected from sucrose, maltose, lactose, lactulose, trehalose, cellobiose, and chitobiose. In some embodiments, the polysaccharides, saccharides, and/or sugar alcohols are added to the microbial composition or a subcomponent thereof exogenously. In some embodiments, the matrix is an amorphous matrix. In some embodiments, the microbial composition or a subcompenent thereof is vitrified. In some embodiments, the microbial composition or a subcompenent thereof is desiccated. In some embodiments, the microbial composition or a subcompenent thereof is lyophilized. In some embodiments, the microbial composition or a subcompenent thereof is spray dried. In some embodiments, the microbial composition or a subcompenent thereof is spray congealed. In some embodiments, the microbial composition is preserved/stabilized by preservation by vaporization. See Harel and Kohavi-Beck (U.S. Pat. No. 8,097,245). See Bronshtein (U.S. Pat. No. 9,469,835).

In some embodiments, the encapsulating compositions comprise at least one layer of encapsulation. In some embodiments, the encapsulating compositions comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 layers of encapsulation/encapsulants.

In some embodiments, the encapsulating compositions comprise at least two layers of encapsulation. In some embodiments, each layer of encapsulation confers a different characteristic to the composition. In some embodiments, no two consecutive layers confer the same characteristic. In some embodiments, at least one layer of the at least two layers of encapsulation confers thermostability, shelf stability, ultraviolet resistance, moisture resistance, dessication resistance, hydrophobicity, hydrophilicity, lipophobicity, lipophilicity, pH stability, acid resistance, and base resistance.

In some embodiments, the encapsulating compositions comprise two layers of encapsulation; the first layer confers thermostability and/or shelf stability, and the second layer provides pH resistance.

In some embodiments, the encapsulating layers confer a timed release of the microbial composition held in the center of the encapsulating layers. In some embodiments, the greater the number of layers confers a greater amount of time before the microbial composition is exposed, post administration.

In some embodiments, the encapsulating shell of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

### Animal Feed

In some embodiments, the microbial products of the present disclosure are mixed with animal feed. In some embodiments, animal feed may be present in various forms such as pellets, capsules, granulated, powdered, liquid, or semi-liquid.

In some embodiments, products of the present disclosure are mixed into the premix at the feed mill (e.g., Cargill or Western Millin), alone as a standalone premix, and/or alongside other feed additives such as MONENSIN, vitamins, etc. In one embodiment, the products of the present disclosure are mixed into the feed at the feed mill. In another embodiment, products of the present disclosure are mixed into the feed itself.

In some embodiments, the feed may be supplemented with water, premix or premixes, forage, fodder, beans (e.g., whole, cracked, or ground), grains (e.g., whole, cracked, or ground), bean- or grain-based oils, bean- or grain-based meals, bean- or grain-based haylage or silage, bean- or grain-based syrups, fatty acids, sugar alcohols (e.g., polyhydric alcohols), commercially available formula feeds, and mixtures thereof.

In some embodiments, the microbial compositions of the present disclosure are mixed into the premix or mash alongside a water additive. In some embodiments, the water additive comprises citric acid monohydrate, trisodium citrate dehydrate, and inulin. In some embodiments, citric acid monohydrate constitutes about 0.05%, 0.075%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.25%, 2.5%, 2.75%, 3.0%, 3.25%, 3.5%, 3.75%, 4.0%, 4.25%, 4.5%, 4.75%, or 5.0% of the water additive. In some embodiments, citric monohydrate constitutes 0.4% of the water additive. In some embodiments, trisodium citrate dehydrate constitutes about 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or about 10% of the water additive. In some embodiments, trisodium citrate dehydrate constitutes about 4.25% of the water additive. In some embodiments, inulin constitutes about 5%, 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, 55%, 60%, 65%, 70%, or 75% of the water additive. In some embodiments, inulin constitutes 28% of the water additive. In some embodiments, the water additive comprises 0.4% citric acid monohydrate, 4.25% trisodium citrate dehydrate, and 28% inulin.

In some embodiments, forage encompasses hay, haylage, and silage. In some embodiments, hays include grass hays (e.g., sudangrass, orchardgrass, or the like), alfalfa hay, and clover hay. In some embodiments, haylages include grass haylages, sorghum haylage, and alfalfa haylage. In some embodiments, silages include maize, oat, wheat, alfalfa, clover, and the like.

In some embodiments, premix or premixes may be utilized in the feed. Premixes may comprise micro-ingredients such as vitamins, minerals, amino acids; chemical preservatives; pharmaceutical compositions such as antibiotics and other medicaments; fermentation products, and other ingredients. In some embodiments, premixes are blended into the feed.

In some embodiments, the feed may include feed concentrates such as soybean hulls, sugar beet pulp, molasses, high protein soybean meal, ground corn, shelled corn, wheat midds, distiller grain, cottonseed hulls, rumen-bypass protein, rumen-bypass fat, and grease. See Luhman (U.S. Publication US20150216817A1), Anderson et al. (U.S. Pat. No. 3,484,243) and Porter and Luhman (U.S. Pat. No. 9,179,694B2) for animal feed and animal feed supplements capable of use in the present compositions and methods.

In some embodiments, feed occurs as a compound, which includes, in a mixed composition capable of meeting the basic dietary needs, the feed itself, vitamins, minerals, amino acids, and other necessary components. Compound feed may further comprise premixes.

In some embodiments, microbial compositions of the present disclosure may be mixed with animal feed, premix, and/or compound feed. Individual components of the animal feed may be mixed with the microbial compositions prior to feeding to ruminants. The microbial compositions of the present disclosure may be applied into or on a premix, into or on a feed, and/or into or on a compound feed.

Methods of Determining Microbial Abundance and Co-Occurrence of Microorganisms with Environmental Parameters

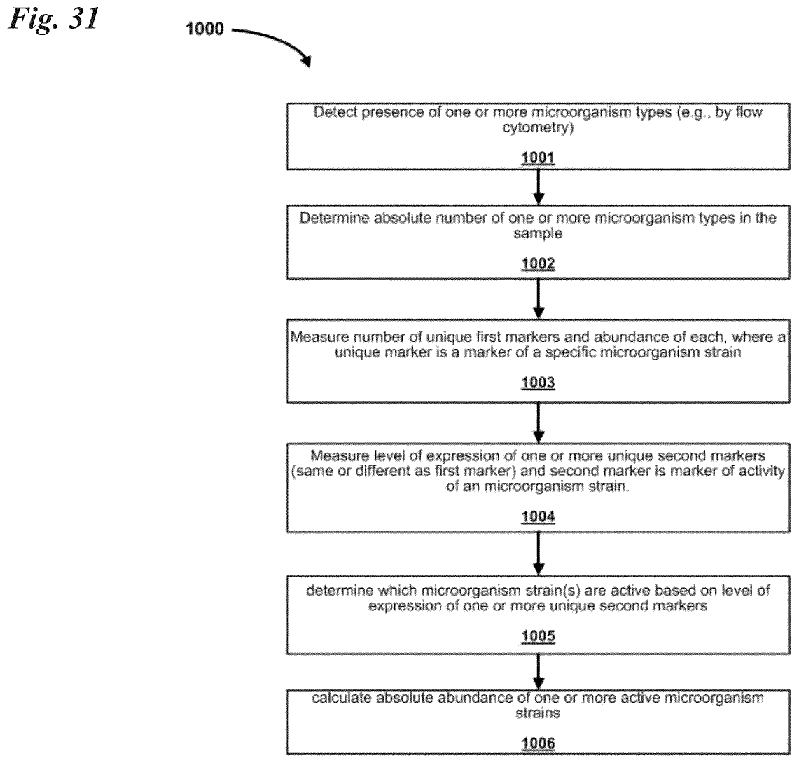

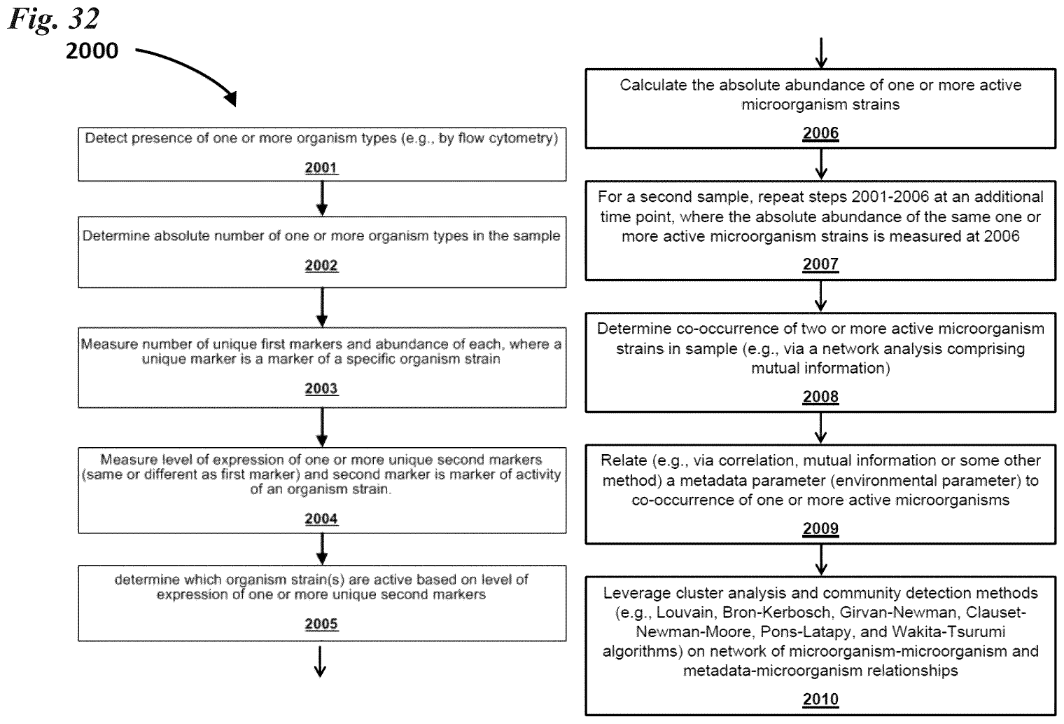

According to the methods provided herein, a sample is processed to detect the presence of one or more microorganism types in the sample (FIG. 31, 1001; FIG. 32, 2001). The absolute number of one or more microorganism types in the sample is determined (FIG. 31, 1002; FIG. 32, 2002). The determination of the presence of the one or more organism types and the absolute number of at least one organism type can be conducted in parallel or serially. For example, in the case of a sample comprising a microbial community comprising bacteria (i.e., one microorganism type) and fungi (i.e., a second microorganism type), the user in one embodiment detects the presence of one or both of the organism types in the sample (FIG. 31, 1001; FIG. 32, 2001). The user, in a further embodiment, determines the absolute number of at least one organism type in the sample—in the case of this example, the number of bacteria, fungi, or combination thereof, in the sample (FIG. 31, 1002; FIG. 32, 2002).

In one embodiment, the sample, or a portion thereof is subjected to flow cytometry (FC) analysis to detect the presence and/or number of one or more microorganism types (FIG. 31, 1001, 1002; FIG. 32, 2001, 2002). In one flow cytometer embodiment, individual microbial cells pass through an illumination zone, at a rate of at least about 300*s⁻¹, or at least about 500*s⁻¹, or at least about 1000*s⁻¹. However, one of ordinary skill in the art will recognize that this rate can vary depending on the type of instrument is employed. Detectors which are gated electronically measure the magnitude of a pulse representing the extent of light scattered. The magnitudes of these pulses are sorted electronically into “bins” or “channels,” permitting the display of histograms of the number of cells possessing a certain quantitative property (e.g., cell staining property, diameter, cell membrane) versus the channel number. Such analysis allows for the determination of the number of cells in each “bin” which in embodiments described herein is an “microorganism type” bin, e.g., a bacteria, fungi, nematode, protozoan, archaca, algae, dinoflagellate, virus, viroid, etc.

In one embodiment, a sample is stained with one or more fluorescent dyes wherein a fluorescent dye is specific to a particular microorganism type, to enable detection via a flow cytometer or some other detection and quantification method that harnesses fluorescence, such as fluorescence microscopy. The method can provide quantification of the number of cells and/or cell volume of a given organism type in a sample. In a further embodiment, as described herein, flow cytometry is harnessed to determine the presence and quantity of a unique first marker and/or unique second marker of the organism type, such as enzyme expression, cell surface protein expression, etc. Two- or three-variable histograms or contour plots of, for example, light scattering versus fluorescence from a cell membrane stain (versus fluorescence from a protein stain or DNA stain) may also be generated, and thus an impression may be gained of the distribution of a variety of properties of interest among the cells in the population as a whole. A number of displays of such multiparameter flow cytometric data are in common use and are amenable for use with the methods described herein.

In one embodiment of processing the sample to detect the presence and number of one or more microorganism types, a microscopy assay is employed (FIG. 31, 1001, 1002). In one embodiment, the microscopy is optical microscopy, where visible light and a system of lenses are used to magnify images of small samples. Digital images can be captured by a charge-couple device (CCD) camera. Other microscopic techniques include, but are not limited to, scanning electron microscopy and transmission electron microscopy. Microorganism types are visualized and quantified according to the aspects provided herein.

In another embodiment of in order to detect the presence and number of one or more microorganism types, the sample, or a portion thereof is subjected to fluorescence microscopy. Different fluorescent dyes can be used to directly stain cells in samples and to quantify total cell counts using an epifluorescence microscope as well as flow cytometry, described above. Useful dyes to quantify microorganisms include but are not limited to acridine orange (AO), 4,6-di-amino-2 phenylindole (DAPI) and 5-cyano-2,3 Dytolyl Tetrazolium Chloride (CTC). Viable cells can be estimated by a viability staining method such as the LIVE/DEAD® Bacterial Viability Kit (Bac-Light™) which contains two nucleic acid stains: the green-fluorescent SYTO 9™ dye penetrates all membranes and the red-fluorescent propidium iodide (PI) dye penetrates cells with damaged membranes. Therefore, cells with compromised membranes will stain red, whereas cells with undamaged membranes will stain green. Fluorescent in situ hybridization (FISH) extends epifluorescence microscopy, allowing for the fast detection and enumeration of specific organisms. FISH uses fluorescent labelled oligonucleotides probes (usually 15-25 basepairs) which bind specifically to organism DNA in the sample, allowing the visualization of the cells using an epifluorescence or confocal laser scanning microscope (CLSM). Catalyzed reporter deposition fluorescence in situ hybridization (CARD-FISH) improves upon the FISH method by using oligonucleotide probes labelled with a horse radish peroxidase (HRP) to amplify the intensity of the signal obtained from the microorganisms being studied. FISH can be combined with other techniques to characterize microorganism communities. One combined technique is high affinity peptide nucleic acid (PNA)-FISH, where the probe has an enhanced capability to penetrate through the Extracellular Polymeric Substance (EPS) matrix. Another example is LIVE/DEAD-FISH which combines the cell viability kit with FISH and has been used to assess the efficiency of disinfection in drinking water distribution systems.

In another embodiment, the sample, or a portion thereof is subjected to Raman micro-spectroscopy in order to determine the presence of a microorganism type and the absolute number of at least one microorganism type (FIG. 31, 1001-1002; FIG. 32, 2001-2002). Raman micro-spectroscopy is a non-destructive and label-free technology capable of detecting and measuring a single cell Raman spectrum (SCRS). A typical SCRS provides an intrinsic biochemical “fingerprint” of a single cell. A SCRS contains rich information of the biomolecules within it, including nucleic acids, proteins, carbohydrates and lipids, which enables characterization of different cell species, physiological changes, and cell phenotypes. Raman microscopy examines the scattering of laser light by the chemical bonds of different cell biomarkers. A SCRS is a sum of the spectra of all the biomolecules in one single cell, indicating a cell's phenotypic profile. Cellular phenotypes, as a consequence of gene expression, usually reflect genotypes. Thus, under identical growth conditions, different microorganism types give distinct SCRS corresponding to differences in their genotypes and can thus be identified by their Raman spectra.

In yet another embodiment, the sample, or a portion thereof is subjected to centrifugation in order to determine the presence of a microorganism type and the number of at least one microorganism type (FIG. 31, 1001-1002; FIG. 32, 2001-2002). This process sediments a heterogeneous mixture by using the centrifugal force created by a centrifuge. More dense components of the mixture migrate away from the axis of the centrifuge, while less dense components of the mixture migrate towards the axis. Centrifugation can allow fractionation of samples into cytoplasmic, membrane and extracellular portions. It can also be used to determine localization information for biological molecules of interest. Additionally, centrifugation can be used to fractionate total microbial community DNA. Different prokaryotic groups differ in their guanine-plus-cytosine (G+C) content of DNA, so density-gradient centrifugation based on G+C content is a method to differentiate organism types and the number of cells associated with each type. The technique generates a fractionated profile of the entire community DNA and indicates abundance of DNA as a function of G+C content. The total community DNA is physically separated into highly purified fractions, each representing a different G+C content that can be analyzed by additional molecular techniques such as denaturing gradient gel electrophoresis (DGGE)/amplified ribosomal DNA restriction analysis (ARDRA) (see discussion herein) to assess total microbial community diversity and the presence/quantity of one or more microorganism types.

In another embodiment, the sample, or a portion thereof is subjected to staining in order to determine the presence of a microorganism type and the number of at least one microorganism type (FIG. 31, 1001-1002; FIG. 32, 2001-2002). Stains and dyes can be used to visualize biological tissues, cells, or organelles within cells. Staining can be used in conjunction with microscopy, flow cytometry or gel electrophoresis to visualize or mark cells or biological molecules that are unique to different microorganism types. In vivo staining is the process of dyeing living tissues, whereas in vitro staining involves dyeing cells or structures that have been removed from their biological context. Examples of specific staining techniques for use with the methods described herein include, but are not limited to: gram staining to determine gram status of bacteria, endospore staining to identify the presence of endospores, Zichl-Neelsen staining, haematoxylin and cosin staining to examine thin sections of tissue, papanicolaou staining to examine cell samples from various bodily secretions, periodic acid-Schiff staining of carbohydrates, Masson's trichome employing a three-color staining protocol to distinguish cells from the surrounding connective tissue, Romanowsky stains (or common variants that include Wright's stain, Jenner's stain, May-Grunwald stain, Leishman stain and Giemsa stain) to examine blood or bone marrow samples, silver staining to reveal proteins and DNA, Sudan staining for lipids and Conklin's staining to detect true endospores. Common biological stains include acridine orange for cell cycle determination; bismarck brown for acid mucins; carmine for glycogen; carmine alum for nuclei; Coomassie blue for proteins; Cresyl violet for the acidic components of the neuronal cytoplasm; Crystal violet for cell walls; DAPI for nuclei; cosin for cytoplasmic material, cell membranes, some extracellular structures and red blood cells; ethidium bromide for DNA; acid fuchsine for collagen, smooth muscle or mitochondria; haematoxylin for nuclei; Hoechst stains for DNA; iodine for starch; malachite green for bacteria in the Gimenez staining technique and for spores; methyl green for chromatin; methylene blue for animal cells; neutral red for Nissl substance; Nile blue for nuclei; Nile red for lipohilic entities; osmium tetroxide for lipids; rhodamine is used in fluorescence microscopy; safranin for nuclei. Stains are also used in transmission electron microscopy to enhance contrast and include phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead (II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, and vanadyl sulfate.

In another embodiment, the sample, or a portion thereof is subjected to mass spectrometry (MS) in order to determine the presence of a microorganism type and the number of at least one microorganism type (FIG. 31, 1001-1002; FIG. 32, 2001-2002). MS, as discussed below, can also be used to detect the presence and expression of one or more unique markers in a sample (FIG. 31, 1003-1004; FIG. 32, 2003-2004). MS is used for example, to detect the presence and quantity of protein and/or peptide markers unique to microorganism types and therefore to provide an assessment of the number of the respective microorganism type in the sample. Quantification can be either with stable isotope labelling or label-free. De novo sequencing of peptides can also occur directly from MS/MS spectra or sequence tagging (produce a short tag that can be matched against a database). MS can also reveal post-translational modifications of proteins and identify metabolites. MS can be used in conjunction with chromatographic and other separation techniques (such as gas chromatography, liquid chromatography, capillary electrophoresis, ion mobility) to enhance mass resolution and determination.

In another embodiment, the sample, or a portion thereof is subjected to lipid analysis in order to determine the presence of a microorganism type and the number of at least one microorganism type (FIG. 31, 1001-1002; FIG. 32, 2001-2002). Fatty acids are present in a relatively constant proportion of the cell biomass, and signature fatty acids exist in microbial cells that can differentiate microorganism types within a community. In one embodiment, fatty acids are extracted by saponification followed by derivatization to give the respective fatty acid methyl esters (FAMEs), which are then analyzed by gas chromatography. The FAME profile in one embodiment is then compared to a reference FAME database to identify the fatty acids and their corresponding microbial signatures by multivariate statistical analyses.

In the aspects of the methods provided herein, the number of unique first makers in the sample, or portion thereof (e.g., sample aliquot) is measured, as well as the abundance of each of the unique first markers (FIG. 31, 1003; FIG. 32, 2003). A unique marker is a marker of a microorganism strain. It should be understood by one of ordinary skill in the art that depending on the unique marker being probed for and measured, the entire sample need not be analyzed. For example, if the unique marker is unique to bacterial strains, then the fungal portion of the sample need not be analyzed. As described above, in some embodiments, measuring the absolute abundance of one or more organism types in a sample comprises separating the sample by organism type, e.g., via flow cytometry.

Any marker that is unique to an organism strain can be employed herein. For example, markers can include, but are not limited to, small subunit ribosomal RNA genes (16S/18S rDNA), large subunit ribosomal RNA genes (23S/25S/28S rDNA), intercalary 5.8S gene, cytochrome c oxidase, beta-tubulin, elongation factor, RNA polymerase, and internal transcribed spacer (ITS).

Ribosomal RNA genes (rDNA), especially the small subunit ribosomal RNA genes, i.e., 18S rRNA genes (18S rDNA) in the case of eukaryotes and 16S rRNA (16S rDNA) in the case of prokaryotes, have been the predominant target for the assessment of organism types and strains in a microbial community. However, the large subunit ribosomal RNA genes, 28S rDNAs, have been also targeted. rDNAs are suitable for taxonomic identification because: (i) they are ubiquitous in all known organisms; (ii) they possess both conserved and variable regions; (iii) there is an exponentially expanding database of their sequences available for comparison. In community analysis of samples, the conserved regions serve as annealing sites for the corresponding universal PCR and/or sequencing primers, whereas the variable regions can be used for phylogenetic differentiation. In addition, the high copy number of rDNA in the cells facilitates detection from environmental samples.

The internal transcribed spacer (ITS), located between the 18S rDNA and 28S rDNA, has also been targeted. The ITS is transcribed but spliced away before assembly of the ribosomes The ITS region is composed of two highly variable spacers, ITS1 and ITS2, and the intercalary 5.8S gene. This rDNA operon occurs in multiple copies in genomes. Because the ITS region does not code for ribosome components, it is highly variable.

In one embodiment, the unique RNA marker can be an mRNA marker, an siRNA marker or a ribosomal RNA marker.

Protein-coding functional genes can also be used herein as a unique first marker. Such markers include but are not limited to: the recombinase A gene family (bacterial RecA, archaca RadA and RadB, eukaryotic Rad51 and Rad57, phage UvsX); RNA polymerase β subunit (RpoB) gene, which is responsible for transcription initiation and elongation; chaperonins. Candidate marker genes have also been identified for bacteria plus archaca: ribosomal protein S2 (rpsB), ribosomal protein S10 (rpsJ), ribosomal protein LI (rplA), translation elongation factor EF-2, translation initiation factor IF-2, metalloendopeptidase, ribosomal protein L22, ffh signal recognition particle protein, ribosomal protein L4/Lle (rplD), ribosomal protein L2 (rplB), ribosomal protein S9 (rpsI), ribosomal protein L3 (rplC), phenylalanyl-tRNA synthetase beta subunit, ribosomal protein L14b/L23e (rpIN), ribosomal protein S5, ribosomal protein S19 (rpsS), ribosomal protein S7, ribosomal protein L16/L10E (rplP), ribosomal protein S13 (rpsM), phenylalanyl-tRNA synthetase a subunit, ribosomal protein L15, ribosomal protein L25/L23, ribosomal protein L6 (rplF), ribosomal protein L11 (rplK), ribosomal protein L5 (rplE), ribosomal protein S12/S23, ribosomal protein L29, ribosomal protein S3 (rpsC), ribosomal protein S11 (rpsK), ribosomal protein L10, ribosomal protein S8, tRNA pseudouridine synthase B, ribosomal protein L18P/L5E, ribosomal protein S15P/S13e, Porphobilinogen deaminase, ribosomal protein S17, ribosomal protein L13 (rplM), phosphoribosylformylglycinamidine cyclo-ligase (rpsE), ribonuclease HII and ribosomal protein L24. Other candidate marker genes for bacteria include: transcription elongation protein NusA (nusA), rpoB DNA-directed RNA polymerase subunit beta (rpoB), GTP-binding protein EngA, rpoC DNA-directed RNA polymerase subunit beta′, priA primosome assembly protein, transcription-repair coupling factor, CTP synthase (pyrG), secY preprotein translocase subunit SecY, GTP-binding protein Obg/CgtA, DNA polymerase I, rpsF 30S ribosomal protein S6, poA DNA-directed RNA polymerase subunit alpha, peptide chain release factor 1, rplI 50S ribosomal protein L9, polyribonucleotide nucleotidyltransferase, tsf elongation factor Ts (tsf), rplQ 50S ribosomal protein L17, IRNA (guanine-N(1)-)-methyltransferase (rplS), rplY probable 50S ribosomal protein L25, DNA repair protein RadA, glucose-inhibited division protein A, ribosome-binding factor A, DNA mismatch repair protein MutL, smpB SsrA-binding protein (smpB), N-acetylglucosaminyl transferase, S-adenosyl-methyltransferase MraW, UDP-N-acetylmuramoylalanine--D-glutamate ligase, rplS 50S ribosomal protein L19, rpIT 50S ribosomal protein L20 (rpIT), ruvA Holliday junction DNA helicase, ruvB Holliday junction DNA helicase B, serS seryl-tRNA synthetase, rplU 50S ribosomal protein L21, rpsR 30S ribosomal protein S18, DNA mismatch repair protein MutS, rpsT 30S ribosomal protein S20, DNA repair protein RecN, frr ribosome recycling factor (frr), recombination protein RecR, protein of unknown function UPF0054, miaA IRNA isopentenyltransferase, GTP-binding protein YchF, chromosomal replication initiator protein DnaA, dephospho-CoA kinase, 16S rRNA processing protein RimM, ATP-cone domain protein, 1-deoxy-D-xylulose 5-phosphate reductoisomerase, 2C-methyl-D-erythritol 2,4-cyclodiphosphate synthase, fatty acid/phospholipid synthesis protein PlsX, tRNA (Ile)-lysidine synthetase, dnaG DNA primase (dnaG), ruvC Holliday junction resolvase, rpsP 30S ribosomal protein S16, Recombinase A recA, riboflavin biosynthesis protein RibF, glycyl-tRNA synthetase beta subunit, trmU tRNA (5-methylaminomethyl-2-thiouridylate)-methyltransferase, rpmI 50S ribosomal protein L35, hemE uroporphyrinogen decarboxylase, Rod shape-determining protein, rpmA 50S ribosomal protein L27 (rpmA), peptidyl-tRNA hydrolase, translation initiation factor IF-3 (infC), UDP-N-acetylmuramyl-tripeptide synthetase, rpmF 50S ribosomal protein L32, rpIL 50S ribosomal protein L7/L12 (rpIL), leuS leucyl-tRNA synthetase, ligA NAD-dependent DNA ligase, cell division protein FtsA, GTP-binding protein TypA, ATP-dependent Clp protease, ATP-binding subunit ClpX, DNA replication and repair protein RecF and UDP-N-acetylenolpyruvoylglucosamine reductase.

Phospholipid fatty acids (PLFAs) may also be used as unique first markers according to the methods described herein. Because PLFAs are rapidly synthesized during microbial growth, are not found in storage molecules and degrade rapidly during cell death, it provides an accurate census of the current living community. All cells contain fatty acids (FAs) that can be extracted and esterified to form fatty acid methyl esters (FAMEs). When the FAMEs are analyzed using gas chromatography-mass spectrometry, the resulting profile constitutes a ‘fingerprint’ of the microorganisms in the sample. The chemical compositions of membranes for organisms in the domains Bacteria and Eukarya are comprised of fatty acids linked to the glycerol by an ester-type bond (phospholipid fatty acids (PLFAs)). In contrast, the membrane lipids of Archaca are composed of long and branched hydrocarbons that are joined to glycerol by an ether-type bond (phospholipid ether lipids (PLELs)). This is one of the most widely used non-genetic criteria to distinguish the three domains. In this context, the phospholipids derived from microbial cell membranes, characterized by different acyl chains, are excellent signature molecules, because such lipid structural diversity can be linked to specific microbial taxa.

As provided herein, in order to determine whether an organism strain is active, the level of expression of one or more unique second markers, which can be the same or different as the first marker, is measured (FIG. 31, 1004; FIG. 32, 2004). Unique first unique markers are described above. The unique second marker is a marker of microorganism activity. For example, in one embodiment, the mRNA or protein expression of any of the first markers described above is considered a unique second marker for the purposes of this invention.

In one embodiment, if the level of expression of the second marker is above a threshold level (e.g., a control level) or at a threshold level, the microorganism is considered to be active (FIG. 31, 1005; FIG. 32, 2005). Activity is determined in one embodiment, if the level of expression of the second marker is altered by at least about 5%, at least about 10%, at least about 15%, at least about 20%, at least about 25%, or at least about 30%, as compared to a threshold level, which in some embodiments, is a control level.

Second unique markers are measured, in one embodiment, at the protein, RNA or metabolite level. A unique second marker is the same or different as the first unique marker.

As provided above, a number of unique first markers and unique second markers can be detected according to the methods described herein. Moreover, the detection and quantification of a unique first marker is carried out according to methods known to those of ordinary skill in the art (FIG. 31, 1003-1004, FIG. 32, 2003-2004).

Nucleic acid sequencing (e.g., gDNA, cDNA, RNA, mRNA) in one embodiment is used to determine absolute abundance of a unique first marker and/or unique second marker. Sequencing platforms include, but are not limited to, Sanger sequencing and high-throughput sequencing methods available from Roche/454 Life Sciences, Illumina/Solexa, Pacific Biosciences, Ion Torrent and Nanopore. The sequencing can be amplicon sequencing of particular DNA or RNA sequences or whole metagenome/transcriptome shotgun sequencing.

Traditional Sanger sequencing (Sanger et al. (1977) DNA sequencing with chain-terminating inhibitors. Proc Natl. Acad. Sci. USA, 74, pp. 5463-5467, incorporated by reference herein in its entirety) relies on the selective incorporation of chain-terminating dideoxynucleotides by DNA polymerase during in vitro DNA replication and is amenable for use with the methods described herein.

In another embodiment, the sample, or a portion thereof is subjected to extraction of nucleic acids, amplification of DNA of interest (such as the rRNA gene) with suitable primers and the construction of clone libraries using sequencing vectors. Selected clones are then sequenced by Sanger sequencing and the nucleotide sequence of the DNA of interest is retrieved, allowing calculation of the number of unique microorganism strains in a sample.

454 pyrosequencing from Roche/454 Life Sciences yields long reads and can be harnessed in the methods described herein (Margulies et al. (2005) Nature, 437, pp. 376-380; U.S. Pat. Nos. 6,274,320; 6,258,568; 6,210,891, each of which is herein incorporated in its entirety for all purposes). Nucleic acid to be sequenced (e.g., amplicons or nebulized genomic/metagenomic DNA) have specific adapters affixed on either end by PCR or by ligation. The DNA with adapters is fixed to tiny beads (ideally, one bead will have one DNA fragment) that are suspended in a water-in-oil emulsion. An emulsion PCR step is then performed to make multiple copies of each DNA fragment, resulting in a set of beads in which each bead contains many cloned copies of the same DNA fragment. Each bead is then placed into a well of a fiber-optic chip that also contains enzymes necessary for the sequencing-by-synthesis reactions. The addition of bases (such as A, C, G, or T) trigger pyrophosphate release, which produces flashes of light that are recorded to infer the sequence of the DNA fragments in each well. About 1 million reads per run with reads up to 1,000 bases in length can be achieved. Paired-end sequencing can be done, which produces pairs of reads, each of which begins at one end of a given DNA fragment. A molecular barcode can be created and placed between the adapter sequence and the sequence of interest in multiplex reactions, allowing each sequence to be assigned to a sample bioinformatically.

Illumina/Solexa sequencing produces average read lengths of about 25 basepairs (bp) to about 300 bp (Bennett et al. (2005) Pharmacogenomics, 6:373-382; Lange et al. (2014). BMC Genomics 15, p. 63; Fadrosh et al. (2014) Microbiome 2, p. 6; Caporaso et al. (2012) ISME J, 6, p. 1621-1624; Bentley et al. (2008) Accurate whole human genome sequencing using reversible terminator chemistry. Nature, 456:53-59). This sequencing technology is also sequencing-by-synthesis but employs reversible dye terminators and a flow cell with a field of oligos attached. DNA fragments to be sequenced have specific adapters on either end and are washed over a flow cell filled with specific oligonucleotides that hybridize to the ends of the fragments. Each fragment is then replicated to make a cluster of identical fragments. Reversible dye-terminator nucleotides are then washed over the flow cell and given time to attach. The excess nucleotides are washed away, the flow cell is imaged, and the reversible terminators can be removed so that the process can repeat and nucleotides can continue to be added in subsequent cycles. Paired-end reads that are 300 bases in length each can be achieved. An Illumina platform can produce 4 billion fragments in a paired-end fashion with 125 bases for each read in a single run. Barcodes can also be used for sample multiplexing, but indexing primers are used.

The SOLID (Sequencing by Oligonucleotide Ligation and Detection, Life Technologies) process is a “sequencing-by-ligation” approach, and can be used with the methods described herein for detecting the presence and abundance of a first marker and/or a second marker (FIG. 31, 1003-1004; FIG. 32, 2003-2004) (Peckham et al. SOLiD™ Sequencing and 2-Base Encoding. San Diego, CA: American Society of Human Genetics, 2007; Mitra et al. (2013) Analysis of the intestinal microbiota using SOLID 16S rRNA gene sequencing and SOLID shotgun sequencing. BMC Genomics, 14 (Suppl 5): S16; Mardis (2008) Next-generation DNA sequencing methods. Annu Rev Genomics Hum Genet, 9:387-402; each incorporated by reference herein in its entirety). A library of DNA fragments is prepared from the sample to be sequenced, and are used to prepare clonal bead populations, where only one species of fragment will be present on the surface of each magnetic bead. The fragments attached to the magnetic beads will have a universal PI adapter sequence so that the starting sequence of every fragment is both known and identical. Primers hybridize to the Pl adapter sequence within the library template. A set of four fluorescently labelled di-base probes compete for ligation to the sequencing primer. Specificity of the di-base probe is achieved by interrogating every Ist and 2nd base in each ligation reaction. Multiple cycles of ligation, detection and cleavage are performed with the number of cycles determining the eventual read length. The SOLID platform can produce up to 3 billion reads per run with reads that are 75 bases long. Paired-end sequencing is available and can be used herein, but with the second read in the pair being only 35 bases long. Multiplexing of samples is possible through a system akin to the one used by Illumina, with a separate indexing run.

The lon Torrent system, like 454 sequencing, is amenable for use with the methods described herein for detecting the presence and abundance of a first marker and/or a second marker (FIG. 31, 1003-1004; FIG. 32, 2003-2004). It uses a plate of microwells containing beads to which DNA fragments are attached. It differs from all of the other systems, however, in the manner in which base incorporation is detected. When a base is added to a growing DNA strand, a proton is released, which slightly alters the surrounding pH. Microdetectors sensitive to pH are associated with the wells on the plate, and they record when these changes occur. The different bases (A, C, G, T) are washed sequentially through the wells, allowing the sequence from each well to be inferred. The lon Proton platform can produce up to 50 million reads per run that have read lengths of 200 bases. The Personal Genome Machine platform has longer reads at 400 bases. Bidirectional sequencing is available. Multiplexing is possible through the standard in-line molecular barcode sequencing.

Pacific Biosciences (PacBio) SMRT sequencing uses a single-molecule, real-time sequencing approach and in one embodiment, is used with the methods described herein for detecting the presence and abundance of a first marker and/or a second marker (FIG. 31, 1003-1004; FIG. 32, 2003-2004). The PacBio sequencing system involves no amplification step, setting it apart from the other major next-generation sequencing systems. In one embodiment, the sequencing is performed on a chip containing many zero-mode waveguide (ZMW) detectors. DNA polymerases are attached to the ZMW detectors and phospholinked dye-labeled nucleotide incorporation is imaged in real time as DNA strands are synthesized. The PacBio system yields very long read lengths (averaging around 4,600 bases) and a very high number of reads per run (about 47,000). The typical “paired-end” approach is not used with PacBio, since reads are typically long enough that fragments, through CCS, can be covered multiple times without having to sequence from each end independently. Multiplexing with PacBio does not involve an independent read, but rather follows the standard “in-line” barcoding model.

In one embodiment, where the first unique marker is the ITS genomic region, automated ribosomal intergenic spacer analysis (ARISA) is used in one embodiment to determine the number and identity of microorganism strains in a sample (FIG. 31, 1003, FIG. 32, 2003) (Ranjard et al. (2003). Environmental Microbiology 5, pp. 1111-1120, incorporated by reference in its entirety for all purposes). The ITS region has significant heterogeneity in both length and nucleotide sequence. The use of a fluorescence-labeled forward primer and an automatic DNA sequencer permits high resolution of separation and high throughput. The inclusion of an internal standard in each sample provides accuracy in sizing general fragments.

In another embodiment, fragment length polymorphism (RFLP) of PCR-amplified rDNA fragments, otherwise known as amplified ribosomal DNA restriction analysis (ARDRA), is used to characterize unique first markers and the abundance of the same in samples (FIG. 31, 1003, FIG. 32, 2003) (Massol-Deya et al. (1995). Mol. Microb. Ecol. Manual. 3.3.2, pp. 1-18, incorporated by reference in its entirety for all purposes). rDNA fragments are generated by PCR using general primers, digested with restriction enzymes, electrophoresed in agarose or acrylamide gels, and stained with ethidium bromide or silver nitrate.

One fingerprinting technique used in detecting the presence and abundance of a unique first marker is single-stranded-conformation polymorphism (SSCP) (Lee et al. (1996). Appl Environ Microbiol 62, pp. 3112-3120; Scheinert et al. (1996). J. Microbiol. Methods 26, pp. 103-117; Schwieger and Tebbe (1998). Appl. Environ. Microbiol. 64, pp. 4870-4876, each of which is incorporated by reference herein in its entirety). In this technique, DNA fragments such as PCR products obtained with primers specific for the 16S rRNA gene, are denatured and directly electrophoresed on a non-denaturing gel. Separation is based on differences in size and in the folded conformation of single-stranded DNA, which influences the electrophoretic mobility. Reannealing of DNA strands during electrophoresis can be prevented by a number of strategies, including the use of one phosphorylated primer in the PCR followed by specific digestion of the phosphorylated strands with lambda exonuclease and the use of one biotinylated primer to perform magnetic separation of one single strand after denaturation. To assess the identity of the predominant populations in a given bioensemble, in one embodiment, bands are excised and sequenced, or SSCP-patterns can be hybridized with specific probes. Electrophoretic conditions, such as gel matrix, temperature, and addition of glycerol to the gel, can influence the separation.

In addition to sequencing based methods, other methods for quantifying expression (e.g., gene, protein expression) of a second marker are amenable for use with the methods provided herein for determining the level of expression of one or more second markers (FIG. 31, 1004; FIG. 32, 2004). For example, quantitative RT-PCR, microarray analysis, linear amplification techniques such as nucleic acid sequence based amplification (NASBA) are all amenable for use with the methods described herein, and can be carried out according to methods known to those of ordinary skill in the art.

In another embodiment, the sample, or a portion thereof is subjected to a quantitative polymerase chain reaction (PCR) for detecting the presence and abundance of a first marker and/or a second marker (FIG. 31, 1003-1004; FIG. 32, 2003-2004). Specific microorganism strains activity is measured by reverse transcription of transcribed ribosomal and/or messenger RNA (rRNA and mRNA) into complementary DNA (cDNA), followed by PCR (RT-PCR).

In another embodiment, the sample, or a portion thereof is subjected to PCR-based fingerprinting techniques to detect the presence and abundance of a first marker and/or a second marker (FIG. 31, 1003-1004; FIG. 32, 2003-2004). PCR products can be separated by electrophoresis based on the nucleotide composition. Sequence variation among the different DNA molecules influences the melting behavior, and therefore molecules with different sequences will stop migrating at different positions in the gel. Thus electrophoretic profiles can be defined by the position and the relative intensity of different bands or peaks and can be translated to numerical data for calculation of diversity indices. Bands can also be excised from the gel and subsequently sequenced to reveal the phylogenetic affiliation of the community members. Electrophoresis methods include, but are not limited to: denaturing gradient gel electrophoresis (DGGE), temperature gradient gel electrophoresis (TGGE), single-stranded-conformation polymorphism (SSCP), restriction fragment length polymorphism analysis (RFLP) or amplified ribosomal DNA restriction analysis (ARDRA), terminal restriction fragment length polymorphism analysis (T-RFLP), automated ribosomal intergenic spacer analysis (ARISA), randomly amplified polymorphic DNA (RAPD), DNA amplification fingerprinting (DAF) and Bb-PEG electrophoresis.

In another embodiment, the sample, or a portion thereof is subjected to a chip-based platform such as microarray or microfluidics to determine the abundance of a unique first marker and/or presence/abundance of a unique second marker (FIG. 31, 1003-1004, FIG. 32, 2003-2004). The PCR products are amplified from total DNA in the sample and directly hybridized to known molecular probes affixed to microarrays. After the fluorescently labeled PCR amplicons are hybridized to the probes, positive signals are scored by the use of confocal laser scanning microscopy. The microarray technique allows samples to be rapidly evaluated with replication, which is a significant advantage in microbial community analyses. In general, the hybridization signal intensity on microarrays is directly proportional to the abundance of the target organism. The universal high-density 16S microarray (PhyloChip) contains about 30,000 probes of 16SrRNA gene targeted to several cultured microbial species and “candidate divisions”. These probes target all 121 demarcated prokaryotic orders and allow simultaneous detection of 8,741 bacterial and archacal taxa. Another microarray in use for profiling microbial communities is the Functional Gene Array (FGA). Unlike PhyloChips, FGAs are designed primarily to detect specific metabolic groups of bacteria. Thus, FGA not only reveal the community structure, but they also shed light on the in situ community metabolic potential. FGA contain probes from genes with known biological functions, so they are useful in linking microbial community composition to ecosystem functions. An FGA termed GeoChip contains >24,000 probes from all known metabolic genes involved in various biogeochemical, ecological, and environmental processes such as ammonia oxidation, methane oxidation, and nitrogen fixation.

A protein expression assay, in one embodiment, is used with the methods described herein for determining the level of expression of one or more second markers (FIG. 31, 1004; FIG. 32, 2004). For example, in one embodiment, mass spectrometry or an immunoassay such as an enzyme-linked immunosorbant assay (ELISA) is utilized to quantify the level of expression of one or more unique second markers, wherein the one or more unique second markers is a protein.

In one embodiment, the sample, or a portion thereof is subjected to Bromodeoxyuridine (BrdU) incorporation to determine the level of a second unique marker (FIG. 31, 1004; FIG. 32, 2004). BrdU, a synthetic nucleoside analog of thymidine, can be incorporated into newly synthesized DNA of replicating cells. Antibodies specific for BRdU can then be used for detection of the base analog. Thus BrdU incorporation identifies cells that are actively replicating their DNA, a measure of activity of a microorganism according to one embodiment of the methods described herein. BrdU incorporation can be used in combination with FISH to provide the identity and activity of targeted cells.

In one embodiment, the sample, or a portion thereof is subjected to microautoradiography (MAR) combined with FISH to determine the level of a second unique marker (FIG. 31, 1004; FIG. 32, 2004). MAR-FISH is based on the incorporation of radioactive substrate into cells, detection of the active cells using autoradiography and identification of the cells using FISH. The detection and identification of active cells at single-cell resolution is performed with a microscope. MAR-FISH provides information on total cells, probe targeted cells and the percentage of cells that incorporate a given radiolabelled substance. The method provides an assessment of the in situ function of targeted microorganisms and is an effective approach to study the in vivo physiology of microorganisms. A technique developed for quantification of cell-specific substrate uptake in combination with MAR-FISH is known as quantitative MAR (QMAR).

In one embodiment, the sample, or a portion thereof is subjected to stable isotope Raman spectroscopy combined with FISH (Raman-FISH) to determine the level of a second unique marker (FIG. 31, 1004; FIG. 32, 2004). This technique combines stable isotope probing, Raman spectroscopy and FISH to link metabolic processes with particular organisms. The proportion of stable isotope incorporation by cells affects the light scatter, resulting in measurable peak shifts for labelled cellular components, including protein and mRNA components. Raman spectroscopy can be used to identify whether a cell synthesizes compounds including, but not limited to: oil (such as alkanes), lipids (such as triacylglycerols (TAG)), specific proteins (such as heme proteins, metalloproteins), cytochrome (such as P450, cytochrome c), chlorophyll, chromophores (such as pigments for light harvesting carotenoids and rhodopsins), organic polymers (such as polyhydroxyalkanoates (PHA), polyhydroxybutyrate (PHB)), hopanoids, steroids, starch, sulfide, sulfate and secondary metabolites (such as vitamin B12).

In one embodiment, the sample, or a portion thereof is subjected to DNA/RNA stable isotope probing (SIP) to determine the level of a second unique marker (FIG. 31, 1004; FIG. 32, 2004). SIP enables determination of the microbial diversity associated with specific metabolic pathways and has been generally applied to study microorganisms involved in the utilization of carbon and nitrogen compounds. The substrate of interest is labelled with stable isotopes (such as 13C or 15N) and added to the sample. Only microorganisms able to metabolize the substrate will incorporate it into their cells. Subsequently, 13C-DNA and 15N-DNA can be isolated by density gradient centrifugation and used for metagenomic analysis. RNA-based SIP can be a responsive biomarker for use in SIP studies, since RNA itself is a reflection of cellular activity.

In one embodiment, the sample, or a portion thereof is subjected to isotope array to determine the level of a second unique marker (FIG. 31, 1004; FIG. 32, 2004). Isotope arrays allow for functional and phylogenetic screening of active microbial communities in a high-throughput fashion. The technique uses a combination of SIP for monitoring the substrate uptake profiles and microarray technology for determining the taxonomic identities of active microbial communities. Samples are incubated with a 14C-labeled substrate, which during the course of growth becomes incorporated into microbial biomass. The 14C-labeled rRNA is separated from unlabeled rRNA and then labeled with fluorochromes. Fluorescent labeled rRNA is hybridized to a phylogenetic microarray followed by scanning for radioactive and fluorescent signals. The technique thus allows simultaneous study of microbial community composition and specific substrate consumption by metabolically active microorganisms of complex microbial communities.

In one embodiment, the sample, or a portion thereof is subjected to a metabolomics assay to determine the level of a second unique marker (FIG. 31, 1004; FIG. 32, 2004). Metabolomics studies the metabolome which represents the collection of all metabolites, the end products of cellular processes, in a biological cell, tissue, organ or organism. This methodology can be used to monitor the presence of microorganisms and/or microbial mediated processes since it allows associating specific metabolite profiles with different microorganisms. Profiles of intracellular and extracellular metabolites associated with microbial activity can be obtained using techniques such as gas chromatography-mass spectrometry (GC-MS). The complex mixture of a metabolomic sample can be separated by such techniques as gas chromatography, high performance liquid chromatography and capillary electrophoresis. Detection of metabolites can be by mass spectrometry, nuclear magnetic resonance (NMR) spectroscopy, ion-mobility spectrometry, electrochemical detection (coupled to HPLC) and radiolabel (when combined with thin-layer chromatography).

According to the embodiments described herein, the presence and respective number of one or more active microorganism strains in a sample are determined (FIG. 31, 1006; FIG. 32, 2006). For example, strain identity information obtained from assaying the number and presence of first markers is analyzed to determine how many occurrences of a unique first marker are present, thereby representing a unique microorganism strain (e.g., by counting the number of sequence reads in a sequencing assay). This value can be represented in one embodiment as a percentage of total sequence reads of the first maker to give a percentage of unique microorganism strains of a particular microorganism type. In a further embodiment, this percentage is multiplied by the number of microorganism types (obtained at step 1002 or 2002, see FIG. 31 and FIG. 32) to give the absolute abundance of the one or more microorganism strains in a sample and a given volume.

The one or more microorganism strains are considered active, as described above, if the level of second unique marker expression at a threshold level, higher than a threshold value, e.g., higher than at least about 5%, at least about 10%, at least about 20% or at least about 30% over a control level.

In another aspect of the invention, a method for determining the absolute abundance of one or more microorganism strains is determined in a plurality of samples (FIG. 32, see in particular, 2007). For a microorganism strain to be classified as active, it need only be active in one of the samples. The samples can be taken over multiple time points from the same source, or can be from different environmental sources (e.g., different animals).

The absolute abundance values over samples are used in one embodiment to relate the one or more active microorganism strains, with an environmental parameter (FIG. 32, 2008). In one embodiment, the environmental parameter is the presence of a second active microorganism strain. Relating the one or more active microorganism strains to the environmental parameter, in one embodiment, is carried out by determining the co-occurrence of the strain and parameter by correlation or by network analysis.

In one embodiment, determining the co-occurrence of one or more active microorganism strains with an environmental parameter comprises a network and/or cluster analysis method to measure connectivity of strains or a strain with an environmental parameter within a network, wherein the network is a collection of two or more samples that share a common or similar environmental parameter. In another embodiment, the network and/or cluster analysis method may be applied to determining the co-occurrence of two or more active microorganism strains in a sample (FIG. 32, 2008). In another embodiment, the network analysis comprises nonparametric approaches including mutual information to establish connectivity between variables. In another embodiment, the network analysis comprises linkage analysis, modularity analysis, robustness measures, betweenness measures, connectivity measures, transitivity measures, centrality measures or a combination thereof (FIG. 32, 2009). In another embodiment, the cluster analysis method comprises building a connectivity model, subspace model, distribution model, density model, or a centroid model and/or using community detection algorithms such as the Louvain, Bron-Kerbosch, Girvan-Newman, Clauset-Newman-Moore, Pons-Latapy, and Wakita-Tsurumi algorithms (FIG. 32, 2010).

In one embodiment, the cluster analysis method is a heuristic method based on modularity optimization. In a further embodiment, the cluster analysis method is the Louvain method. Sec, e.g., the method described by Blondel et al. (2008). Fast unfolding of communities in large networks. Journal of Statistical Mechanics: Theory and Experiment, Volume 28 Oct. 2008, incorporated by reference herein in its entirety for all purposes.

In another embodiment, the network analysis comprises predictive modeling of network through link mining and prediction, collective classification, link-based clustering, relational similarity, or a combination thereof. In another embodiment, the network analysis comprises differential equation based modeling of populations. In another embodiment, the network analysis comprises Lotka-Volterra modeling.

In one embodiment, relating the one or more active microorganism strains to an environmental parameter (e.g., determining the co-occurrence) in the sample comprises creating matrices populated with linkages denoting environmental parameter and microorganism strain associations.

In one embodiment, the multiple sample data obtained at step 2007 (e.g., over two or more samples which can be collected at two or more time points where each time point corresponds to an individual sample), is compiled. In a further embodiment, the number of cells of each of the one or more microorganism strains in each sample is stored in an association matrix (which can be in some embodiments, an abundance matrix). In one embodiment, the association matrix is used to identify associations between active microorganism strains in a specific time point sample using rule mining approaches weighted with association (e.g., abundance) data. Filters are applied in one embodiment to remove insignificant rules.

In one embodiment, the absolute abundance of one or more, or two or more active microorganism strains is related to one or more environmental parameters (FIG. 32, 2008), e.g., via co-occurrence determination. Environmental parameters are chosen by the user depending on the sample(s) to be analyzed and are not restricted by the methods described herein. The environmental parameter can be a parameter of the sample itself, e.g., pH, temperature, amount of protein in the sample. Alternatively, the environmental parameter is a parameter that affects a change in the identity of a microbial community (i.e., where the “identity” of a microbial community is characterized by the type of microorganism strains and/or number of particular microorganism strains in a community), or is affected by a change in the identity of a microbial community. In one embodiment, the environmental parameter is the presence, activity, and/or abundance of a second microorganism strain in the microbial community, present in the same sample.

In some embodiments described herein, an environmental parameter is referred to as a metadata parameter.

Other examples of metadata parameters include but are not limited to genetic information from the host from which the sample was obtained (e.g., DNA mutation information), sample pH, sample temperature, expression of a particular protein or mRNA, nutrient conditions (e.g., level and/or identity of one or more nutrients) of the surrounding environment/ecosystem), susceptibility or resistance to disease, onset or progression of disease, susceptibility or resistance of the sample to toxins, efficacy of xenobiotic compounds (pharmaceutical drugs), biosynthesis of natural products, or a combination thereof.

For example, according to one embodiment, microorganism strain number changes are calculated over multiple samples according to the method of FIG. 32 (i.e., at 2001-2007). Strain number changes of one or more active strains over time is compiled (e.g., one or more strains that have initially been identified as active according to step 2006), and the directionality of change is noted (i.e., negative values denoting decreases, positive values denoting increases). The number of cells over time is represented as a network, with microorganism strains representing nodes and the abundance weighted rules representing edges. Markov chains and random walks are leveraged to determine connectivity between nodes and to define clusters. Clusters in one embodiment are filtered using metadata in order to identify clusters associated with desirable metadata (FIG. 32, 2008).

In a further embodiment, microorganism strains are ranked according to importance by integrating cell number changes over time and strains present in target clusters, with the highest changes in cell number ranking the highest.

Network and/or cluster analysis method in one embodiment, is used to measure connectivity of the one or more strains within a network, wherein the network is a collection of two or more samples that share a common or similar environmental parameter. In one embodiment, network analysis comprises linkage analysis, modularity analysis, robustness measures, betweenness measures, connectivity measures, transitivity measures, centrality measures, or a combination thereof. In another embodiment, network analysis comprises predictive modeling of network through link mining and prediction, social network theory, collective classification, link-based clustering, relational similarity, or a combination thereof. In another embodiment, network analysis comprises differential equation based modeling of populations. In yet another embodiment, network analysis comprises Lotka-Volterra modeling.

Cluster analysis method comprises building a connectivity model, subspace model, distribution model, density model, or a centroid model.

Network and cluster based analysis, for example, to carry out method step 2008 of FIG. 32, can be carried out via a module. As used herein, a module can be, for example, any assembly, instructions and/or set of operatively-coupled electrical components, and can include, for example, a memory, a processor, electrical traces, optical connectors, software (executing in hardware) and/or the like.

In some embodiments, a network and/or cluster analysis method is used to measure connectivity of the one or more strains within a network, wherein the network is a collection of two or more samples that share a common or similar environmental parameter. In one embodiment, network analysis comprises linkage analysis, modularity analysis, robustness measures, betweenness measures, connectivity measures, transitivity measures, centrality measures, or a combination thereof. In another embodiment, network analysis comprises predictive modeling of network through link mining and prediction, social network theory, collective classification, link-based clustering, relational similarity, or a combination thereof. In another embodiment, network analysis comprises mutual information, maximal information coefficient (MIC) calculations, or other nonparametric methods between variables to establish connectivity. In another embodiment, network analysis comprises differential equation based modeling of populations. In yet another embodiment, network analysis comprises Lotka-Volterra modeling.

The environmental parameter can be a parameter of the sample itself, e.g., pH, temperature, amount of protein in the sample. Alternatively, the environmental parameter is a parameter that affects a change in the identity of a microbial community (i.e., where the “identity” of a microbial community is characterized by the type of microorganism strains and/or number of particular microorganism strains in a community), or is affected by a change in the identity of a microbial community. For example, in some embodiments, an environmental parameter is the food intake of an animal or the amount of eggs produced. In some embodiments, the environmental parameter is the presence, activity and/or abundance of a second microorganism strain in the microbial community, present in the same sample. In some embodiments, an environmental parameter is referred to as a metadata parameter.

Other examples of metadata parameters include but are not limited to genetic information from the host from which the sample was obtained (e.g., DNA mutation information), sample pH, sample temperature, expression of a particular protein or mRNA, nutrient conditions (e.g., level and/or identity of one or more nutrients) of the surrounding environment/ecosystem), susceptibility or resistance to disease, onset or progression of disease, susceptibility or resistance of the sample to toxins, efficacy of xenobiotic compounds (pharmaceutical drugs), biosynthesis of natural products, or a combination thereof.

The term maximal information coefficient or “MIC” refers to a type of nonparametric analysis that identifies a score between active microbial strains of the present disclosure and at least one measured metadata (e.g., increase in weight). The results from the nonparametric analysis are pooled to create a list of all relationships and their corresponding MIC scores. If the relationship scores below a given threshold, the relationship is deemed/identified as irrelevant. If the relationship is above a given threshold, the relationship deemed/identified as relevant, and is further subject to network analysis. Methods of determining MIC scores are further described in U.S. Pat. No. 9,540,676, which is hereby incorporated by reference in its entirety.

The following code fragment shows an exemplary methodology for MIC analysis, according to one embodiment:

```
Read total list of relationships file as links
threshold = 0.8
for i in range(len(links)):
  if links >= threshold
    multiplier[i] = 1
  else
    multiplier[i] = 0
end if
links_temp = multiplier*links
final_links = links_temp[links_temp != 0]
savetxt(output_file,final_links)
output_file.close( )
```

With regard to MIC scores, a cut-off based on this score is used to define useful and non-useful microorganisms with respect to the improvement of specific traits. The point at which the data points on the curve move transition from the log scale to the linear scale (with regard to the slope) is the inflection point. The organisms with MIC scores that fall below the inflection point are generally non-useful, while the organisms with MIC scores that are found above the inflection point are generally useful, as it pertains to the specific characteristic being evaluated for the MIC score.

In some embodiments, the compositions of the present disclosure comprise one or more bacteria that have a MIC score of at least about 0.1, 0.15, 0.2, 0.25, 0.3, 0.35, 0.4, 0.45, 0.5, 0.55, 0.6, 0.7, 0.75, 0.8, 0.85, 0.9, or 0.95. In some embodiments, the isolated bacteria selected for inclusion in the microbial compositions described herein comprise a MIC score of at least 0.2.

Based on the output of the network analysis, active strains are selected for preparing products (e.g., ensembles, aggregates, and/or other synthetic groupings) containing the selected strains. The output of the network analysis can also be used to inform the selection of strains for further product composition testing. Thresholds can be, depending on the implementation and application: (1) empirically determined (e.g., based on distribution levels, setting a cutoff at a number that removes a specified or significant portion of low level reads); (2) any non-zero value; (3) percentage/percentile based; (4) only strains whose normalized second marker (i.e., activity) reads is greater than normalized first marker (cell count) reads; (5) log 2 fold change between activity and quantity or cell count; (6) normalized second marker (activity) reads is greater than mean second marker (activity) reads for entire sample (and/or sample set); and/or any magnitude threshold described above in addition to a statistical threshold (i.e., significance testing). The following example provides thresholding detail for distributions of RNA-based second marker measurements with respect to DNA-based first marker measurements, according to one embodiment.

### Microbial Culture Techniques

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores acrobically/anaerobically, cellular morphology, carbon source metabolism, acid/base production, enzyme secretion, metabolic secretions, etc.) and to reduce the likelihood of working with a culture which has become contaminated.

For example, for microbes of the disclosure, biologically pure isolates can be obtained through repeated subculture of biological samples, each subculture followed by streaking onto solid media to obtain individual colonies or colony forming units. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze dried liquid formulations and cultures stored long term at −70° C. in solutions containing glycerol are contemplated for use in providing formulations of the present disclosure.

The microbes of the disclosure can be propagated in a liquid medium under aerobic conditions, or alternatively anaerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the microbes include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 g/L. Preferably, glucose, starch, and/or peptone is contained in the medium as a major carbon source, at a concentration of 0.1-5% (W/V).

Examples of suitable nitrogen sources which can be used for growing the bacterial strains of the present disclosure include, but are not limited to, amino acids, yeast extract, tryptone, beef extract, peptone, potassium nitrate, ammonium nitrate, ammonium chloride, ammonium sulfate, ammonium phosphate, ammonia, or combinations thereof. The amount of nitrogen source varies according to the type of nitrogen source, typically between 0.1 g/L to 30 g/L.

The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 g/L to 10 g/L. Examples of specially required substances include, but are not limited to, vitamins, nucleic acids, yeast extract, peptone, meat extract, malt extract, dried yeast, and combinations thereof.

Cultivation can be effected at a temperature, which allows the growth of the microbial strains, essentially, between 20° C. and 46° C. In some aspects, a temperature range is 30° C.-39° C. For optimal growth, in some embodiments, the medium can be adjusted to pH 6.0-7.4. It will be appreciated that commercially available media may also be used to culture the microbial strains, such as Nutrient Broth or Nutrient Agar available from Difco, Detroit, MI. It will be appreciated that cultivation time may differ depending on the type of culture medium used and the concentration of sugar as a major carbon source.

In some aspects, cultivation lasts between 8-96 hours. Microbial cells thus obtained are isolated using methods which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described herein above. In some aspects, microbial multi-strain cultures may be obtained by propagating two or more of the strains described hereinabove. It will be appreciated that the microbial strains may be cultured together when compatible culture conditions can be employed.

### Methods of Use

In some embodiments, the present disclosure provides a method of treating and/or preventing colic in an equine comprising administering a microbial composition described herein to an equine in need thereof in an amount effective to prevent and/or treat colic in the equine administered the microbial composition compared to an equine that was not administered the microbial composition.

In some embodiments, the microbial composition is administered to the equine daily for at least 1, 2, 3, 4, 5, 6, 7, 8, 9, 10 days or longer. In some embodiments, the microbial composition is administered to the equine daily for at least 1, 2, 3, 4, 5, 6, 7, 8, 9, 10, 11, 12, 13, 14, 15, 16, 17, 18, 19, 20 weeks or longer. In some embodiments, the microbial composition is administered to the equine daily for at least 1, 2, 3, 4, 5, 6, 7, 8, 9, 10, 11, 12 months or longer.

In some embodiments, the microbial compositions described herein are administered by fecal microbiota transplant via nasogastric intubation, fecal enema, direct injection of microbial suspension into intestines or colon during surgery, as a liquid formulation or bolus injection, as a small pills administered with food, as a powder sprinkled on feed, or as an in-feed pellet. In some embodiments, the microbial compositions are administered with one or more additional therapeutic agents or interventions. For example, in some embodiments, the microbial compositions are administered with an antibiotic, a proton pump inhibitor, and/or food. In some embodiments, the microbial compositions are administered after the administration of an antibiotic, a proton pump inhibitor, and/or food. In such embodiments, the administration of antibiotics, proton pump inhibitors with or just prior to the compositions described herein raises the pH of the equine stomach, therefore enabling the microbes present in the composition to persist for longer periods of time after administration. In some embodiments, the pH of the stomach is increased by at least 0.2, at least 0.4, at least 0.6, at least 0.8, at least 1, at least 1.2, at least 1.4, at least 1.6, at least 1.8, at least 2, at least 2.2, at least 2.4, at least 2.6, at least 2.8, at least 3, at least 3.2, at least 3.4, at least 3.6, at least 3.8, at least 4, at least 4.2, at least 4.4, at least 4.6, at least 4.8, at least 5, at least 5.2, at least 5.4, at least 5.6, at least 5.8, at least 6, at least 6.2, at least 6.4, at least 6.6, at least 6.8, or at least 7. In some embodiments, the microbial compositions are administered before, during, or after a surgical procedure.

In some embodiments, compositions of the present disclosure are administered to competitively exclude microbial pathogens from causing a disease state in equines. In some embodiments, administration of the compositions described herein prevents pathogenic microbes from outcompeting the non-pathogenic microbes present in the composition in the stomach and/or gastrointestinal tract of the equine. In some embodiments, compositions of the present disclosure competitively bind molecules of the glycocalyx/extracellular matrix of the gut cell walls to preclude or competitively inhibit pathogens from adhering to lectins and other molecules such as collagens (particularly types-III, IV, and V), gelatin, fibrinogen, laminin, and vitronectin. Pathogen adherence to these molecules are believed to contribute to the virulence of the pathogens.

In some embodiments, administration of compositions of the present disclosure in a decrease in the binding of pathogenic microbes to the glycocalyx/extracellular matrix of the cells of the equine gastrointestinal tract. In some embodiments, the compositions of the present disclosure result in the binding of the administered microbes to the glycocalyx/extracellular matrix, preventing pathogenic microbes from adhering to the glycocalyx/extracellular matrix and preventing pathogenic disease. In some embodiments, the compositions of the present disclosure result in the chemical modification of the molecules of the glycocalyx/extracellular matrix by the administered microbial composition, preventing pathogenic microbes from adhering to the glycocalyx/extracellular matrix and preventing pathogenic disease. In some embodiments, the molecules bound or chemically modified by the administered microbes are selected from lectins, collagens, gelatins, fibrinogens, laminins, and vitronectins.

In some embodiments, the administration of microbial compositions of the present disclosure to equines stimulate the production of B cells. In some embodiments, the administration of microbial compositions of the present disclosure to equines result in an increase of one or more types of B cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the administration of microbial compositions of the present disclosure to equines activates B cells. In some embodiments, administration of microbial compositions of the present disclosure to equines result in an increase in activation of one or more types of B cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, B cells are selected from regulatory B cells, B-1 cells, B-2 cells, marginal zone B cells, follicular B cells, memory B cells, plasma cells, and plasmablasts.

In some embodiments, the administration of microbial compositions of the present disclosure to equines stimulate the production of T cells. In some embodiments, the administration of microbial compositions of the present disclosure to equines result in an increase of one or more types of T cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the administration of microbial compositions of the present disclosure to equines activates T cells. In some embodiments, administration of microbial compositions of the present disclosure to equines result in an increase in activation of one or more types of T cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, T cells are selected from γδ (gamma delta) T cells, αβ (alpha beta) T cells, natural killer T cells, regulatory T cells, memory T cells, cytotoxic T cells, helper T cells, and effector T cells.

In some embodiments, the administration of microbial compositions of the present disclosure to equines activates antigen-presenting cells. In some embodiments, administration of microbial compositions of the present disclosure to equines results in an increase in activation of one or more types of antigen-presenting cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, antigen-presenting cells are selected from dendritic cells, macrophages, B cells, or innate lymphoid cells.

In some embodiments, the administration of microbial compositions of the present disclosure to equines results in an increase in the number of isolated lymphoid follicles (ILFs). In some embodiments, the administration of microbial compositions of the present disclosure to equines results in an increase of isolated lymphoid follicles by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the administration of microbial compositions of the present disclosure result in the modulation of the gene expression of mucins, tight junction polypeptides, and cytokines. In some embodiments, the modulation of the gene expression of mucins, tight junction polypeptides, and cytokines results in an increase of the gene expression of said molecules. In some embodiments, the modulation of the gene expression of mucins, tight junction polypeptides, and cytokines results in a decrease of the gene expression of said molecules.

In some embodiments, administration of the microbial compositions of the present disclosure results in a decrease in the expression of mucins. In some embodiments, the mucins are selected from MUC1, MUC2, MUC4, MUC5AC, MUC5B, MUC6, MUC13, and MUC16.

In some embodiments, administration of the microbial compositions of the present disclosure results in a decrease in the expression of cytokines. In some embodiments, the cytokines are selected from granulocyte-macrophage stimulating factor (GM-CSF), IL-IRA, IL-1a, IL-1B, IL-2, IL-4, IL-6, IL-10, IL-11, IL-12, IL-13, IL-17A, IL-17D, IL-17E, IL-17F, IL-18, IL-22, IL-23, tumor necrosis factor (TNF), interferon beta (IFN-β), IFN-γ, and IFN-2.

In some embodiments, the administration of microbial compositions of the present disclosure result in a decrease of gut inflammation in equines, as measured by the serum levels of inflammation markers. In some embodiment, the inflammation markers are selected from al-acid glycoprotein (AGP), IL-8, IL-1B, IL-17A, IL-17F, transforming growth factor (TGF-β4), fatty acid-binding protein (FABP2), C-reactive protein, haptoglobin, ceruloplasmin, hemopexin, and serum amyloid A.

In some embodiments, the methods provided herein prevent or reduce one or more symptoms of colic in an equine. For example, in some embodiments, the methods prevent or reduce one or more symptoms selected from abdominal pain, stomach irritation, rolling, kicking at stomach, distension of GI organs, and decreased eating. In some embodiments, the methods provided herein reduce the frequency with which colic occurs in an equine. For example, in some embodiments, the methods provided herein decrease the frequency of colic episodes in an equine administered the compositions described herein compared to the frequency of colic episodes observed in an equine that has not been administered the compositions described herein.

### FURTHER NUMBERED EMBODIMENTS

Further numbered embodiments of the present disclosure are provided as follows:

Embodiment 1. A microbial composition comprising: one or more bacteria with a 16S nucleic acid sequence that is at least 97% identical to a nucleic acid sequence selected from SEQ ID NOs: 1-574; and a carrier suitable for equine administration.

Embodiment 2. The microbial composition of Embodiment 1, comprising one or more bacteria with a 16S nucleic acid sequence that is at least 97%, at least 98%, or at least 99% identical to a nucleic acid sequence selected from the group consisting of SEQ ID NO: 5, SEQ ID NO: 141, SEQ ID NO: 319, SEQ ID NO: 426, and SEQ ID NO: 475.

Embodiment 3. The microbial composition of Embodiment 1, comprising one or more bacteria with a 16S nucleic acid sequence comprising or consisting of a nucleic acid sequence selected from the group consisting of SEQ ID NO: 5, SEQ ID NO: 141, SEQ ID NO: 319, SEQ ID NO: 426, and SEQ ID NO: 475.

Embodiment 4. The microbial composition of Embodiment 1, comprising one or more bacteria with a 16S nucleic acid sequence that is at least 97%, at least 98%, or at least 99% identical to a nucleic acid sequence selected from the group consisting of SEQ ID NO: 11, SEQ ID NO: 142, SEQ ID NO: 320, SEQ ID NO: 433, and SEQ ID NO: 476.

Embodiment 5. The microbial composition of Embodiment 1, comprising one or more bacteria with a 16S nucleic acid sequence comprising or consisting of a nucleic acid sequence selected from the group consisting of SEQ ID NO: 11, SEQ ID NO: 142, SEQ ID NO: 320, SEQ ID NO: 433, and SEQ ID NO: 476.

Embodiment 6. The microbial composition of Embodiment 1, comprising two, three, four, five, or more bacteria with a 16S nucleic acid sequence that is at least 97% identical to a nucleic acid sequence selected from SEQ ID NOs: 1-574.

Embodiment 7. The microbial composition of Embodiment 1, comprising two, three, four, or five bacteria with a 16S nucleic acid sequence that is at least 97%, at least 98%, or at least 99% identical to a nucleic acid sequence selected from the group consisting of SEQ ID NO: 5, SEQ ID NO: 141, SEQ ID NO: 319, SEQ ID NO: 426, and SEQ ID NO: 475.

Embodiment 8. The microbial composition of Embodiment 1, comprising two, three, four, or five bacteria with a 16S nucleic acid sequence comprising or consisting of a nucleic acid sequence selected from the group consisting of SEQ ID NO: 5, SEQ ID NO: 141, SEQ ID NO: 319, SEQ ID NO: 426, and SEQ ID NO: 475.

Embodiment 9. The microbial composition of Embodiment 1, comprising two, three, four, or five bacteria with a 16S nucleic acid sequence that is at least 97%, at least 98%, or at least 99% identical to a nucleic acid sequence selected from the group consisting of SEQ ID NO: 11, SEQ ID NO: 142, SEQ ID NO: 320, SEQ ID NO: 433, and SEQ ID NO: 476.

Embodiment 10. The microbial composition of Embodiment 1, comprising two, three, four, or five bacteria with a 16S nucleic acid sequence comprising or consisting of a nucleic acid sequence selected from the group consisting of SEQ ID NO: 11, SEQ ID NO: 142, SEQ ID NO: 320, SEQ ID NO: 433, and/or SEQ ID NO: 476.

Embodiment 11. A microbial composition comprising: one or more bacterium selected from a Clostridium spp. bacterium; a Streptococcus spp. bacterium; an Escheria spp. bacterium; and an Atlantibacter spp. bacterium; and a carrier suitable for equine administration.

Embodiment 12. A microbial composition comprising: one or more bacterium selected from a Clostridium butyricum bacterium; a Streptococcus equinis bacterium; an Escheria coli bacterium; a Clostridium maximum bacterium; and an Atlantibacter hermannii bacterium; and a carrier suitable for equine administration.

Embodiment 13. The microbial composition of Embodiment 12, wherein: the Clostridium butyricum bacterium comprises a 16S nucleic acid sequence that is at least 97%, 98%, or 99% identical to one of SEQ ID NOs: 5-13; the Streptococcus equinis bacterium comprises a 16S nucleic acid sequence that is at least 97%, 98%, or 99% identical to one of SEQ ID NOs: 143-150; theEscheria coli bacterium comprises a 16S nucleic acid sequence that is at least 97%, 98%, or 99% identical to one of SEQ ID NOs: 321-328; the Clostridium maximum bacterium comprises a 16S nucleic acid sequence that is at least 97%, 98%, or 99% identical to one of SEQ ID NOs: 430-437; and/or the Atlantibacter hermannii bacterium comprises a 16S nucleic acid sequence that is at least 97%, 98%, or 99% identical to one of SEQ ID NOs: 480-486.

Embodiment 14. The microbial composition of Embodiment 12, wherein: the Clostridium butyricum bacterium comprises a 16S nucleic acid sequence comprising or consisting of one of SEQ ID NOs: 5-13; the Streptococcus equinis bacterium comprises a 16S nucleic acid sequence comprising or consisting of one of SEQ ID NOs: 143-150; theEscheria coli bacterium comprises a 16S nucleic acid sequence comprising or consisting of one of SEQ ID NOs: 321-328; the Clostridium maximum bacterium comprises a 16S nucleic acid sequence comprising or consisting of one of SEQ ID NOs: 430-437; and/or the Atlantibacter hermannii bacterium comprises a 16S nucleic acid sequence comprising or consisting of one of SEQ ID NOs: 480-486.

Embodiment 15. The microbial composition of any one of Embodiments 12-14, wherein: the Clostridium butyricum bacterium comprises a 16S nucleic acid sequence that is at least 97%, 98%, or 99% identical to SEQ ID NO: 5 or SEQ ID NO: 11; the Streptococcus equinis bacterium comprises a 16S nucleic acid sequence that is at least 97%, 98%, or 99% identical to SEQ ID NO: 141 or SEQ ID NO: 142; theEscheria coli bacterium comprises a 16S nucleic acid sequence that is at least 97%, 98%, or 99% identical to SEQ ID NO: 319 or SEQ ID NO: 320; the Clostridium maximum bacterium comprises a 16S nucleic acid sequence that is at least 97%, 98%, or 99% identical to SEQ ID NO: 426 or SEQ ID NO: 433; and/or the Atlantibacter hermannii bacterium comprises a 16S nucleic acid sequence that is at least 97%, 98%, or 99% identical SEQ ID NO: 475 or SEQ ID NO: 476.

Embodiment 16. The microbial composition of any one of Embodiments 12-14, wherein: the Clostridium butyricum bacterium comprises a 16S nucleic acid sequence comprising or consisting of SEQ ID NO: 5 or SEQ ID NO: 11; the Streptococcus equinis bacterium comprises a 16S nucleic acid sequence comprising or consisting of SEQ ID NO: 141 or SEQ ID NO: 142; theEscheria coli bacterium comprises a 16S nucleic acid sequence comprising or consisting of SEQ ID NO: 319 or SEQ ID NO: 320; the Clostridium maximum bacterium comprises a 16S nucleic acid sequence comprising or consisting of SEQ ID NO: 426 or SEQ ID NO: 433; and/or the Atlantibacter hermannii bacterium comprises a 16S nucleic acid sequence comprising or consisting of SEQ ID NO: 475 or SEQ ID NO: 476.

Embodiment 17. The microbial composition of any one of Embodiments 1-16, wherein the one or more bacteria has a MIC score of at least about 0.2.

Embodiment 18. The microbial composition of any one of Embodiments 1-17, wherein the equine is a domesticated equine or a wild equine.

Embodiment 19. The microbial composition of any one of Embodiments 1-18, wherein the equine is selected from a horse, a zebra, a mule, and a donkey.

Embodiment 20. The microbial composition of any one of Embodiments 1-19, wherein the carrier comprises a solidification agent and a sweeting agent.

Embodiment 21. The microbial composition of Embodiment 20, wherein the solidification agent is selected from xantham gum, agar, and gelatin.

Embodiment 22. The microbial composition of Embodiment 20, wherein the sweeting agent is selected from corn syrup, molasses, cane molasses, brewer's yeast, and honey.

Embodiment 23. The microbial composition of any one of Embodiments 1-22, wherein the composition is formulated as a gel, a liquid, a powder, a tablet, a capsule, a pill, a feed additive, a food ingredient, a food supplement, a water additive, a heat-stabilized additive, a moisture-stabilized additive, a pelleted feed additive, a pre-pelleted applied feed additive, a post-pelleted applied feed additive, or a spray additive.

Embodiment 24. The microbial composition of any one of Embodiments 1-23 wherein the composition is formulated for administration by injection, direct application to target organ, bolus administration, oral administration (such as with or as part of food), fecal enema, fecal microbiota transplant via nasogastric intubation

Embodiment 25. The microbial composition of any one of Embodiments 1-24, comprising the one or more bacteria in an amount effective to treat one or more symptoms of colic in an equine or to reduce the frequency of colic episodes.

Embodiment 26. A method for preventing and/or treating colic in an equine comprising administering the microbial composition of any one of Embodiments 1-25 to an equine in need thereof in an amount effective to prevent and/or treat colic in the equine administered the microbial composition compared to an equine that was not administered the microbial composition.

Embodiment 27. The method of Embodiment 26, wherein the equine is a domesticated equine or a wild equine.

Embodiment 28. The method of Embodiment 26 or Embodiment 27, wherein the equine is selected from a horse, a zebra, a mule, and a donkey.

Embodiment 29. The method of any one of Embodiments 26-28, wherein the microbial composition is administered daily for at least 1, 2, 3, 4, 5, 6, 7 days, or longer.

Embodiment 30. The method of any one of Embodiments 26-28, wherein the microbial composition is administered daily for at least 1 week, at least 2 weeks, at least 3 weeks, at least 4 weeks, at least 5 weeks, at least 6 weeks, at least 7 weeks, at least 8 weeks, or longer.

Embodiment 31. The method of any one of Embodiments 26-28, wherein the microbial composition is administered daily for at least 1 month, at least 2 months, at least 3 months, at least 4 months, at least 5 months, at least 6 months, or longer.

Embodiment 32. The method of any one of Embodiments 26-31, wherein the microbial composition is administered to the equine with an antibiotic, a proton pump inhibitor, and/or food.

Embodiment 33. The method of any one of Embodiments 26-31, wherein the microbial composition is administered to the equine after administration of an antibiotic, a proton pump inhibitor, and/or food.

Embodiment 34. The method of any one of Embodiments 26-33, wherein the administration of the microbial composition reduces one or more symptoms of colic selected from abdominal pain, stomach irritation, rolling, kicking at stomach, distension of GI organs, and decreased eating.

Embodiment 35. The method of any one of Embodiments 26-33, wherein the administration of the microbial composition reduces the frequency of colic episodes in an equine administered the microbial composition compared to an equine that has not been administered the microbial composition.

### EXAMPLES

The present disclosure is further illustrated by reference to the following Experimental Data and Examples. However, it should be noted that these Experimental Data and Examples, like the embodiments described above, are illustrative and are not to be construed as restricting the scope of the disclosure in any way.

### EXAMPLE 1: FORMULATION OF MICROBIAL COMPOSITIONS FOR ADMINISTRATION TO EQUINES

An equine microbial composition (comprising AscusEQ_4F (SEQ ID NO: 11), AscusEQ_61A (SEQ ID NO: 320), AscusEQ_140A (SEQ ID NO: 142), AscusEQ_414G (SEQ ID NO: 433), and AscusEQ_109A (SEQ ID NO: 476), referred to as Ascus Equine) was formulated for administration to equines. Briefly, the equine product is administered as a sweet paste delivered orally. The dose therefore needs high viscosity to delay oxygen permeation during administration and to provide time for the equine patient to consume the majority of the dose without loss of composition. Sodium alginate encapsulation of microbes was used to assist in delivery to hindgut by protecting cells against variable pH/low pH within equine gastric contents.

Solidification and sweetening: Gelatin and xantham gum tested for solidification viscosity at the following concentrations:
(a) Gelatin 0.1%-1.4%
(b) Xantham Gum 0.2%-2.0%
(c) Agar 0.25%-2.5%.

Concentrations of xantham gum higher than 1.4% gelled adequately. Agar concentrations of 1% were ideal. Other potential food grade thickening agents suitable for use during the solidification process include: starches, alginin, guar gum, collagen, pectin, and carboxymethyl cellulose. The solidified mixture is sweetened with corn syrup, molasses, cane molasses, brewer's yeast, and/or honey to be made palatable to horse. Salt (e.g. sodium chloride) also improves palatability.

Solidification/Sweetening carrier solution formulation: The solidification and sweetening agents are mixed together to form a carrier solution comprising water, cane molasses, sodium chloride, and 1.0% agar. A fluid gel carrier solution (Table 7) was created and then immediately post-autoclave, transferred to an anaerobic chamber and allowed to solidify while mixing by stir bar. Once solidified, the media was blended with an autoclaved overhead mixer until the correct liquid consistency is obtained. Using a serological pipet controller, 80 mLs aliquots were aliquoted into fluid gel carrier serum bottles.

TABLE 7
Fluid Gel Carrier Solution

| Component | g/L |
|---|---|
| Agar | 10.0 |
| NaCl | 2.0 |
| Molasses (Cane Sugar) | 1.5 |
| Deionized Water | Q.S. |

Place the components into a beaker 2× larger than the volume being made.
Use a stir-bar to mix. Do not remove the stir-bar.
pH to 7.2 ± 0.1
Cover the beaker with foil and autoclave at 121° C. for 15 minutes.
Immediately cycle into anaerobic chamber (15% CO₂, 5% H₂, 80% N₂)
Place on stir-plate and let solidify.
Once solid, blend with an upright mixer and autoclaved blades.

Microbial solution formulation: The microbial cells are prepared and stored separately. Individual strains are inoculated into anaerobic bottles and grown for 24 hours. Cells are enumerated and centrifuged to remove fermentation broth. Cells are resuspended in PBS and centrifuged to wash cell pellet. Cells are resuspended in a soy peptone/dextrose suspension solution for long term storage at 4° C.

Five equine product strains (AscusEQ_4F (SEQ ID NO: 11), AscusEQ_61A (SEQ ID NO: 320), AscusEQ_140A (SEQ ID NO: 142), AscusEQ_414G (SEQ ID NO: 433), and AscusEQ_109A (SEQ ID NO: 476) were inoculated into separate anaerobic tryptic soy broth bottles and incubated anaerobically for 24 hours. After incubation, cell concentrations were enumerated by Petroff-Hausser counting chamber. The appropriate amount of culture was then centrifuged at 4,300×g for 20 minutes at 4° C. Supernatants were decanted and cell pellets washed via resuspension in anaerobic PBS followed by centrifugation at 4,300×g for 20 minutes at 4° C. Supernatants were again decanted and cells resuspended in stability solution (Table 8). Strains were then combined and aliquoted in 20 mL aliquots into microbial solution serum bottles.

TABLE 8
Stability solution

| Component | g/L |
|---|---|
| NaCl | 8.0 |
| KCl | 0.2 |
| Na₂HPO₄ | 1.44 |
| KH₂PO₄ | 0.24 |
| K₂HPO₄ | 0.24 |
| Soy Peptone | 1.0 |
| Dextrose | 1.0 |
| Deionized Water | Q.S. |

pH to 7.2 ± 0.1
Pour contents into an appropriate autoclavable container
Autoclave at 121° C. for 15 minutes

Composition Administration: When ready to administer, sanitize the top of the microbial solution serum bottle with an alcohol wipe and withdraw 20 mL using a syringe (20 or 30 mL syringe) and needle (18G). Sanitize the top of the fluid gel carrier bottle and inject the 20 mL microbial solution into the fluid gel. Mix the two solutions by shaking vigorously for approximately 10 seconds. Remove the aluminum crimp seal and remove the stopper (Contents will be under slight pressure due to the 20 mL microbial solution addition). Remove the plunger from a 50+mL catheter tip syringe and shake 50 mLs of the solution into the syringe. Administer orally by inserting the nozzle of the syringe into the interdental space and depositing the appropriate amount. Table 9 provides the final composition per 50 mL dose.

TABLE 9
Composition components per 50 mL dose

| Component | CAS # | Concentration (per 50 mLs) | |
|---|---|---|---|
| AscusEQ_4F | N/A | 1.0 × 10⁹ | Cells |
| AscusEQ_61A | N/A | 1.0 × 10⁹ | Cells |
| AscusEQ_140A | N/A | 1.0 × 10⁹ | Cells |
| AscusEQ_414G | N/A | 1.0 × 10⁹ | Cells |
| AscusEQ_109A | N/A | 1.0 × 10⁹ | Cells |
| Agar | 9002-18-0 | 0.8 | grams |
| NaCl | 7647-14-5 | 0.32 | grams |
| Molasses (Sugar Cane) | N/A | 0.12 | grams |
| Na₂HPO₄ | 7558-79-4 | 28.8 | milligrams |
| Soy Peptone | N/A | 20.0 | milligrams |
| Dextrose | 50-99-7 | 20.0 | milligrams |
| KH₂PO₄ | 7778-77-0 | 4.8 | milligrams |
| K₂HPO₄ | 7758-11-4 | 4.8 | milligrams |
| KCl | 7447-40-7 | 4.0 | milligrams |

### Example 2: Case Studies with Microbial Composition Administration

Experiments were performed to gauge the efficacy of the microbial compositions described herein in reducing recurrent equine colic and to track microbial community changes.

Each horse enrolled in the study received one daily dose of an equine microbial composition (comprising AscusEQ_4F (SEQ ID NO: 11), AscusEQ_61A (SEQ ID NO: 320), AscusEQ_140A (SEQ ID NO: 142), AscusEQ_414G (SEQ ID NO: 433), and AscusEQ_109A (SEQ ID NO: 476), referred to as Ascus Equine) for 14 days. Fecal samples were collected daily from enrolled horses during the 14-day administration. Daily fecal sampling continued for 7 days after the final dose of the Ascus Equine microbial composition. An overview of Ascus Equine formulations, storage, and preparation conditions are provided below in Table 10.

TABLE 10
Summary of Equine Microbial Composition
Formulations and Testing Conditions

| Test article/Compound | |
|---|---|
| name: | Ascus Equine |
| Formulations: | Doses are packaged in two separate bottles. |
| | One fluid gel carrier (80 mL) and one microbial |
| | suspension in salt solution (20 mL). |
| Potency/concentration: | 50 mL per animal per day |
| Manufacturer: | Ascus Biosciences |
| Storage: | Both the liquid gel carrier and the microbial |
| | suspension are refrigerated at 4° C. until |
| | day of use. |
| Preparation: | 20 ml of microbial suspension are withdrawn |
| | using a needle and syringe, injected into the |
| | fluid gel carrier, and mixed thoroughly by |
| | shaking. The stopper is removed from fluid |
| | gel carrier bottle and 50 mL is dispensed into |
| | dosing syringe. The mixture is administered |
| | orally to horses. |
| Use conditions: | Dose to be prepared and administered by vet |
| | personnel at a dose of 50 mL per horse per day. |

Health Observations were performed by a vet throughout the 14-day administration period and on follow-up sampling dates. Additional observations were made if necessary depending on the needs of the patient. If additional colic episodes occur in the 60-day period following administration, additional unscheduled sample(s) were taken when the horse was admitted for colic evaluation.

Seven patients were treated according to the protocol outlined above. The initial health status of the patients is as follows:

(a) Patient #1: Experienced several episodes of colic in the past. Although not actively colicking at the start of microbe administration, the patient's fecal microbiome revealed that the patient was in a transient colic state, which explains the reoccuring colic episodes.

(b) Patient #2: Not actively colicking, and did not have frequent colic episodes in the past. Machine learning revealed that the patient was in a healthy state at the onset of microbe administration.

(c) Patient #3: Not actively colicking, and did not have frequent colic episodes in the past. Machine learning revealed that the patient was in a healthy state at the onset of microbe administration.

(d) Patient #4: Experienced several episodes of colic in the past. Actively colicking at the start of microbe administration.

(e) Patient #5: Experienced several episodes of colic in the past. Although not actively colicking at the start of microbe administration, the patient's fecal microbiome revealed that the patient was in a transient colic state, which explains the reoccuring colic episodes.

(f) Patient #6: Not actively colicking, and did have frequent colic episodes in the past. Machine learning revealed that the patient was in a transient, non-colicking/asymptomatic state at the onset of microbe administration.

(g Patient #7: Not actively colicking, and did not have frequent colic episodes in the past. Machine learning revealed that the patient was in a transient, non-colicking/asymptomatic state at the onset of microbe administration.

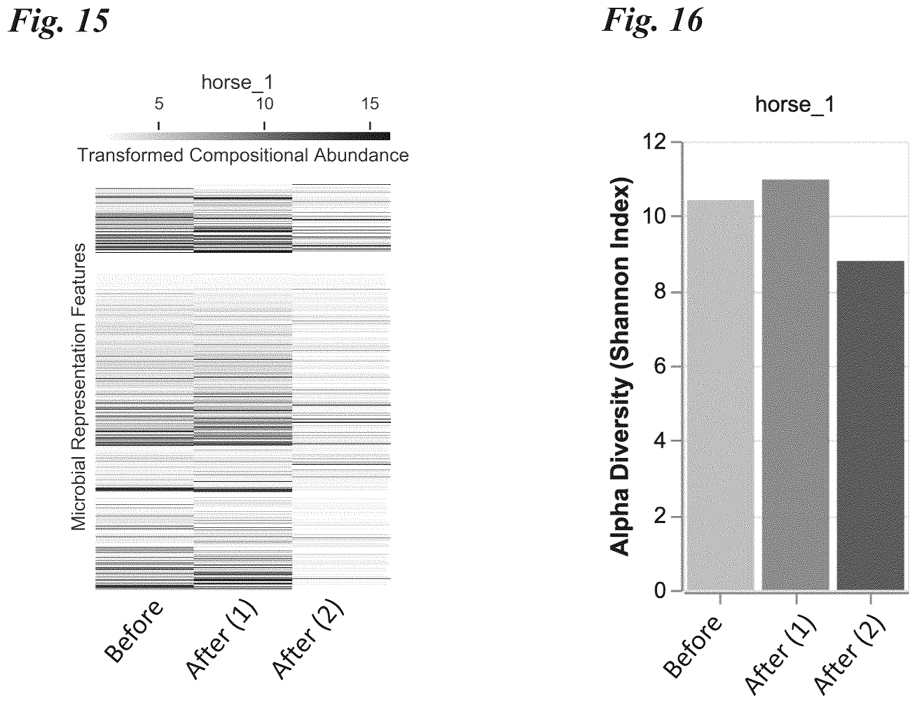

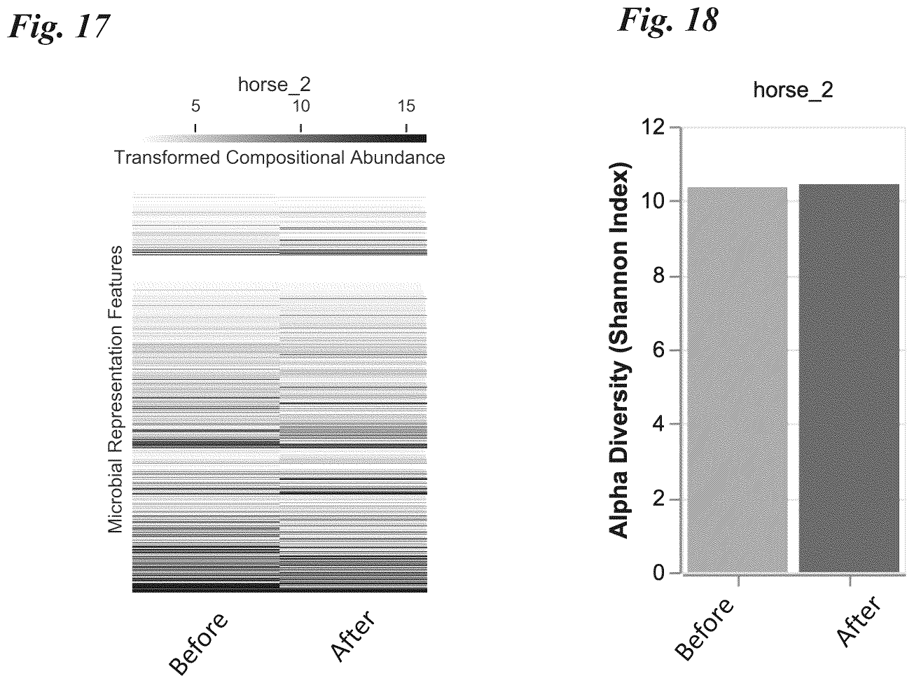

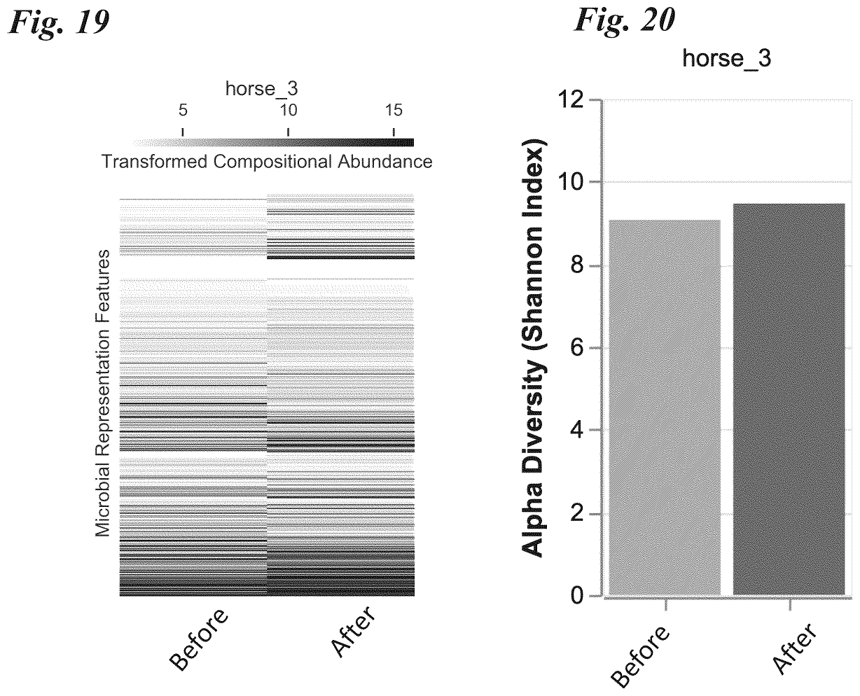

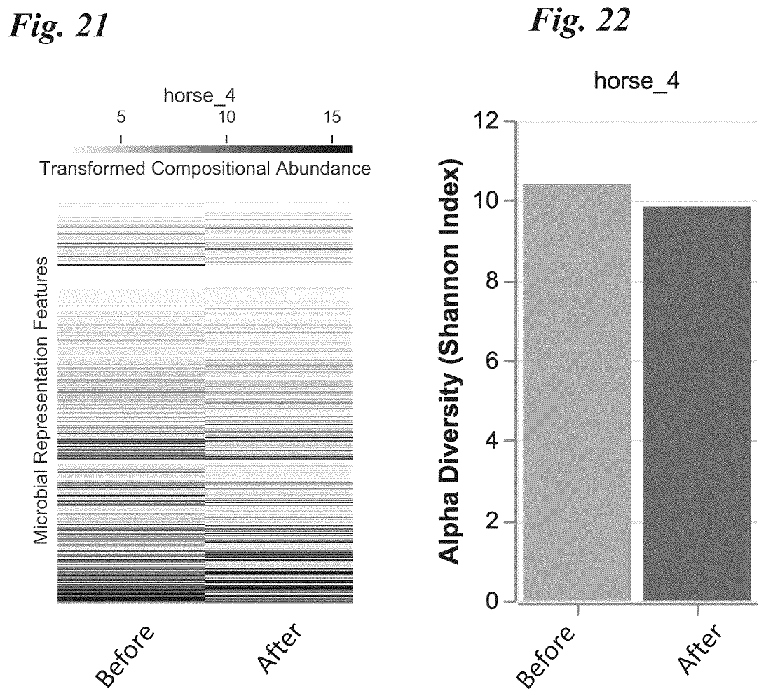

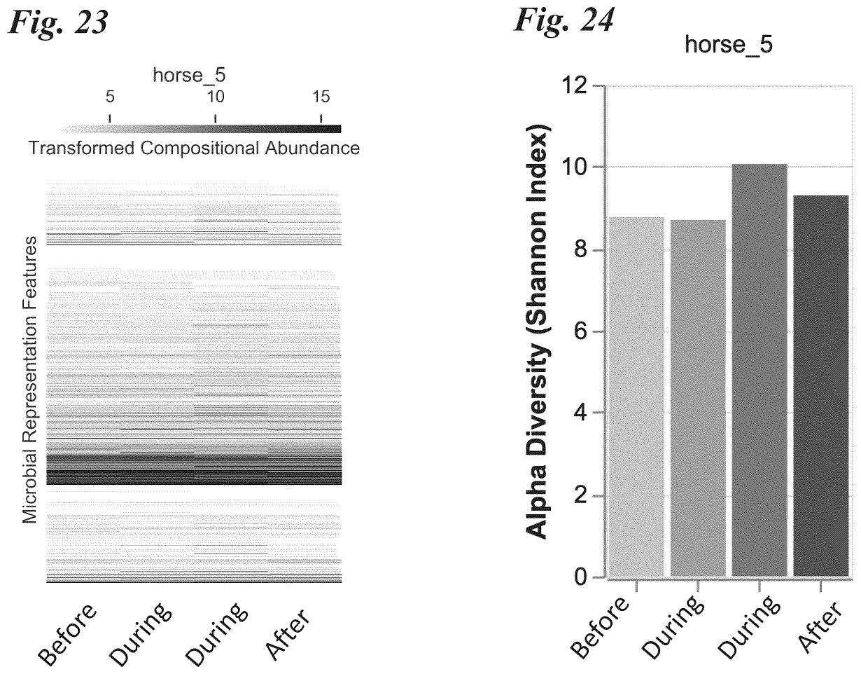

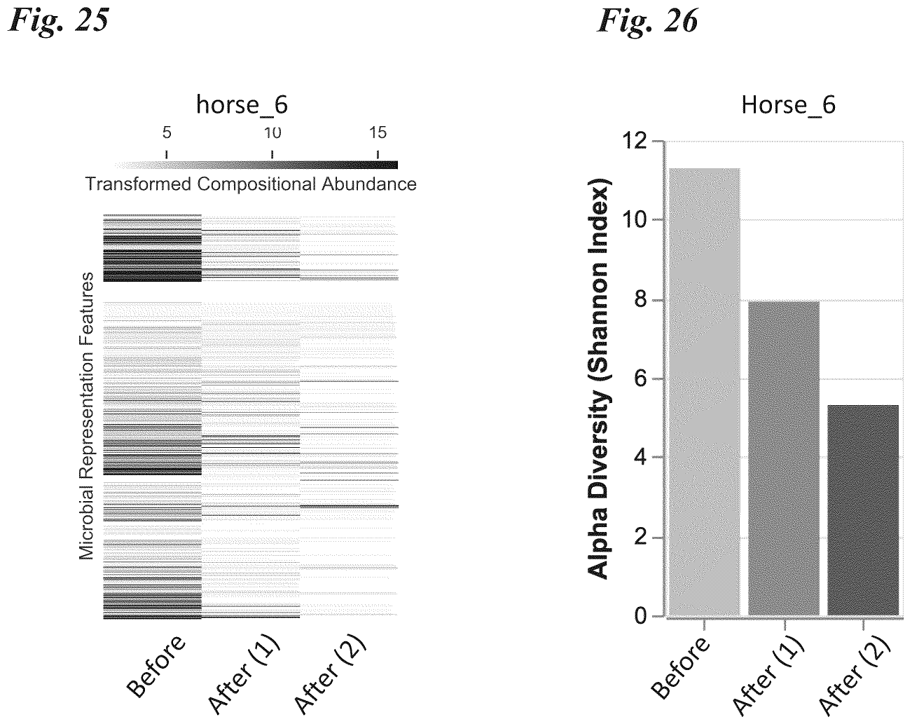

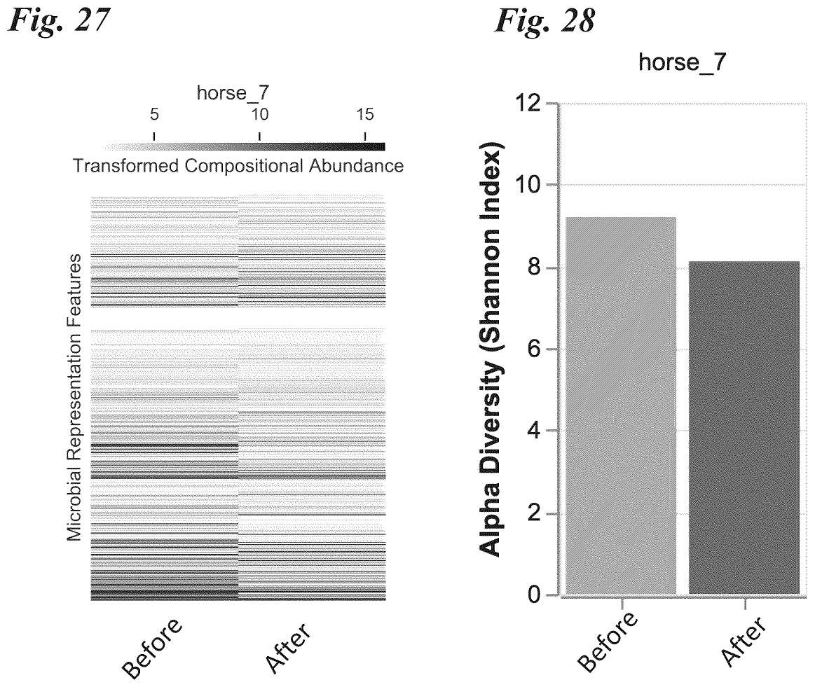

Administration of Ascus Equine to all 7 patients did not cause any abnormal health observations. No colic events were noted during the administration period nor follow up period. The pre-administration fecal microbiomes of the patients were compared to the post-administration fecal microbiome to assess efficacy of native microorganisms in shifting the overall microbial community towards a more healthy state. Previous studies have found that reduced alpha diversity is a common characteristic of healthy microbiomes. FIG. 16 (Patient #1), FIG. 18 (Patient #2), FIG. 20 (Patient #3), FIG. 22 (Patient #4), FIG. 24 (Patient #5), FIG. 26, (Patient #6) and FIG. 28 (Patient #7) depict the alpha diversity of the patients' fecal microbiome prior to Ascus Equine administration (left) and after Ascus Equine administration (right). Some patients have intermediary samples represented as well—these samples represent the microbiome during the administration period. The microbiome composition for each patient is depicted as heat maps in FIG. 15 (Patient #1), FIG. 17 (Patient #2), FIG. 19 (Patient #3), FIG. 21 (Patient #4), FIG. 23 (Patient #5), FIG. 25, (Patient #6) and FIG. 27 (Patient #7). Microbes are represented on the y-axis, and time is on the x-axis (left is pre-administration, right is post-administration, samples in between are during administration period).

Patients can be loosely categorized into the following groups:
(a) Healthy patients with no history of colic: Patient 2, Patient 3, Patient 7
(b) Healthy patients with a history of colic: Patient 1, Patient 5, Patient 6
(c) Actively colicing patients: Patient 4.

In healthy patients with no history of colic who were not actively colicing, little change was observed in alpha diversity after administration of microorganisms. However, the composition of the microbiome was found to shift even closer to the “healthy” state that is more healthy-associated microbes were observed in their fecale microbiomes. Although these animals were not colicing, the post-administration state of the fecal microbiome was more optimal than the pre-administration state.

In healthy/asymptomatic patients who did have a history of colic, many of the pre-administration samples were found to resemble a more colic-like microbiome. This suggests that the patients were in a transient state, and thus more likely to develop a symptomatic colic episode. After administration of microorganisms, patients' fecal microbiomes generally exhibited a reduction in alpha diversity, suggesting that the post-administration state of the fecal microbiome is more optimal than the pre-administration state. Similarly, the composition of their microbiomes because to have increased abundance of healthy-associated microbes. Patient #5 did not exhibit a clear decrease in alpha diversity, however, it's possible that the microorganisms needed to be administered for longer than 2 weeks since this particular patient experienced very frequent and very severe colic episodes. The shift in alpha diversity suggests that the microbiome was beginning to change towards the end of the administration period, and potentially needed additional time to fully exert its impact.

Actively colicing patients saw the largest improvement in state. Patient #4 was experiencing gas colic at the time of administration. After microorganisms were administered, a decrease in alpha diversity was observed, as well as a clear shift in the fecal microbiome towards a more healthy state (healthy-associated microorganisms increased in abundace). No additional colic episodes were observed in the follow-up period. In this case, the post-administration state of the fecal microbiome was more optimal than the pre-administration state. No additional colicing symptoms were observed in the patient, and the patient is likely to have fewer reoccuring cases of colic since the microbiome has been pushed towards a healthier, more stable state.

Collectively, the fecal microbiome data obtained from this case study suggests that administration of Ascus Equine for a 2-week period can shift the microbiome of a horse that experiences frequent colic episodes or a horse that is actively colicing towards a more stable microbiome that causes fewer/no colic episodes.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A microbial composition comprising:
a. one or more bacteria comprising a 16S nucleic acid sequence that is at least 97% identical to any one of SEQ ID NOs: 1-575; and
b. a carrier suitable for equine administration.

2. The microbial composition of claim 1, comprising one or more bacteria comprising a 16S nucleic acid sequence that is at least 98% identical, or at least 99% identical to any one of SEQ ID NOs: 1-575.

3. The microbial composition of claim 1, comprising one or more bacteria comprising a 16S nucleic acid sequence of any one of SEQ ID NOs: 1-575.

4. The microbial composition of claim 1, comprising one or more bacteria comprising a 16S nucleic acid sequence that is at least 97% identical SEQ ID NO: 11, SEQ ID NO: 142, SEQ ID NO: 320, SEQ ID NO: 433, or SEQ ID NO: 476.

5. The microbial composition of claim 1, comprising one or more bacteria comprising a 16S nucleic acid sequence of SEQ ID NO: 11, SEQ ID NO: 142, SEQ ID NO: 320, SEQ ID NO: 433, and or SEQ ID NO: 476.

6. The microbial composition of claim 1, comprising one or more bacteria comprising a 16S nucleic acid sequence consisting of any one of SEQ ID NOs: 1-575.

7. (canceled)

8. The microbial composition of claim 1, comprising one or more bacteria comprising a 16S nucleic acid sequence that is at least 98% identical to SEQ ID NO: 11, SEQ ID NO: 142, SEQ ID NO: 320, SEQ ID NO: 433, or SEQ ID NO: 476.

9. The microbial composition of claim 1, comprising one or more bacteria comprising a 16S nucleic acid sequence that is at least 99% identical to SEQ ID NO: 11, SEQ ID NO: 142, SEQ ID NO: 320, SEQ ID NO: 433, or SEQ ID NO: 476.

10. The microbial composition of claim 1, comprising one or more bacteria comprising a 16S nucleic acid sequence consisting of SEQ ID NO: 11, SEQ ID NO: 142, SEQ ID NO: 320, SEQ ID NO: 433, or SEQ ID NO: 476.

11. (canceled)

12. (canceled)

13. (canceled)

14. (canceled)

15. The microbial composition of claim 1, wherein the microbial composition comprises:
a. a bacterium comprising a 16S nucleic acid sequence that is at least 97% identical to SEQ ID NO: 11;
b. a bacterium comprising a 16S nucleic acid sequence that is at least 97% identical to SEQ ID NO: 142;
c. a bacterium comprising a 16S nucleic acid sequence that is at least 97% identical to SEQ ID NO: 320;
d. a bacterium comprising a 16S nucleic acid sequence that is at least 97% identical to SEQ ID NO: 433; and/or
e. a bacterium comprising a 16S nucleic acid sequence that is at least 97% identical to SEQ ID NO: 476.

16. The microbial composition of claim 1, wherein the microbial composition comprises:
a. a bacterium comprising a 16S nucleic acid sequence of SEQ ID NO: 11;
b. a bacterium comprises a 16S nucleic acid sequence of SEQ ID NO: 142;
c. a bacterium comprising a 16S nucleic acid sequence of SEQ ID NO: 320;
d. a bacterium comprises a 16S nucleic acid sequence of SEQ ID NO: 433; and/or
e. a bacterium comprises a 16S nucleic acid sequence of SEQ ID NO: 476.

17. (canceled)

18. (canceled)

19. (canceled)

20. The microbial composition of claim 1, wherein the carrier comprises a solidification agent or a sweeting agent.

21. The microbial composition of claim 20, wherein the solidification agent is selected from xantham gum, agar, and gelatin; and the sweeting agent is selected from corn syrup, molasses, cane molasses, brewer's yeast, and honey.

22. (canceled)

23. The microbial composition of claim 1, wherein the composition is formulated as a gel, a liquid, a powder, a tablet, a capsule, a pill, a feed additive, a food ingredient, a food supplement, a water additive, a heat-stabilized additive, a moisture-stabilized additive, a pelleted feed additive, a pre-pelleted applied feed additive, a post-pelleted applied feed additive, or a spray additive.

24. The microbial composition of claim 1 wherein the composition is formulated for administration by injection, direct application to target organ, bolus administration, oral administration (such as with or as part of food), fecal enema, or fecal microbiota transplant via nasogastric intubation.

25. (canceled)

26. A method for preventing and/or treating colic in an equine comprising administering the microbial composition of claim 1 to an equine in need thereof in an amount effective to prevent and/or treat colic in the equine administered the microbial composition compared to an equine that was not administered the microbial composition.

27. The method of claim 26, wherein the equine is a domesticated equine, a wild equine, a horse, a zebra, a mule, or a donkey.

28. (canceled)

29. (canceled)

30. The method of claim 26, wherein the microbial composition is administered daily for at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, or longer.

31. (canceled)

32. The method of claim 26, wherein the microbial composition is administered to the equine with an antibiotic, a proton pump inhibitor, and/or food or after administration of an antibiotic, a proton pump inhibitor, and/or food.

33. (canceled)

34. The method of claim 26, wherein the administration of the microbial composition reduces one or more symptoms of colic selected from abdominal pain, stomach irritation, rolling, kicking at stomach, distension of gastrointestinal organs, and decreased eating in the equine; and/or the administration of the microbial composition reduces the frequency of colic episodes in the equine.

35. (canceled)


SEQUENCE LISTING (575 sequences)

SEQ ID NO: 1 - DNA, 225 bp, Arcanobacterium hippocolea
  source 1..225: mol_type = genomic DNA
agagtttgatcatggctcaggacgaacgctggcggcgtgcttaacacatgcaagtcgaacgatgaagcggaaagttttctttttgtggattagtggcgaacgggtgagtaatacgtgagtaacctgccctcttctctgggataagccttggaaacggggtctaatactggatattctgctttaaccgcatggtttttgttggaaagggtttctggtgggggatgg

SEQ ID NO: 2 - DNA, 225 bp, Ihubacter massiliensis
  source 1..225: mol_type = genomic DNA
agagtttgatcctggctcaggatgaacgctggcggcgtgcctaacacatgcaagtcgagcgagaagctgatgattgacacttcggttgagagaatcagtggaaagcggcggacgggtgagtaacgcgtaggcaacctgccctttgcagagggatagcctcgggaaaccgggattaaaacctcatgatgctgtatgtccgcatgggcagacggtcaaagatttatc

SEQ ID NO: 3 - DNA, 225 bp, Peptoniphilus asaccharolyticus
  source 1..225: mol_type = genomic DNA
agagtttgatcatggctcaggacgaacgctggcggcgcgcctaacacatgcaagtcgagcgatgaatcgcacacagaacccttcggggtgaggtgagcgaggattagcggcggacgggtgagtaacacgtgagcaacctgcctttgacacggggatagccaccggaaacggtgattaatacccgataacatcgaattgacacctgtcgaatcgatcaaagtgcct

SEQ ID NO: 4 - DNA, 225 bp, Anaerococcus vaginalis
  source 1..225: mol_type = genomic DNA
agagtttgatcctggctcaggattaacgctggcggcgtgcataacacatgcaagtcgaacgatgacgttttatggaaatcttcggatagaaataaaaaagattagtggcgaacgggtgagtaacacgtgagtaacctgccttacacaaggggatagccgttggaaacgacgaataataccctatgaagtactaagatcgcatgataatagtaccaaagtgcttag

SEQ ID NO: 5 - DNA, 225 bp, Clostridium butyricum
  source 1..225: mol_type = genomic DNA
agagtttgatcatggctcaggacgaacgctggcggcgtgcttaacacatgcaagtcgagcgatgaagctccttcgggagcggattagcggcggacgggtgagtaacacgtgggtaacctgcctcatagaggggaatagcctttcgaaaggaagattaataccgcataagattgtagtgccgcatggcatagcaattaaaggagtaatccgctatgagatggaccc

SEQ ID NO: 6 - DNA, 225 bp, Clostridium butyricum
  source 1..225: mol_type = genomic DNA
agatttgatcatggctcaggacgaacgctggcggcgtgcttaacacatgcaagtcgagcgatgaagctccttcgggagtggattagcggcggacgggtgagtaacacgtgggtaacctgcctcatagaggggaatagccttccgaaaggaagattaataccgcataagattgtagtgctgcatggcatagcaattaaaggagtaatccgctatgagatggacccg

SEQ ID NO: 7 - DNA, 225 bp, Clostridium butyricum
  source 1..225: mol_type = genomic DNA
agagtttgatcatggctcaggacgaacgctggcggcgcgcttaacgcatgcaagtcgagcgatgaggctccttcgggagcggattagcggcggacgggtgagtaacacgtgggtaacctgcctcatagaggggaatagcctttcgaaaggaagattaataccgcataagattgtagtgccgcatggcatagcaattaaaggagtaatccgctatgagatggaccc

SEQ ID NO: 8 - DNA, 225 bp, Clostridium butyricum
  source 1..225: mol_type = genomic DNA
agagtttgatcatggctcaggacgaacgctggcggcgtgcttaacacatgcaagtcgagcgatgaagctccttcgggagtggaatagcggcggacgggtgagtaacacgtgggtaacctgcctcatagaggggaatagcctttcgaaaggaagattaataccgcataagattgtagtgccgcatggcatagcaattaaaggagtaatccgctatgagatggaccc

SEQ ID NO: 9 - DNA, 225 bp, Clostridium butyricum
  source 1..225: mol_type = genomic DNA
agagtttgatcatggctcaggacgaacgctggcggcgtgcttaacacatgcaagtcgagcgatgaagctccttcgggagtggattagcggcggacgggtgagtaacacgtgggtaacctgcctcatagaggggaatagcctttcgaaaggaagattaataccgcataagattgtagtgccgcatggcatagcaattaaaggagtaatccgctatgagatggaccc

SEQ ID NO: 10 - DNA, 225 bp, Clostridium butyricum
  source 1..225: mol_type = genomic DNA
agagtttgatcatggctcaggacgaacgctggcggcgtgcttaacacatgcaagtcgagcgatgaagctccttcgggagtggattagcggcggacgggtgagtaacacgtgggtaacctgcctcatagaggggaatagcctttcgaaaggaagattaataccgcataagattgtagtaccgcatggtacagcaattaaaggagtaatccgctatgagatggaccc

SEQ ID NO: 11 - DNA, 225 bp, Clostridium butyricum; identical to SEQ ID NO: 5
  source 1..225: mol_type = genomic DNA

SEQ ID NO: 12 - DNA, 225 bp, Clostridium butyricum
  source 1..225: mol_type = genomic DNA
agagtttgatcctggctcaggacgaacgctggcggcgtgcttaacacatgcaagtcgagcgatgaagctccttcgggagtggattagcggcggacgggtgagtaacacgtgggtaacctgcctcatagaggggaatagcctttcgaaaggaagattaataccgcataagattgtagtaccgcatggtacagcaattaaaggagtaatccgctatgagatggaccc

SEQ ID NO: 13 - DNA, 225 bp, Clostridium butyricum
  source 1..225: mol_type = genomic DNA
agagtttgatcatggctcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcatttaacacatgttagatgcttgaaagaagcaattgcttcactagta

SEQ ID NO: 14 - DNA, 225 bp, Terrisporobacter mayombei
  source 1..225: mol_type = genomic DNA
agagtttgatcctggctcaggatgaacgctggcggcgtgcctaacacatgcaagtcgagcgatcttcttcggaagagagcggcggacgggtgagtaacgcgtgggtaacctgcctcatacacatggataacataccgaaaggtatgctaatacaagatgatataagagattcgcatggatttcttatcaaagctccggcggtatgagatggacccgcgtctgatt

SEQ ID NO: 15 - DNA, 225 bp, Terrisporobacter mayombei
  source 1..225: mol_type = genomic DNA
agagtttgatcatggctcaggatgaacgctggcggcgtgcctaacacatgcaagtcgagcgatcttcttcggaagagagcggcggacgggtgagtaacgcgtgggtaacctgcctcatacacatggataacataccgaaaggtatgctaatacacgataatataagagattcacatggatttcttatcaaagctccggcggtatgagatggacccgcgtctgatt

SEQ ID NO: 16 - DNA, 225 bp, Terrisporobacter mayombei
  source 1..225: mol_type = genomic DNA
agagtttgatcctggctcaggatgaacgctggcggcgtgcctaacacatgcaagtcgagcgatcttcttcggaagagagcggcggacgggtgagtaacgcgtgggtaacctgcctcatacacatggataacataccgaaaggtatgctaatacacgataatataagagattcacatggatttcttatcaaagctccggcggtatgagatggacccgcgtctgatt

SEQ ID NO: 17 - DNA, 225 bp, Terrisporobacter mayombei
  source 1..225: mol_type = genomic DNA
agagtttgatcatggctcaggatgaacgctggcggcgtgcctaacacatgcaagtcgagcgatcttcttcggaagagagcggcggacgggtgagtaacgcgtgggtaacctgcctcatacacatggataacataccgaaaggtatgctaatacaagatgatataagagattcgcatggatttcttatcaaagctccggcggtatgagatggacccgcgtctgatt

SEQ ID NO: 18 - DNA, 225 bp, Terrisporobacter mayombei
  source 1..225: mol_type = genomic DNA
agagtttgatcctggctcaggatgaacgctggcggcgtgcctaacacatgcaagtcgagcgatcttcttcggaagagagcggcggacgggtgagtaacgcgtgggtaacctgcctcatacacatggataacataccgaaaggtatgctaatacaagataatataagaacttcacatgtagttcttatcaaagctccggcggtatgagatggacccgcgtctgatt

SEQ ID NO: 19 - DNA, 225 bp, Mogibacterium pumilum
  source 1..225: mol_type = genomic DNA
agagtttgatcatggctcaggatgaacgctggcggcgtgcctaatacatgcaagtcgagcgagatgcttatgattgaaacttcggtcgaaagattaagcggaaagcggcggacgggtgagtaacgcgtaggcaacctgcccttagcagagggatagccattggaaacgatgattaaaacctcataacatcgtagctacacatgtagcatcggtcaaagatttatc

SEQ ID NO: 20 - DNA, 225 bp, Mogibacterium pumilum
  source 1..225: mol_type = genomic DNA
agagtttgatcctggctcaggatgaacgctggcggcgtgcctaatacatgcaagtcgagcgagatgcttatgattgaaacttcggtcgaaagattaagcggaaagcggcggacgggtgagtaacgcgtaggcaacctgcccttagcagagggatagccattggaaacgatgattaaaacctcataacatcgtagctacacatgtagcaacggtcaaagatttatc

SEQ ID NO: 21 - DNA, 225 bp, Mogibacterium pumilum
  source 1..225: mol_type = genomic DNA
agagtttgatcctggctcaggatgaacgctggcggcgtgcctaatacatgcaagtcgagcgagatgctcatgattgaaacttcggtcgaaagattgagcggaaagcggcggacgggtgagtaacgcgtaggcaacctgcccttagcagagggatagccattggaaacgatgattaaaacctcataacatcgtagcaacacatgttgcaacggtcaaagatttatc

SEQ ID NO: 22 - DNA, 225 bp, Mogibacterium pumilum
  source 1..225: mol_type = genomic DNA
agagtttgatcatggctcaggatgaacgctggcggcgtgcctaatacatgcaagtcgagcgagaagctcatgattgaaacttcggtcgaaagattgagtggaaagcggcggacgggtgagtaacgcgtaggcaacctgcccttagcagagggatagccattggaaacgatgattaaaacctcataacatcgtagctacacatgtagcatcggtcaaagatttatc

SEQ ID NO: 23 - DNA, 225 bp, Mogibacterium pumilum
  source 1..225: mol_type = genomic DNA
agagtttgatcctggctcaggatgaacgctggcggcgtgcctaatacatgcaagtcgagcgagaagctcatgattgaaacttcggtcgaaagattgagtggaaagcggcggacgggtgagtaacgcgtaggcaacctgcccttagcagagggatagccattggaaacgatgattaaaacctcataacatcgtagctacacatgtagcatcggtcaaagatttatc

SEQ ID NO: 24 - DNA, 225 bp, Arcanobacterium phocae
  source 1..225: mol_type = genomic DNA
agagtttgatcctggctcaggacgaacgctggcggcgtgcttaacacatgcaagtcgaacgatgaagcctagcttgctgggtggattagtggcgaacgggtgagtaatacgtgagtaacctgccctcttctttgggataagccttggaaacggggtctaatactggatattctgcaatgatcgcatggttgttgttggaaaggttttttctggtgggggatgggc

SEQ ID NO: 25 - DNA, 225 bp, Catabacter hongkongensis
  source 1..225: mol_type = genomic DNA
agagtttgatcatggctcaggacgaacgctggcggcgtgcttaacacatgcaagtcgaacgaggatgttggcttcggctgacattcgagtggcggacgggtgagtaacgcgtgagcaatctgtctcagagagggggataacaactggaaacagttgctaataccgcataagaccacgggagggcatccttctgaggtaaaagcagcaatgcgctttgaggtgagc

SEQ ID NO: 26 - DNA, 225 bp, Catabacter hongkongensis
  source 1..225: mol_type = genomic DNA
agagtttgatcctggctcaggacgaacgctggcggcgtgcttaacacatgcaagtcgaacgaggatgttggcttcggccgacattcgagtggcggacgggtgagtaacgcgtgagcaatctgtctcagagagggggataacaactggaaacagttgctaataccgcataagaccacgggagggcatccttctgaggtaaaagcagcaatgcgctttgaggtgagc

SEQ ID NO: 27 - DNA, 225 bp, Parabacteroides distasonis
  source 1..225: mol_type = genomic DNA
agagtttgatcctggctcaggatgaacgctagcgacaggcttaacacatgcaagtcgaggggcagcacagggtagcaatacttgggtggcgaccggcgcacgggtgagtaacgcgtatgcaacttacctatcagagggggatagcccggcgaaagtcggattaataccccatgaaacaggggtcccgcatgggaatatttgttaaagattcatcgctgatagata

SEQ ID NO: 28 - DNA, 225 bp, Finegoldia magna
  source 1..225: mol_type = genomic DNA
agagtttgatcctggctcaggacgaacgctggcggcgtgcttaacacatgcaagtcgaacgggatttagtaaacagaaacctcggtggaagattactaatgagagtggcgaacgggtgagtaacgcgtgagcaacctgcctatgacagtgggatagcctcgggaaaccgggattaataccgcataaaatcgtagaaacacatgtttcagcggtcaaagatttatc

SEQ ID NO: 29 - DNA, 225 bp, Clostridium nitritogenes
  source 1..225: mol_type = genomic DNA
agagtttgatcatggctcaggacgaacgctggcggcgtgcctaacacatgcaagtcgagcgatgaagtttccttcgggaaatggattagcggcggacgggtgagtaacacgtgggtaacctgcctcatagaggggaatagcctcccgaaagggagattaataccgcataacattacagtttcgcatgaaacagtaattaaaggagcaatccgctatgagatggac

SEQ ID NO: 30 - DNA, 225 bp, Clostridium nitritogenes; identical to SEQ ID NO: 29
  source 1..225: mol_type = genomic DNA

SEQ ID NO: 31 - DNA, 225 bp, Clostridium nitritogenes
  source 1..225: mol_type = genomic DNA
agagtttgatcatggctcaggacgaacgctggcggcgtgcctaacacatgcaagtcgagcgatgaagtttccttcgggaaatggattagcggcggacgggtgagtaacacgtgggtaacctgcctcatagaggggaatagcctcccgaaagggagattaataccgcataatattacagtttcgcatgaaacagtaattaaaggagcaatccgctatgagatggac

SEQ ID NO: 32 - DNA, 225 bp, Clostridium nitritogenes
  source 1..225: mol_type = genomic DNA
agagtttgatcctggctcaggacgaacgctggcggcgtgcctaacacatgcaagtcgagcgatgaagtttccttcgggaaatggattagcggcggacgggtgagtaacacgtgggtaacctgcctcatagaggggaatagcctcccgaaagggagattaataccgcataatattacagtttcgcatgaaacagtaattaaaggagcaatccgctatgagatggac

SEQ ID NO: 33 - DNA, 225 bp, Clostridium nitritogenes
  source 1..225: mol_type = genomic DNA
agatttgctcatggctcaggacgaacgctggcggcgtgcctaacacatgcaagtcgagcgatgaagtttccttcgggaaatggattagcggcggacgggtgagtaacacgtgggtaacctgcctcatagaggggaatagcctcccgaaagggagattaataccgcataatattacagtttcgcatgaaacagtaattaaaggagcaatccgctatgagatggacc

SEQ ID NO: 34 - DNA, 225 bp, Clostridium nitritogenes
  source 1..225: mol_type = genomic DNA
agagtttgatcctggctcaggacgaacgctggcggcgtgcctaacacatgcaagtcgagcgatgaagtttccttcgggaaatggattagcggcggacgggtgagtaacacgtgggtaacctgcctcatagaggggaatagcctcccgaaagggagattaataccgcataatattacagtttcgcatgagacagtaattaaaggagcaatccgctatgagatggac

SEQ ID NO: 35 - DNA, 225 bp, Clostridium nitritogenes
  source 1..225: mol_type = genomic DNA
agatttgatcctggctcaggacgaacgctggcggcgtgcctaacacatgcaagtcgagcgatgaagtttccttcgggaaacggattagcggcggacgggtgagtaacacgtgggtaacctgcctcatagaggggaatagcctcccgaaagggagattaataccgcataatattacagtttcgcatgaaacagtaattaaaggagcaatccgctatgagatggacc

SEQ ID NO: 36 - DNA, 225 bp, Clostridium nitritogenes
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattagatagcatttgataatgattaaagatttattgctgatggatggacatgcgttgga

SEQ ID NO: 37 - DNA, 225 bp, Corynebacterium glaucum
  source 1..225: mol_type = genomic DNA
agagtttgatcatggctcaggatgaacgctggcggcgtgcttaacacatgcaagtcgaacggaaaggcctcagcttgctggggtgctcgagtggcgaacgggtgagtaacacgtaggtgatctgccctgcactgtgggataagcctgggaaactgggtctaataccgcataggaccatgatgttgtgattgtggtggaaaggttttttctggtgtgggatgagct

SEQ ID NO: 38 - DNA, 225 bp, unidentified
  misc_feature 1..225: note = Oribacterium sp.
  source 1..225: mol_type = genomic DNA
agagtttgatcatggctcaggatgaacgctggcggcgtgcctaacacatgcaagtcgaacggtgtcatgatgaaacctggtgattcatgacatagtggcggacgggtgagtaacgcgtgggtaacctgccttctggagggggatagcagctggaaacggctggtaataccgcataagcgcacagctccgcatggagcagtgtgaaaagatttatcgccagaagat

SEQ ID NO: 39 - DNA, 225 bp, unidentified
  misc_feature 1..225: note = Oribacterium sp.
  source 1..225: mol_type = genomic DNA
agagtttgatcctggctcaggatgaacgctggcggcgtgcctaacacatgcaagtcgaacggtgtcatgatgaaacctagtgattcatgacatagtggcggacgggtgagtaacgcgtgggtaacctgccttctggagggggatagcagctggaaacggctggtaataccgcataagcgcacagctccgcatggagcagtgtgaaaagatttatcgccagaagat

SEQ ID NO: 40 - DNA, 225 bp, unidentified
  misc_feature 1..225: note = Oribacterium sp.
  source 1..225: mol_type = genomic DNA
agagtttgatcatggctcaggatgaacgctggcggcgtgcctaacacatgcaagtcgaacggtgtcatgatgaaacctagtgattcatgacatagtggcggacgggtgagtaacgcgtgggtaacctgccttctggagggggatagcagctggaaacggctggtaataccgcataagcgcacagctccgcatggagcagtgtgaaaagatttatcgccagaagat

SEQ ID NO: 41 - DNA, 225 bp, Lachnospiraceae bacterium
  source 1..225: mol_type = genomic DNA
agagtttgatcctggctcaggatgaacgctggcggcgtgcttaacacatgcaagtcgaacggaaacttattgaaacctagtgatataagttttagtggcggacgggtgagtaacgcgtgggtaacctgccttgtactgggggacaacagttggaaacggctgctaataccgcataagaccacagagtcgcatgactcaggggtaaaaactccggtggtacaagat

SEQ ID NO: 42 - DNA, 225 bp, Lachnospiraceae bacterium
  source 1..225: mol_type = genomic DNA
agagtttgatcatggctcaggatgaacgctggcggcgtgcttaacacatgcaagtcgaacggaaacttattgaaacctagtgatataagttttagtggcggacgggtgagtaacgcgtgggtaacctgccttgtactgggggacaacagttggaaacggctgctaataccgcataagaccacagagtcgcatgactcaggggtaaaaactccggtggtacaagat

SEQ ID NO: 43 - DNA, 225 bp, Lachnospiraceae bacterium
  source 1..225: mol_type = genomic DNA
agagtttgatcatggctcaggatgaacgctggcggcgtgcttaacacatgcaagtcgaacggaaacttattgaaacctagtgatataagttttagtggcggacgggtgagtaacgcgtgggtaacctgcctcgtactgggggacaacagttggaaacgactgctaataccgcataagaccacagagtcgcatgactcaggggtaaaaactccggtggtacgagat

SEQ ID NO: 44 - DNA, 225 bp, Lachnospiraceae bacterium
  source 1..225: mol_type = genomic DNA
agagtttgatcctggctcaggatgaacgctggcggcgtgcttaacacatgcaagtcgaacggaaacttattgaaacctagtgatataagttttagtggcggacgggtgagtaacgcgtgggtaacctgcctcgtactgggggacaacagttggaaacgactgctaataccgcataagaccacagagtcgcatgactcaggggtaaaaactccggtggtacgagat

SEQ ID NO: 45 - DNA, 225 bp, Lachnospiraceae bacterium; identical to SEQ ID NO: 13
  source 1..225: mol_type = genomic DNA

SEQ ID NO: 46 - DNA, 225 bp, Lachnospiraceae bacterium; identical to SEQ ID NO: 41
  source 1..225: mol_type = genomic DNA

SEQ ID NO: 47 - DNA, 225 bp, Lachnospiraceae bacterium
  source 1..225: mol_type = genomic DNA
agagtttgatcctggctcaggatgaacgctggcggcgtgcttaacacatgcaagtcgaacggacttatctttaagcttgctttttgataagttagtggcggacgggtgagtaacgcgtgggtaacctgcctcgtactgggggacaacagttggaaacgactgctaataccgcataagaccacagagtcgcatgactcaggggtaaaaactccggtggtacgagat

SEQ ID NO: 48 - DNA, 225 bp, Lachnospiraceae bacterium
  source 1..225: mol_type = genomic DNA
agagtttgatcatggctcaggatgaacgctggcggcgtgcttaacacatgcaagtcgaacggacttatctttaagcttgctttttgataagttagtggcggacgggtgagtaacgcgtgggtaacctgcctcgtactgggggacaacagttggaaacgactgctaataccgcataagaccacagagccgcatggctcaggggtaaaaactccggtggtacgagat

SEQ ID NO: 49 - DNA, 225 bp, Lachnospiraceae bacterium
  source 1..225: mol_type = genomic DNA
agagtttgatcatggctcaggatgaacgctggcggcgtgcttaacacatgcaagtcgaacggacttatctttaagcttgctttttgataagttagtggcggacgggtgagtaacgcgtgggtaacctgcctcgtactgggggacaacagttggaaacgactgctaataccgcataagaccacagagtcgcatgactcaggggtaaaaactccggtggtacgagat

SEQ ID NO: 50 - DNA, 225 bp, Lachnospiraceae bacterium; identical to SEQ ID NO: 47
  source 1..225: mol_type = genomic DNA

SEQ ID NO: 51 - DNA, 225 bp, Lachnospiraceae bacterium
  source 1..225: mol_type = genomic DNA
agagtttgatcctggctcaggatgaacgctggcggcgtgcttaacacatgcaagtcgaacggacttatctttaagcttgctttttgataagttagtggcggacgggtgagtaacgcgtgggtaacctgcctcgtactgggggacaacagttggaaacgactgctaataccgcataagaccacagagccgcatggctcaggggtaaaaactccggtggtacgagat

SEQ ID NO: 52 - DNA, 225 bp, Odoribacter splanchnicus
  source 1..225: mol_type = genomic DNA
agagtttgatcctggctcaggatgaacgctagcgacaggcctaacacatgcaagtcgaggggcatcgcgggtagcaatacctggcggcgaccggcgcacgggtgcgtaacaggtgtgcaatctgtccatagtcgggggatagcccagcgaaagctgaattaacactccatgtgagtgagagtcgcatgactttcacttgaaacgtaaggactatgggtgagcacg

SEQ ID NO: 53 - DNA, 225 bp, Odoribacter splanchnicus
  source 1..225: mol_type = genomic DNA
agagtttgatcctggctcaggatgaacgctagcggcaggcttaacacatgcaagtcgaggggcagcgcaggtagcaataccgggcggcgaccggcgcacgggtgcgtaacaggtgtgcaatctgtccgtagtcgggggatagcccagcgaaagctgaattaacactccatgtgagtgagagtcgcatgactttcacttgaaacgtaaggactatgggtgagcacg

SEQ ID NO: 54 - DNA, 225 bp, Odoribacter splanchnicus
  source 1..225: mol_type = genomic DNA
agagtttgatcatggctcaggatgaacgctagcgacaggcctaacacatgcaagtcgaggggcagcgcgggtagcaatacctggcggcgaccggcgcacgggtgcgtaacaggtgtgcaatctgtccatagttgggggatagcccagcgaaagctgaattaacactccatgtgagtgggagtcgcatgactttcacttgaaacgtaaggactatgggtgagcacg

SEQ ID NO: 55 - DNA, 225 bp, Odoribacter splanchnicus
  source 1..225: mol_type = genomic DNA
agagtttgatcatggctcaggatgaacgctagcgacaggcctaacacatgcaagtcgaggggcatcgcgggtagcaatacctggcggcgaccggcgcacgggtgcgtaacaggtgtgcaatctgtccatagtcgggggatagcccagcgaaagctgaattaacactccatgtgagtgagagttgcatgactttcacttgaaacgtaaggactatgggtgagcacg

SEQ ID NO: 56 - DNA, 225 bp, Odoribacter splanchnicus
  source 1..225: mol_type = genomic DNA
agagtttgatcatggctcaggatgaacgctagcgacaggcctaacacatgcaagtcgaggggcatcgcgggtagcaatacctggcggcgaccggcgcacgggtgcgtaacaggtgtgcaatctgtccatagtcgggggatagcccagcgaaagctgaattaacactccatgtgagtgagagtcgcatgactttcacttgaaacgtaaggactatgggtgagcacg

SEQ ID NO: 57 - DNA, 225 bp, Odoribacter splanchnicus
  source 1..225: mol_type = genomic DNA
agagtttgatcatggctcaggatgaacgctagcgacaggcctaacacatgcaagtcgaggggcatcgcgggtagcaatacctggcggcgaccggcgcacgggcgcgtaacaggtgtgcaatctgtccatagtcgggggatagcccagcgaaagctgaattaacactccatgtgagtgagagttgcatgactttcacttgaaacgtaaggactatgggtgagcacg

SEQ ID NO: 58 - DNA, 225 bp, Odoribacter splanchnicus
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgatatcgtttgaaagcatttgataatgattaaagatttatcgctgatggatggacatgcgttgga

SEQ ID NO: 59 - DNA, 225 bp, Bacteroides thetaiotaomicron
  source 1..225: mol_type = genomic DNA
agagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatc

SEQ ID NO: 60 - DNA, 225 bp, unidentified
  misc_feature 1..225: note = Peptoniphilus sp.
  source 1..225: mol_type = genomic DNA
agagtttgatcatggctcaggacgaacgctggcggcgcgcctaacacatgcaagtcgagcgatgaaagtttgacggaggtcttcggaccgaagacaaacaggattagcggcgaacgggtgagtaacgcgtgaggaacctacctttcacaacgggatagcctcgggaaaccgggattaataccgtatgacacagtgcattcgcatgaacgtactgttaaagtgtta

SEQ ID NO: 61 - DNA, 225 bp, Intestinimonas butyriciproducens
  source 1..225: mol_type = genomic DNA
agagtttgatcctggctcaggatgaacgctggcggcgtgcttaacacatgcaagtcgaacggagcacccctgacggagttttcggacaacgaaagggaatgcttagtggcggacgggtgagtaacgcgtgagcaacctgccttggagtggggaataacagccggaaacggctgctaataccgcatgatgtatctggatcgcatggttctggataccaaagattta

SEQ ID NO: 62 - DNA, 225 bp, Terrisporobacter glycolicus
  source 1..225: mol_type = genomic DNA
agagtttgatcatggctcaggatgaacgctggcggcgtgcctaacacatgcaagtcgagcgattcacttcggtgaagagcggcggacgggtgagtaacgcgtgggtaacctgcctcatacacatggataacataccgaaaggtatgctaatacaggataatataagagattcgcatggatttcttatcaaagctccggcggtatgagatggacccgcgtctgatt

SEQ ID NO: 63 - DNA, 225 bp, Terrisporobacter glycolicus
  source 1..225: mol_type = genomic DNA
agagtttgatcctggctcaggatgaacgctggcggcgtgcctaacacatgcaagtcgagcgattctcttcggagaagagcggcggacgggtgagtaacgcgtgggtaacctgcctcatacacatggataacataccgaaaggtatgctaatacaggataacataagagattcacatggatttcttatcaaagctctggcggtatgagatggacccgcgtctgatt

SEQ ID NO: 64 - DNA, 225 bp, Terrisporobacter glycolicus; identical to SEQ ID NO: 34
  source 1..225: mol_type = genomic DNA

SEQ ID NO: 65 - DNA, 225 bp, Terrisporobacter glycolicus; identical to SEQ ID NO: 13
  source 1..225: mol_type = genomic DNA

SEQ ID NO: 66 - DNA, 225 bp, Terrisporobacter glycolicus
  source 1..225: mol_type = genomic DNA
agagtttgatcctggctcaggatgaacgctggcggcgtgcctaacacatgcaagtcgagcgattcacttcggtgaagagcggcggacgggtgagtaacgcgtgggtaacctgcctcatacacatggataacataccgaaaggtatgctaatacaggataatataagagattcgcatggatttcttatcgaagcttttgcggtatgagatggacccgcgtctgatt

SEQ ID NO: 67 - DNA, 225 bp, Terrisporobacter glycolicus
  source 1..225: mol_type = genomic DNA
agagtttgatcatggctcaggatgaacgctggcggcgtgcctaacacatgcaagtcgagcgattcacttcggtgaagagcggcggacgggtgagtaacgcgtgggtaacctgcctcatacacatggataacataccgaaaggtatgctaatacaggataatataagagattcgcatggatttcttatcgaagcttttgcggtatgagatggacccgcgtctgatt

SEQ ID NO: 68 - DNA, 225 bp, Terrisporobacter glycolicus
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgaaagcatttgataacgattaaagatttatcgctgatggatggacatgcgttgga

SEQ ID NO: 69 - DNA, 225 bp, Terrisporobacter glycolicus; identical to SEQ ID NO: 56
  source 1..225: mol_type = genomic DNA

SEQ ID NO: 70 - DNA, 225 bp, Porphyromonas canoris
  source 1..225: mol_type = genomic DNA
agagtttgatcatggctcaggatgaacgctagcgataggcttaacacatgcaagtcgaggggtagcatattggcagcttgctgtcaatgatgacgaccggcggatgggtgcgtaacgcgtatgcaacttacctattagcgggggataacccggagaaattcggactaatacgccatattttccagaagttgcctgatttttggaagaaagatttatcactaatag

SEQ ID NO: 71 - DNA, 225 bp, unidentified
  misc_feature 1..225: note = Peptococcus sp.
  source 1..225: mol_type = genomic DNA
agagtttgatcctggctcaggacaaacgctggcggcatgcctaacacatgcaagtcgaacggacggaggttacggaacttttcggagggaagtgacccgagttagtggcggatgggtgagtaacgcgtgggtaacctgccttatagtggggaataacattgagaaatcattgctaataccgcataatattctcgtgtcgcatgacaagagaaggaaagacggcct

SEQ ID NO: 72 - DNA, 225 bp, Eubacterium barkeri
  source 1..225: mol_type = genomic DNA
agagtttgatcatggctcaggacgaacgctggcggtatgcttaacacatgcaagtcgaacgagaagcttttgatggatccttcgggagaaatgagaagcggaaagtggcgaacgggtgagtaacgcgtgggtaacctacctcatggaaagggatagccccgggaaactgggaataaaaccttatagatattgtattcgcatggatacagtaagaaaactccggtg

SEQ ID NO: 73 - DNA, 225 bp, Eubacterium barkeri
  source 1..225: mol_type = genomic DNA
agagtttgatcctggctcaggacgaacgctggcggtatgcttaacacatgcaagtcgaacgagaagctttttatggatccttcgggagaaatgaggagcagaaagtggcgaacgggtgagtaacgcgtgggtaacctacctcatggaaagggatagccccgggaaactgggaataaaaccttatagatattgtattcgcatggatgcagtaagaaaactccggtg

SEQ ID NO: 74 - DNA, 225 bp, Eubacterium barkeri
  source 1..225: mol_type = genomic DNA
agagtttgatcatggctcaggacgaacgctggcggtatgcttaacacatgcaagtcgaacgagaagcttttgatggatccttcgggagaaatgagaagcggaaagtggcgaacgggtgagtaacgcgtgggtaacctacctcatggaaagggatagccccgggaaactgggaataaaaccttatagatattgtattcgcatggatgcagtaagaaaactccggtg

SEQ ID NO: 75 - DNA, 225 bp, Eubacterium barkeri
  source 1..225: mol_type = genomic DNA
agagtttgatcctggctcaggacgaacgctggcggtatgcttaacacatgcaagtcgaacgagaagcttttgatggatccttcgggagaaatgagaagcggaaagtggcgaacgggtgagtaacgcgtgggtaacctacctcatggaaagggatagccccgggaaactgggaataaaaccttatagatattgtaatcgcatggatacagtaagaaaactccggtg

SEQ ID NO: 76 - DNA, 225 bp, Eubacterium barkeri; identical to SEQ ID NO: 72
  source 1..225: mol_type = genomic DNA

SEQ ID NO: 77 - DNA, 225 bp, Lachnospiraceae bacterium
  source 1..225: mol_type = genomic DNA
agagtttgatcatggctcaggatgaacgctggcggcgtgcttaacacatgcaagtcgaacggacttattatgaagcctagcgattgataagttagtggcggacgggtgagtaacgcgtgggtaacctgcctcgtactgggggacaacagttggaaacgactgctaataccgcataagcgcacgggattgcatgatccagtgtgaaaaactccggtggtacgagat

SEQ ID NO: 78 - DNA, 225 bp, Lachnospiraceae bacterium; identical to SEQ ID NO: 77
  source 1..225: mol_type = genomic DNA

SEQ ID NO: 79 - DNA, 225 bp, Monoglobus pectinilyticus
  source 1..225: mol_type = genomic DNA
agagtttgatcctggctcaggacgaacgctggcggcgtgcctaacacatgcaagtcgagcgggaaatttcaaaatgaaacttcggtcgaattttgaaatggagagcggcggacgggtgagtaacgcgtgagtaacctgcccttcggaatgggataacacatcgaaaggtgtgctaataccgtataacataactttaccgcatgatagagttatcaaagatttatc

SEQ ID NO: 80 - DNA, 225 bp, Cellulosilyticum ruminicola
  source 1..225: mol_type = genomic DNA
agagtttgatcatggctcaggatgaacgctggcggcgtgcttaacacatgcaagtcgaacgaacggaggagagcttgctctccaaagttagtggcggacgggtgagtaacgcgtgggtaacctgcctcatgcagggggataacgtttggaaacgaacgctaataccgcataaactatggattaccgcatggtaattatagcaaagatttatcggcatgagatgga

SEQ ID NO: 81 - DNA, 225 bp, Cellulosilyticum ruminicola
  source 1..225: mol_type = genomic DNA
agagtttgatcatggctcaggatgaacgctggcggcgtgcttaacacatgcaagtcgaacgaacggaggagtgcttgcactccaaagttagtggcggacgggtgagtaacgcgtgggtaacctgcctcatgcagggggataacgtttggaaacgaacgctaataccgcataaactatggattaccgcatggtaattatagcaaagatttatcggcatgagatgga

SEQ ID NO: 82 - DNA, 225 bp, Cellulosilyticum ruminicola
  source 1..225: mol_type = genomic DNA
agagtttgatcatggctcaggatgaacgctggcggcgtgcttaacacatgcaagtcgaacgaacggagaagagcttgctcttcgaagttagtggcggacgggtgagtaacgcgtgggtaacctgcctcatgcagggggataacgtttggaaacgaacgctaataccgcataaactatggattaccgcatggtaattatagcaaagatttatcggcatgagatgga

SEQ ID NO: 83 - DNA, 225 bp, Cellulosilyticum ruminicola
  source 1..225: mol_type = genomic DNA
agagtttgatcctggctcaggatgaacgctggcggcgtgcttaacacatgcaagtcgaacgaacagaggagtgcttgcactccgaagttagtggcggacgggtgagtaacgcgtgggtaacctgcctcatgcagggggataacgtttggaaacgaacgctaataccgcataaactatggattaccgcatggtaattatagcaaagatttatcggcatgagatgga

SEQ ID NO: 84 - DNA, 225 bp, Cellulosilyticum ruminicola
  source 1..225: mol_type = genomic DNA
agagtttgatcctggctcaggatgaacgctggcggcgtgcttaacacatgcaagtcgaacgaacggagaagagcttgctcttcgaagttagtggcggacgggtgagtaacgcgtgggtaacctgcctcatgcagggggataacgtttggaaacgaacgctaataccgcataaactatggattaccgcatggtaattatagcaaagatttatcggcatgagatgga

SEQ ID NO: 85 - DNA, 225 bp, Erysipelotrichaceae bacterium
  source 1..225: mol_type = genomic DNA
agagtttgatcatggctcaggatgaacgctggcggcgtgcttaatacatgcaagtcgaacggacaaaatcagtaagcttgcttattgatcgagttagtggcgaacgggtgagtaatacgtaggcaacctgccctaaagtgggggatagcagttggaaacgactattaataccgcataggtaatcggatggcatcatctgattattaaagacgcgtttgcgtcgct

SEQ ID NO: 86 - DNA, 225 bp, Erysipelotrichaceae bacterium
  source 1..225: mol_type = genomic DNA
agagtttgatcctggctcaggatgaacgctggcggcgtgcttaatacatgcaagtcgaacggacaaagtcaataagcttgcttattgatcgagttagtggcgaacgggtgagtaatacgtaggcaacctaccctaaagtgggggatagcagttggaaacgactattaataccgcataggtaatcggaaggcatcttctgattattaaagacgcgtttgcgtcgct

SEQ ID NO: 87 - DNA, 225 bp, Erysipelotrichaceae bacterium
  source 1..225: mol_type = genomic DNA
agagtttgatcatggctcaggatgaacgctggcggcgtgcttaatacatgcaagtcgaacggacaaagtcagtaagcttgcttactgatagagttagtggcgaacgggtgagtaatacgtaggcaacctgccctaaagtgggggatagcagttggaaacgactattaataccgcataggtaatcggatggcatcatctgattattaaagacgcgtttgcgtcgct

SEQ ID NO: 88 - DNA, 225 bp, Erysipelotrichaceae bacterium; identical to SEQ ID NO: 85
  source 1..225: mol_type = genomic DNA

SEQ ID NO: 89 - DNA, 225 bp, Erysipelotrichaceae bacterium
  source 1..225: mol_type = genomic DNA
agagtttgatcctggctcaggatgaacgctggcggcgtgcttaatacatgcaagtcgaacggacaaaatcaataagcttgcttattgatcgagttagtggcgaacgggtgagtaatacgtaggcaacctgccctaaagtgggggatagcagttggaaacgactattaataccgcataggtaatcggatggcatcatctgattattaaagacgcgtttgcgtcgct

SEQ ID NO: 90 - DNA, 225 bp, Erysipelotrichaceae bacterium
  source 1..225: mol_type = genomic DNA
agagtttgatcctggctcaggatgaacgctggcggcgtgcttaatacatgcaagtcgaacggacaaagtcagtaagcttgcttactgatagagttagtggcgaacgggtgagtaatacgtaggcaacctgccctaaagtgggggatagcagttggaaacgactattaataccgcataggtaatcggatggcatcatctgattattaaagacgcgtttgcgtcgct

SEQ ID NO: 91 - DNA, 225 bp, Erysipelotrichaceae bacterium
  source 1..225: mol_type = genomic DNA
agagtttgatcatggctcaggatgaacgctggcggcgtgcttaatacatgcaagtcgaacggacaaaatcaataagcttgcttattgatcgagttagtggcgaacgggtgagtaatacgtaggcaacctgccctaaagtgggggatagcagttggaaacgactattaataccgcataggtaatcggatggcatcatctgattattaaagacgcgtttgcgtcgct

SEQ ID NO: 92 - DNA, 225 bp, Erysipelotrichaceae bacterium
  source 1..225: mol_type = genomic DNA
agagtttgatcatggctcaggatgaacgctggcggcgtgcttaatacatgcaagtcgaacggacaaagtcaataagcttgcttattgatcgagttagtggcgaacgggtgagtaatacgtaggcaacctaccctaaagtgggggatagcagttggaaacgactattaataccgcataggtaatcggaaggcatcttctgattattaaagacgcgtttgcgtcgct

SEQ ID NO: 93 - DNA, 225 bp, Howardella ureilytica
  source 1..225: mol_type = genomic DNA
agagtttgatcctggctcaggatgaacgctggcggcgtgcctaacacatgcaagtcgaacggagattattcgctgccaaaccttcgggtgaagcttgagtaatcttagtggcggacgggtgagtaacgcgtgggcaacctgccttacacagggggatagcagttggaaacgactggtaataccgcataccttggggaattcgcatgtttacctcaagaaaacttt

SEQ ID NO: 94 - DNA, 225 bp, Howardella ureilytica
  source 1..225: mol_type = genomic DNA
agagtttgatcatggctcaggatgaacgctggcggcgtgcctaacacatgcaagtcgaacggagattattcgctgccaaaccttcgggtgaagcttgagtaatcttagtggcggacgggtgagtaacgcgtgggcaacctgccttacacagggggatagcagttggaaacgactggtaataccgcataccttggggaattcgcatgtttacctcaagaaaacttt

SEQ ID NO: 95 - DNA, 225 bp, Howardella ureilytica; identical to SEQ ID NO: 93
  source 1..225: mol_type = genomic DNA

SEQ ID NO: 96 - DNA, 225 bp, Negativicoccus massiliensis
  source 1..225: mol_type = genomic DNA
agagtttgatcatggctcaggacgaacgctggcggcgtgcctaacacatgcaagtcgaacggggtgacgagaaagcttgcttttttggaacctagtggcgaacgggtgagtaacgcgtaaacaacctacctttaggacggggacaacagacggaaacgactgctaataccggatagactctcgaatcgagtgagacgaagaggaaaggtggcctctcgaagaagc

SEQ ID NO: 97 - DNA, 225 bp, Butyrivibrio fibrisolvens
  source 1..225: mol_type = genomic DNA
agagtttgatcatggctcaggatgaacgctggcggcgtgcctaacacatgcaagtcgaacggacctgatttgtcggagacttcggtcaaagatgatgactggttagtggcggacgggtgagtaacgcgtgggtaacctgcctctaagagggggataacagagagaaatcactgctaataccgcataagcgcacagtaccgcatggtacagtgtgaaaagatttat

SEQ ID NO: 98 - DNA, 225 bp, Butyrivibrio fibrisolvens; identical to SEQ ID NO: 97
  source 1..225: mol_type = genomic DNA

SEQ ID NO: 99 - DNA, 225 bp, Butyrivibrio fibrisolvens
  source 1..225: mol_type = genomic DNA
agagtttgatcctggctcaggatgaacgctggcggcgtgcctaacacatgcaagtcgaacggacctgatttgtcagagacttcggtcgaagatgatgactggttagtggcggacgggtgagtaacgcgtgggtaacctgcctctaagagggggataacagagagaaatcactgctaataccgcataagcgcacagtaccgcatggtacagtgtgaaaagattcat

SEQ ID NO: 100 - DNA, 225 bp, Butyrivibrio fibrisolvens
  source 1..225: mol_type = genomic DNA
agagtttgatcctggctcaggatgaacgctggcggcgtgcctaacacatgcaagtcgaacggacctgattcgtcggagacttcggtcgaagatgatgactggttagtggcggacgggtgagtaacgcgtgggtaacctgcctctaagagggggataacagagagaaatcactgctaataccgcataagcgcacagtaccgcatggtacagtgtgaaaagatttat

SEQ ID NO: 101 - DNA, 225 bp, Butyrivibrio fibrisolvens
  source 1..225: mol_type = genomic DNA
agagtttgatcatggctcaggatgaacgctggcggcgtgcctaacacatgcaagtcgaacggacctgatttgtcggagacttcggtcatagatgatgactggttagtggcggacgggtgagtaacgcgtgggtaacctgcctctaagagggggataacagagagaaatcactgctaataccgcataagcgcacagtaccgcatggtacagtgtgaaaagattcat

SEQ ID NO: 102 - DNA, 225 bp, Butyrivibrio fibrisolvens
  source 1..225: mol_type = genomic DNA
agagtttgatcctggctcaggatgaacgctagctacaggcttaacacatgcaagtcgaggggcagcatgagattagcttgctaatcttgatggcgaccggcgcacgggtgagtaacgcgtatccaaccttccctttactcagggatagcccggtgaaaaccggattaacacctgatagtttcacatgacggcatcagactgtgaataaaggttacggtaaaggat

SEQ ID NO: 103 - DNA, 225 bp, Peptostreptococcus anaerobius
  source 1..225: mol_type = genomic DNA
agagtttgatcatggctcaggatgaacgctggcggcgtgcctaacacatgcaagtcgagcgcgtcttatggtgcttgcaccgataagatgagcggcggacgggtgagtaacgcgtgggtaacctgccctatacacatggataacatactgaaaagtttactaatacatgataaagtatatattcggcatcgaagatataccaaagcgtaagcggtataggatgga

SEQ ID NO: 104 - DNA, 225 bp, Lachnospiraceae bacterium
  source 1..225: mol_type = genomic DNA
agagtttgatcatggctcaggatgaacgctggcggcgtgcttaacacatgcaagtcgaacggacttaatagagagcttgctcgaaattaagttagtggcggacgggtgagtaacgcgtggataacctgcctcgtactgggggacaacagttggaaacgactgctaataccgcataagcgcacggttccgcatggaacagtgtgaaaaactccggtggtacgagat

SEQ ID NO: 105 - DNA, 225 bp, Lachnospiraceae bacterium
  source 1..225: mol_type = genomic DNA
agagtttgatcctggctcaggatgaacgctggcggcgtgcttaacacatgcaagtcgaacggacttaatagagagcttgctcgaaattaagttagtggcggacgggtgagtaacgcgtggataacctgcctcgtactgggggacaacagttggaaacgactgctaataccgcataagcgcacggttctgcatggaacagtgtgaaaaactccggtggtacgagat

SEQ ID NO: 106 - DNA, 225 bp, Lachnospiraceae bacterium
  source 1..225: mol_type = genomic DNA
agagtttgatcatggctcaggatgaacgctagctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatggtttcacatgacggcatcagactgtgaataaaggttacggtaaaggat

SEQ ID NO: 107 - DNA, 225 bp, Lachnospiraceae bacterium; identical to SEQ ID NO: 104
  source 1..225: mol_type = genomic DNA

SEQ ID NO: 108 - DNA, 225 bp, Lachnospiraceae bacterium
  source 1..225: mol_type = genomic DNA
agagtttgatcatggctcaggatgaacgctggcggcgtgcttaacacatgcaagtcgaacggacttaatcgagagcttgctcgagattaagttagtggcggacgggtgagtaacgcgtggataacctgcctcgtactgggggacaacagttggaaacgactgctaataccgcataagcgcacgattccgcatggaacagtgtgaaaaactccggtggtacgagat

SEQ ID NO: 109 - DNA, 225 bp, Lachnospiraceae bacterium
  source 1..225: mol_type = genomic DNA
agagtttgatcctggctcaggatgaacgctggcggcgtgcttaacacatgcaagtcgaacggacttaatcgagagcttgctcgagattaagttagtggcggacgggtgagtaacgcgtggataacctgcctcgtactgggggacaacagttggaaacgactgctaataccgcataagcgcacgattccgcatggaacagtgtgaaaaactccggtggtacgagat

SEQ ID NO: 110 - DNA, 225 bp, unidentified
  misc_feature 1..225: note = Anaerocolumna sp.
  source 1..225: mol_type = genomic DNA
agagtttgatcatggctcaggatgaacgctggcggcgtgcttaacacatgcaagtcgaacgaagctctctagacggcgacttcggtcaaagtatagagatgacttagtggcggacgggtgagtaacgcgtgggtaacctaccttaagcagggggacaacagttggaaacgactgctaataccgcataagaccacactaccgcatggtagaggggtaaaagattta

SEQ ID NO: 111 - DNA, 225 bp, Frisingicoccus caecimuris
  source 1..225: mol_type = genomic DNA
agagtttgatcatggctcaggatgaacgctggcggcgtgcttaacacatgcaagtcgagcgaagcactttgactgatccttcgggtgatttcactgtgactgagcggcggacgggtgagtaacgcgtgggtaacctgcctcatacagggggataacagttagaaatgactgctaataccgcataagcgcacagtaccgcatggtacagtgtgaaaaactccggtg

SEQ ID NO: 112 - DNA, 225 bp, Frisingicoccus caecimuris; identical to SEQ ID NO: 13
  source 1..225: mol_type = genomic DNA

SEQ ID NO: 113 - DNA, 225 bp, Frisingicoccus caecimuris
  source 1..225: mol_type = genomic DNA
agagtttgatcctggctcaggatgaacgctagcggcaggcttaacacatgcaagtcgaggggcagcgcggggtagcaatactctggcggcgaccggcggaagggtgcgtaacgcgtgagcaacttgcccgtatcagggggataagcattggaaacgatgtctaacaccccgtaacagtagacaccgcatgatgtcttcttgaaatcctcggaggatacggatggg

SEQ ID NO: 114 - DNA, 225 bp, Frisingicoccus caecimuris
  source 1..225: mol_type = genomic DNA
agagtttgatcctggctcaggatgaacgctggcggcgtgcttaacacatgcaagtcgagcgaagcactttgactgatccttcgggtgatttcactgtgactgagcggcggacgggtgagtaacgcgtgggtaacctgcctcatacagggggataacagttagaaatgactgctaataccgcataagcgcacagtaccgcatggtacagtgtgaaaaactccggtg

SEQ ID NO: 115 - DNA, 225 bp, Frisingicoccus caecimuris
  source 1..225: mol_type = genomic DNA
agagtttgatcatggctcaggacgaacgctggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcggccgaccgcatggttgaccgatgaaagatggcctctacatgtaagctatcgccgga

SEQ ID NO: 116 - DNA, 225 bp, Frisingicoccus caecimuris
  source 1..225: mol_type = genomic DNA
agagtttgatcctggctcaggatgaacgctggcggcgtgcttaacacatgcaagtcgagcgaagcactttgattgatccttcgggtgatatcactgtgactgagcggcggacgggtgagtaacgcgtgggtaacctgcctcatacagggggataacagttagaaatgactgctaataccgcataagcgcacagtaccgcatggtacagtgtgaaaaactccggtg

SEQ ID NO: 117 - DNA, 225 bp, Frisingicoccus caecimuris; identical to SEQ ID NO: 111
  source 1..225: mol_type = genomic DNA

SEQ ID NO: 118 - DNA, 225 bp, Frisingicoccus caecimuris
  source 1..225: mol_type = genomic DNA
agagtttgatcatggctcaggatgaacgctggcggcgtgcttaacacatgcaagtcgagcgaagcactctggttgatccttcgggtgattccattgtgactgagcggcggacgggtgagtaacgcgtgggtaacctgcctcatacagggggataacagttagaaatgactgctaataccgcataagcgcacagtaccgcatggtacagtgtgaaaagctccggtg

SEQ ID NO: 119 - DNA, 225 bp, Frisingicoccus caecimuris
  source 1..225: mol_type = genomic DNA
agagtttgatcctggctcaggatgaacgctggcggcgtgcttaacacatgcaagtcgagcgaagcactttggttgatccttcgggtgattccattgtgactgagcggcggacgggtgagtaacgcgtgggtaacctgcctcatacagggggataacagttagaaatgactgctaataccgcataagcgcacagtaccgcatggtacagtgtgaaaaactccggtg

SEQ ID NO: 120 - DNA, 225 bp, Ruminiclostridium thermocellum
  source 1..225: mol_type = genomic DNA
agagtttgatcatggctcaggacgaacgctggcggcgtgcttaacacatgcaagtcgagcggggataagagcttcggtttttattctagcggcggacgggtgagtaacgcgtgagcaatctgtcccagacagggggataacacttggaaacaggtgctaataccgcataagaccacgagaggacatccttttgaggtaaaaggagaaatccggtttggggtgagc

SEQ ID NO: 121 - DNA, 225 bp, Ruminiclostridium thermocellum
  source 1..225: mol_type = genomic DNA
agagtttgatcatggctcaggacgaacgctggcggcgtgcttaacacatgcaagtcgagcggggataagagcttcggtttttattctagcggcggacgggtgagtaacgcgtgagcaatctgtcccagacagggggataacacttggaaacaggtgctaataccgcataagaccacgaggcgacatcgccttgaggtaaaaggagcaatccggtttggggtgagc

SEQ ID NO: 122 - DNA, 225 bp, unidentified
  misc_feature 1..225: note = Spiroplasma sp.
  source 1..225: mol_type = genomic DNA
agagtttgatcctggctcaggatgaacgctggcggcgtgcctaatacatgcaagtcgaacgaggtaattcgttaccgagtggcgaacgggtgagtaacacgtagataatctgccttttagtttgggatacccaggggaaactttggctaataccggatacgaggttgagagacatctcttaattttgaaaggggcttatgcctcgctagaagatgagtctgcggc

SEQ ID NO: 123 - DNA, 225 bp, unidentified
  misc_feature 1..225: note = Spiroplasma sp.
  source 1..225: mol_type = genomic DNA
agagtttgatcatggctcaggatgaacgctggcggcgtgcctaatacatgcaagtcgaacgaggtaattcgttaccgagtggcgaacgggtgagtaacacgtagataatctgccttttagtttgggatacccagaggaaactttggctaataccggatacgaggttgagagacatctcttaattttgaaaggggcttatgccccgctagaagatgagtctgcgtc

SEQ ID NO: 124 - DNA, 225 bp, Finegoldia magna
  source 1..225: mol_type = genomic DNA
agagtttgatcctggctcaggacgaacgctggcggcgtgcttaacacatgcaagtcgaacgggatttagtaaacagaaacctcggtggaagattactaatgagagtggcgaacgggtgagtaacgcgtgagcaacctgcctatgacagtgggatagcctcgggaaaccgggattaataccgcataaaatcgtagaaacacatgtttcaacggtcaaagatttatc

SEQ ID NO: 125 - DNA, 225 bp, Algoriphagus ratkowskyi
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgaaagcatttgataatgattaaagatttatcgctgatggatggacatgcgttgga

SEQ ID NO: 126 - DNA, 225 bp, Algoriphagus ratkowskyi
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgatatcatttgatagcatttgagaatgattaaagatttatcgctgatggatggacatgcgttgga

SEQ ID NO: 127 - DNA, 225 bp, Algoriphagus ratkowskyi; identical to SEQ ID NO: 125
  source 1..225: mol_type = genomic DNA

SEQ ID NO: 128 - DNA, 225 bp, Algoriphagus ratkowskyi
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgagagcatttgataatgattaaagatttatcgctgatggatggacatgcgttgga

SEQ ID NO: 129 - DNA, 225 bp, Algoriphagus ratkowskyi; identical to SEQ ID NO: 13
  source 1..225: mol_type = genomic DNA

SEQ ID NO: 130 - DNA, 225 bp, Algoriphagus ratkowskyi
  source 1..225: mol_type = genomic DNA
agatttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgaaagcatttgataacgattaaagatttatcgctgatggatggacatgcgttggat

SEQ ID NO: 131 - DNA, 225 bp, Algoriphagus ratkowskyi
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgaaagcatttgataacgattaaagatttatcgctgatggatggacatgcgttgga

SEQ ID NO: 132 - DNA, 225 bp, Algoriphagus ratkowskyi; identical to SEQ ID NO: 126
  source 1..225: mol_type = genomic DNA

SEQ ID NO: 133 - DNA, 225 bp, Prevotella copri
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgatatcgtttgaaagcatttgataatgattaaagatttatcgctgatggatggacatgcgttgga

SEQ ID NO: 134 - DNA, 225 bp, Prevotella copri; identical to SEQ ID NO: 133
  source 1..225: mol_type = genomic DNA

SEQ ID NO: 135 - DNA, 225 bp, Prevotella copri
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccggatgacatcgtttgatagcatttgagaatgattaaagatttatcgctgatggatggacatgcgttgga

SEQ ID NO: 136 - DNA, 225 bp, Prevotella copri
  source 1..225: mol_type = genomic DNA
agagtttgatcatggctcaggatgaacgctagcgacaggcctaacacatgcaagtcgagcggcagcacctcgggtggcgaccggcgcacgggtgagtaacgcgtatgtaacctgcccatcagtttgggatagcccagtgaaaactggattaataccgaatgatatcgtttgaaagcatttgataacgattaaagatttatcgctgatggatggacatgcgttgga

SEQ ID NO: 137 - DNA, 225 bp, Prevotella copri; identical to SEQ ID NO: 13
  source 1..225: mol_type = genomic DNA

SEQ ID NO: 138 - DNA, 225 bp, Prevotella copri; identical to SEQ ID NO: 130
  source 1..225: mol_type = genomic DNA

SEQ ID NO: 139 - DNA, 225 bp, Prevotella copri; identical to SEQ ID NO: 58
  source 1..225: mol_type = genomic DNA

SEQ ID NO: 140 - DNA, 225 bp, Prevotella copri; identical to SEQ ID NO: 126
  source 1..225: mol_type = genomic DNA

SEQ ID NO: 141 - DNA, 225 bp, Streptococcus equinus
  source 1..225: mol_type = genomic DNA
agagtttgatcatggctcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataatagcatttaacacatgttagatgcttgaaagaagcaattgcttcactagta

SEQ ID NO: 142 - DNA, 225 bp, Streptococcus equinus; identical to SEQ ID NO: 141
  source 1..225: mol_type = genomic DNA

SEQ ID NO: 143 - DNA, 225 bp, Streptococcus equinus
  source 1..225: mol_type = genomic DNA
agagtttgatcatggctcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcttttaactcatgttaggagcttgaaagaagcaattgcttcactagta

SEQ ID NO: 144 - DNA, 225 bp, Streptococcus equinus; identical to SEQ ID NO: 13
  source 1..225: mol_type = genomic DNA

SEQ ID NO: 145 - DNA, 225 bp, Streptococcus equinus
  source 1..225: mol_type = genomic DNA
agagtttgatcatggctcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcatttaacacatgttagatgtttgaaagaagcaattgcttcactagta

SEQ ID NO: 146 - DNA, 225 bp, Streptococcus equinus
  source 1..225: mol_type = genomic DNA
agagtttgatcatggctcaggacgaacgctggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcggtcgaccgcatggttgaccgatgaaagatggcctctacatgtaagctatcgccgga

SEQ ID NO: 147 - DNA, 225 bp, Streptococcus equinus; identical to SEQ ID NO: 13
  source 1..225: mol_type = genomic DNA

SEQ ID NO: 148 - DNA, 225 bp, Streptococcus equinus
  source 1..225: mol_type = genomic DNA
agagtttgatcatggctcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcttttaactcatgttagatgcttgaaagaagcaattgcttcactagta

SEQ ID NO: 149 - DNA, 225 bp, Prevotella copri; identical to SEQ ID NO: 58
  source 1..225: mol_type = genomic DNA

SEQ ID NO: 150 - DNA, 225 bp, Prevotella copri
  source 1..225: mol_type = genomic DNA
agagtttgatcctggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaaggactttccgcatggagagttcttgaaagatggcctctatttataagctatcgctagagg

SEQ ID NO: 151 - DNA, 225 bp, Prevotella copri
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccggatgatatcatttgaaagcatttgataatgattaaagatttatcgctgatggatggacatgcgttgga

SEQ ID NO: 152 - DNA, 225 bp, Prevotella copri; identical to SEQ ID NO: 136
  source 1..225: mol_type = genomic DNA

SEQ ID NO: 153 - DNA, 225 bp, Prevotella copri; identical to SEQ ID NO: 13
  source 1..225: mol_type = genomic DNA

SEQ ID NO: 154 - DNA, 225 bp, Prevotella copri; identical to SEQ ID NO: 130
  source 1..225: mol_type = genomic DNA

SEQ ID NO: 155 - DNA, 225 bp, Prevotella copri; identical to SEQ ID NO: 58
  source 1..225: mol_type = genomic DNA

SEQ ID NO: 156 - DNA, 225 bp, Prevotella copri; identical to SEQ ID NO: 126
  source 1..225: mol_type = genomic DNA

SEQ ID NO: 157 - DNA, 225 bp, Duodenibacillus massiliensis
  source 1..225: mol_type = genomic DNA
agagtttgatcatggctcagattgaacgctggcggcatgctttacacatgcaagtcgaacggcagcacagggagcttgctcctgggtggcgagtggcgaacgggtgagtaatacatcggaacgtgtccgaatgtgggggacaactatccgaaaggatagctaataccgcataatacctgagggtgaaagtgggggaccgcaaggcctcacgcatacggagcggcc

SEQ ID NO: 158 - DNA, 225 bp, Duodenibacillus massiliensis
  source 1..225: mol_type = genomic DNA
aagagttgatcctggctcagattgaacgctggcggcatgctttacacatgcaagtcgaacggcagcacagggagcttgctcctgggtggcgagtggcgaacgggtgagtaatacatcggaacgtgtccgaatgtgggggacaactatccgaaaggatagctaataccgcataatacctgagggtgaaagtgggggaccgcaaggcctcacgcatacggagcggcc

SEQ ID NO: 159 - DNA, 225 bp, Duodenibacillus massiliensis; identical to SEQ ID NO: 157
  source 1..225: mol_type = genomic DNA

SEQ ID NO: 160 - DNA, 225 bp, Duodenibacillus massiliensis
  source 1..225: mol_type = genomic DNA
agatttgatcctggctcagattgaacgctggcggcatgctttacacatgcaagtcgaacggcagcacagggagcttgctcctgggtggcgagtggcgaacgggtgagtaatacatcggaacgtgtccgaatgtgggggacaactatccgaaaggatagctaataccgcataatacctgagggtgaaagtgggggaccgcaaggcctcacgcatacggagcggccg

SEQ ID NO: 161 - DNA, 225 bp, Duodenibacillus massiliensis
  source 1..225: mol_type = genomic DNA
agagtttgatcctggctcagattgaacgctggcggcatgctttacacatgcaagtcgaacggcagcacagggagcttgctcctgggtggcgagtggcgaacgggtgagtaatacatcggaacgtgtccgaatgtgggggacaactatccgaaaggatagctaataccgcataatacctgagggtgaaagtgggggaccgcaaggcctcacgcatacggagcggcc

SEQ ID NO: 162 - DNA, 225 bp, Bacteroidales bacterium
  source 1..225: mol_type = genomic DNA
agagtttgatcctggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacagtgaggacatcttcgttgtttgaaagtttcgacagatacggatt

SEQ ID NO: 163 - DNA, 225 bp, Bacteroidales bacterium
  source 1..225: mol_type = genomic DNA
agagtttgatcctggctcaggatgaacgctagcgacaggcctaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacagtgagggcatcttcgttgtttgaaagtttcgacagatacggatt

SEQ ID NO: 164 - DNA, 225 bp, Bacteroidales bacterium; identical to SEQ ID NO: 162
  source 1..225: mol_type = genomic DNA

SEQ ID NO: 165 - DNA, 225 bp, Bacteroidales bacterium
  source 1..225: mol_type = genomic DNA
agagtttgatcatggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacagtgaggacatcttcgttgtttgaaagtttcgacagatacggatt

SEQ ID NO: 166 - DNA, 225 bp, Bacteroidales bacterium
  source 1..225: mol_type = genomic DNA
agagtttgatcatggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacagtgaggacatcttcgttgtttgaaagttacgacagatacggatt

SEQ ID NO: 167 - DNA, 225 bp, Bacteroidales bacterium
  source 1..225: mol_type = genomic DNA
agagtttgatcctggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacattggagacatctccggagtttgaaagtttcgacagatacggatt

SEQ ID NO: 168 - DNA, 225 bp, Bacteroidales bacterium; identical to SEQ ID NO: 13
  source 1..225: mol_type = genomic DNA

SEQ ID NO: 169 - DNA, 225 bp, Bacteroidales bacterium
  source 1..225: mol_type = genomic DNA
agagtttgatcctggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaaccatggagacatcttcgttgtttgaaagtttcgacagatacggatt

SEQ ID NO: 170 - DNA, 225 bp, Bacteroidia bacterium
  source 1..225: mol_type = genomic DNA
agagtttgatcctggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacaggggaggcatcttctttgtttgaaagtttcgacagatacggatt

SEQ ID NO: 171 - DNA, 225 bp, Bacteroidia bacterium; identical to SEQ ID NO: 170
  source 1..225: mol_type = genomic DNA

SEQ ID NO: 172 - DNA, 225 bp, Bacteroidia bacterium
  source 1..225: mol_type = genomic DNA
agagtttgacatggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacaggggaggcatcttctttgtttgaaagtttcgacagatacggattg

SEQ ID NO: 173 - DNA, 225 bp, Bacteroidia bacterium
  source 1..225: mol_type = genomic DNA
agatttgatcatggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacaggggaggcatcttctttgtttgaaagtttcgacagatacggattg

SEQ ID NO: 174 - DNA, 225 bp, Bacteroidia bacterium
  source 1..225: mol_type = genomic DNA
agaggtttgatcatggctcaggatgaacgctagcggcaggcttaacacaggcaagtagaggggcagcataagggtagcaatatctttgatggcgaccggcggaagggtgcgtaacgcgtgagcaacatgcccgtatccgggggataaccgagagaaatttcgactaataccccataacaacaggggaggcatcttctttgtttgaaagtttcgacagatacggat

SEQ ID NO: 175 - DNA, 225 bp, Bacteroidia bacterium
  source 1..225: mol_type = genomic DNA
agagtttgatcatggctcaggatgaacgctggcggcacgcctaacacatgcaagtcgaggggcagcataagggtagcaatatctttgatggcgaccggcggaagggtgcataacgcgtgagcaacatgcccgtatccgggggataaccgagagaaatttcgactaataccccataacaacaggggaggcatcttctttgtttgaaagtttcgacagatacggatt

SEQ ID NO: 176 - DNA, 225 bp, Bacteroidia bacterium
  source 1..225: mol_type = genomic DNA
agatttgatcctggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacaggggaggcatcttctttgtttgaaagtttcgacagatacggattg

SEQ ID NO: 177 - DNA, 225 bp, Bacteroidia bacterium
  source 1..225: mol_type = genomic DNA
agagtttgatcctggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacagaggaggcatcttctttgtttgaaagtttcgacagatacggatt

SEQ ID NO: 178 - DNA, 225 bp, Cecembia lonarensis
  source 1..225: mol_type = genomic DNA
agagtttgatcctggctcaggatgaacgctagcgacaggcctaacacatgcaagtcgagggagatccccttcgggggtgagaccggcgcacgggtgagtaacgcgtatgtaacctgcccataagtgggggatagcccagcgaaagctggattaataccgcataatatccgagactgacatctttttcggattaaagatttattgcttatggatggacatgcgttg

SEQ ID NO: 179 - DNA, 225 bp, Cecembia lonarensis
  source 1..225: mol_type = genomic DNA
agagtttgatcctggctcaggatgaacgctagcgacaggcctaacacatgcaagtcgagggagatccccttcgggggtgagaccggcgcacgggtgagtaacgcgtatgtaacctgcccataagtgggggatagcccagtgaaaactggattaataccgcataataccggagactgacatctttttctggttaaagatttattgcttatggatggacatgcgttg

SEQ ID NO: 180 - DNA, 225 bp, Cecembia lonarensis
  source 1..225: mol_type = genomic DNA
agagtttgatcatggctcaggatgaacgctagcgacaggcctaacacatgcaagtcgagggagatccccttcgggggtgagaccggcgcacgggtgagtaacgcgtatgtaacctgcccataagtgggggatagcccagcgaaagctggattaataccgcataatatccgagactgacatctttttcggattaaagatttattgcttatggatggacatgcgttg

SEQ ID NO: 181 - DNA, 225 bp, Cecembia lonarensis
  source 1..225: mol_type = genomic DNA
agagtttgatcatggctcaggatgaacgctagcgacaggcctaacacatgcaagtcgagggagatccccttctggggtgagaccggcgcacgggtgagtaacgcgtatgtaacctgcccataagtgggtgatagcccagcgaaagctggattaataccgcataatatccgagactgacatctttttcggattaaagatttattgcttacggatggacatgcgttg

SEQ ID NO: 182 - DNA, 225 bp, Cecembia lonarensis
  source 1..225: mol_type = genomic DNA
agagttgctcatggctcaggatgaacgctagcgacaggcctaacacatgcaagtcgagggagattcccttcggggatgagaccggcgcacgggtgagtaacgcgtatgtaacctgcccataagtgggggatagcccagcgaaagctggattaataccgcataatatccgagactgacatctttttcggattaaagatttattgcttatggatggacatgcgttgg

SEQ ID NO: 183 - DNA, 225 bp, Cecembia lonarensis
  source 1..225: mol_type = genomic DNA
agagtttgatcatggctcaggatgaacgctagcgacaggcctaacacatgcaagtcgagggagattcccttcggggatgagaccggcgcacgggtgagtaacgcgtatgtaacctgcccataagtgggggatagcccagcgaaagctggattaataccgcataatatccgagactgacatctttttcggattaaagatttattgcttatggatggacatgcgttg

SEQ ID NO: 184 - DNA, 225 bp, Cecembia lonarensis; identical to SEQ ID NO: 178
  source 1..225: mol_type = genomic DNA

SEQ ID NO: 185 - DNA, 225 bp, Cecembia lonarensis
  source 1..225: mol_type = genomic DNA
agagtttgatcatggctcaggatgaacgctagcgacaggcctaacacatgcaagtcgagggagatccccttcaggggtgagaccggcgcacgggtgagtaacgcgtatgtaacctgcccataagtgggggatagcccagcgaaagctggattaataccgcataatatccgagactgacatctttttcggattaaagatttattgcttatggatggacatgcgttg

SEQ ID NO: 186 - DNA, 225 bp, Bacteroidia bacterium
  source 1..225: mol_type = genomic DNA
agagtttgatcctggctcaggatgaacgctagcggcaggcttaacacatgcaagtcgaggggcagcagcggggagtagcaatacactccggctggcgaccggcgcaagggtgcgtaacgcgtgagcaacatgcccgtatcagggacataaccggtggaaacgccgcctaatttcccataacatgtgaagaggcatctctttatattgaaagattcgtcggatacg

SEQ ID NO: 187 - DNA, 225 bp, Bacteroidia bacterium
  source 1..225: mol_type = genomic DNA
agagtttgatcctggctcaggatgaacgctagcggcaggcttaacacatgcaagtcgaggcgcagcagcggggagtagcaatacactccggctggcgaccggcgcaagggtgcgtaacgcgtgagcaacatacccgtatcagggacataaccggtggaaacgccgcctaatttcccataacatgtgaagaggcatctctttatattgaaagattcgtcggatacg

SEQ ID NO: 188 - DNA, 225 bp, Bacteroidia bacterium
  source 1..225: mol_type = genomic DNA
agagtttgatcatggctcaggatgaacgctagcggcaggcttaacacatgcaagtcgaggggcagcagcagggagtagcaatacaccttggctggcgaccggcgcaagggtgcgtaacgcgtgagcaacatgcccgtatcagggacataaccggtggaaacgccgcctaatttcccataatatgtgaagaggcatctctttatattgaaagattcgtcggatacg

SEQ ID NO: 189 - DNA, 225 bp, Bacteroidia bacterium; identical to SEQ ID NO: 186
  source 1..225: mol_type = genomic DNA

SEQ ID NO: 190 - DNA, 225 bp, Bacteroidia bacterium
  source 1..225: mol_type = genomic DNA
agagtttgatcctggctcaggatgaacgctagcggcaggcttaacacatgcaagtcgaggggcagcagcagggagtagcaatacaccttggctggcgaccggcgcaagggtgcgtaacgcgtgagcaacatgcccgtatcagggacataaccggtggaaacgccgcctaatttcccataacatgtgaagaggcatctctttatattgaaagattcgtcggatacg

SEQ ID NO: 191 - DNA, 225 bp, Bacteroidia bacterium; identical to SEQ ID NO: 13
  source 1..225: mol_type = genomic DNA

SEQ ID NO: 192 - DNA, 225 bp, Bacteroidia bacterium
  source 1..225: mol_type = genomic DNA
agagtttgatcatggctcaggatgaacgctagcggcaggcttaacacatgcaagtcgaggggcagcagcagggagtagcaatacaccttggctggcgaccggcgcaagggtgcgtaacgcgtgagcaacatgcccgtatcagggacataaccggtggaaacgccgcctaatttcccataacatgtgaagaggcatctctttatattgaaagattcgtcggatacg

SEQ ID NO: 193 - DNA, 225 bp, Bacteroidia bacterium; identical to SEQ ID NO: 115
  source 1..225: mol_type = genomic DNA

SEQ ID NO: 194 - DNA, 225 bp, Bacteroidales bacterium; identical to SEQ ID NO: 113
  source 1..225: mol_type = genomic DNA

SEQ ID NO: 195 - DNA, 225 bp, Bacteroidales bacterium
  source 1..225: mol_type = genomic DNA
agagtttgatcatggctcaggatgaacgctagcgacaggcttaacacatgcaagtcgaggggcagcgcggcggtagcaataccgctggcggcgaccggcggaagggtgcgtaacgcgtgagcaacttgcccgtatcagggggataagcattggaaacgatgtctaacaccccgtaacagtagacaccgcatgatgtcttcttgaaatcctcggaggatacggatg

SEQ ID NO: 196 - DNA, 225 bp, Bacteroidales bacterium
  source 1..225: mol_type = genomic DNA
agagtttgatcatggctcaggatgaacgctagcggcaggcttaacacatgcaagtcgaggggcagcgcggggtagcaatactctggcggcgaccggcggaagggtgcgtaacgcgtgagcaacttgcccgtatcagggggataagcattggaaacgatgtctaacaccccgtaacaggagacaccgcatgatgtcttcttgaaatcctcggaggatacggatggg

SEQ ID NO: 197 - DNA, 225 bp, Bacteroidales bacterium; identical to SEQ ID NO: 113
  source 1..225: mol_type = genomic DNA

SEQ ID NO: 198 - DNA, 225 bp, Bacteroidales bacterium
  source 1..225: mol_type = genomic DNA
agagtttgatcctggctcaggatgaacgctagcggcaggcttaacacatgcaagtcgaggggcagcagggagtagcaatactctggcggcgaccggcggaagggtgcgtaacgcgtgagcaacttgcccgtatcagggggataagcattggaaacgatgtctaacaccccgtaacagtagacaccgcatgatgtcttcttgaaatcctcggaggatacggatggg

SEQ ID NO: 199 - DNA, 225 bp, Bacteroidales bacterium
  source 1..225: mol_type = genomic DNA
agagtttgatcatggctcaggatgaacgctagcggcaggcttaacacatgcaagtcgaggggcagcgcggggtagcaatactctggcggcgaccggcggaagggtgcgtaacgcgtgagcaacttgcccgtatcagggggataagcattggaaacgatgtctaacaccccgtaacagtagacaccgcatgatgtcttcttgaaatcctcggaggatacggatggg

SEQ ID NO: 200 - DNA, 225 bp, Clostridium maximum
  source 1..225: mol_type = genomic DNA
agagtttgatcctggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttaataatcaaaggagtaatccgctataagatgggc

SEQ ID NO: 201 - DNA, 225 bp, Clostridium maximum
  source 1..225: mol_type = genomic DNA
agagtttgatcatggtcaggatgaacgctggcggcgtgcttaacacatgcaagtcgagcgagaaaagcttcttcggaagtttttctagcggcggacgggtgagtaacacgtgggcaacctgccttatagaggggaatagccttccgaaaggaagattaataccgcataacattattgaaaggcatcttttgataatcaaaggagcaatccgctataagatgggcc

SEQ ID NO: 202 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcgggaatttttctagcggcggacgggtgagtaacacgtgggcaacctgccttatagaggggaatagccttccgaaaggaagattaataccgcataacattattgaaaggcatcttttaataatcaaaggagcaatccgctataagatgggc

SEQ ID NO: 203 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcgggaatttttctagcggcggacgggtgagtaacacgtgggcaacctgccttatagaggggaatagccttccgaaaggaagattaataccgcataatattattgaaaggcatcttttaataatcaaaggagcaatccgctataagatgggc

SEQ ID NO: 204 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttaataatcaaaggagtaatccgctataagatgggc

SEQ ID NO: 205 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttaataatcaaaggagcaattcgctataagatgggc

SEQ ID NO: 206 - DNA, 225 bp, Clostridium maximum; identical to SEQ ID NO: 200
  source 1..225: mol_type = genomic DNA

SEQ ID NO: 207 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttaataatcaaaggagcaatccgctataagatgggc

SEQ ID NO: 208 - DNA, 225 bp, Finegoldia magna; identical to SEQ ID NO: 28
  source 1..225: mol_type = genomic DNA

SEQ ID NO: 209 - DNA, 225 bp, Bacteroidales bacterium; identical to SEQ ID NO: 177
  source 1..225: mol_type = genomic DNA

SEQ ID NO: 210 - DNA, 225 bp, Bacteroidales bacterium
  source 1..225: mol_type = genomic DNA
agagtttgatcatggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacagaggaggcatcttctttgtttgaaagtttcgacagatacggatt

SEQ ID NO: 211 - DNA, 225 bp, Bacteroidales bacterium; identical to SEQ ID NO: 172
  source 1..225: mol_type = genomic DNA

SEQ ID NO: 212 - DNA, 225 bp, Bacteroidales bacterium; identical to SEQ ID NO: 173
  source 1..225: mol_type = genomic DNA

SEQ ID NO: 213 - DNA, 225 bp, Bacteroidales bacterium; identical to SEQ ID NO: 175
  source 1..225: mol_type = genomic DNA

SEQ ID NO: 214 - DNA, 225 bp, Bacteroidales bacterium
  source 1..225: mol_type = genomic DNA
agagtttgatcctggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacagaggaggcatctcctttgcttgaaagtttcgacagatacggatt

SEQ ID NO: 215 - DNA, 225 bp, Bacteroidales bacterium; identical to SEQ ID NO: 176
  source 1..225: mol_type = genomic DNA

SEQ ID NO: 216 - DNA, 225 bp, Bacteroidales bacterium; identical to SEQ ID NO: 177
  source 1..225: mol_type = genomic DNA

SEQ ID NO: 217 - DNA, 225 bp, Bacteroidales bacterium
  source 1..225: mol_type = genomic DNA
agagtttgatcctggctcaggatgaacgctagcggcaggcttaacacatgcaagtcgaggggcagcgcggggtagcaatactctggcggcgaccggcggaagggtgcgtaacgcgtgagcaacattcccgtatcagggagataaccggaggaaactccgcctaatatcccataacaacagtggggacatccccgttgtttgaaagattcgtcggatacggattgg

SEQ ID NO: 218 - DNA, 225 bp, Bacteroidales bacterium
  source 1..225: mol_type = genomic DNA
agagtttgatcatggctcaggatgaacgctagcggcaggcttaacacatgcaagtcgaggggcagcgcggagtagcaatactctggcggcgaccggcggaagggtgcgtaacgcgtgagcaacattcccgtatcagggagataaccggaggaaactccgcctaatatcccataacaacagtggggacatccccgttgtttgaaagattcgtcggatacggattgg

SEQ ID NO: 219 - DNA, 225 bp, Bacteroidales bacterium; identical to SEQ ID NO: 217
  source 1..225: mol_type = genomic DNA

SEQ ID NO: 220 - DNA, 225 bp, Bacteroidales bacterium; identical to SEQ ID NO: 13
  source 1..225: mol_type = genomic DNA

SEQ ID NO: 221 - DNA, 225 bp, unidentified; identical to SEQ ID NO: 104
  misc_feature 1..225: note = Parvimonas sp.
  source 1..225: mol_type = genomic DNA

SEQ ID NO: 222 - DNA, 225 bp, Bacteroidales bacterium
  source 1..225: mol_type = genomic DNA
agagtttgatcctggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacaggggaggcatctcctttgcttgaaagtttcgacagatacggatt

SEQ ID NO: 223 - DNA, 225 bp, Bacteroidales bacterium
  source 1..225: mol_type = genomic DNA
agagtttgatcatggctcaggatgaacgctagcggcaggcttaacacatgcaagtcgaggggcagcataagggtagcaatatctttgatggcgaccggcggaagggtgcgtaacgcgtgagcaacatgcccgtatccgggggataaccgagagaaatttcgactaataccccataacaacagaggaggcatctcctttgcttgaaagtttcgacagatacggatt

SEQ ID NO: 224 - DNA, 225 bp, Bacteroidales bacterium; identical to SEQ ID NO: 172
  source 1..225: mol_type = genomic DNA

SEQ ID NO: 225 - DNA, 225 bp, Bacteroidales bacterium; identical to SEQ ID NO: 173
  source 1..225: mol_type = genomic DNA

SEQ ID NO: 226 - DNA, 225 bp, Bacteroidales bacterium; identical to SEQ ID NO: 214
  source 1..225: mol_type = genomic DNA

SEQ ID NO: 227 - DNA, 225 bp, Bacteroidales bacterium; identical to SEQ ID NO: 176
  source 1..225: mol_type = genomic DNA

SEQ ID NO: 228 - DNA, 225 bp, Bacteroidales bacterium; identical to SEQ ID NO: 177
  source 1..225: mol_type = genomic DNA

SEQ ID NO: 229 - DNA, 225 bp, Bacteroidia bacterium
  source 1..225: mol_type = genomic DNA
agagtttgatcctggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaaccatggagacatcttcgaggtttgaaagtttcgacagatacggatt

SEQ ID NO: 230 - DNA, 225 bp, Bacteroidia bacterium
  source 1..225: mol_type = genomic DNA
agagtttgatcatggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaacattggagacatctccggagtttgaaagtttcgacagatacggatt

SEQ ID NO: 231 - DNA, 225 bp, Bacteroidia bacterium; identical to SEQ ID NO: 229
  source 1..225: mol_type = genomic DNA

SEQ ID NO: 232 - DNA, 225 bp, Bacteroidia bacterium
  source 1..225: mol_type = genomic DNA
agagtttgatcatggctcaggatgaacgctagcggcaggcttaacacatgcaagtcgaggggcagcgtgattgtagcaatacgattggcggcgaccggcggaagggtgcgtaacgcgtgagcaacatgcccgtatccgggggataaccgagagaaatttcgactaataccccataacaaccatggagacatcttcaaggtttgaaagttacgacagatacggatt

SEQ ID NO: 233 - DNA, 225 bp, Bacteroidia bacterium
  source 1..225: mol_type = genomic DNA
agagtttgatcctggctcaggatgaacgctagcggcaggcttaacacatgcaagtcgaggggcagcgtgattgtagcaatacgattgacggcgaccggcggaagggtgcgtaacgcgtgagcaacatgcccgtatccgggggataaccgagagaaatttcgactaataccccataacaactatggagacatctccggagtttgaaagttacgacagatacggatt

SEQ ID NO: 234 - DNA, 225 bp, Bacteroidia bacterium
  source 1..225: mol_type = genomic DNA
agagtttgatcatggctcaggatgaacgctagctacaggcttaacacatgcaagtcgagcgggaccctttcgggggtctagcggcaaacgggtgagtaacacgtatccaacctttcccttacttggggatagcctttagaaatgaagagtaatacccgatgtggtgcgattatcgcatgttcattgcaccaaagattcatcggtaaggggtggggatgcgttcca

SEQ ID NO: 235 - DNA, 225 bp, Bacteroidia bacterium; identical to SEQ ID NO: 13
  source 1..225: mol_type = genomic DNA

SEQ ID NO: 236 - DNA, 225 bp, Bacteroidia bacterium; identical to SEQ ID NO: 169
  source 1..225: mol_type = genomic DNA

SEQ ID NO: 237 - DNA, 225 bp, unidentified
  misc_feature 1..225: note = Acidaminococcus sp.
  source 1..225: mol_type = genomic DNA
agagtttgatcctggctcaggacgaacgctggcggcatgcctaacacatgcaagtcgaacggggtttcttcggaaacctagtggcgaacgggtgagtaacgcgtaggcaacctgccctccggatggggacaacattccgaaaggagtgctaataccgaatgtgaagcggttatcgcatgatgaccgtttgaaagatggcctctatttataagctatcgccggggg

SEQ ID NO: 238 - DNA, 225 bp, unidentified
  misc_feature 1..225: note = Acidaminococcus sp.
  source 1..225: mol_type = genomic DNA
agagtttgatcatggctcaggacgaacgctggcggcatgcctaacacatgcaagtcgaacggggcttcttcggaagccaagtggcgaacgggtgagtaacgcgtaggcaacctgccctccggatggggacaacattccgaaaggagtgctaataccgaatgtgaagcggttatcgcatgatgactgcttgaaagatggcctctatttataagctatcgccggggg

SEQ ID NO: 239 - DNA, 225 bp, unidentified; identical to SEQ ID NO: 237
  misc_feature 1..225: note = Acidaminococcus sp.
  source 1..225: mol_type = genomic DNA

SEQ ID NO: 240 - DNA, 225 bp, unidentified
  misc_feature 1..225: note = Acidaminococcus sp.
  source 1..225: mol_type = genomic DNA
agagtttgatcctggctcaggacgaacgctggcggcatgcctaacacatgcaagtcgaacggggcttcttcggaagccaagtggcgaacgggtgagtaacgcgtaggcaacctgccctccggatggggacaacattccgaaaggagtgctaataccgaatgtgaagcggttatcgcatgatgaccgtttgaaagatggcctctatttataagctatcgccggggg

SEQ ID NO: 241 - DNA, 225 bp, unidentified
  misc_feature 1..225: note = Acidaminococcus sp.
  source 1..225: mol_type = genomic DNA
agagtttgatcatggctcaggacgaacgctggcggcatgcctaacacatgcaagtcgaacggggtttcttcggaaacctagtggcgaacgggtgagtaacgcgtaggcaacctgccctccggatggggacaacattccgaaaggagtgctaataccgaatgtgaagcggttatcgcatgatgaccgcttgaaagatggcctctatttataagctatcgccggggg

SEQ ID NO: 242 - DNA, 225 bp, unidentified
  misc_feature 1..225: note = Acidaminococcus sp.
  source 1..225: mol_type = genomic DNA
agagtttgatcctggctcaggacgaacgctggcggcatgcctaacacatgcaagtcgaacggggtttcttcggaaacctagtggcgaacgggtgagtaacgcgtaggcaacctgccctccggatggggacaacattccgaaaggagtgctaataccgaatgtgaagcggttatcgcatgatgaccgcttgaaagatggcctctatttataagctatcgccggggg

SEQ ID NO: 243 - DNA, 225 bp, unidentified
  misc_feature 1..225: note = Acidaminococcus sp.
  source 1..225: mol_type = genomic DNA
agagtttgatcctggctcaggacgaacgctggcggcatgcctaacacatgcaagtcgaacggggcttcttcggaagccaagtggcgaacgggtgagtaacgcgtaggcaacctgccctccggatggggacaacattccgaaaggagtgctaataccgaatgtgaagcggttatcgcatgatgactgcttgaaagatggcctctatttataagctatcgccggggg

SEQ ID NO: 244 - DNA, 225 bp, unidentified
  misc_feature 1..225: note = Acidaminococcus sp.
  source 1..225: mol_type = genomic DNA
agagtttgatcatggctcaggacgaacgctggcggcatgcctaacacatgcaagtcgaacggggcttcttcggaagccaagtggcgaacgggtgagtaacgcgtaggcaacctgccctccggatggggacaacattccgaaaggagtgctaataccgaatgtgaagcggttatcgcatgatgaccgtttgaaagatggcctctatttataagctatcgccggggg

SEQ ID NO: 245 - DNA, 225 bp, Bacteroidales bacterium
  source 1..225: mol_type = genomic DNA
agagtttgatcatggctcaggatgaacgctagcggcaggcttaacacatgcaagtcgaggggcagcggggagtagcaatactctgccggcgaccggcggaagggtgcgtaacacgtgagcaacatgcccgtatccgggggataagcgttggaaacgacgtctaacatcccataacaacgaccgggacatcccggatgtttgaaaactgaggtggatacggattgg

SEQ ID NO: 246 - DNA, 225 bp, Bacteroidales bacterium
  source 1..225: mol_type = genomic DNA
agagtttgatcctggctcaggatgaacgctagcggcaggcttaacacatgcaagtcgaggggcagcggggagtagcaatactctgccggcgaccggcggaagggtgcgtaacacgtgagcaacatgcccgtatccgggggataagcgttggaaacgacgtctaacatcccataacaacgtccgggacatcccggatgtttgaaaactgaggtggatacggattgg

SEQ ID NO: 247 - DNA, 225 bp, Bacteroidales bacterium; identical to SEQ ID NO: 245
  source 1..225: mol_type = genomic DNA

SEQ ID NO: 248 - DNA, 225 bp, Bacteroidales bacterium
  source 1..225: mol_type = genomic DNA
agagtttgatcctggctcaggatgaacgctagcggcaggcttaacacatgcaagtcgaggggcagcggggagtagcaatactctgccggcgaccggcggaagggtgcgtaacacgtgagcaacatgcccgtatccgggggataagcgttggaaacgacgtctaacatcccataacaacgaccgggacatcccggatgtttgaaaactgaggtggatacggattgg

SEQ ID NO: 249 - DNA, 225 bp, Streptococcus orisasini
  source 1..225: mol_type = genomic DNA
agagtttgatcctggctcaggacgaacgctggcggcgtgcctaatacatgcaagtggaacgcagtgaattcaccggagcttgctccaccgaattgactgagtcgcgaacgggtgagtaacgcgtaggtaacctgccttgtagcgggggataactattggaaacgatagctaataccgcataacaacagatgacacatgttagctgtttgaaaggagcaattgctc

SEQ ID NO: 250 - DNA, 225 bp, Bacteroides graminisolvens
  source 1..225: mol_type = genomic DNA
agagtttgatcctggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcacttccacgaaagcgtaacggtgaaggatgggg

SEQ ID NO: 251 - DNA, 225 bp, Bacteroides graminisolvens; identical to SEQ ID NO: 250
  source 1..225: mol_type = genomic DNA

SEQ ID NO: 252 - DNA, 225 bp, Bacteroides graminisolvens
  source 1..225: mol_type = genomic DNA
agatttgatcatggctcaggatgaacgctagcgacaggcctaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcacttccacgaaagcgtaacggtgaaggatgggga

SEQ ID NO: 253 - DNA, 225 bp, Bacteroides graminisolvens
  source 1..225: mol_type = genomic DNA
agagtttgatcctggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtgggagagttacatggcgctcccacgaaagcgtaacggtgaaggatgggg

SEQ ID NO: 254 - DNA, 225 bp, Bacteroides graminisolvens
  source 1..225: mol_type = genomic DNA
agagtttgatcatggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtgggagagttacatggcgctcccacgaaagcgtaacggtgaaggatgggg

SEQ ID NO: 255 - DNA, 225 bp, Bacteroides graminisolvens
  source 1..225: mol_type = genomic DNA
agagtttgatcatggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcacttccacgaaagcgtaacggtgaaggatgggg

SEQ ID NO: 256 - DNA, 225 bp, Bacteroides graminisolvens
  source 1..225: mol_type = genomic DNA
agagtttgatcctggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtgggagagttacatggcgctcccacgaaagcattacggtgaaggatgggg

SEQ ID NO: 257 - DNA, 225 bp, Bacteroides graminisolvens
  source 1..225: mol_type = genomic DNA
agagtttgatcatggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcgcttccacgaaagcgtaacggtgaaggatgggg

SEQ ID NO: 258 - DNA, 225 bp, Bacteroidales bacterium; identical to SEQ ID NO: 199
  source 1..225: mol_type = genomic DNA

SEQ ID NO: 259 - DNA, 225 bp, Bacteroidales bacterium
  source 1..225: mol_type = genomic DNA
agagtttgatcctggctcaggatgaacgctagcggcaggcttaacacatgcaagtcgaggggcagcgcggggtagcaatactctggcggcgaccggcggaagggtgcgtaacgcgtgagcaacttgcccgtatcagggggataagcattggaaacgatgtctaacaccccgtaacaggagacaccgcatgatgtcttcttgaaatcctcggaggatacggatggg

SEQ ID NO: 260 - DNA, 225 bp, Bacteroidales bacterium; identical to SEQ ID NO: 113
  source 1..225: mol_type = genomic DNA

SEQ ID NO: 261 - DNA, 225 bp, Bacteroidales bacterium; identical to SEQ ID NO: 198
  source 1..225: mol_type = genomic DNA

SEQ ID NO: 262 - DNA, 225 bp, Bacteroidales bacterium; identical to SEQ ID NO: 199
  source 1..225: mol_type = genomic DNA

SEQ ID NO: 263 - DNA, 225 bp, Parabacteroides distasonis; identical to SEQ ID NO: 36
  source 1..225: mol_type = genomic DNA

SEQ ID NO: 264 - DNA, 225 bp, Parabacteroides distasonis
  source 1..225: mol_type = genomic DNA
agagtttgatcatggctcaggatgaacgctagctacaggcttaacacatgcaagtcgaggggcagcacttcgggtggcgaccggcgcacgggtgagtaacgcgtttgtaacctgcccatcagtgagggatagcccagtgaaaactggattaataccgcatgatatcattagatagcatttgaaaatgattaaagatttattgctgatggatggacatgcgttgga

SEQ ID NO: 265 - DNA, 225 bp, Parabacteroides distasonis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattagatagcatttgaaaatgattaaagatttattgctgatggatggacatgcgtagga

SEQ ID NO: 266 - DNA, 225 bp, Parabacteroides distasonis
  source 1..225: mol_type = genomic DNA
agagtttgatcctggctcaggatgaacgctagcggcaggcttaacacatgcaagtcgaggggcagcacttcgggcggcgaccggcgcacgggtgagtaacgcgtatgtaacctgcccatcagtgagggatagcccagtgaaaactggattaataccgcatgatatcattagatagcatttgaaaatgattaaagatttattgctgatggatggacatgcgttgga

SEQ ID NO: 267 - DNA, 225 bp, Parabacteroides distasonis
  source 1..225: mol_type = genomic DNA
agagtttgatcctggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattggatagcatttgataatgattaaagatttattgctgatggatggacatgcgttgga

SEQ ID NO: 268 - DNA, 225 bp, Parabacteroides distasonis; identical to SEQ ID NO: 36
  source 1..225: mol_type = genomic DNA

SEQ ID NO: 269 - DNA, 225 bp, Parabacteroides distasonis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattagatagcatttgaaaatgattaaagatttattgctgatggatggacatgcgttgga

SEQ ID NO: 270 - DNA, 225 bp, Parabacteroides distasonis; identical to SEQ ID NO: 126
  source 1..225: mol_type = genomic DNA

SEQ ID NO: 271 - DNA, 225 bp, Bacteroides coprophilus
  source 1..225: mol_type = genomic DNA
agagtttgatcatggctcaggatgaacgctagctacaggcctaacacatgcaagtcgaggggcagcgggaaataagcttgcttattttgccggcgaccggcgcacgggtgcgtaacgcgtatcgaacctgccctctactcggggatagccttgcgaaagtaagattaatacccgatggtctcaaactaagacatctttatttgagtaaagattcatcggtagagg

SEQ ID NO: 272 - DNA, 225 bp, Bacteroides coprophilus
  source 1..225: mol_type = genomic DNA
agagtttgatcatggctcaggatgaacgctagctacaggcctaacacatgcaagtcgaggggcagcgggaataaagcttgctttatttgccggcgaccggcgcacgggtgcgtaacgcgtatcgaacctgccctctactcggggatagccttgcgaaagtaagattaatacccgatggtctcaaagtaagacatctttatttgagtaaagattcatcggtagagg

SEQ ID NO: 273 - DNA, 225 bp, Bacteroides coprophilus; identical to SEQ ID NO: 13
  source 1..225: mol_type = genomic DNA

SEQ ID NO: 274 - DNA, 225 bp, Bacteroides coprophilus
  source 1..225: mol_type = genomic DNA
agagtttgatcatggctcaggatgaacgctagctacaggcctaacacatgcaagacgaggggcagcgggaaataagcttgcttatcttgccggcgaccggcgcacgggtgcgtaacgcgtatcgaacctgccctctactcggggatagccttgcgaaagtaagattaatacccgatggtctcaaagtaagacatctttatttgagtaaagattcatcggtagagg

SEQ ID NO: 275 - DNA, 225 bp, Bacteroides coprophilus
  source 1..225: mol_type = genomic DNA
agagtttgatcctggctcaggatgaacgctagctacaggcctaacacatgcaagtcgaggggcagcgggaaataagcttgcttattttgccggcgaccggcgcacgggtgcgtaacgcgtatcgaacctgccctctactcggggatagccttgcgaaagtaagattaatacccgatggtctcaaagtaagacatctttatttgagtaaagattcatcggtagagg

SEQ ID NO: 276 - DNA, 225 bp, Bacteroides coprophilus
  source 1..225: mol_type = genomic DNA
agagtttgatcctggctcaggatgaacgctagctacaggcctaacacatgcaagtcgaggggcagcgggaaataagcttgcttattttgccggcgaccggcgcacgggtgcgtaacgcgtatcgaacctgccctctactcggggatagccttgcgaaagtaagattaatacccgatggtctcaaactaagacatctttatttgagtaaagattcatcggtagagg

SEQ ID NO: 277 - DNA, 225 bp, Bacteroides coprophilus; identical to SEQ ID NO: 13
  source 1..225: mol_type = genomic DNA

SEQ ID NO: 278 - DNA, 225 bp, Acidaminococcus fermentans
  source 1..225: mol_type = genomic DNA
agagtttgatcctggctcaggacgaacgctggcggcatgcctaacacatgcaagtcgaacggggcttcttcggaagccaagtggcgaacgggtgagtaacgcgtaggcaacctgccctccggatggggacaacatcccgaaaggggtgctaataccgaatgagaccctgctgtcgcatgacagcaggatgaaagacggcctctatttataagctgtcgccggagg

SEQ ID NO: 279 - DNA, 225 bp, Bacteroidales bacterium; identical to SEQ ID NO: 106
  source 1..225: mol_type = genomic DNA

SEQ ID NO: 280 - DNA, 225 bp, Bacteroidales bacterium
  source 1..225: mol_type = genomic DNA
agagtttgatcctggctcaggatgaacgctagctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatggtttcacatgacggcatcagactgtgaataaaggttacggtaaaggat

SEQ ID NO: 281 - DNA, 225 bp, Bacteroidales bacterium; identical to SEQ ID NO: 102
  source 1..225: mol_type = genomic DNA

SEQ ID NO: 282 - DNA, 225 bp, Bacteroidales bacterium; identical to SEQ ID NO: 106
  source 1..225: mol_type = genomic DNA

SEQ ID NO: 283 - DNA, 225 bp, Bacteroidales bacterium
  source 1..225: mol_type = genomic DNA
agagtttgatcctggctcaggatgaacgctagctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatgggttcacatgacggcaacagactgtgaataaaggttacggtaaaggat

SEQ ID NO: 284 - DNA, 225 bp, Bacteroidales bacterium
  source 1..225: mol_type = genomic DNA
agagtttgatcatggctcaggatgaacgctagctacaggcttaacacatgcaagtcgaggggcagcatgagattagcttgctaatcttgatggcgaccggcgcacgggtgagtaacgcgtatccaaccttccctttactcagggatagcccggtgaaaaccggattaacacctgatggtttcacatgacggcatcagactgtgaataaaggttacggtaaaggat

SEQ ID NO: 285 - DNA, 225 bp, Bacteroidales bacterium
  source 1..225: mol_type = genomic DNA
agatttgatcatggctcaggatgaacgctagctacaggcttaacacatgcaagtcgaggggcagcatgagattagcttgctaatcttgatggcgaccggcgcacgggtgagtaacgcgtatccaactttccctttactcagggatagcccggtgaaaaccggattaacacctgatggtttcacatgacggcatcagactgtgaataaaggttacggtaaaggatg

SEQ ID NO: 286 - DNA, 225 bp, Bacteroidales bacterium
  source 1..225: mol_type = genomic DNA
agatttgatcctggctcaggatgaacgctagctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatgggttcacatgacggcatcagactgtgaataaaggttacggtaaaggatg

SEQ ID NO: 287 - DNA, 225 bp, unidentified
  misc_feature 1..225: note = Streptococcus sp.
  source 1..225: mol_type = genomic DNA
agagtttgatcatggctcaggacgaacgctggcggcgtgcctaatacatgcaagtagaacgctgaagtctggtgcttgcactagacggatgagttgcgaacgggtgagtaacgcgtaggtaacctaccttatagcgggggataactattggaaacgatagctaataccgcataacaggtgttaacacatgttaatatcttgaaagggacaactgtctcactataa

SEQ ID NO: 288 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaaggattttccgcatggagagttcttgaaagatggcctctatttataagctatcgctagagg

SEQ ID NO: 289 - DNA, 225 bp, unidentified; identical to SEQ ID NO: 288
  misc_feature 1..225: note = Phascolarctobacterium sp.
  source 1..225: mol_type = genomic DNA

SEQ ID NO: 290 - DNA, 225 bp, unidentified
  misc_feature 1..225: note = Phascolarctobacterium sp.
  source 1..225: mol_type = genomic DNA
agagtttgatcctggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaaggatcttccgcatggagggtctttgaaagatggcctctatttataagctatcgctagagg

SEQ ID NO: 291 - DNA, 225 bp, unidentified
  misc_feature 1..225: note = Phascolarctobacterium sp.
  source 1..225: mol_type = genomic DNA
agagtttgatcctggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaaggattttccgcatggagagttcttgaaagatggcctctatttataagctatcgctagagg

SEQ ID NO: 292 - DNA, 225 bp, unidentified; identical to SEQ ID NO: 146
  misc_feature 1..225: note = Phascolarctobacterium sp.
  source 1..225: mol_type = genomic DNA

SEQ ID NO: 293 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggagctacttcagtagcttagtggcgaacgggtgagtaacgcgtaggcaacctgccctctagatggggacaacattccgaaaggagtgctaataccgaatgtgaaggactttccgcatggagagttcttgaaagatggcctctatttataagctatcgctagagg

SEQ ID NO: 294 - DNA, 225 bp, unidentified; identical to SEQ ID NO: 133
  misc_feature 1..225: note = Phascolarctobacterium sp.
  source 1..225: mol_type = genomic DNA

SEQ ID NO: 295 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaagaactttccgcatggagggttcttgaaagatggcctctatttataagctatcgctagagg

SEQ ID NO: 296 - DNA, 225 bp, Bacteroides vulgatus
  source 1..225: mol_type = genomic DNA
agagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacg

SEQ ID NO: 297 - DNA, 225 bp, unidentified
  misc_feature 1..225: note = Phascolarctobacterium sp.
  source 1..225: mol_type = genomic DNA
agagtttgatcctggctcaggacgaacgctggcggcatgcctaacacatgcaagtcgaacggagcttcttcggaagcttagtggcgaacgggtgagtaacgcgtaggcaacctgccctctagatggggacaacattccgaaaggggtgctaataccgaatgtgacggagctatcgcatgacagttccgtgaaagatggcctctatttataagctatcgctagagg

SEQ ID NO: 298 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggagcttcttcggaagcttagtggcgaacgggtgagtagcgcgtaggcaacctgccctctagatggggacaacattccgaaaggggtgctaataccgaatgtgacggaactatcgcatgacagttccgtgaaagatggcctctatttataagctatcgctagagg

SEQ ID NO: 299 - DNA, 225 bp, unidentified
  misc_feature 1..225: note = Phascolarctobacterium sp.
  source 1..225: mol_type = genomic DNA
agagtttgatcatggctcaggacgaacgctggcggcgtgcctaacacatgcaagtcgaacggagcttcttcggaagcttagtggcgaacgggtgagtaacgcgtaggcaacctgccctctagatggggacaacattccgaaaggggtgttaataccgaatgtgacggaactatcgcatgacagttccgtgaaagatggcctctatttataagctatcgctagagg

SEQ ID NO: 300 - DNA, 225 bp, unidentified; identical to SEQ ID NO: 150
  misc_feature 1..225: note = Phascolarctobacterium sp.
  source 1..225: mol_type = genomic DNA

SEQ ID NO: 301 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggagcttcttcggaagcttagtggcgaacgggtgagtaacgcgtaggcaacctgccctctagatggggacaacattccgaaaggggtgctaataccgaatgtgacggagctatcgcatgacagttccgtgaaagatggcctctatttataagctatcgctagagg

SEQ ID NO: 302 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggagcttcttcggaagcttagtggcgaacgggtgagtaacgcgtaggcaacctgccctctagatggggacaacattccgaaaggggtgctaataccgaatgtgacggagctgtcgcatgacagttccgtgaaagatggcctctatttataagctatcgctagagg

SEQ ID NO: 303 - DNA, 225 bp, unidentified; identical to SEQ ID NO: 13
  misc_feature 1..225: note = Phascolarctobacterium sp.
  source 1..225: mol_type = genomic DNA

SEQ ID NO: 304 - DNA, 225 bp, Streptococcus orisasini
  source 1..225: mol_type = genomic DNA
agagtttgatcatggctcaggacgaacgctggcggcgtgcctaatacatgcaagtggaacgcagtgaattcaccggagcttgctccaccgaattgactgagtcgcgaacgggtgagtaacgcgtaggtaacctgccttgtagcgggggataactattggaaacgatagctaataccgcataacaacagatgacacatgttagctgtttgaaaggagcaattgctc

SEQ ID NO: 305 - DNA, 225 bp, Bacteroides graminisolvens
  source 1..225: mol_type = genomic DNA
agagtttgatcctggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcacttccacgaaagcattacggtgaaggatgggg

SEQ ID NO: 306 - DNA, 225 bp, Bacteroides graminisolvens; identical to SEQ ID NO: 250
  source 1..225: mol_type = genomic DNA

SEQ ID NO: 307 - DNA, 225 bp, Bacteroides graminisolvens; identical to SEQ ID NO: 253
  source 1..225: mol_type = genomic DNA

SEQ ID NO: 308 - DNA, 225 bp, Bacteroides graminisolvens; identical to SEQ ID NO: 254
  source 1..225: mol_type = genomic DNA

SEQ ID NO: 309 - DNA, 225 bp, Bacteroides graminisolvens; identical to SEQ ID NO: 256
  source 1..225: mol_type = genomic DNA

SEQ ID NO: 310 - DNA, 225 bp, Bacteroides graminisolvens; identical to SEQ ID NO: 257
  source 1..225: mol_type = genomic DNA

SEQ ID NO: 311 - DNA, 225 bp, unidentified; identical to SEQ ID NO: 146
  misc_feature 1..225: note = Phascolarctobacterium sp.
  source 1..225: mol_type = genomic DNA

SEQ ID NO: 312 - DNA, 225 bp, unidentified
  misc_feature 1..225: note = Phascolarctobacterium sp.
  source 1..225: mol_type = genomic DNA
agagtttgatcctggctcaggacgaacgctggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcggtccaccgcatggcggaccgatgaaagatggcctctacatgtaagctatcgccgga

SEQ ID NO: 313 - DNA, 225 bp, unidentified; identical to SEQ ID NO: 146
  misc_feature 1..225: note = Phascolarctobacterium sp.
  source 1..225: mol_type = genomic DNA

SEQ ID NO: 314 - DNA, 225 bp, unidentified; identical to SEQ ID NO: 58
  misc_feature 1..225: note = Phascolarctobacterium sp.
  source 1..225: mol_type = genomic DNA

SEQ ID NO: 315 - DNA, 225 bp, unidentified; identical to SEQ ID NO: 13
  misc_feature 1..225: note = Phascolarctobacterium sp.
  source 1..225: mol_type = genomic DNA

SEQ ID NO: 316 - DNA, 225 bp, unidentified; identical to SEQ ID NO: 13
  misc_feature 1..225: note = Phascolarctobacterium sp.
  source 1..225: mol_type = genomic DNA

SEQ ID NO: 317 - DNA, 225 bp, unidentified; identical to SEQ ID NO: 135
  misc_feature 1..225: note = Phascolarctobacterium sp.
  source 1..225: mol_type = genomic DNA

SEQ ID NO: 318 - DNA, 225 bp, unidentified; identical to SEQ ID NO: 115
  misc_feature 1..225: note = Phascolarctobacterium sp.
  source 1..225: mol_type = genomic DNA

SEQ ID NO: 319 - DNA, 225 bp, Escherichia coli
  source 1..225: mol_type = genomic DNA
agagtttgatcatggctcagattgaacgctggcggcaggcctaacacatgcaagtcgaacggtaacaggaagcagcttgctgctttgctgacgagtggcggacgggtgagtaatgtctgggaaactgcctgatggcgggggataactactggaaacggtagctaataccgcataacgtcgcaagaccaaagagggggaccttcgggcctcttgccatcggatgtg

SEQ ID NO: 320 - DNA, 225 bp, Escherichia coli; identical to SEQ ID NO: 319
  source 1..225: mol_type = genomic DNA

SEQ ID NO: 321 - DNA, 225 bp, Escherichia coli; identical to SEQ ID NO: 13
  source 1..225: mol_type = genomic DNA

SEQ ID NO: 322 - DNA, 225 bp, Escherichia coli; identical to SEQ ID NO: 146
  source 1..225: mol_type = genomic DNA

SEQ ID NO: 323 - DNA, 225 bp, Escherichia coli
  source 1..225: mol_type = genomic DNA
agagtttgatcctggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtg

SEQ ID NO: 324 - DNA, 225 bp, Escherichia coli
  source 1..225: mol_type = genomic DNA
agagtttgatcctggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtg

SEQ ID NO: 325 - DNA, 225 bp, Escherichia coli; identical to SEQ ID NO: 13
  source 1..225: mol_type = genomic DNA

SEQ ID NO: 326 - DNA, 225 bp, Escherichia coli; identical to SEQ ID NO: 13
  source 1..225: mol_type = genomic DNA

SEQ ID NO: 327 - DNA, 225 bp, Bacteroidetes bacterium
  source 1..225: mol_type = genomic DNA
agagtttgatcatggctcaggatgaacgctagcggcaggcttaacacatgcaagtcgaggggcagcatggggagtagcaatacaccctgatggcgaccggcgcaagggtgcgtaacgcgtgagcaacatgcccgtatctgggagataaccggtggaaacgccgcctaatatcccataacaacagaagcagcattgcttttgtttgaaagatacgttggatacgga

SEQ ID NO: 328 - DNA, 225 bp, Bacteroidetes bacterium; identical to SEQ ID NO: 327
  source 1..225: mol_type = genomic DNA

SEQ ID NO: 329 - DNA, 225 bp, Bacteroidetes bacterium
  source 1..225: mol_type = genomic DNA
agagtttgatcatggctcaggatgaacgctagctacaggcttaacacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggg

SEQ ID NO: 330 - DNA, 225 bp, Bacteroidetes bacterium
  source 1..225: mol_type = genomic DNA
agagtttgatcctggctcaggatgaacgctagcggcaggcttaacacatgcaagtcgaggggcagcatggggagtagcaatacaccctgatggcgaccggcgcaagggtgcgtaacgcgtgagcaacatgcccgtatctgggagataaccggtggaaacgccgcctaatatcccataacagcaggagcagcattgcttttgtttgaaagatacgttggatacgga

SEQ ID NO: 331 - DNA, 225 bp, Bacteroidetes bacterium
  source 1..225: mol_type = genomic DNA
agagtttgatcatggctcaggatgaacgctagcggcaggcttaacacatgcaagtcgaggggcagcatggggagtagcaatacaccctgatggcgaccggcgcaagggtgcgtaacgcgtgagcaacatgcccgtatctgggagataaccggtggaaacgccgcctaatatcccataacagcagaagcagcattgcttttgtttgaaagattcgttggatacgga

SEQ ID NO: 332 - DNA, 225 bp, unidentified
  misc_feature 1..225: note = Phascolarctobacterium sp.
  source 1..225: mol_type = genomic DNA
agagtttgatcctggctcaggacgaacgctggcggcatgcctaacacatgcaagtcgaacggggactatttcggtagtcctagtggcgaacgggtgagtaacgcgtaggcaacctgccctccggatggggacaacatcccgaaaggggtgctaataccgagtacgatccgggaatcgcatgattcccggatgaaagatggcctctacatgtaagctatcgccgga

SEQ ID NO: 333 - DNA, 225 bp, unidentified
  misc_feature 1..225: note = Phascolarctobacterium sp.
  source 1..225: mol_type = genomic DNA
agatttgatcatggctcaggacgaacgctggcggcatgcctaacacatgcaagtcgaacggggactatttcggtagtcctagtggcgaacgggtgagtaacgcgtaggcaacctgccctccggatggggacaacatcccgaaaggggtgctaataccgagtacgatccgggaatcgcatgattcccggatgaaagatggcctctacatgtaagctatcgccggag

SEQ ID NO: 334 - DNA, 225 bp, unidentified
  misc_feature 1..225: note = Phascolarctobacterium sp.
  source 1..225: mol_type = genomic DNA
agatttgatcctggctcaggacgaacgctggcggcatgcctaacacatgcaagtcgaacggggactatttcggtagtcctagtggcgaacgggtgagtaacgcgtaggcaacctgccctccggatggggacaacatcccgaaaggggtgctaataccgagtacgatccgggaatcgcatgattcccggatgaaagatggcctctacatgtaagctatcgccggag

SEQ ID NO: 335 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggggactatttaggtagtcctagtggcgaacgggtgagtaacgcgtaggcaacctgccctccggatggggacaacatcccgaaaggggtgctaataccgagtacgatccgggaatcgcatgattcccggatgaaagatggcctctacatgtaagctatcgccgga

SEQ ID NO: 336 - DNA, 225 bp, unidentified; identical to SEQ ID NO: 332
  misc_feature 1..225: note = Phascolarctobacterium sp.
  source 1..225: mol_type = genomic DNA

SEQ ID NO: 337 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggggactatttcggtagtcctagtggcgaacgggtgagtaacgcgtaggcaacctgccctccggatggggacaacatcccgaaaggggtgctaataccgagtacgatccgggaatcgcatgattcccggatgaaagatggcctctacatgtaagctatcgccgga

SEQ ID NO: 338 - DNA, 225 bp, unidentified
  misc_feature 1..225: note = Alloprevotella sp.
  source 1..225: mol_type = genomic DNA
agagtttgatcctggctcaggatgaacgctagctacaggcttaacacatgcaagtcgaggggcagcgggagggtagcttgctactcttgccggcgaccggcgcacgggtgcgtaacgcgtatccaatctacccatgtctcggggatagccttgcgaaagtaagattaatacccgatggcttcggttcctcgcatgaggtgccgaataaagattcatcggacatgg

SEQ ID NO: 339 - DNA, 225 bp, unidentified
  misc_feature 1..225: note = Alloprevotella sp.
  source 1..225: mol_type = genomic DNA
agagtttgatcctggctcaggatgaacgctagctacaggcttaacacatgcaagtcgaggggcagcgggagagtagcttgctgctcttgccggcgaccggcgcacgggtgcgtaacgcgtatccaatctacccacgtctcggggatagccttgcgaaagtaagattaatacccgatggcttcggttcctcgcatgaggtgccgaataaagattcatcggacatgg

SEQ ID NO: 340 - DNA, 225 bp, unidentified
  misc_feature 1..225: note = Alloprevotella sp.
  source 1..225: mol_type = genomic DNA
agagtttgatcatggctcaggatgaacgctagctacaggcttaacacatgcaagtcgaggggcagcgggagagtagcttgctactcttgccggcgaccggcgcacgggtgcgtaacgcgtatccaatctacccatgtctcggggatagccttgcgaaagtaagattaatacccgatggcttcggtcccccgcatgaggggccgaataaagattcatcggacatgg

SEQ ID NO: 341 - DNA, 225 bp, unidentified
  misc_feature 1..225: note = Alloprevotella sp.
  source 1..225: mol_type = genomic DNA
agagtttgatcctggctcaggatgaacgctagctacaggcttaacacatgcaagtcgaggggcagcgggagagtagcttgctactcttgccggcgaccggcgcacgggtgcgtaacgcgtatccaatctacccatgtctcggggatagccttgcgaaagtaagattaatacccgatggcttcggtcccccgcatgaggggccgaataaagattcatcggacatgg

SEQ ID NO: 342 - DNA, 225 bp, unidentified
  misc_feature 1..225: note = Alloprevotella sp.
  source 1..225: mol_type = genomic DNA
agagtttgatcctggctcaggatgaacgctagctacaggcttaacacatgcaagtcgaggggcagcgggagagtagcttgctactcttgccggcgaccggcgcacgggtgcgtaacgcgtatccaatctacccatgtctcggggatagccctgcgaaagtaagattaatacccgatggcttcggtcctccgcatgaggggccgaataaagattcatcggacatgg

SEQ ID NO: 343 - DNA, 225 bp, unidentified; identical to SEQ ID NO: 13
  misc_feature 1..225: note = Alloprevotella sp.
  source 1..225: mol_type = genomic DNA

SEQ ID NO: 344 - DNA, 225 bp, unidentified; identical to SEQ ID NO: 13
  misc_feature 1..225: note = Alloprevotella sp.
  source 1..225: mol_type = genomic DNA

SEQ ID NO: 345 - DNA, 225 bp, unidentified; identical to SEQ ID NO: 13
  misc_feature 1..225: note = Alloprevotella sp.
  source 1..225: mol_type = genomic DNA

SEQ ID NO: 346 - DNA, 225 bp, Muribaculaceae bacterium
  source 1..225: mol_type = genomic DNA
agagtttgatcctggctcaggatgaacgctagcgacaggcttaacacatgcaagtcgaggggcagcgagagggtagcaatacccttgtcggcgaccggcgcactggtgagtaacacgtatgcgacctgccccctacaggggaataacccgtagaaatgcggcctaataccccatagtcccggaagctgcatggcctccgggggaaaggtgagccggtaggggatg

SEQ ID NO: 347 - DNA, 225 bp, Muribaculaceae bacterium
  source 1..225: mol_type = genomic DNA
agagtttgatcatggctcaggatgaacgctagcgacaggcttaacacatgcaagtcgaggggcagcgagagggtagcaatacccttgtcggcgaccggcgcactggtgagtaacacgtatgcgacctgccccctacaggggaataacccgtagaaatgcggcctaataccccatagtcccggaagctgcatggctctcgggggaaaggtgagccggtaggggatg

SEQ ID NO: 348 - DNA, 225 bp, Bacteroidia bacterium
  source 1..225: mol_type = genomic DNA
agagtttgatcatggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctctgggggcatcctcggggggataaggaagcgattcggccgcggacgggcatg

SEQ ID NO: 349 - DNA, 225 bp, Bacteroidia bacterium
  source 1..225: mol_type = genomic DNA
agatttgatcatggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctcggggggcatccttcgggggataaggaagcgattcggccgcggacgggcatgc

SEQ ID NO: 350 - DNA, 225 bp, Bacteroidia bacterium
  source 1..225: mol_type = genomic DNA
agagtttgatcctggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctctgggggcatcctttgggggataaggaagcgattcggccgtggacgggcatg

SEQ ID NO: 351 - DNA, 225 bp, Bacteroidia bacterium
  source 1..225: mol_type = genomic DNA
agagtttgatcctggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctctgggggcatcctcggggggataaggaagcgattcggccgtggacgggcatg

SEQ ID NO: 352 - DNA, 225 bp, Bacteroidia bacterium
  source 1..225: mol_type = genomic DNA
agagtttgatcctggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctctgggggcatcctcggggggataaggaagcgattcggccgcggacgggcatg

SEQ ID NO: 353 - DNA, 225 bp, Bacteroidia bacterium; identical to SEQ ID NO: 348
  source 1..225: mol_type = genomic DNA

SEQ ID NO: 354 - DNA, 225 bp, Bacteroidia bacterium
  source 1..225: mol_type = genomic DNA
agagtttgatcatggctcaggatgaacgctagcggcaggcctaatacatgcaagtcggacggtaagggcccttcggggctcgagagtggcgcacgggtgcgtaacgcgtatgcaaccaacccacggccgggggatagccggtggaaacgccggataataccccatggttccctcggggggcatccttcgggggataaggaagcgattcggccgcggacgggcatg

SEQ ID NO: 355 - DNA, 225 bp, Bacteroides massiliensis
  source 1..225: mol_type = genomic DNA
agagtttgatcatggctcaggacgaacgctggcggcgtgcttaacacatgcaagtcgaacgggatttagtaaacagaaacctcggtggaagattactaatgagagtggcgaacgggtgagtaacgcgtgagcaacctgcctatgacagtgggatagcctcgggaaaccgggattaataccgcataaaatcgtagaaacacatgtttcaacggtcaaagatttatc

SEQ ID NO: 356 - DNA, 225 bp, Bacteroides massiliensis
  source 1..225: mol_type = genomic DNA
agagtttgatcatggctcaggatgaacgctagctacaggcttaacacatgcaagtagaggggcagcatggggcagcaatgccccgatggcgacctgcggatgggtgagtaacacgtatccaacctgcccggcactggaggatagcctcccgaaagggaaattaacactccatgccgtaccgtcgggacatcccttcggtatgaaagttttacggtgtcggatggg

SEQ ID NO: 357 - DNA, 225 bp, Bacteroides massiliensis
  source 1..225: mol_type = genomic DNA
agagtttgatcctggctcaggatgaacgctagctacaggcttaacacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggg

SEQ ID NO: 358 - DNA, 225 bp, Bacteroides massiliensis
  source 1..225: mol_type = genomic DNA
agagtttgatcctggctcaggatgaacgctagctacaggcttaacacatgcaagtagaggggcagcatggggcagcaatgccccgatggcgacctgcggatgggtgagtaacacgtatccaacctgcccggcactggaggatagcctcccgaaagggaaattaacactccatgccgtaccgtcgggacatcccttcggtatgtaagttttacggtgtcggatggg

SEQ ID NO: 359 - DNA, 225 bp, Bacteroides massiliensis
  source 1..225: mol_type = genomic DNA
agagtttgatcatggctcagggtgaacgctagctacaggcttaacacatgcaagtagaggggcagcatggggcagcaatgccccgatggcgacctgcggatgggtgagtaacacgtatccaacctgcccggcactggaggatagcctcccgaaagggaaattaacactccatgccgtaccgtcgggacatcccttcggtatgaaagttttacggtgtcggatggg

SEQ ID NO: 360 - DNA, 225 bp, Bacteroides massiliensis; identical to SEQ ID NO: 356
  source 1..225: mol_type = genomic DNA

SEQ ID NO: 361 - DNA, 225 bp, Oscillibacter valericigenes
  source 1..225: mol_type = genomic DNA
agagtttgatcctggctcaggacgaacgctggcggcgtgcttaacacatgcaagtcgaacgagaatctgtggattgaggattcgtccaaatgaaacagaggaaagtggcggacgggtgagtaacgcgtgaggaacctgcctttcagagggggacaacagttggaaacgactgctaataccgcataacgcatattggtcgcatggtcgatatgccaaagatttatc

SEQ ID NO: 362 - DNA, 225 bp, Oscillibacter valericigenes
  source 1..225: mol_type = genomic DNA
agagtttgatcctggctcaggacgaacgctggcggcgtgcttaacacatgcaagtcgaacgagaatctgtggatcgaggattcgtccaagtgaagcagaggaaagtggcggacgggtgagtaacgcgtgaggaacctgcctttcagagggggacaacagttggaaacgactgctaataccgcataacgcatattggtcgcatggtcgatatgccaaagatttatc

SEQ ID NO: 363 - DNA, 225 bp, Oscillibacter valericigenes; identical to SEQ ID NO: 361
  source 1..225: mol_type = genomic DNA

SEQ ID NO: 364 - DNA, 225 bp, Oscillibacter valericigenes
  source 1..225: mol_type = genomic DNA
agagtttgatcatggctcaggatgaacgctggcggcgtgcttaacacatgcaagtcgaacgagaatctgtggatcgaggattcgtccaagtgaagcagaggaaagtggcggacgggtgagtaacgcgtgaggaacctgcctttcagagggggacaacagttggaaacgactgctaataccgcataacgcatattggtcgcatggtcgatatgccaaagatttatc

SEQ ID NO: 365 - DNA, 225 bp, Oscillibacter valericigenes
  source 1..225: mol_type = genomic DNA
agagtttgatcatggctcaggacgaacgctggcggcgtgcttaacacatgcaagtcgaacgagaatctgtggattgaggattcgtccaaatgaaacagaggaaagtggcggacgggtgagtaacgcgtgaggaacctgcctttcagagggggacaacagttggaaacgactgctaataccgcataacgcatattggtcgcatggtcgatatgccaaagatttatc

SEQ ID NO: 366 - DNA, 225 bp, Oscillibacter valericigenes; identical to SEQ ID NO: 13
  source 1..225: mol_type = genomic DNA

SEQ ID NO: 367 - DNA, 225 bp, Oscillibacter valericigenes
  source 1..225: mol_type = genomic DNA
agagtttgatcctggctcaggacgaccgctggcggcgtgcttaacacatgcaagtcgaacgagaatctgtggattgaggattcgtccaaatgaaacagaggaaagtggcggacgggtgagtaacgcgtgaggaacctgcctttcagagggggacaacagttggaaacgactgctaataccgcataacgcatattggtcgcatggtcgatatgccaaagaattatc

SEQ ID NO: 368 - DNA, 225 bp, Oscillibacter valericigenes
  source 1..225: mol_type = genomic DNA
agagtttgatcatggctcaggacgaacgctggcggcgtgcttaacacatgcaagtcgaacgagaatctgtggattgaggattcgtccaaatgaagcagaggacagtggcggacgggtgagtaacgcgtgaggaacctgcctttcagagggggacaacagttggaaacgactgctaataccgcataacgcatattggtcgcatggccgatatgccaaagatttatc

SEQ ID NO: 369 - DNA, 225 bp, TM7 bacterium
  source 1..225: mol_type = genomic DNA
agagtttgatcctggctcaggatgaatgctggcggcgtgcctaatacatgcaagtcgagcggtaacagggcttcaccgtttccttgacacacctggccaaagtgcaggttgttagattcgagggaacagtgaagctgctgacgagcggcggacggctgagtaacgcgtgggaacataccccaaactgagggataacgcaccgaaaggtgtgctaataccgcatat

SEQ ID NO: 370 - DNA, 225 bp, TM7 bacterium
  source 1..225: mol_type = genomic DNA
agagtttgatcctggctcaggatgaatgctggcggcgtgcctaatacatgcaagtcgagcggtaacagggcttcaccgtttccttgacacacctgaccattgcgtaggttgttagattcgagggaacagtgaagctgctgacgagcggcggacggctgagtaacgcgtgggaacataccccaaactgagggataacgcaccgaaaggtgtgctaataccgcatgt

SEQ ID NO: 371 - DNA, 225 bp, TM7 bacterium
  source 1..225: mol_type = genomic DNA
agagtttgatcatggctcaggatgaatgctggcggcgtgcctaatacatgcaagtcgagcggtaacagggcttcaccgtttccttgacacacctggccaaagtgcaggttgttagattcgagggaacagtgaagctgctgacgagcggcggacggctgagtaacgcgtgggaacataccccaaactgagggataacgcaccgaaaggtgtgctaataccgcatat

SEQ ID NO: 372 - DNA, 225 bp, TM7 bacterium; identical to SEQ ID NO: 369
  source 1..225: mol_type = genomic DNA

SEQ ID NO: 373 - DNA, 225 bp, Phascolarctobacterium succinatutens
  source 1..225: mol_type = genomic DNA
agagtttgatcctggctcaggacgaacgctggcggcatgcctaacacatgcaagtcgaacggagaagaccagcaccaagttcattcttggtgccggcagcgtcaagaatctgcgaagcagattctttaggccaacaaacatgccaacactcagggtgagtttggtgctggttttcttagtggcgaacgggtgagtaacgcgtaggcaacctgccctttagatggg

SEQ ID NO: 374 - DNA, 225 bp, Phascolarctobacterium succinatutens; identical to SEQ ID NO: 13
  source 1..225: mol_type = genomic DNA

SEQ ID NO: 375 - DNA, 225 bp, Phascolarctobacterium succinatutens
  source 1..225: mol_type = genomic DNA
agagtttgatcatggctcaggacgaacgctggcggcatgcctaacacatgcaagtcgaacggagaagaccagcaccaagttcattcttggtgccggcagcgtcaagaatctgcgaagcagattcttcaggccaacaaacatgccaacactcagggtgagtttggtgctggttttcttagtggcgaacgggtgagtaacgcgtaggcaacctgccctttagatggg

SEQ ID NO: 376 - DNA, 225 bp, Phascolarctobacterium succinatutens; identical to SEQ ID NO: 373
  source 1..225: mol_type = genomic DNA

SEQ ID NO: 377 - DNA, 225 bp, Phascolarctobacterium succinatutens; identical to SEQ ID NO: 150
  source 1..225: mol_type = genomic DNA

SEQ ID NO: 378 - DNA, 225 bp, Phascolarctobacterium succinatutens
  source 1..225: mol_type = genomic DNA
agagtttgatcatggctcaggacgaacgctggcggcatgcctaacacatgcaagtcgaacggagaagaccagcaccaagttcattcttggtgccggcggcgtcaagaatctgcggagcagattcttcaggccaacaaacatgccaacactcagggtgagtttggtgctggttttcttagtggcgaacgggtgagtaacgcgtaggcaacctgccctttagatggg

SEQ ID NO: 379 - DNA, 225 bp, Bacteroides graminisolvens
  source 1..225: mol_type = genomic DNA
agagtttgatcatggctcaggatgaacgctagctacaggcttaacacatgcaagtcgaggggcatcgcggtgcagcaatgcactggcggcgaccggcgcacgggtgagtaacgcgtatccgacctggccttcactcgggtatagcccctcgaaagagggattaatccccgatatgtggtagagttacatggcacttccacgaaagcattacggtgaaggatgggg

SEQ ID NO: 380 - DNA, 225 bp, Bacteroides graminisolvens; identical to SEQ ID NO: 253
  source 1..225: mol_type = genomic DNA

SEQ ID NO: 381 - DNA, 225 bp, Bacteroides graminisolvens; identical to SEQ ID NO: 254
  source 1..225: mol_type = genomic DNA

SEQ ID NO: 382 - DNA, 225 bp, Bacteroides graminisolvens; identical to SEQ ID NO: 255
  source 1..225: mol_type = genomic DNA

SEQ ID NO: 383 - DNA, 225 bp, Bacteroides graminisolvens; identical to SEQ ID NO: 256
  source 1..225: mol_type = genomic DNA

SEQ ID NO: 384 - DNA, 225 bp, Bacteroides graminisolvens; identical to SEQ ID NO: 257
  source 1..225: mol_type = genomic DNA

SEQ ID NO: 385 - DNA, 225 bp, Alphaproteobacteria bacterium
  source 1..225: mol_type = genomic DNA
agagtttgatcctggctcagaacgaacgctggcggcaggtcttaggcatgcaagtcgaacgagtgaagcagggcttgccctgtggagcgagtggcgcacgagtgagtaacgcgtgggaaactgcccattactggggaataacgtttggaaacgaacgctaataccgcatacgccggaaacgggaaagatttatcggtaatggatgtgcccgcgttggattagctt

SEQ ID NO: 386 - DNA, 225 bp, Alphaproteobacteria bacterium
  source 1..225: mol_type = genomic DNA
agagtttgatcctggctcagaacgaacgctggcggcaggtcttaggcatgcaagtcgaacgagtgaagcagggcttgccctgtggatcgagtggcgcacgagtgagtaacgcgtgggaaactgcccatcactggggaataacgtttggaaacgaacgctaataccgcatacgccggaaacgggaaagatttatcggtgatggatgtgcccgcgttggattagctt

SEQ ID NO: 387 - DNA, 225 bp, Alphaproteobacteria bacterium
  source 1..225: mol_type = genomic DNA
agagtttgatcatggctcagaacgaacgctggcggcaggtcttaggcatgcaagtcgaacgagtgaagcagggcttgccctgtggatcgagtggcgcacgagtgagtaacgcgtgggaaactgcccatcactggggaataacgtttggaaacgaacgctaataccgcatacgccggaaacgggaaagatttatcggtgatggatgtgcccgcgttggattagctt

SEQ ID NO: 388 - DNA, 225 bp, Alphaproteobacteria bacterium
  source 1..225: mol_type = genomic DNA
agagtttgatcatggctcagaacgaacgctggcggcaggtcttaggcatgcaagtcgaacgagtgaagcagggcttgccctgtggagcgagtggcgcacgagtgagtaacgcgtgggaaactgcccatcactggggaataacgtttggaaacgaacgctaataccgcatacgcccgcaaggggaaagatttatcggtgatggatgtgcccgcgttggattagctt

SEQ ID NO: 389 - DNA, 225 bp, Alphaproteobacteria bacterium
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgatagcatttgagaacgattaaagatttatcgctgatggatggacatgcgttgga

SEQ ID NO: 390 - DNA, 225 bp, Alphaproteobacteria bacterium; identical to SEQ ID NO: 146
  source 1..225: mol_type = genomic DNA

SEQ ID NO: 391 - DNA, 225 bp, Alphaproteobacteria bacterium
  source 1..225: mol_type = genomic DNA
agagtttgatcatggctcagaacgaacgctggcggcaggtcttaggcatgcaagtcgaacgagtgaagcagggcttgccctgtggagcgagtggcgcacgagtgagtaacgcgtgggaaactgcccattactggggaataacgtttggaaacgaacgctaataccgcatacgccggaaacgggaaagatttatcggtaatggatgtgcccgcgttggattagctt

SEQ ID NO: 392 - DNA, 225 bp, Alphaproteobacteria bacterium
  source 1..225: mol_type = genomic DNA
agagtttgatcatggctcagaacgaacgctggcggcaggtcttaggcatgcaagtcgaacgagtgaagcagggcttgccctgtggagcgagtggcgcacgagtgagtaacgcgtgggaaactgcccattactggggaataacgtttggaaccgaacgctaataccgcatacgccggaaacgggaaagatttatcggtaatggatgtgcccgcgttggattagctt

SEQ ID NO: 393 - DNA, 225 bp, unidentified
  misc_feature 1..225: note = Parvimonas sp.
  source 1..225: mol_type = genomic DNA
agagtttgatcatggctcaggacgaacgctggcggcgtgcttaacacatgcaagtcgaacgagaatttagcggactaatctttcgggagaaggaagttgaaggaaagtggcgaacgggtgagtaacacgtgagcaacctaccttacacaaagggatagccactggaaacggtgattaataccatatgagaccacaatcctgcatgggatgggggttaaagattta

SEQ ID NO: 394 - DNA, 225 bp, Cecembia lonarensis
  source 1..225: mol_type = genomic DNA
agagtttgatcctggctcaggatgaacgctagcgacaggcctaacacatgcaagtcgagggagattcccttcggggatgagaccggcgcacgggtgagtaacgcgtatgtaacctgcccataagtgggggatagcccagcgaaagctggattaataccgcataatatccgagactgacatctttttcggattaaagatttattgcttatggatggacatgcgttg

SEQ ID NO: 395 - DNA, 225 bp, Cecembia lonarensis
  source 1..225: mol_type = genomic DNA
agatttgatcatggctcaggatgaacgctagcgacaggcctaacacatgcaagtcgagggagattcccttcggggatgagaccggcgcatgggtgagtaacgcgtatgtaacctgcccataagtgggggatagcccagcgaaagctggattaataccgcataatatccgagactgacatctttttcggattaaagatttattgcttatggatggacatgcgttgg

SEQ ID NO: 396 - DNA, 225 bp, Cecembia lonarensis; identical to SEQ ID NO: 394
  source 1..225: mol_type = genomic DNA

SEQ ID NO: 397 - DNA, 225 bp, Cecembia lonarensis; identical to SEQ ID NO: 181
  source 1..225: mol_type = genomic DNA

SEQ ID NO: 398 - DNA, 225 bp, Cecembia lonarensis; identical to SEQ ID NO: 182
  source 1..225: mol_type = genomic DNA

SEQ ID NO: 399 - DNA, 225 bp, Cecembia lonarensis; identical to SEQ ID NO: 183
  source 1..225: mol_type = genomic DNA

SEQ ID NO: 400 - DNA, 225 bp, Cecembia lonarensis; identical to SEQ ID NO: 178
  source 1..225: mol_type = genomic DNA

SEQ ID NO: 401 - DNA, 225 bp, Cecembia lonarensis; identical to SEQ ID NO: 185
  source 1..225: mol_type = genomic DNA

SEQ ID NO: 402 - DNA, 225 bp, Streptococcus pluranimalium
  source 1..225: mol_type = genomic DNA
agagtttgatcctggctcaggacgaacgctggcggcgtgcctaatacatgcaagtggaacgcaattgattctcccgtagcttgctacatttgaattgattgagtcgcgaacgggtgagtaacgcgtaggtaacctaccttatagcgggggataactattggaaacgatagctaataccgcataagatttggcaacacatgttgttaatttaaaaggagcaacagc

SEQ ID NO: 403 - DNA, 225 bp, unidentified
  misc_feature 1..225: note = Phascolarctobacterium sp.
  source 1..225: mol_type = genomic DNA
agagtttgatcctggctcaggacgaacgctggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcggccgaccgcatggttgaccgatgaaagatggcctctacatgtaagctatcgccgga

SEQ ID NO: 404 - DNA, 225 bp, unidentified
  misc_feature 1..225: note = Phascolarctobacterium sp.
  source 1..225: mol_type = genomic DNA
agagtttgatcatggctcaggacgaacgctggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcgatcgaccgcatggttgaccgatgaaagatggcctctacatgtaagctatcgccgga

SEQ ID NO: 405 - DNA, 225 bp, unidentified
  misc_feature 1..225: note = Phascolarctobacterium sp.
  source 1..225: mol_type = genomic DNA
agatttgatcctggctcaggatgaacgttggcggcatgcctaacacatgcaagtcgaacggagactatttcggtagtcttagtggcgaacgggtgagtaacgcgtaggcaacctgccctccggatggggacaacatcccgaaaggggtgctaataccgagtacgatcggtcgaccgcatggttgaccgatgaaagatggcctctacatgtaagctatcgccggag

SEQ ID NO: 406 - DNA, 225 bp, unidentified
  misc_feature 1..225: note = Phascolarctobacterium sp.
  source 1..225: mol_type = genomic DNA
agagtttgatcctggctcaggacgaacgctggcggcatgcctaacacatgcaagtcgagcggagactatttcggtagtcttagtggcgaacgggtgagtaacgcgtaggcaacctgccttccgaatggggacaacatcccgaaaggggtgctaataccgagtacgatcggccgaccgcatggttggccgatgaaagatggcctctacatgtaagctatcgccgga

SEQ ID NO: 407 - DNA, 225 bp, unidentified; identical to SEQ ID NO: 146
  misc_feature 1..225: note = Phascolarctobacterium sp.
  source 1..225: mol_type = genomic DNA

SEQ ID NO: 408 - DNA, 225 bp, unidentified; identical to SEQ ID NO: 13
  misc_feature 1..225: note = Phascolarctobacterium sp.
  source 1..225: mol_type = genomic DNA

SEQ ID NO: 409 - DNA, 225 bp, unidentified; identical to SEQ ID NO: 135
  misc_feature 1..225: note = Phascolarctobacterium sp.
  source 1..225: mol_type = genomic DNA

SEQ ID NO: 410 - DNA, 225 bp, unidentified; identical to SEQ ID NO: 115
  misc_feature 1..225: note = Phascolarctobacterium sp.
  source 1..225: mol_type = genomic DNA

SEQ ID NO: 411 - DNA, 225 bp, Bacteroidia bacterium; identical to SEQ ID NO: 190
  source 1..225: mol_type = genomic DNA

SEQ ID NO: 412 - DNA, 225 bp, Bacteroidia bacterium
  source 1..225: mol_type = genomic DNA
agagtttgatcctggctcaggatgaacgctagcggcaggcttaacacatgcaagtcgaggggcagcagcagagagtagcaatacaatttggctggcgaccggcgcaagggtgcgtaacgcgtgagcaacatgcccgtatcagggacataaccggtggaaacgccgcctaatttcccataacatgtgaagaggcatctctttatattgaaagattcgtcggatacg

SEQ ID NO: 413 - DNA, 225 bp, Bacteroidia bacterium; identical to SEQ ID NO: 188
  source 1..225: mol_type = genomic DNA

SEQ ID NO: 414 - DNA, 225 bp, Bacteroidia bacterium; identical to SEQ ID NO: 186
  source 1..225: mol_type = genomic DNA

SEQ ID NO: 415 - DNA, 225 bp, Bacteroidia bacterium; identical to SEQ ID NO: 190
  source 1..225: mol_type = genomic DNA

SEQ ID NO: 416 - DNA, 225 bp, Bacteroidia bacterium; identical to SEQ ID NO: 13
  source 1..225: mol_type = genomic DNA

SEQ ID NO: 417 - DNA, 225 bp, Bacteroidia bacterium; identical to SEQ ID NO: 192
  source 1..225: mol_type = genomic DNA

SEQ ID NO: 418 - DNA, 225 bp, Bacteroidia bacterium; identical to SEQ ID NO: 115
  source 1..225: mol_type = genomic DNA

SEQ ID NO: 419 - DNA, 225 bp, Bacteroides graminisolvens; identical to SEQ ID NO: 255
  source 1..225: mol_type = genomic DNA

SEQ ID NO: 420 - DNA, 225 bp, Bacteroides graminisolvens; identical to SEQ ID NO: 252
  source 1..225: mol_type = genomic DNA

SEQ ID NO: 421 - DNA, 225 bp, Bacteroides graminisolvens; identical to SEQ ID NO: 253
  source 1..225: mol_type = genomic DNA

SEQ ID NO: 422 - DNA, 225 bp, Bacteroides graminisolvens; identical to SEQ ID NO: 254
  source 1..225: mol_type = genomic DNA

SEQ ID NO: 423 - DNA, 225 bp, Bacteroides graminisolvens; identical to SEQ ID NO: 255
  source 1..225: mol_type = genomic DNA

SEQ ID NO: 424 - DNA, 225 bp, Bacteroides graminisolvens; identical to SEQ ID NO: 256
  source 1..225: mol_type = genomic DNA

SEQ ID NO: 425 - DNA, 225 bp, Bacteroides graminisolvens; identical to SEQ ID NO: 257
  source 1..225: mol_type = genomic DNA

SEQ ID NO: 426 - DNA, 225 bp, Clostridium maximum; identical to SEQ ID NO: 207
  source 1..225: mol_type = genomic DNA

SEQ ID NO: 427 - DNA, 225 bp, Clostridium maximum; identical to SEQ ID NO: 201
  source 1..225: mol_type = genomic DNA

SEQ ID NO: 428 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcgggaatttttctagcggcggacgggtgagtaacacgtgggcaacctgccttatagaggggaatagccttccgaaaggaagattaataccgcataacattattgaaaggcatcttttaataatcaaaggagtaatccgctataagatgggc

SEQ ID NO: 429 - DNA, 225 bp, Clostridium maximum
  source 1..225: mol_type = genomic DNA
agatttgatcctggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttaataatcaaaggagtaatccgctataagatgggcc

SEQ ID NO: 430 - DNA, 225 bp, Clostridium maximum; identical to SEQ ID NO: 205
  source 1..225: mol_type = genomic DNA

SEQ ID NO: 431 - DNA, 225 bp, Clostridium maximum
  source 1..225: mol_type = genomic DNA
agagtttgatcatggctcaggatgaacgctggcggcgtgcttaacacatgcaagtcgagcgagaaaaatttcttcggaagtttttctagcggcggacgggtgagtaacacgtgggcaacctgccttatagaggggaatagccttccgaaaggaagattaataccgcataacattattgaaaggcatcttttgataatcaaaggagcaatccacttaaggaggggc

SEQ ID NO: 432 - DNA, 225 bp, Clostridium maximum; identical to SEQ ID NO: 200
  source 1..225: mol_type = genomic DNA

SEQ ID NO: 433 - DNA, 225 bp, Clostridium maximum; identical to SEQ ID NO: 207
  source 1..225: mol_type = genomic DNA

SEQ ID NO: 434 - DNA, 225 bp, Bacteroidia bacterium
  source 1..225: mol_type = genomic DNA
agagtttgatcatggctcaggatgaacgctagcggcaggcctaacacatgcaagtcgaggggcagcggggagtagcaatactctgccggcgaccggcggaagggtgcgtaacgcgtgagcaacttgcccgtatcagggacataaccggtggaaacgccgactaatttcccataacaacaagcgccgcatggcgcgtgtttgaaagattcgtcggatacggatggg

SEQ ID NO: 435 - DNA, 225 bp, Bacteroidia bacterium; identical to SEQ ID NO: 434
  source 1..225: mol_type = genomic DNA

SEQ ID NO: 436 - DNA, 225 bp, Bacteroidia bacterium
  source 1..225: mol_type = genomic DNA
agagtttgatcatggctcaggatgaacgctagcggcaggcctaacacatgcaagtcgaggggcagcggggagtagcaatactctgccggcgaccggcggaagggtgcgtaacgcgtgagcaacttgcccgtatcagggacataaccggtggaaacgccgattaatttcccataacaacaagcgccgcatggcgcgtgtttgaaagattcgtcggatacggatggg

SEQ ID NO: 437 - DNA, 225 bp, Bacteroidia bacterium
  source 1..225: mol_type = genomic DNA
agagtttgatcctggctcaggatgaacgctagcggcaggcctaacacatgcaagtcgaggggcagcggggagtagcaatactctgccggcgaccggcggaagggtgcgtaacgcgtgagcaacttgcccgtatcagggacataaccggtggaaacgccgactaatttcccataacaacaagcgccgcatgacgcgtgtttgaaagattcgtcggatacggatggg

SEQ ID NO: 438 - DNA, 225 bp, Bacteroides mediterraneensis
  source 1..225: mol_type = genomic DNA
agagtttgatcatggctcaggatgaacgctagctacaggcttaacacatgcaagtcgtgcggtgcgctttcgggcgcacagcggcaaacgggtgagtaacacgtatccaacctggcccttacttggggatagcccctcgaaagagagattaatacccgatgttctggaagtcccacatgccacttccagcaaagctccggcggtaagggatggggatgcgctcca

SEQ ID NO: 439 - DNA, 225 bp, Bacteroides mediterraneensis
  source 1..225: mol_type = genomic DNA
agatttgatcctggctcaggatgaacgctagctacaggcttaacacatgcaagtcgtgcggtgcgctttcgggcgcacagcggcaaacgggtgagtaacacgtatccaacctggcccttacttggggatagcccctcgaaagagagattaatacccgatgttctggaagtcccacatgccacttccagcaaagctccggcggtaagggatggggatgcgctccat

SEQ ID NO: 440 - DNA, 225 bp, Bacteroides mediterraneensis
  source 1..225: mol_type = genomic DNA
agagtttgatcctggctcaggatgaacgctagctacaggcttaacacgtgcaagtcgtgcggtgcgctttcgggcgcacagcggcaaacgggtgagtaacacgtatccaacctggcccttacttggggatagcccctcgaaagagagattaatacccgatgttctggaagtcccacatgccacttccagcaaagcttcggcggtaagggatggggatgcgctcca

SEQ ID NO: 441 - DNA, 225 bp, Bacteroides mediterraneensis; identical to SEQ ID NO: 438
  source 1..225: mol_type = genomic DNA

SEQ ID NO: 442 - DNA, 225 bp, Bacteroides mediterraneensis
  source 1..225: mol_type = genomic DNA
agagtttgatcatggctcaggatgaacgctagctacaggcttaacacatgcaagtcgtgcggtgcgctttcgggtgcacagcggcaaacgggtgagtaacacgtatccaacctggcccttacttggggatagcccctcgaaagagagattaatacccgatgttctggaagtcccacatgccacttccagcaaagctccggcggtaagggatggggatgcgctcca

SEQ ID NO: 443 - DNA, 225 bp, Bacteroides mediterraneensis
  source 1..225: mol_type = genomic DNA
agagtttgatcatggctcaggatgaacgctagctacaggcttaacacatgcaagtcgtgcggtgcgctttcgggcgcgcagcggcaaacgggtgagtaacacgtatccaacctggcccttacttggggatagcccctcgcaagagagattaatacccgatgttctggaagtcccacatgccacttccagcaaagctccggcggtaagggatggggatgcgctcca

SEQ ID NO: 444 - DNA, 225 bp, unidentified
  misc_feature 1..225: note = Phascolarctobacterium sp.
  source 1..225: mol_type = genomic DNA
agagtttgatcctggctcaggacaaacgctggcggcatgcctaacacatgcaagtcgaacggagctacttcggtagcttagtggcgaacgggtgagtaacgcgtaggcaacctgccctctagatggggacaacattccgaaaggagtgctaataccgaatgtgaaggactttccgcatggagagttcttgaaagatggcctctatttataagctatcgctagagg

SEQ ID NO: 445 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggagctacttcggtagcttagtggcgaacgggtgagtaacgcgtaggcaacctgccctctagatggggacaacattccgaaaggagtgctaataccgaatgtgaagaattttccgcatggagagttcttgaaagatggcctctatttataagctatcgctagagg

SEQ ID NO: 446 - DNA, 225 bp, unidentified; identical to SEQ ID NO: 150
  misc_feature 1..225: note = Phascolarctobacterium sp.
  source 1..225: mol_type = genomic DNA

SEQ ID NO: 447 - DNA, 225 bp, unidentified; identical to SEQ ID NO: 148
  misc_feature 1..225: note = Phascolarctobacterium sp.
  source 1..225: mol_type = genomic DNA

SEQ ID NO: 448 - DNA, 225 bp, unidentified
  misc_feature 1..225: note = Phascolarctobacterium sp.
  source 1..225: mol_type = genomic DNA
agagtttgatcctggctcaggacaaacgctggcggcatgcctaacacatgcaagtcgaacggagctacttcggtagcttagtggcgtacgggtgagtaacgcgtaggcaacctgccctctagatggggacaacattccgaaaggagtgctaataccgaatgtgaaggactttccgcatggagagttcttgaaagatggcctctatttataagctatcgctagagg

SEQ ID NO: 449 - DNA, 225 bp, unidentified; identical to SEQ ID NO: 291
  misc_feature 1..225: note = Phascolarctobacterium sp.
  source 1..225: mol_type = genomic DNA

SEQ ID NO: 450 - DNA, 225 bp, unidentified; identical to SEQ ID NO: 146
  misc_feature 1..225: note = Phascolarctobacterium sp.
  source 1..225: mol_type = genomic DNA

SEQ ID NO: 451 - DNA, 225 bp, unidentified; identical to SEQ ID NO: 293
  misc_feature 1..225: note = Phascolarctobacterium sp.
  source 1..225: mol_type = genomic DNA

SEQ ID NO: 452 - DNA, 225 bp, Bacteroides massiliensis
  source 1..225: mol_type = genomic DNA
agagtttgatcctggctcaggatgaacgctagctacaggcttaacacatgcaagtagaggggcagcatggggcagcaatgccccgatggcgacctgcggatgggtgagtaacacgtatccaacctgcccggcactggaggatagcctcccgaaagggaaattaacactccatgccgtaccgtcgggacatcccttcggtatgaaagttttacggtgtcggatggg

SEQ ID NO: 453 - DNA, 225 bp, Bacteroides massiliensis; identical to SEQ ID NO: 357
  source 1..225: mol_type = genomic DNA

SEQ ID NO: 454 - DNA, 225 bp, Bacteroides massiliensis
  source 1..225: mol_type = genomic DNA
agagtttgatcatggctcaggatgaacgctagctacaggcttaacacatgcaagtagaggggcagcatggggcagcaatgccccgatggtgacctgcggatgggtgagtaacacgtatccaacctgcccggcaccggaggatagcctcccgaaagggaaattaacactccatgccgtaccgtcgggacatcccttcggtatgaaagttttacggtgtcggatggg

SEQ ID NO: 455 - DNA, 225 bp, Bacteroides massiliensis; identical to SEQ ID NO: 452
  source 1..225: mol_type = genomic DNA

SEQ ID NO: 456 - DNA, 225 bp, Bacteroides massiliensis; identical to SEQ ID NO: 359
  source 1..225: mol_type = genomic DNA

SEQ ID NO: 457 - DNA, 225 bp, Bacteroides massiliensis; identical to SEQ ID NO: 356
  source 1..225: mol_type = genomic DNA

SEQ ID NO: 458 - DNA, 225 bp, Algoriphagus taiwanensis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgagagcatttgataatgattaaagatttatcgctgatggatggacatgcgttgga

SEQ ID NO: 459 - DNA, 225 bp, Algoriphagus taiwanensis; identical to SEQ ID NO: 151
  source 1..225: mol_type = genomic DNA

SEQ ID NO: 460 - DNA, 225 bp, Algoriphagus taiwanensis; identical to SEQ ID NO: 128
  source 1..225: mol_type = genomic DNA

SEQ ID NO: 461 - DNA, 225 bp, Algoriphagus taiwanensis; identical to SEQ ID NO: 13
  source 1..225: mol_type = genomic DNA

SEQ ID NO: 462 - DNA, 225 bp, Algoriphagus taiwanensis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgagaactggattaatatcggatgacatcgtttgatagcatttgagaacgattaaagatttatcgctgatggatggacatgcgttgga

SEQ ID NO: 463 - DNA, 225 bp, Algoriphagus taiwanensis; identical to SEQ ID NO: 131
  source 1..225: mol_type = genomic DNA

SEQ ID NO: 464 - DNA, 225 bp, Algoriphagus taiwanensis; identical to SEQ ID NO: 126
  source 1..225: mol_type = genomic DNA

SEQ ID NO: 465 - DNA, 225 bp, Bacteroidales bacterium; identical to SEQ ID NO: 280
  source 1..225: mol_type = genomic DNA

SEQ ID NO: 466 - DNA, 225 bp, Bacteroidales bacterium
  source 1..225: mol_type = genomic DNA
agagtttgatcatggctcaggatgaacgctagcggcaggcttaacacatgcaagtcgaggggcagcatgagattagcttgctaatcttgatggcgaccggcgcacgggtgagtaacgcgtatccaaccttccctttactcagggatagcccggtgaaaaccggattaacacctgatggtttcacatgacggcatcagactgtgaataaaggttacggtaaaggat

SEQ ID NO: 467 - DNA, 225 bp, Bacteroidales bacterium
  source 1..225: mol_type = genomic DNA
agagtttgatcaggctcaggatgaacgctagcggcaggcttaacacatgcaagtcgaggggcagcatgagattagcttgctaatcttgatggcgaccggcgcacgggtgagtaacgcgtatccaaccttccctttactcagggatagcccggtgaaaaccggattaacacctgatggtttcacatgacggcatcagactgtgaataaaggttacggtaaaggatg

SEQ ID NO: 468 - DNA, 225 bp, Bacteroidales bacterium; identical to SEQ ID NO: 280
  source 1..225: mol_type = genomic DNA

SEQ ID NO: 469 - DNA, 225 bp, Bacteroidales bacterium
  source 1..225: mol_type = genomic DNA
agagtttgatcctggctcaggatgaacgctagctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatgggttcacatgacggcatcagactgtgaataaaggttacggtaaaggat

SEQ ID NO: 470 - DNA, 225 bp, Bacteroidales bacterium; identical to SEQ ID NO: 283
  source 1..225: mol_type = genomic DNA

SEQ ID NO: 471 - DNA, 225 bp, Bacteroidales bacterium; identical to SEQ ID NO: 284
  source 1..225: mol_type = genomic DNA

SEQ ID NO: 472 - DNA, 225 bp, Bacteroidales bacterium; identical to SEQ ID NO: 286
  source 1..225: mol_type = genomic DNA

SEQ ID NO: 473 - DNA, 225 bp, Bacteroidales bacterium; identical to SEQ ID NO: 248
  source 1..225: mol_type = genomic DNA

SEQ ID NO: 474 - DNA, 225 bp, Parabacteroides distasonis
  source 1..225: mol_type = genomic DNA
agagtttgatcatggctcaggatgaacgctagcgacaggcttaacacatgcaagtcgaggggcagcacagggtagcaatacttgggtggcgaccggcgcacgggtgagtaacgcgtatgcaacttacctatcagagggggatagcccggcgaaagtcggattaataccccatgaaacaggggtcccgcatgggaatatttgttaaagattcatcgctgatagata

SEQ ID NO: 475 - DNA, 225 bp, Atlantibacter hermannii
  source 1..225: mol_type = genomic DNA
agagtttgatcatggctcagattgaacgctggcggcaggcctaacacatgcaagtcgaacggtaacaggaagcagcttgctgcttcgctgacgagtggcggacgggtgagtaatgtctggggatctgcctgatggagggggataactactggaaacggtagctaataccgcataacgtcgcaagaccaaagagggggaccttcgggcctcttgccatcagatgaa

SEQ ID NO: 476 - DNA, 225 bp, Atlantibacter hermannii; identical to SEQ ID NO: 475
  source 1..225: mol_type = genomic DNA

SEQ ID NO: 477 - DNA, 225 bp, Atlantibacter hermannii
  source 1..225: mol_type = genomic DNA
agagtttgatcctggctcagattgaacgctggcggcaggcctaacacatgcaagtcgaacggtaacaggaagcagcttgctgctttgctgacgagtggcggacgggtgagtaatgtctggggatctgcctgatggagggggataactactggaaacggtagctaataccgcataacgtcgcaagaccaaagagggggaccttcgggcctcttgccatcagatgaa

SEQ ID NO: 478 - DNA, 225 bp, Atlantibacter hermannii
  source 1..225: mol_type = genomic DNA
agagtttgatcctggctcagattgaacgctggcggcaggcctaacacatgcaagtcgaacggtaacaggaagcagcttgctgcttcgctgacgagtggcggacgggtgagtaatgtctggggatctgcctgatggagggggataactactggaaacggtagctaataccgcataacgtcgcaagaccaaagagggggaccttcgggcctcttgccatcagatgaa

SEQ ID NO: 479 - DNA, 225 bp, Atlantibacter hermannii
  source 1..225: mol_type = genomic DNA
agagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcagatgtg

SEQ ID NO: 480 - DNA, 225 bp, Atlantibacter hermannii
  source 1..225: mol_type = genomic DNA
agagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtg

SEQ ID NO: 481 - DNA, 225 bp, Atlantibacter hermannii
  source 1..225: mol_type = genomic DNA
agagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataatgtcgcaagaccaaagagggggaccttcgggcctcttgccatcggatgtg

SEQ ID NO: 482 - DNA, 225 bp, Atlantibacter hermannii
  source 1..225: mol_type = genomic DNA
agagtttgatcatggctcaggatgaacgctagcggcaggcttaacacatgcaagtcgaggggcagcggggagtagcaatactctgccggcgaccggcggaagggtgcgtaacacgtgagcaacatacccgtatccgggggataagcgttggaaacgacgtctaacatcccataacaacgaccgggacatcccggatgtttgaaaactgaggtggatacggattgg

SEQ ID NO: 483 - DNA, 225 bp, Atlantibacter hermannii; identical to SEQ ID NO: 245
  source 1..225: mol_type = genomic DNA

SEQ ID NO: 484 - DNA, 225 bp, Atlantibacter hermannii; identical to SEQ ID NO: 248
  source 1..225: mol_type = genomic DNA

SEQ ID NO: 485 - DNA, 225 bp, Prevotella copri
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtttgggatagcccagtgaaaactggattaataccgaatgacatcgtttgaaagcatttgataatgattaaagatttatcgctgatggatggacatgcgttgga

SEQ ID NO: 486 - DNA, 225 bp, Prevotella copri; identical to SEQ ID NO: 485
  source 1..225: mol_type = genomic DNA

SEQ ID NO: 487 - DNA, 225 bp, Prevotella copri; identical to SEQ ID NO: 135
  source 1..225: mol_type = genomic DNA

SEQ ID NO: 488 - DNA, 225 bp, Prevotella copri
  source 1..225: mol_type = genomic DNA
agagtttgatcctggctcaggatgaacgctagctacaggcttaacacatgcaagtcgaggggcagcacttcgggtggcgaccggcgcacgggtgagtaacgcgtatgtaacctgcccatcagtttgggatagcccagtgaaaactggattaataccgaatgatatcgtttgaaagcatttgataacgattaaagatttatcgctgatggatggacatgcgttgga

SEQ ID NO: 489 - DNA, 225 bp, Prevotella copri; identical to SEQ ID NO: 13
  source 1..225: mol_type = genomic DNA

SEQ ID NO: 490 - DNA, 225 bp, Prevotella copri; identical to SEQ ID NO: 130
  source 1..225: mol_type = genomic DNA

SEQ ID NO: 491 - DNA, 225 bp, Prevotella copri; identical to SEQ ID NO: 462
  source 1..225: mol_type = genomic DNA

SEQ ID NO: 492 - DNA, 225 bp, Prevotella copri; identical to SEQ ID NO: 131
  source 1..225: mol_type = genomic DNA

SEQ ID NO: 493 - DNA, 225 bp, Bacteroidales bacterium; identical to SEQ ID NO: 469
  source 1..225: mol_type = genomic DNA

SEQ ID NO: 494 - DNA, 225 bp, Bacteroidales bacterium
  source 1..225: mol_type = genomic DNA
agagtttgatcctggctcaggatgaacgctagctacaggcttaacacatgcaagtcgaggggcagcatgacaggagcttgcttctgttgatggcgaccggcgcacgggtgagtaacgcgtatccaaccttccctttactctgggatagcccggtgaaaaccggattaacaccggatgggttcacatgacggcatcagactgtgaataaaggttacggtaaaggat

SEQ ID NO: 495 - DNA, 225 bp, Bacteroidales bacterium; identical to SEQ ID NO: 106
  source 1..225: mol_type = genomic DNA

SEQ ID NO: 496 - DNA, 225 bp, Bacteroidales bacterium; identical to SEQ ID NO: 469
  source 1..225: mol_type = genomic DNA

SEQ ID NO: 497 - DNA, 225 bp, Bacteroidales bacterium
  source 1..225: mol_type = genomic DNA
agagtttgatcatggctcaggatgaacgctagctacaggcttaacacatgcaagtcgaggggcagcatgagattagcttgctaatcttgatggcgaccggcgcacgggtgagtaacgcgtatccaaccttccctttactcagggatagcccggtgaaaaccggattaacacctgatagtttcacatgacggcatcagactgtgaataaaggttacggtaaaggat

SEQ ID NO: 498 - DNA, 225 bp, Bacteroidales bacterium; identical to SEQ ID NO: 283
  source 1..225: mol_type = genomic DNA

SEQ ID NO: 499 - DNA, 225 bp, Bacteroidales bacterium; identical to SEQ ID NO: 284
  source 1..225: mol_type = genomic DNA

SEQ ID NO: 500 - DNA, 225 bp, Bacteroidales bacterium; identical to SEQ ID NO: 286
  source 1..225: mol_type = genomic DNA

SEQ ID NO: 501 - DNA, 225 bp, Prevotella oris; identical to SEQ ID NO: 329
  source 1..225: mol_type = genomic DNA

SEQ ID NO: 502 - DNA, 225 bp, Prevotella oris
  source 1..225: mol_type = genomic DNA
aagagttgtcctggctcaggatgaacgctagctacaggcttaatacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggga

SEQ ID NO: 503 - DNA, 225 bp, Prevotella oris
  source 1..225: mol_type = genomic DNA
agagtttgtcatggctccggatgaacgctagctacaggcttaacacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggga

SEQ ID NO: 504 - DNA, 225 bp, Prevotella oris; identical to SEQ ID NO: 329
  source 1..225: mol_type = genomic DNA

SEQ ID NO: 505 - DNA, 225 bp, Prevotella oris; identical to SEQ ID NO: 357
  source 1..225: mol_type = genomic DNA

SEQ ID NO: 506 - DNA, 225 bp, Prevotella oris
  source 1..225: mol_type = genomic DNA
agatttgatcctggctcaggatgaacgctagctacaggcttaacacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggga

SEQ ID NO: 507 - DNA, 225 bp, Prevotella oris
  source 1..225: mol_type = genomic DNA
agatttgatcatggctcaggatgaacgctagctacaggcttaacacatgcaagtcgaggggtaacatggagggagcttgcttcctccgatgacgaccggcgaatgggtgagtaacgcgtatccaacctgcccctttcccacggatagcccttagaaatgaggattaatacgtgatggcatccggcaatcgcatgagcgccggattaaagattcatcggaaaggga

SEQ ID NO: 508 - DNA, 225 bp, Bacteroides caecicola; identical to SEQ ID NO: 234
  source 1..225: mol_type = genomic DNA

SEQ ID NO: 509 - DNA, 225 bp, Bacteroides caecicola
  source 1..225: mol_type = genomic DNA
agatttgatcctggctcaggatgaacgctagctacaggcttaacacatgcaagtcgagcgggaccctttcgggggtctagcggcaaacgggtgagtaacacgtatccaacctttcccttacttggggatagcctttagaaatgaagagtaatacccgatgtggtgcgattatcgcatgttcattgcaccaaagattcatcggtaaggggtggggatgcgttccat

SEQ ID NO: 510 - DNA, 225 bp, Bacteroides caecicola; identical to SEQ ID NO: 234
  source 1..225: mol_type = genomic DNA

SEQ ID NO: 511 - DNA, 225 bp, Bacteroides caecicola
  source 1..225: mol_type = genomic DNA
agagtttgatcctggctcaggatgaacgctagctacaggcttaacacatgcaagtcgagcgggaccctttcgggggtctagcggcaaacgggtgagtaacacgtatccaacctttcccttacttggggatagcctttagaaatgaagagtaatacccgatgtggtgcgattatcgcatgttcattgcaccaaagattcatcggtaaggggtggggatgcgttcca

SEQ ID NO: 512 - DNA, 225 bp, Algoriphagus ratkowskyi; identical to SEQ ID NO: 68
  source 1..225: mol_type = genomic DNA

SEQ ID NO: 513 - DNA, 225 bp, Algoriphagus ratkowskyi; identical to SEQ ID NO: 389
  source 1..225: mol_type = genomic DNA

SEQ ID NO: 514 - DNA, 225 bp, Algoriphagus ratkowskyi; identical to SEQ ID NO: 68
  source 1..225: mol_type = genomic DNA

SEQ ID NO: 515 - DNA, 225 bp, Algoriphagus ratkowskyi; identical to SEQ ID NO: 135
  source 1..225: mol_type = genomic DNA

SEQ ID NO: 516 - DNA, 225 bp, Algoriphagus ratkowskyi
  source 1..225: mol_type = genomic DNA
agagtttgatcctggctcaggacgaacgctggcgacaggcctaacacatgcaagtcgaggggcagcacttcgggtggcgaccggcgcacgggtgagtaacgcgtatgtaacctgcccatcagtttgggatagcccagtgaaaactggattaataccggatgacatcgtttgaaagcatttgataatgattaaagatttatcgctgatggatggacatgcgttgga

SEQ ID NO: 517 - DNA, 225 bp, Algoriphagus ratkowskyi; identical to SEQ ID NO: 128
  source 1..225: mol_type = genomic DNA

SEQ ID NO: 518 - DNA, 225 bp, Algoriphagus ratkowskyi; identical to SEQ ID NO: 462
  source 1..225: mol_type = genomic DNA

SEQ ID NO: 519 - DNA, 225 bp, Algoriphagus ratkowskyi; identical to SEQ ID NO: 131
  source 1..225: mol_type = genomic DNA

SEQ ID NO: 520 - DNA, 225 bp, Parabacteroides distasonis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattagatagcatttgataatgattaaagatttattgctgatggatggacatgcgttgga

SEQ ID NO: 521 - DNA, 225 bp, Parabacteroides distasonis; identical to SEQ ID NO: 264
  source 1..225: mol_type = genomic DNA

SEQ ID NO: 522 - DNA, 225 bp, Parabacteroides distasonis; identical to SEQ ID NO: 266
  source 1..225: mol_type = genomic DNA

SEQ ID NO: 523 - DNA, 225 bp, Parabacteroides distasonis
  source 1..225: mol_type = genomic DNA
agaggttgatcctggctcaggatgaacgctagcgacaggcctaacacatgcaagtcgaggggcagcacttcgggtggcgaccggcgcacgggtgagtaacgcgtatgtaacctgcccatcagtgagggatagcccagtgaaaactggattaataccgcatgatatcattagatagcatttgaaaatgattaaagatttattgctgatggatggacatgcgttgga

SEQ ID NO: 524 - DNA, 225 bp, Parabacteroides distasonis; identical to SEQ ID NO: 13
  source 1..225: mol_type = genomic DNA

SEQ ID NO: 525 - DNA, 225 bp, Parabacteroides distasonis; identical to SEQ ID NO: 267
  source 1..225: mol_type = genomic DNA

SEQ ID NO: 526 - DNA, 225 bp, Parabacteroides distasonis
  source 1..225: mol_type = genomic DNA
agagtttgatcatggctcaggatgaacgctagcgacaggcctaacacatgcaagtcgaggggcagcacttcgggtggcgaccggcgcacgggtgagtaacgcgtatgtaacctgcccgtcagtgagggatagcccagtgaaaactggattaataccgcatgatgtcatatgatagcatttgattatgattaaagatttatcgctgatggatggacatgcgttgga

SEQ ID NO: 527 - DNA, 225 bp, Parabacteroides distasonis; identical to SEQ ID NO: 269
  source 1..225: mol_type = genomic DNA

SEQ ID NO: 528 - DNA, 225 bp, Helcococcus ovis
  source 1..225: mol_type = genomic DNA
agagtttgatcctggctcaggacgaacgctggcggcgtgcttaacacatgcaagttgaacgagaaattaatcttcgatttcttcggagtgaaaagattgatggaaagtagcgaacgggtgagtaacacgtgagaaacctgcctttcacaaagggatagcctcgggaaaccgggattaataccttatgacacataacattcgcatgaatgatatgttaaagaattt

SEQ ID NO: 529 - DNA, 225 bp, Bacteroidales bacterium
  source 1..225: mol_type = genomic DNA
agagtttgatcctggctcaggatgaacgctagcggcaggcttaacacatgcaagtcgaggggcagcgcggagtagcaatactctggcggcgaccggcggaagggtgcgtaacgcgtgagcaacatgcccgtcttcgggagataaccgatggaaacgtcgcctaatatcccataacaacattggaggcatcttcgaagtttgaaagctacggcgaagacggattgg

SEQ ID NO: 530 - DNA, 225 bp, Bacteroidales bacterium
  source 1..225: mol_type = genomic DNA
agagtttgatcatggctcaggatgaacgctagcggcaggcttaacacatgcaagtcgaggggcagcgcggtgtagcaatacactggcggcgaccggcggaagggtgcgtaacgcgtgagcaacatgcccgtcttcgggagataaccgatggaaacgtcgcctaatatcccataacaacattggaggcatctccgaagtttgaaagctacggcgaagacggattgg

SEQ ID NO: 531 - DNA, 225 bp, Bacteroidales bacterium
  source 1..225: mol_type = genomic DNA
agagtttgatcctggctcaggatgaacgctagcggcaggcttaacacatgcaagtcgaggggcagcgcggagtagcaatactctggcggcgaccggcggaagggtgcgtaacgcgtgagcaacatgcccgtcttcgggagataaccgatggaaacgtcgcctaatatcccataacaacattggaggcatctccggagtttgaaagctacggcgaagacggattgg

SEQ ID NO: 532 - DNA, 225 bp, Bacteroidales bacterium; identical to SEQ ID NO: 529
  source 1..225: mol_type = genomic DNA

SEQ ID NO: 533 - DNA, 225 bp, Bacteroidales bacterium
  source 1..225: mol_type = genomic DNA
agagtttgatcctggctcaggatgaacgctagcggcaggcttaacacatgcaagtcgaggggcagcgcggagtagcaatactctggcggcgaccggcggaagggtgcgtaacgcgtgagcaacatgcccgtcttcgggagataaccgatggaaacgtcgcctaatatcccataacaacattggaggcatctccgaagtttgaaagctacggcgaagacggattgg

SEQ ID NO: 534 - DNA, 225 bp, Bacteroidales bacterium
  source 1..225: mol_type = genomic DNA
agagtttgatcctggctcaggatgaacgctagcggcaggcttaacacatgcaagtcgaggggcagcggggagtagcaatactctggcggcgaccggcggaagggtgcgtaacgcgtgagcaacatgcccgtcttcgggagataaccgatggaaacgtcgcctaatatcccataacaacattggaggcatctccggagtttgaaagctacggcgaagacggattgg

SEQ ID NO: 535 - DNA, 225 bp, Alphaproteobacteria bacterium
  source 1..225: mol_type = genomic DNA
agagtttgatcctggctcagaacgaacgctggcggcaggtcttaggcatgcaagtcgaacgagcgaagcagggcttgccctgtggaacgagtggcgcacgagtgagtaacgcgtgggaaactgcccactactggggaataacgtctggaaacggacgctaataccgcatacgccggaaacgggaaagatttatcggtggtggatgtgcccgcgttggattagctt

SEQ ID NO: 536 - DNA, 225 bp, Alphaproteobacteria bacterium
  source 1..225: mol_type = genomic DNA
agagtttgatcatggctcagaacgaacgctggcggcaggtcttaggcatgcaagtcgaacgagcgaagcagggcttgccctgtggaacgagtggcgcacgagtgagtaacgcgtgggaaactgcccaccactggggaataacgtctggaaacggacgctaataccgcatacgccggaaacgggaaagatttatcggtggtggatgtgcccgcgttggattagctt

SEQ ID NO: 537 - DNA, 225 bp, Alphaproteobacteria bacterium; identical to SEQ ID NO: 535
  source 1..225: mol_type = genomic DNA

SEQ ID NO: 538 - DNA, 225 bp, Alphaproteobacteria bacterium
  source 1..225: mol_type = genomic DNA
agagtttgatcctggctcagaacgaacgctggcggcaggtcttaggcatgcaagtcgaacgagcgaagcagggcttgccctgtggaacgagtggcgcacgagtgagtaacgcgtgggaaactgcccattactggggaataacgtttggaaacgaacgctaataccgcatacgccggaaacgggaaagatttatcggtaatggatgtgcccgcgttggattagctt

SEQ ID NO: 539 - DNA, 225 bp, Alphaproteobacteria bacterium
  source 1..225: mol_type = genomic DNA
agagtttgatcatggctcagaacgaacgctggcggcaggtcttaggcatgcaagtcgaacgagcgaagcagggcttgccctgtggaacgagtggcgcacgagtgagtaacgcgtgggaaactgcccactactggggaataacgtctggaaacggacgctaataccgcatacgccggaaacgggaaagatttatcggtggtggatgtgcccgcgttggattagctt

SEQ ID NO: 540 - DNA, 225 bp, Alphaproteobacteria bacterium
  source 1..225: mol_type = genomic DNA
agagtttgatcatggctcagaacgaacgctggcggcaggtcttaggcatgcaagtcgaacgagcgaagcagggcttgccctgtggaacgagtggcgcacgagtgagtaacgcgtgggaaactgcccattactggggaataacgtctggaaacggacgctaataccgcatacgcccgcaaggggaaagatttatcggtgatggatgtgcccgcgttggattagctt

SEQ ID NO: 541 - DNA, 225 bp, Anaerocella delicata
  source 1..225: mol_type = genomic DNA
agagtttgatcatggctcaggatgaacgctagcggcaggcttaacacatgcaagtcgaggggcatcgagattgtagcaatacaattgtcggcgaccggcggacgggtgcgtaacgcgtatgcaacctgccttatacagggggataacccagagaaatttggactaataccccataagcttattacaggcatctgtagaaagtgaaagattaatcggtataagatg

SEQ ID NO: 542 - DNA, 225 bp, Anaerocella delicata
  source 1..225: mol_type = genomic DNA
agagtttgatcctggctcaggatgaacgctagcggcaggcttaacacatgcaagtcgaggggcatcgagattgtagcaatacaattgtcggcgaccggcggacgggtgcgtaacgcgtatgcaacctgccttatacagggggataaccaagagaaatttggactaataccccataagcttattacaggcatctgtagaaagtgaaagattaatcggtataagatg

SEQ ID NO: 543 - DNA, 225 bp, Anaerocella delicata
  source 1..225: mol_type = genomic DNA
agagtttgatcctggctcaggatgaacgctagcggcaggcttaacacatgcaagtcgaggggcatcgagattgtagcaatacaattgtcggcgaccggcggacgggtgcgtaacgcgtatgcaacctgccttatacagggggataacccagagaaatttggactaataccccataagcttattacaggcatctgtagaaagtgaaagattaatcggtataagatg

SEQ ID NO: 544 - DNA, 225 bp, Anaerocella delicata; identical to SEQ ID NO: 541
  source 1..225: mol_type = genomic DNA

SEQ ID NO: 545 - DNA, 225 bp, Alphaproteobacteria bacterium; identical to SEQ ID NO: 539
  source 1..225: mol_type = genomic DNA

SEQ ID NO: 546 - DNA, 225 bp, Alphaproteobacteria bacterium
  source 1..225: mol_type = genomic DNA
agagtttgatcctggctcagaacgaacgctggcggcaggtcttaggcatgcaagtcgaacgagcgaagcagggcttgccctgtggaacgagtggcgcacgagtgagtaacgcgtgggaaactgcccaccactggggaataacgtctggaaacggacgctaataccgcatacgccggaaacgggaaagatttatcggtggtggatgtgcccgcgttggattagctt

SEQ ID NO: 547 - DNA, 225 bp, Alphaproteobacteria bacterium; identical to SEQ ID NO: 535
  source 1..225: mol_type = genomic DNA

SEQ ID NO: 548 - DNA, 225 bp, Alphaproteobacteria bacterium; identical to SEQ ID NO: 538
  source 1..225: mol_type = genomic DNA

SEQ ID NO: 549 - DNA, 225 bp, Alphaproteobacteria bacterium; identical to SEQ ID NO: 539
  source 1..225: mol_type = genomic DNA

SEQ ID NO: 550 - DNA, 225 bp, Alphaproteobacteria bacterium; identical to SEQ ID NO: 540
  source 1..225: mol_type = genomic DNA

SEQ ID NO: 551 - DNA, 225 bp, unidentified; identical to SEQ ID NO: 284
  misc_feature 1..225: note = Prevotella sp.
  source 1..225: mol_type = genomic DNA

SEQ ID NO: 552 - DNA, 225 bp, unidentified
  misc_feature 1..225: note = Prevotella sp.
  source 1..225: mol_type = genomic DNA
agagtttgatcctggctcaggatgaacgctggctacaggcttaacacatgcaagtcgaggggcagcatgagattagcttgctaatcttgatggcgaccggcgcacgggtgagtaacgcgtatccaaccttccctttactctgggatagcccggtgaaaaccggattaacaccggatgggtccacatgacggcatcagactgtgaataaaggttacggtaaaggat

SEQ ID NO: 553 - DNA, 225 bp, unidentified; identical to SEQ ID NO: 280
  misc_feature 1..225: note = Prevotella sp.
  source 1..225: mol_type = genomic DNA

SEQ ID NO: 554 - DNA, 225 bp, unidentified; identical to SEQ ID NO: 106
  misc_feature 1..225: note = Prevotella sp.
  source 1..225: mol_type = genomic DNA

SEQ ID NO: 555 - DNA, 225 bp, unidentified; identical to SEQ ID NO: 497
  misc_feature 1..225: note = Prevotella sp.
  source 1..225: mol_type = genomic DNA

SEQ ID NO: 556 - DNA, 225 bp, unidentified; identical to SEQ ID NO: 283
  misc_feature 1..225: note = Prevotella sp.
  source 1..225: mol_type = genomic DNA

SEQ ID NO: 557 - DNA, 225 bp, unidentified; identical to SEQ ID NO: 284
  misc_feature 1..225: note = Prevotella sp.
  source 1..225: mol_type = genomic DNA

SEQ ID NO: 558 - DNA, 225 bp, unidentified; identical to SEQ ID NO: 285
  misc_feature 1..225: note = Prevotella sp.
  source 1..225: mol_type = genomic DNA

SEQ ID NO: 559 - DNA, 225 bp, unidentified; identical to SEQ ID NO: 291
  misc_feature 1..225: note = Phascolarctobacterium sp.
  source 1..225: mol_type = genomic DNA

SEQ ID NO: 560 - DNA, 225 bp, unidentified
  misc_feature 1..225: note = Phascolarctobacterium sp.
  source 1..225: mol_type = genomic DNA
agagtttgatcctggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaagagttttccgcatggagagttcttgaaagatggcctctatttataagctatcgctagagg

SEQ ID NO: 561 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaagagttttccgcatggagagttcttgaaagatggcctctatttataagctatcgctagagg

SEQ ID NO: 562 - DNA, 225 bp, unidentified; identical to SEQ ID NO: 291
  misc_feature 1..225: note = Phascolarctobacterium sp.
  source 1..225: mol_type = genomic DNA

SEQ ID NO: 563 - DNA, 225 bp, unidentified; identical to SEQ ID NO: 146
  misc_feature 1..225: note = Phascolarctobacterium sp.
  source 1..225: mol_type = genomic DNA

SEQ ID NO: 564 - DNA, 225 bp, unidentified; identical to SEQ ID NO: 133
  misc_feature 1..225: note = Phascolarctobacterium sp.
  source 1..225: mol_type = genomic DNA

SEQ ID NO: 565 - DNA, 225 bp, unidentified; identical to SEQ ID NO: 295
  misc_feature 1..225: note = Phascolarctobacterium sp.
  source 1..225: mol_type = genomic DNA

SEQ ID NO: 566 - DNA, 225 bp, unidentified; identical to SEQ ID NO: 115
  misc_feature 1..225: note = Phascolarctobacterium sp.
  source 1..225: mol_type = genomic DNA

SEQ ID NO: 567 - DNA, 225 bp, unidentified
  misc_feature 1..225: note = Phascolarctobacterium sp.
  source 1..225: mol_type = genomic DNA
agagtttgatcatggctcaggacaaacgctggcggcatgcctaacacatgcaagtcgaacggggttacttcggtaacctagtggcgaacgggtgagtaacgcgtaggcaacctgccctctagatggggacaacattccgaaaggagtgctaataccgaatgtgaagaactttccgcatggagagttcttgaaagatggcctctatttataagctatcgctagagg

SEQ ID NO: 568 - DNA, 225 bp, Bacteroidales bacterium
  source 1..225: mol_type = genomic DNA
agagtttgatcatggctcaggatgaacgctagcggcaggcttaacacatgcaagtcgaggggcagcgcggagtagcaatactttggcggcgaccggcggaagggtgcgtaacgcgtgagcaacatgcccgtctttgggagataaccgatggaaacgtcgcctaatatcccataacaactgaggaggcatctttttagtttgaaagtttcgatgaagacggattgg

SEQ ID NO: 569 - DNA, 225 bp, Bacteroidales bacterium; identical to SEQ ID NO: 148
  source 1..225: mol_type = genomic DNA

SEQ ID NO: 570 - DNA, 225 bp, Bacteroidales bacterium; identical to SEQ ID NO: 291
  source 1..225: mol_type = genomic DNA

SEQ ID NO: 571 - DNA, 225 bp, Bacteroidales bacterium; identical to SEQ ID NO: 146
  source 1..225: mol_type = genomic DNA

SEQ ID NO: 572 - DNA, 225 bp, Bacteroidales bacterium; identical to SEQ ID NO: 293
  source 1..225: mol_type = genomic DNA

SEQ ID NO: 573 - DNA, 225 bp, Bacteroidales bacterium; identical to SEQ ID NO: 133
  source 1..225: mol_type = genomic DNA

SEQ ID NO: 574 - DNA, 225 bp, Bacteroidales bacterium; identical to SEQ ID NO: 295
  source 1..225: mol_type = genomic DNA

SEQ ID NO: 575 - DNA, 225 bp, Bacteroidales bacterium; identical to SEQ ID NO: 115
  source 1..225: mol_type = genomic DNA